SEQUENCE LISTING (45 sequences)

SEQ ID NO: 1 - DNA, 265 bp, synthetic construct
  misc_feature 1..265: note = Beta-globin promoter
  source 1..265: mol_type = other DNA
aagcaatagatggctctgccctgacttttatgcccagccctggctcctgccctccctgctcctgggagtagattggccaaccctagggtgtggctccacagggtgaggtctaagtgatgacagccgtacctgtccttggctcttctggcactggcttaggagttggacttcaaaccctcagccctccctctaagatatatctcttggccccataccatcagtacaaattgctactaaaaacatcctcctttgcaagtgtatttac

SEQ ID NO: 2 - DNA, 44 bp, synthetic construct
  misc_feature 1..44: note = Beta-globin 5' UTR
  source 1..44: mol_type = other DNA
tgtttgaggttgctagtgaacacagttgtgtcagaagcaaatgt

SEQ ID NO: 3 - DNA, 424 bp, synthetic construct
  misc_feature 1..424: note = Beta-globin 3' UTR
  source 1..424: mol_type = other DNA
catgaggacagctaaaacaataagtaatgtaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagc

SEQ ID NO: 4 - DNA, 37 bp, synthetic construct
  misc_feature 1..37: note = Alpha-globin 5' UTR
  source 1..37: mol_type = other DNA
ggtgggttctctctgagtctgtggggaccagaagagt

SEQ ID NO: 5 - DNA, 111 bp, synthetic construct
  misc_feature 1..111: note = Alpha-globin 3' UTR
  source 1..111: mol_type = other DNA
tgccgcccactcagactttattcaaagaccacgggggtacgggtgcaggaaggggaggaggggctggggggaggcccaaggggcaagaagcatggccaccgaggctccagc

SEQ ID NO: 6 - DNA, 37 bp, synthetic construct; identical to SEQ ID NO: 4
  misc_feature 1..37: note = Alpha-globin 2 5' UTR
  source 1..37: mol_type = other DNA

SEQ ID NO: 7 - DNA, 110 bp, synthetic construct
  misc_feature 1..110: note = Alpha-globin 2 3' UTR
  source 1..110: mol_type = other DNA
tgctgcccactcagactttattcaaagaccaggaagggccggtgcaaggaggggaggagggcccgttgggaggcccagcgggcaggaggaacggctaccgaggctccagc

SEQ ID NO: 8 - DNA, 695 bp, synthetic construct
  misc_feature 1..695: note = Alpha-globin A1 gene
  source 1..695: mol_type = other DNA
ttaacggtatttggaggtcagcacggtgctcacagaagccaggaacttgtccagggaggcgtgcaccgcaggggtgaactcggcggggaggtgggcggccagggtcaccagcaggcagtggcttaggagctgtgcagagaagagggtcagtggggccgagggcccaggcccgcagccgccgcctgcgctacacctcccgcaacccgcgtgatcctctgccctgcgaggaaggcgccatctcgcccctcgacccagatcgctcccggcccgccgctcaccttgaagttgaccgggtccacccgaagcttgtgcgcgtgcaggtcgctcagggcggacagcgcgttgggcatgtcgtccacgtgcgccacggcgttggtcagcgcgtcggccaccttcttgccgtggcccttaacctgggcagagccgtggctcaggtcgaagtgcgggaagtaggtcttggtggtggggaaggacaggaacatcctgcggggagaagcagagtgaggggtggggtttgggtccggggccaggacggttgagggtggcctgtgggtccgggcgggcgaggagcccgggtcggagcaggggagggagcctcacctctccagggcctccgcaccatactcgccagcgtgcgcgccgaccttaccccaggcggccttgacgttggtcttgtcggcaggagacagcaccat

SEQ ID NO: 9 - DNA, 688 bp, synthetic construct
  misc_feature 1..688: note = Alpha-globin A2 gene
  source 1..688: mol_type = other DNA
ttaacggtatttggaggtcagcacggtgctcacagaagccaggaacttgtccagggaggcgtgcaccgcaggggtgaactcggcggggaggtgggcggccagggtcaccagcaggcagtggcttaggagctgtgcagagaagagggtcagtgcggcccaggcccgcagccgccgcctgcgctacacctcccgcaacccgcgtgatcctctgccctgagaggaaggcgccatctcgcccctcgacccagatcgctcccggcccgccgctcaccttgaagttgaccgggtccacccgaagcttgtgcgcgtgcaggtcgctcagggcggacagcgcgttgggcatgtcgtccacgtgcgccacggcgttggtcagcgcgtcggccaccttcttgccgtggcccttaacctgggcagagccgtggctcaggtcgaagtgcgggaagtaggtcttggtggtggggaaggacaggaacatcctgcggggagaagcagagtgaggggtggggtttgggtccggggccaggacggttgagggtggcctgtgggtccgggcgggcgaggagcccgggtcggagcaggggagggagcctcacctctccagggcctccgcaccatactcgccagcgtgcgcgccgaccttaccccaggcggccttgacgttggtcttgtcggcaggagacagcaccat

SEQ ID NO: 10 - DNA, 256 bp, synthetic construct
  misc_feature 1..256: note = HS40 alpha-globin regulatory element
  source 1..256: mol_type = other DNA
ctctcagataaacaggagggggaggcagcaccagaggttgtaatcagcagtaccattcacccctttcccttgtcccacctccacccccacaggatgactcagcagtcctgttggcctccagaagcactgagtcatggttggcccagttatctgctccctcaagtgttttcacagacccccacagctgcatgcctccacccttggcttgggcagatgtgcttgcctggctgactgtggtccctgataggttccagag

SEQ ID NO: 11 - DNA, 1179 bp, synthetic construct
  misc_feature 1..1179: note = Core locus control region (LCR)
  source 1..1179: mol_type = other DNA
tacgtatatgtgtatatatatatatatattcaggaaataatatattctagaatatgtcacattctgtctcaggcatccattttctttatgatgccgtttgaggtggagttttagtcaggtggtcagcttctccttttttttgccatctgccctgtaagcatcctgctggggacccagataggagtcatcactctaggctgagaacatctgggcacacaccctaagcctcagcatgactcatcatgactcagcattgctgtgcttgagccagaaggtttgcttagaaggttacacagaaccagaaggcgggggtggggcactgaccccgacaggggcctggccagaactgctcatgcttggactatgggaggtcactaatggagacacacagaaatgtaacaggaactaaggaaaaactgaagctttgggggtataggggagcagtcccatgtagtagtagaatgaaaaatgctgctatgctgtgcctcccccacctttcccatgtctgccctctactcatggtctatctctcctggctcctgggagtcatggactccacccagcaccaccaacctgacctaaccacctatctgagcctgccagcctataacccatctgggccctgatagctggtggccagccctgaccccaccccaccctccctggaacctctgatagacacatctggcacaccagctcgcaaagtcaccgtgagggtcttgtgtttgctgagtcaaaattccttgaaatccaagtccttagagactcccaggcttggattcaaagctcctgactttctgtctagtgtatgtgcagtgagccccttttcctctaactgaaagaaggaaaaaaaaatggaacccaaaatattctacatagtttccatgtcacagccagggctgggcagtctcctgttatttcttttaaaataaatatatcatttaaatgcataaataagcaaaccctgctcgggaatgggagggagagtctctggagtccaccccttctcggccctggctctgcagatagtgctatcaaagccctgacagagccctgcccattgctgggccttggagtgagtcagcctagtagagaggcagggcaagccatctcatagctgctgagtgggagagagaaaagggctcattgtctataaactcaggtcatggctattcttat

SEQ ID NO: 12 - DNA, 2623 bp, synthetic construct
  misc_feature 1..2623: note = GLOBE beta globin locus control region (LCR)
  source 1..2623: mol_type = other DNA
ccttttgccacctagctgtccaggggtgccttaaaatggcaaacaaggtttgttttcttttcctgttttcatgccttcctcttccatatccttgtttcatattaatacatgtgtatagatcctaaaaatctatacacatgtattaataaagcctgattctgccgcttctaggtatagaggccacctgcaagataaatatttgattcacaataactaatcattctatggcaattgataacaacaaatatatatatatatatatatacgtatatgtgtatatatatatatatatattcaggaaataatatattctagaatatgtcacattctgtctcaggcatccattttctttatgatgccgtttgaggtggagttttagtcaggtggtcagcttctccttttttttgccatctgccctgtaagcatcctgctggggacccagataggagtcatcactctaggctgagaacatctgggcacacaccctaagcctcagcatgactcatcatgactcagcattgctgtgcttgagccagaaggtttgcttagaaggttacacagaaccagaaggcgggggtggggcactgaccccgacaggggcctggccagaactgctcatgcttggactatgggaggtcactaatggagacacacagaaatgtaacaggaactaaggaaaaactgaagcttatttaatcagagatgaggatgctggaagggatagagggagctgagcttgtaaaaagtatagtaatcattcagcaaatggttttgaagcacctgctggatgctaaacactattttcagtgcttgaatcataaataagaataaaacatgtatcttattccccacaagagtccaagtaaaaaataacagttaattataatgtgctctgtcccccaggctggagtgcagtggcacgatctcagctcactgcaacctccgcctcccgggttcaagcaattctcctgcctcagccaccctaatagctgggattacaggtgcacaccaccatgccaggctaatttttgtactttttgtagaggcagggtatcaccatgttgtccaagatggtcttgaactcctgagctccaagcagtccacccacctcagcctcccaaagtgctgggattacaggtgtgagacaccatgcccagattttccatatttaatagaggtatttatgggatgggggaaaagaatgtttctctcactgtggattattttagagagtggagaatggtcaagatttttttaaaaattaagaaaacataagttggaccttgagaaatgaaaatttatttttttgttggaggatacccattctctatctcccatcagggcaagctgtaaggaactggctaagacacagtgagacagagtgacttagtcttagaggccccactggtacgacggtcaccaagctttcattaaaaaaagtctaaccagctgcattcgactttgactgcagcagctggttagaaggttctactggaggagggtcccagcccattgctaaattaacatcaggctctgagactggcagtatatctctaacagtggttgatgctatcttctggaacttgcctgctacattgagaccactgacccatacataggaagcccatagctctgtcctgaactgttaggccactggtccagagagtgtgcatctcctttgatcctcataataaccctatgagatagacacaattattactcttactttatagatgatgatcctgaaaacataggagtcaaggcacttgcccctagctgggggtataggggagcagtcccatgtagtagtagaatgaaaaatgctgctatgctgtgcctcccccacctttcccatgtctgccctctactcatggtctatctctcctggctcctgggagtcatggactccacccagcaccaccaacctgacctaaccacctatctgagcctgccagcctataacccatctgggccctgatagctggtggccagccctgaccccaccccaccctccctggaacctctgatagacacatctggcacaccagctcgcaaagtcaccgtgagggtcttgtgtttgctgagtcaaaattccttgaaatccaagtccttagagactcctgctcccaaatttacagtcatagacttcttcatggctgtctcctttatccacagaatgattcctttgcttcattgccccatccatctgatcctcctcatcagtgcagcacagggcccatgagcagtagctgcagagtctcacataggtctggcactgcctctgacatgtccgaccttaggcaaatgcttgactcttctgagctcagtcttgtcatggcaaaataaagataataatagtgtttttttatggagttagcgtgaggatggaaaacaatagcaaaattgattagactataaaaggtctcaacaaatagtagtagattttatcatccattaatccttccctctcctctcttactcatcccatcacgtatgcctcttaattttcccttacctataataagagttattcctcttattatattcttcttatagtgattctggatattaaagtgggaatgaggggcaggccactaacgaagaagatgtttctcaaagaagc

SEQ ID NO: 13 - DNA, 2175 bp, synthetic construct
  misc_feature 1..2175: note = GLOBE beta globin locus control region (LCR) containing HS3
  source 1..2175: mol_type = other DNA
ccttttgccacctagctgtccaggggtgccttaaaatggcaaacaaggtttgttttcttttcctgttttcatgccttcctcttccatatccttgtttcatattaatacatgtgtatagatcctaaaaatctatacacatgtattaataaagcctgattctgccgcttctaggtatagaggccacctgcaagataaatatttgattcacaataactaatcattctatggcaattgataacaacaaatatatatatatatatatatacgtatatgtgtatatatatatatatatattcaggaaataatatattctagaatatgtcacattctgtctcaggcatccattttctttatgatgccgtttgaggtggagttttagtcaggtggtcagcttctccttttttttgccatctgccctgtaagcatcctgctggggacccagataggagtcatcactctaggctgagaacatctgggcacacaccctaagcctcagcatgactcatcatgactcagcattgctgtgcttgagccagaaggtttgcttagaaggttacacagaaccagaaggcgggggtggggcactgaccccgacaggggcctggccagaactgctcatgcttggactatgggaggtcactaatggagacacacagaaatgtaacaggaactaaggaaaaactgaagcttatttaatcagagatgaggatgctggaagggatagagggagctgagcttgtaaaaagtatagtaatcattcagcaaatggttttgaagcacctgctggatgctaaacactattttcagtgcttgaatcataaataagaataaaacatgtatcttattccccacaagagtccaagtaaaaaataacagttaattataatgtgctctgtcccccaggctggagtgcagtggcacgatctcagctcactgcaacctccgcctcccgggttcaagcaattctcctgcctcagccaccctaatagctgggattacaggtgcacaccaccatgccaggctaatttttgtactttttgtagaggcagggtatcaccatgttgtccaagatggtcttgaactcctgagctccaagcagtccacccacctcagcctcccaaagtgctgggattacaggtgtgagacaccatgcccagattttccatatttaatagaggtatttatgggatgggggaaaagaatgtttctctcactgtggattattttagagagtggagaatggtcaagatttttttaaaaattaagaaaacataagttggaccttgagaaatgaaaatttatttttttgttggaggatacccattctctatctcccatcagggcaagctgtaaggaactggctaagacacagtgagacagagtgacttagtcttagaggccccactggtacgacggtcacctgggggtataggggagcagtcccatgtagtagtagaatgaaaaatgctgctatgctgtgcctcccccacctttcccatgtctgccctctactcatggtctatctctcctggctcctgggagtcatggactccacccagcaccaccaacctgacctaaccacctatctgagcctgccagcctataacccatctgggccctgatagctggtggccagccctgaccccaccccaccctccctggaacctctgatagacacatctggcacaccagctcgcaaagtcaccgtgagggtcttgtgtttgctgagtcaaaattccttgaaatccaagtccttagagactcccaggcttggattcaaagctcctgactttctgtctagtgtatgtgcagtgagccccttttcctctaactgaaagaaggaaaaaaaaatggaacccaaaatattctacatagtttccatgtcacagccagggctgggcagtctcctgttatttcttttaaaataaatatatcatttaaatgcataaataagcaaaccctgctcgggaatgggagggagagtctctggagtccaccccttctcggccctggctctgcagatagtgctatcaaagccctgacagagccctgcccattgctgggccttggagtgagtcagcctagtagagaggcagggcaagccatctcatagctgctgagtgggagagagaaaagggctcattgtctataaactcaggtcatggctattcttat

SEQ ID NO: 14 - DNA, 210 bp, synthetic construct
  misc_feature 1..210: note = Core alpha-globin promoter
  source 1..210: mol_type = other DNA
gccggcccgcgagcgcgccagggtttatgcttggggcgcgggggcacgcccggccgggcggcgctcattggctggcgcggagcccggggcgcggcctggaccgcaggggagtcccgggcggggcctgcgcggggccgggcggcgggctcggcctcccctgggggtgcacgcggggcgggggccaggacgtctccaccctccacccgccac

SEQ ID NO: 15 - DNA, 425 bp, synthetic construct
  misc_feature 1..425: note = Hypersensitivity site 2 (HS2)
  source 1..425: mol_type = other DNA
tacgtatatgtgtatatatatatatatattcaggaaataatatattctagaatatgtcacattctgtctcaggcatccattttctttatgatgccgtttgaggtggagttttagtcaggtggtcagcttctccttttttttgccatctgccctgtaagcatcctgctggggacccagataggagtcatcactctaggctgagaacatctgggcacacaccctaagcctcagcatgactcatcatgactcagcattgctgtgcttgagccagaaggtttgcttagaaggttacacagaaccagaaggcgggggtggggcactgaccccgacaggggcctggccagaactgctcatgcttggactatgggaggtcactaatggagacacacagaaatgtaacaggaactaaggaaaaactgaagctt

SEQ ID NO: 16 - DNA, 344 bp, synthetic construct
  misc_feature 1..344: note = Hypersensitivity site 3 (HS3)
  source 1..344: mol_type = other DNA
tgggggtataggggagcagtcccatgtagtagtagaatgaaaaatgctgctatgctgtgcctcccccacctttcccatgtctgccctctactcatggtctatctctcctggctcctgggagtcatggactccacccagcaccaccaacctgacctaaccacctatctgagcctgccagcctataacccatctgggccctgatagctggtggccagccctgaccccaccccaccctccctggaacctctgatagacacatctggcacaccagctcgcaaagtcaccgtgagggtcttgtgtttgctgagtcaaaattccttgaaatccaagtccttagagactcc

SEQ ID NO: 17 - DNA, 410 bp, synthetic construct
  misc_feature 1..410: note = Hypersensitivity site 4 (HS4)
  source 1..410: mol_type = other DNA
caggcttggattcaaagctcctgactttctgtctagtgtatgtgcagtgagccccttttcctctaactgaaagaaggaaaaaaaaatggaacccaaaatattctacatagtttccatgtcacagccagggctgggcagtctcctgttatttcttttaaaataaatatatcatttaaatgcataaataagcaaaccctgctcgggaatgggagggagagtctctggagtccaccccttctcggccctggctctgcagatagtgctatcaaagccctgacagagccctgcccattgctgggccttggagtgagtcagcctagtagagaggcagggcaagccatctcatagctgctgagtgggagagagaaaagggctcattgtctataaactcaggtcatggctattcttat

SEQ ID NO: 18 - DNA, 18 bp, synthetic construct
  misc_feature 1..18: note = Tag for PCR
  source 1..18: mol_type = other DNA
tgtgctgctctgcggcga

SEQ ID NO: 19 - DNA, 853 bp, synthetic construct
  misc_feature 1..853: note = Alpha-globin gene with tag
  source 1..853: mol_type = other DNA
tgctgcccactcagactttattcaaagaccaggaagggccggtgcaaggaggggaggagggcccgttgggaggcccagcgggcaggaggaacggctaccgaggctccagctgtgctgctctgcggcgattaacggtatttggaggtcagcacggtgctcacagaagccaggaacttgtccagggaggcgtgcaccgcaggggtgaactcggcggggaggtgggcggccagggtcaccagcaggcagtggcttaggagctgtgcagagaagagggtcagtgcggcccaggcccgcagccgccgcctgcgctacacctcccgcaacccgcgtgatcctctgccctgagaggaaggcgccatctcgcccctcgacccagatcgctcccggcccgccgctcaccttgaagttgaccgggtccacccgaagcttgtgcgcgtgcaggtcgctcagggcggacagcgcgttgggcatgtcgtccacgtgcgccacggcgttggtcagcgcgtcggccaccttcttgccgtggcccttaacctgggcagagccgtggctcaggtcgaagtgcgggaagtaggtcttggtggtggggaaggacaggaacatcctgcggggagaagcagagtgaggggtggggtttgggtccggggccaggacggttgagggtggcctgtgggtccgggcgggcgaggagcccgggtcggagcaggggagggagcctcacctctccagggcctccgcaccatactcgccagcgtgcgcgccgaccttaccccaggcggccttgacgttggtcttgtcggcaggagacagcaccatggtgggttctctctgagtctgtggggaccagaagagt

SEQ ID NO: 20 - DNA, 594 bp, synthetic construct
  misc_feature 1..594: note = Alpha-globin cDNA with tag
  source 1..594: mol_type = other DNA
tgctgcccactcagactttattcaaagaccaggaagggccggtgcaaggaggggaggagggcccgttgggaggcccagcgggcaggaggaacggctaccgaggctccagctgtgctgctctgcggcgattaacggtatttggaggtcagcacggtgctcacagaagccaggaacttgtccagggaggcgtgcaccgcaggggtgaactcggcggggaggtgggcggccagggtcaccagcaggcagtggcttaggagcttgaagttgaccgggtccacccgaagcttgtgcgcgtgcaggtcgctcagggcggacagcgcgttgggcatgtcgtccacgtgcgccacggcgttggtcagcgcgtcggccaccttcttgccgtggcccttaacctgggcagagccgtggctcaggtcgaagtgcgggaagtaggtcttggtggtggggaaggacaggaacatcctctccagggcctccgcaccatactcgccagcgtgcgcgccgaccttaccccaggcggccttgacgttggtcttgtcggcaggagacagcaccatggtgggttctctctgagtctgtggggaccagaagagt

SEQ ID NO: 21 - DNA, 973 bp, synthetic construct
  misc_feature 1..973: note = Hba-a1 gene with TAG
  source 1..973: mol_type = other DNA
tcaaagaccaagaggtacaggtgcaagggagagaagaagggcatggccagaaggcaagccccgcagaaggcagctgtgctgctctgcggcgattaacggtacttggaggtcagcacggtgctcacagaggcaaggaatttgtccagagaggcatgcaccgcgggggtgaaatcggcagggtggtggctagccaaggtcaccagcaggcagtggctcaggagctgcggagacaaagtggacaccctgatgcctctgctccccttcctgggaccactatgttccctgccttgggcacgaggacccccaagcccttcctaggggtcccagatgccgcctgccaggtcccagcgcataccttgaagttgacgggatccacacgcagcttgtgggcatgcaggtcgctcagagcagacagggcaccgggcaggtcatcgaggtggcctgcagcattggccagagcatcggcgaccttcttgccgtgacccttgacctgggcagagccgtggcttacatcaaagtgagggaagtaggtcttggtggtggggaagctagcaaacatcctgggagaaggagagaagagaagttgaggtcacagaaaagcatagttagaagctgcccactgagcgagtgccaggtccatattggatcctgtgatccttacagatcaaggtcctgttctcacctttccagggcttcagctccatattcagcaccatggccaccaatcttcccccaggcagccttgatgttgcttttgtcttccccagagagcaccatggtttcttcctgagtctgtcagaatcagaagtgtcttggacctgcagcaagtagcacttatatgccctccaagggcgtgtcctccaggcagggtggacacgcccttggagcagttctcattggctggtttgtgcttgctctacccagtaaaacttgttagggcccctgagattctggggctgggttgtgcttcccc

SEQ ID NO: 22 - DNA, 15 bp, synthetic construct
  misc_feature 1..15: note = Central polypurine tract (cPPT)
  source 1..15: mol_type = other DNA
aaaagaaaagggggg

SEQ ID NO: 23 - DNA, 269 bp, synthetic construct
  misc_feature 1..269: note = Rev response element (RRE)
  source 1..269: mol_type = other DNA
agtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaa

SEQ ID NO: 24 - DNA, 138 bp, synthetic construct
  misc_feature 1..138: note = Packaging signal (Psi)
  source 1..138: mol_type = other DNA
tcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggag

SEQ ID NO: 25 - DNA, 180 bp, synthetic construct
  misc_feature 1..180: note = 5' LTR
  source 1..180: mol_type = other DNA
gggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagc

SEQ ID NO: 26 - DNA, 149 bp, synthetic construct
  misc_feature 1..149: note = 3' LTR
  source 1..149: mol_type = other DNA
tggaagggctaattcactcccaacgaagacaagatctgctttttgcttgtactgggtctctctggttagaccagatctgagcctgggagctctctggctaactagggaacccactgcttaagcctcaataaagcttgccttgagtgctt

SEQ ID NO: 27 - DNA, 8585 bp, synthetic construct
  misc_feature 1..8585: note = Lentiviral vector
  source 1..8585: mol_type = other DNA
acgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatagcacctagatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacgggggggtcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgatctcgacacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagacatgaggacagctaaaacaataagtaatgtaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttaacggtatttggaggtcagcacggtgctcacagaagccaggaacttgtccagggaggcgtgcaccgcaggggtgaactcggcggggaggtgggcggccagggtcaccagcaggcagtggcttaggagctgtgcagagaagagggtcagtgcggcccaggcccgcagccgccgcctgcgctacacctcccgcaacccgcgtgatcctctgccctgagaggaaggcgccatctcgcccctcgacccagatcgctcccggcccgccgctcaccttgaagttgaccgggtccacccgaagcttgtgcgcgtgcaggtcgctcagggcggacagcgcgttgggcatgtcgtccacgtgcgccacggcgttggtcagcgcgtcggccaccttcttgccgtggcccttaacctgggcagagccgtggctcaggtcgaagtgcgggaagtaggtcttggtggtggggaaggacaggaacatcctgcggggagaagcagagtgaggggtggggtttgggtccggggccaggacggttgagggtggcctgtgggtccgggcgggcgaggagcccgggtcggagcaggggagggagcctcacctctccagggcctccgcaccatactcgccagcgtgcgcgccgaccttaccccaggcggccttgacgttggtcttgtcggcaggagacagcaccatggtggctgtttgaggttgctagtgaacacagttgtgtcagaagcaaatgtaagcaatagatggctctgccctgacttttatgcccagccctggctcctgccctccctgctcctgggagtagattggccaaccctagggtgtggctccacagggtgaggtctaagtgatgacagccgtacctgtccttggctcttctggcactggcttaggagttggacttcaaaccctcagccctccctctaagatatatctcttggccccataccatcagtacaaattgctactaaaaacatcctcctttgcaagtgtatttactacgtatatgtgtatatatatatatatattcaggaaataatatattctagaatatgtcacattctgtctcaggcatccattttctttatgatgccgtttgaggtggagttttagtcaggtggtcagcttctccttttttttgccatctgccctgtaagcatcctgctggggacccagataggagtcatcactctaggctgagaacatctgggcacacaccctaagcctcagcatgactcatcatgactcagcattgctgtgcttgagccagaaggtttgcttagaaggttacacagaaccagaaggcgggggtggggcactgaccccgacaggggcctggccagaactgctcatgcttggactatgggaggtcactaatggagacacacagaaatgtaacaggaactaaggaaaaactgaagctttgggggtataggggagcagtcccatgtagtagtagaatgaaaaatgctgctatgctgtgcctcccccacctttcccatgtctgccctctactcatggtctatctctcctggctcctgggagtcatggactccacccagcaccaccaacctgacctaaccacctatctgagcctgccagcctataacccatctgggccctgatagctggtggccagccctgaccccaccccaccctccctggaacctctgatagacacatctggcacaccagctcgcaaagtcaccgtgagggtcttgtgtttgctgagtcaaaattccttgaaatccaagtccttagagactcccaggcttggattcaaagctcctgactttctgtctagtgtatgtgcagtgagccccttttcctctaactgaaagaaggaaaaaaaaatggaacccaaaatattctacatagtttccatgtcacagccagggctgggcagtctcctgttatttcttttaaaataaatatatcatttaaatgcataaataagcaaaccctgctcgggaatgggagggagagtctctggagtccaccccttctcggccctggctctgcagatagtgctatcaaagccctgacagagccctgcccattgctgggccttggagtgagtcagcctagtagagaggcagggcaagccatctcatagctgctgagtgggagagagaaaagggctcattgtctataaactcaggtcatggctattcttattaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgttggttttttgtgtgtgcattgtctgagtaggtgtcattctattctggggggtggggtggggcagcacagcaagggggaggattgggaagacaatagcaggcatgctggggatgcggtgggctctatggagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctctcactacttctggaatagctcagaggccgaggcggcctcggcctctgcataaataaaaaaaattagtcgaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggg

SEQ ID NO: 28 - DNA, 8266 bp, synthetic construct
  misc_feature 1..8266: note = Lentiviral vector
  source 1..8266: mol_type = other DNA
acgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatagcacctagatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacgggggggtcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgatctcgacacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagatgccgcccactcagactttattcaaagaccacgggggtacgggtgcaggaaggggaggaggggctggggggaggcccaaggggcaagaagcatggccaccgaggctccagcttaacggtatttggaggtcagcacggtgctcacagaagccaggaacttgtccagggaggcgtgcaccgcaggggtgaactcggcggggaggtgggcggccagggtcaccagcaggcagtggcttaggagctgtgcagagaagagggtcagtggggccgagggcccaggcccgcagccgccgcctgcgctacacctcccgcaacccgcgtgatcctctgccctgcgaggaaggcgccatctcgcccctcgacccagatcgctcccggcccgccgctcaccttgaagttgaccgggtccacccgaagcttgtgcgcgtgcaggtcgctcagggcggacagcgcgttgggcatgtcgtccacgtgcgccacggcgttggtcagcgcgtcggccaccttcttgccgtggcccttaacctgggcagagccgtggctcaggtcgaagtgcgggaagtaggtcttggtggtggggaaggacaggaacatcctgcggggagaagcagagtgaggggtggggtttgggtccggggccaggacggttgagggtggcctgtgggtccgggcgggcgaggagcccgggtcggagcaggggagggagcctcacctctccagggcctccgcaccatactcgccagcgtgcgcgccgaccttaccccaggcggccttgacgttggtcttgtcggcaggagacagcaccatggtgggttctctctgagtctgtggggaccagaagagtaagcaatagatggctctgccctgacttttatgcccagccctggctcctgccctccctgctcctgggagtagattggccaaccctagggtgtggctccacagggtgaggtctaagtgatgacagccgtacctgtccttggctcttctggcactggcttaggagttggacttcaaaccctcagccctccctctaagatatatctcttggccccataccatcagtacaaattgctactaaaaacatcctcctttgcaagtgtatttactacgtatatgtgtatatatatatatatattcaggaaataatatattctagaatatgtcacattctgtctcaggcatccattttctttatgatgccgtttgaggtggagttttagtcaggtggtcagcttctccttttttttgccatctgccctgtaagcatcctgctggggacccagataggagtcatcactctaggctgagaacatctgggcacacaccctaagcctcagcatgactcatcatgactcagcattgctgtgcttgagccagaaggtttgcttagaaggttacacagaaccagaaggcgggggtggggcactgaccccgacaggggcctggccagaactgctcatgcttggactatgggaggtcactaatggagacacacagaaatgtaacaggaactaaggaaaaactgaagctttgggggtataggggagcagtcccatgtagtagtagaatgaaaaatgctgctatgctgtgcctcccccacctttcccatgtctgccctctactcatggtctatctctcctggctcctgggagtcatggactccacccagcaccaccaacctgacctaaccacctatctgagcctgccagcctataacccatctgggccctgatagctggtggccagccctgaccccaccccaccctccctggaacctctgatagacacatctggcacaccagctcgcaaagtcaccgtgagggtcttgtgtttgctgagtcaaaattccttgaaatccaagtccttagagactcccaggcttggattcaaagctcctgactttctgtctagtgtatgtgcagtgagccccttttcctctaactgaaagaaggaaaaaaaaatggaacccaaaatattctacatagtttccatgtcacagccagggctgggcagtctcctgttatttcttttaaaataaatatatcatttaaatgcataaataagcaaaccctgctcgggaatgggagggagagtctctggagtccaccccttctcggccctggctctgcagatagtgctatcaaagccctgacagagccctgcccattgctgggccttggagtgagtcagcctagtagagaggcagggcaagccatctcatagctgctgagtgggagagagaaaagggctcattgtctataaactcaggtcatggctattcttattaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgttggttttttgtgtgtgcattgtctgagtaggtgtcattctattctggggggtggggtggggcagcacagcaagggggaggattgggaagacaatagcaggcatgctggggatgcggtgggctctatggagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctctcactacttctggaatagctcagaggccgaggcggcctcggcctctgcataaataaaaaaaattagtcgaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggg

SEQ ID NO: 29 - DNA, 8258 bp, synthetic construct
  misc_feature 1..8258: note = Lentiviral vector
  source 1..8258: mol_type = other DNA
acgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatagcacctagatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacgggggggtcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgatctcgacacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagatgctgcccactcagactttattcaaagaccaggaagggccggtgcaaggaggggaggagggcccgttgggaggcccagcgggcaggaggaacggctaccgaggctccagcttaacggtatttggaggtcagcacggtgctcacagaagccaggaacttgtccagggaggcgtgcaccgcaggggtgaactcggcggggaggtgggcggccagggtcaccagcaggcagtggcttaggagctgtgcagagaagagggtcagtgcggcccaggcccgcagccgccgcctgcgctacacctcccgcaacccgcgtgatcctctgccctgagaggaaggcgccatctcgcccctcgacccagatcgctcccggcccgccgctcaccttgaagttgaccgggtccacccgaagcttgtgcgcgtgcaggtcgctcagggcggacagcgcgttgggcatgtcgtccacgtgcgccacggcgttggtcagcgcgtcggccaccttcttgccgtggcccttaacctgggcagagccgtggctcaggtcgaagtgcgggaagtaggtcttggtggtggggaaggacaggaacatcctgcggggagaagcagagtgaggggtggggtttgggtccggggccaggacggttgagggtggcctgtgggtccgggcgggcgaggagcccgggtcggagcaggggagggagcctcacctctccagggcctccgcaccatactcgccagcgtgcgcgccgaccttaccccaggcggccttgacgttggtcttgtcggcaggagacagcaccatggtgggttctctctgagtctgtggggaccagaagagtaagcaatagatggctctgccctgacttttatgcccagccctggctcctgccctccctgctcctgggagtagattggccaaccctagggtgtggctccacagggtgaggtctaagtgatgacagccgtacctgtccttggctcttctggcactggcttaggagttggacttcaaaccctcagccctccctctaagatatatctcttggccccataccatcagtacaaattgctactaaaaacatcctcctttgcaagtgtatttactacgtatatgtgtatatatatatatatattcaggaaataatatattctagaatatgtcacattctgtctcaggcatccattttctttatgatgccgtttgaggtggagttttagtcaggtggtcagcttctccttttttttgccatctgccctgtaagcatcctgctggggacccagataggagtcatcactctaggctgagaacatctgggcacacaccctaagcctcagcatgactcatcatgactcagcattgctgtgcttgagccagaaggtttgcttagaaggttacacagaaccagaaggcgggggtggggcactgaccccgacaggggcctggccagaactgctcatgcttggactatgggaggtcactaatggagacacacagaaatgtaacaggaactaaggaaaaactgaagctttgggggtataggggagcagtcccatgtagtagtagaatgaaaaatgctgctatgctgtgcctcccccacctttcccatgtctgccctctactcatggtctatctctcctggctcctgggagtcatggactccacccagcaccaccaacctgacctaaccacctatctgagcctgccagcctataacccatctgggccctgatagctggtggccagccctgaccccaccccaccctccctggaacctctgatagacacatctggcacaccagctcgcaaagtcaccgtgagggtcttgtgtttgctgagtcaaaattccttgaaatccaagtccttagagactcccaggcttggattcaaagctcctgactttctgtctagtgtatgtgcagtgagccccttttcctctaactgaaagaaggaaaaaaaaatggaacccaaaatattctacatagtttccatgtcacagccagggctgggcagtctcctgttatttcttttaaaataaatatatcatttaaatgcataaataagcaaaccctgctcgggaatgggagggagagtctctggagtccaccccttctcggccctggctctgcagatagtgctatcaaagccctgacagagccctgcccattgctgggccttggagtgagtcagcctagtagagaggcagggcaagccatctcatagctgctgagtgggagagagaaaagggctcattgtctataaactcaggtcatggctattcttattaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgttggttttttgtgtgtgcattgtctgagtaggtgtcattctattctggggggtggggtggggcagcacagcaagggggaggattgggaagacaatagcaggcatgctggggatgcggtgggctctatggagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctctcactacttctggaatagctcagaggccgaggcggcctcggcctctgcataaataaaaaaaattagtcgaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggg

SEQ ID NO: 30 - DNA, 8514 bp, synthetic construct
  misc_feature 1..8514: note = Lentiviral vector
  source 1..8514: mol_type = other DNA
acgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatagcacctagatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacgggggggtcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgatctcgacacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagatgctgcccactcagactttattcaaagaccaggaagggccggtgcaaggaggggaggagggcccgttgggaggcccagcgggcaggaggaacggctaccgaggctccagcttaacggtatttggaggtcagcacggtgctcacagaagccaggaacttgtccagggaggcgtgcaccgcaggggtgaactcggcggggaggtgggcggccagggtcaccagcaggcagtggcttaggagctgtgcagagaagagggtcagtgcggcccaggcccgcagccgccgcctgcgctacacctcccgcaacccgcgtgatcctctgccctgagaggaaggcgccatctcgcccctcgacccagatcgctcccggcccgccgctcaccttgaagttgaccgggtccacccgaagcttgtgcgcgtgcaggtcgctcagggcggacagcgcgttgggcatgtcgtccacgtgcgccacggcgttggtcagcgcgtcggccaccttcttgccgtggcccttaacctgggcagagccgtggctcaggtcgaagtgcgggaagtaggtcttggtggtggggaaggacaggaacatcctgcggggagaagcagagtgaggggtggggtttgggtccggggccaggacggttgagggtggcctgtgggtccgggcgggcgaggagcccgggtcggagcaggggagggagcctcacctctccagggcctccgcaccatactcgccagcgtgcgcgccgaccttaccccaggcggccttgacgttggtcttgtcggcaggagacagcaccatggtgggttctctctgagtctgtggggaccagaagagtaagcaatagatggctctgccctgacttttatgcccagccctggctcctgccctccctgctcctgggagtagattggccaaccctagggtgtggctccacagggtgaggtctaagtgatgacagccgtacctgtccttggctcttctggcactggcttaggagttggacttcaaaccctcagccctccctctaagatatatctcttggccccataccatcagtacaaattgctactaaaaacatcctcctttgcaagtgtatttacctctcagataaacaggagggggaggcagcaccagaggttgtaatcagcagtaccattcacccctttcccttgtcccacctccacccccacaggatgactcagcagtcctgttggcctccagaagcactgagtcatggttggcccagttatctgctccctcaagtgttttcacagacccccacagctgcatgcctccacccttggcttgggcagatgtgcttgcctggctgactgtggtccctgataggttccagagtacgtatatgtgtatatatatatatatattcaggaaataatatattctagaatatgtcacattctgtctcaggcatccattttctttatgatgccgtttgaggtggagttttagtcaggtggtcagcttctccttttttttgccatctgccctgtaagcatcctgctggggacccagataggagtcatcactctaggctgagaacatctgggcacacaccctaagcctcagcatgactcatcatgactcagcattgctgtgcttgagccagaaggtttgcttagaaggttacacagaaccagaaggcgggggtggggcactgaccccgacaggggcctggccagaactgctcatgcttggactatgggaggtcactaatggagacacacagaaatgtaacaggaactaaggaaaaactgaagctttgggggtataggggagcagtcccatgtagtagtagaatgaaaaatgctgctatgctgtgcctcccccacctttcccatgtctgccctctactcatggtctatctctcctggctcctgggagtcatggactccacccagcaccaccaacctgacctaaccacctatctgagcctgccagcctataacccatctgggccctgatagctggtggccagccctgaccccaccccaccctccctggaacctctgatagacacatctggcacaccagctcgcaaagtcaccgtgagggtcttgtgtttgctgagtcaaaattccttgaaatccaagtccttagagactcccaggcttggattcaaagctcctgactttctgtctagtgtatgtgcagtgagccccttttcctctaactgaaagaaggaaaaaaaaatggaacccaaaatattctacatagtttccatgtcacagccagggctgggcagtctcctgttatttcttttaaaataaatatatcatttaaatgcataaataagcaaaccctgctcgggaatgggagggagagtctctggagtccaccccttctcggccctggctctgcagatagtgctatcaaagccctgacagagccctgcccattgctgggccttggagtgagtcagcctagtagagaggcagggcaagccatctcatagctgctgagtgggagagagaaaagggctcattgtctataaactcaggtcatggctattcttattaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgttggttttttgtgtgtgcattgtctgagtaggtgtcattctattctggggggtggggtggggcagcacagcaagggggaggattgggaagacaatagcaggcatgctggggatgcggtgggctctatggagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctctcactacttctggaatagctcagaggccgaggcggcctcggcctctgcataaataaaaaaaattagtcgaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggg

SEQ ID NO: 31 - DNA, 9702 bp, synthetic construct
  misc_feature 1..9702: note = Lentiviral vector
  source 1..9702: mol_type = other DNA
acgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatagcacctagatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacgggggggtcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgatctcgacacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagatgctgcccactcagactttattcaaagaccaggaagggccggtgcaaggaggggaggagggcccgttgggaggcccagcgggcaggaggaacggctaccgaggctccagcttaacggtatttggaggtcagcacggtgctcacagaagccaggaacttgtccagggaggcgtgcaccgcaggggtgaactcggcggggaggtgggcggccagggtcaccagcaggcagtggcttaggagctgtgcagagaagagggtcagtgcggcccaggcccgcagccgccgcctgcgctacacctcccgcaacccgcgtgatcctctgccctgagaggaaggcgccatctcgcccctcgacccagatcgctcccggcccgccgctcaccttgaagttgaccgggtccacccgaagcttgtgcgcgtgcaggtcgctcagggcggacagcgcgttgggcatgtcgtccacgtgcgccacggcgttggtcagcgcgtcggccaccttcttgccgtggcccttaacctgggcagagccgtggctcaggtcgaagtgcgggaagtaggtcttggtggtggggaaggacaggaacatcctgcggggagaagcagagtgaggggtggggtttgggtccggggccaggacggttgagggtggcctgtgggtccgggcgggcgaggagcccgggtcggagcaggggagggagcctcacctctccagggcctccgcaccatactcgccagcgtgcgcgccgaccttaccccaggcggccttgacgttggtcttgtcggcaggagacagcaccatggtgggttctctctgagtctgtggggaccagaagagtaagcaatagatggctctgccctgacttttatgcccagccctggctcctgccctccctgctcctgggagtagattggccaaccctagggtgtggctccacagggtgaggtctaagtgatgacagccgtacctgtccttggctcttctggcactggcttaggagttggacttcaaaccctcagccctccctctaagatatatctcttggccccataccatcagtacaaattgctactaaaaacatcctcctttgcaagtgtatttacccttttgccacctagctgtccaggggtgccttaaaatggcaaacaaggtttgttttcttttcctgttttcatgccttcctcttccatatccttgtttcatattaatacatgtgtatagatcctaaaaatctatacacatgtattaataaagcctgattctgccgcttctaggtatagaggccacctgcaagataaatatttgattcacaataactaatcattctatggcaattgataacaacaaatatatatatatatatatatacgtatatgtgtatatatatatatatatattcaggaaataatatattctagaatatgtcacattctgtctcaggcatccattttctttatgatgccgtttgaggtggagttttagtcaggtggtcagcttctccttttttttgccatctgccctgtaagcatcctgctggggacccagataggagtcatcactctaggctgagaacatctgggcacacaccctaagcctcagcatgactcatcatgactcagcattgctgtgcttgagccagaaggtttgcttagaaggttacacagaaccagaaggcgggggtggggcactgaccccgacaggggcctggccagaactgctcatgcttggactatgggaggtcactaatggagacacacagaaatgtaacaggaactaaggaaaaactgaagcttatttaatcagagatgaggatgctggaagggatagagggagctgagcttgtaaaaagtatagtaatcattcagcaaatggttttgaagcacctgctggatgctaaacactattttcagtgcttgaatcataaataagaataaaacatgtatcttattccccacaagagtccaagtaaaaaataacagttaattataatgtgctctgtcccccaggctggagtgcagtggcacgatctcagctcactgcaacctccgcctcccgggttcaagcaattctcctgcctcagccaccctaatagctgggattacaggtgcacaccaccatgccaggctaatttttgtactttttgtagaggcagggtatcaccatgttgtccaagatggtcttgaactcctgagctccaagcagtccacccacctcagcctcccaaagtgctgggattacaggtgtgagacaccatgcccagattttccatatttaatagaggtatttatgggatgggggaaaagaatgtttctctcactgtggattattttagagagtggagaatggtcaagatttttttaaaaattaagaaaacataagttggaccttgagaaatgaaaatttatttttttgttggaggatacccattctctatctcccatcagggcaagctgtaaggaactggctaagacacagtgagacagagtgacttagtcttagaggccccactggtacgacggtcaccaagctttcattaaaaaaagtctaaccagctgcattcgactttgactgcagcagctggttagaaggttctactggaggagggtcccagcccattgctaaattaacatcaggctctgagactggcagtatatctctaacagtggttgatgctatcttctggaacttgcctgctacattgagaccactgacccatacataggaagcccatagctctgtcctgaactgttaggccactggtccagagagtgtgcatctcctttgatcctcataataaccctatgagatagacacaattattactcttactttatagatgatgatcctgaaaacataggagtcaaggcacttgcccctagctgggggtataggggagcagtcccatgtagtagtagaatgaaaaatgctgctatgctgtgcctcccccacctttcccatgtctgccctctactcatggtctatctctcctggctcctgggagtcatggactccacccagcaccaccaacctgacctaaccacctatctgagcctgccagcctataacccatctgggccctgatagctggtggccagccctgaccccaccccaccctccctggaacctctgatagacacatctggcacaccagctcgcaaagtcaccgtgagggtcttgtgtttgctgagtcaaaattccttgaaatccaagtccttagagactcctgctcccaaatttacagtcatagacttcttcatggctgtctcctttatccacagaatgattcctttgcttcattgccccatccatctgatcctcctcatcagtgcagcacagggcccatgagcagtagctgcagagtctcacataggtctggcactgcctctgacatgtccgaccttaggcaaatgcttgactcttctgagctcagtcttgtcatggcaaaataaagataataatagtgtttttttatggagttagcgtgaggatggaaaacaatagcaaaattgattagactataaaaggtctcaacaaatagtagtagattttatcatccattaatccttccctctcctctcttactcatcccatcacgtatgcctcttaattttcccttacctataataagagttattcctcttattatattcttcttatagtgattctggatattaaagtgggaatgaggggcaggccactaacgaagaagatgtttctcaaagaagctaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgttggttttttgtgtgtgcattgtctgagtaggtgtcattctattctggggggtggggtggggcagcacagcaagggggaggattgggaagacaatagcaggcatgctggggatgcggtgggctctatggagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctctcactacttctggaatagctcagaggccgaggcggcctcggcctctgcataaataaaaaaaattagtcgaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggg

SEQ ID NO: 32 - DNA, 9254 bp, synthetic construct
  misc_feature 1..9254: note = Lentiviral vector
  source 1..9254: mol_type = other DNA
acgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatagcacctagatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacgggggggtcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgatctcgacacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagatgctgcccactcagactttattcaaagaccaggaagggccggtgcaaggaggggaggagggcccgttgggaggcccagcgggcaggaggaacggctaccgaggctccagcttaacggtatttggaggtcagcacggtgctcacagaagccaggaacttgtccagggaggcgtgcaccgcaggggtgaactcggcggggaggtgggcggccagggtcaccagcaggcagtggcttaggagctgtgcagagaagagggtcagtgcggcccaggcccgcagccgccgcctgcgctacacctcccgcaacccgcgtgatcctctgccctgagaggaaggcgccatctcgcccctcgacccagatcgctcccggcccgccgctcaccttgaagttgaccgggtccacccgaagcttgtgcgcgtgcaggtcgctcagggcggacagcgcgttgggcatgtcgtccacgtgcgccacggcgttggtcagcgcgtcggccaccttcttgccgtggcccttaacctgggcagagccgtggctcaggtcgaagtgcgggaagtaggtcttggtggtggggaaggacaggaacatcctgcggggagaagcagagtgaggggtggggtttgggtccggggccaggacggttgagggtggcctgtgggtccgggcgggcgaggagcccgggtcggagcaggggagggagcctcacctctccagggcctccgcaccatactcgccagcgtgcgcgccgaccttaccccaggcggccttgacgttggtcttgtcggcaggagacagcaccatggtgggttctctctgagtctgtggggaccagaagagtaagcaatagatggctctgccctgacttttatgcccagccctggctcctgccctccctgctcctgggagtagattggccaaccctagggtgtggctccacagggtgaggtctaagtgatgacagccgtacctgtccttggctcttctggcactggcttaggagttggacttcaaaccctcagccctccctctaagatatatctcttggccccataccatcagtacaaattgctactaaaaacatcctcctttgcaagtgtatttacccttttgccacctagctgtccaggggtgccttaaaatggcaaacaaggtttgttttcttttcctgttttcatgccttcctcttccatatccttgtttcatattaatacatgtgtatagatcctaaaaatctatacacatgtattaataaagcctgattctgccgcttctaggtatagaggccacctgcaagataaatatttgattcacaataactaatcattctatggcaattgataacaacaaatatatatatatatatatatacgtatatgtgtatatatatatatatatattcaggaaataatatattctagaatatgtcacattctgtctcaggcatccattttctttatgatgccgtttgaggtggagttttagtcaggtggtcagcttctccttttttttgccatctgccctgtaagcatcctgctggggacccagataggagtcatcactctaggctgagaacatctgggcacacaccctaagcctcagcatgactcatcatgactcagcattgctgtgcttgagccagaaggtttgcttagaaggttacacagaaccagaaggcgggggtggggcactgaccccgacaggggcctggccagaactgctcatgcttggactatgggaggtcactaatggagacacacagaaatgtaacaggaactaaggaaaaactgaagcttatttaatcagagatgaggatgctggaagggatagagggagctgagcttgtaaaaagtatagtaatcattcagcaaatggttttgaagcacctgctggatgctaaacactattttcagtgcttgaatcataaataagaataaaacatgtatcttattccccacaagagtccaagtaaaaaataacagttaattataatgtgctctgtcccccaggctggagtgcagtggcacgatctcagctcactgcaacctccgcctcccgggttcaagcaattctcctgcctcagccaccctaatagctgggattacaggtgcacaccaccatgccaggctaatttttgtactttttgtagaggcagggtatcaccatgttgtccaagatggtcttgaactcctgagctccaagcagtccacccacctcagcctcccaaagtgctgggattacaggtgtgagacaccatgcccagattttccatatttaatagaggtatttatgggatgggggaaaagaatgtttctctcactgtggattattttagagagtggagaatggtcaagatttttttaaaaattaagaaaacataagttggaccttgagaaatgaaaatttatttttttgttggaggatacccattctctatctcccatcagggcaagctgtaaggaactggctaagacacagtgagacagagtgacttagtcttagaggccccactggtacgacggtcacctgggggtataggggagcagtcccatgtagtagtagaatgaaaaatgctgctatgctgtgcctcccccacctttcccatgtctgccctctactcatggtctatctctcctggctcctgggagtcatggactccacccagcaccaccaacctgacctaaccacctatctgagcctgccagcctataacccatctgggccctgatagctggtggccagccctgaccccaccccaccctccctggaacctctgatagacacatctggcacaccagctcgcaaagtcaccgtgagggtcttgtgtttgctgagtcaaaattccttgaaatccaagtccttagagactcccaggcttggattcaaagctcctgactttctgtctagtgtatgtgcagtgagccccttttcctctaactgaaagaaggaaaaaaaaatggaacccaaaatattctacatagtttccatgtcacagccagggctgggcagtctcctgttatttcttttaaaataaatatatcatttaaatgcataaataagcaaaccctgctcgggaatgggagggagagtctctggagtccaccccttctcggccctggctctgcagatagtgctatcaaagccctgacagagccctgcccattgctgggccttggagtgagtcagcctagtagagaggcagggcaagccatctcatagctgctgagtgggagagagaaaagggctcattgtctataaactcaggtcatggctattcttattaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgttggttttttgtgtgtgcattgtctgagtaggtgtcattctattctggggggtggggtggggcagcacagcaagggggaggattgggaagacaatagcaggcatgctggggatgcggtgggctctatggagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctctcactacttctggaatagctcagaggccgaggcggcctcggcctctgcataaataaaaaaaattagtcgaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggg

SEQ ID NO: 33 - DNA, 8203 bp, synthetic construct
  misc_feature 1..8203: note = Lentiviral vector
  source 1..8203: mol_type = other DNA
acgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatagcacctagatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacgggggggtcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgatctcgacacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagatgctgcccactcagactttattcaaagaccaggaagggccggtgcaaggaggggaggagggcccgttgggaggcccagcgggcaggaggaacggctaccgaggctccagcttaacggtatttggaggtcagcacggtgctcacagaagccaggaacttgtccagggaggcgtgcaccgcaggggtgaactcggcggggaggtgggcggccagggtcaccagcaggcagtggcttaggagctgtgcagagaagagggtcagtgcggcccaggcccgcagccgccgcctgcgctacacctcccgcaacccgcgtgatcctctgccctgagaggaaggcgccatctcgcccctcgacccagatcgctcccggcccgccgctcaccttgaagttgaccgggtccacccgaagcttgtgcgcgtgcaggtcgctcagggcggacagcgcgttgggcatgtcgtccacgtgcgccacggcgttggtcagcgcgtcggccaccttcttgccgtggcccttaacctgggcagagccgtggctcaggtcgaagtgcgggaagtaggtcttggtggtggggaaggacaggaacatcctgcggggagaagcagagtgaggggtggggtttgggtccggggccaggacggttgagggtggcctgtgggtccgggcgggcgaggagcccgggtcggagcaggggagggagcctcacctctccagggcctccgcaccatactcgccagcgtgcgcgccgaccttaccccaggcggccttgacgttggtcttgtcggcaggagacagcaccatggtgggttctctctgagtctgtggggaccagaagagtgccggcccgcgagcgcgccagggtttatgcttggggcgcgggggcacgcccggccgggcggcgctcattggctggcgcggagcccggggcgcggcctggaccgcaggggagtcccgggcggggcctgcgcggggccgggcggcgggctcggcctcccctgggggtgcacgcggggcgggggccaggacgtctccaccctccacccgccactacgtatatgtgtatatatatatatatattcaggaaataatatattctagaatatgtcacattctgtctcaggcatccattttctttatgatgccgtttgaggtggagttttagtcaggtggtcagcttctccttttttttgccatctgccctgtaagcatcctgctggggacccagataggagtcatcactctaggctgagaacatctgggcacacaccctaagcctcagcatgactcatcatgactcagcattgctgtgcttgagccagaaggtttgcttagaaggttacacagaaccagaaggcgggggtggggcactgaccccgacaggggcctggccagaactgctcatgcttggactatgggaggtcactaatggagacacacagaaatgtaacaggaactaaggaaaaactgaagctttgggggtataggggagcagtcccatgtagtagtagaatgaaaaatgctgctatgctgtgcctcccccacctttcccatgtctgccctctactcatggtctatctctcctggctcctgggagtcatggactccacccagcaccaccaacctgacctaaccacctatctgagcctgccagcctataacccatctgggccctgatagctggtggccagccctgaccccaccccaccctccctggaacctctgatagacacatctggcacaccagctcgcaaagtcaccgtgagggtcttgtgtttgctgagtcaaaattccttgaaatccaagtccttagagactcccaggcttggattcaaagctcctgactttctgtctagtgtatgtgcagtgagccccttttcctctaactgaaagaaggaaaaaaaaatggaacccaaaatattctacatagtttccatgtcacagccagggctgggcagtctcctgttatttcttttaaaataaatatatcatttaaatgcataaataagcaaaccctgctcgggaatgggagggagagtctctggagtccaccccttctcggccctggctctgcagatagtgctatcaaagccctgacagagccctgcccattgctgggccttggagtgagtcagcctagtagagaggcagggcaagccatctcatagctgctgagtgggagagagaaaagggctcattgtctataaactcaggtcatggctattcttattaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgttggttttttgtgtgtgcattgtctgagtaggtgtcattctattctggggggtggggtggggcagcacagcaagggggaggattgggaagacaatagcaggcatgctggggatgcggtgggctctatggagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctctcactacttctggaatagctcagaggccgaggcggcctcggcctctgcataaataaaaaaaattagtcgaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggg

SEQ ID NO: 34 - DNA, 8266 bp, synthetic construct; identical to SEQ ID NO: 28
  misc_feature 1..8266: note = Lentiviral vector
  source 1..8266: mol_type = other DNA

SEQ ID NO: 35 - DNA, 9647 bp, synthetic construct
  misc_feature 1..9647: note = Lentiviral vector
  source 1..9647: mol_type = other DNA
acgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatagcacctagatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacgggggggtcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgatctcgacacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagatgctgcccactcagactttattcaaagaccaggaagggccggtgcaaggaggggaggagggcccgttgggaggcccagcgggcaggaggaacggctaccgaggctccagcttatctatacttgctcgtgagtacagtgctcacagaagccaggaacttgtccagggaggcgtgcaccgcaggggtgaactcggcggggaggtgggcggccagggtcaccagcaggcagtggcttaggagctgtgcagagaagagggtcagtgcggcccaggcccgcagccgccgcctgcgctacacctcccgcaacccgcgtgatcctctgccctgagaggaaggcgccatctcgcccctcgacccagatcgctcccggcccgccgctcaccttgaagttgaccgggtccacccgaagcttgtgcgcgtgcaggtcgctcagggcggacagcgcgttgggcatgtcgtccacgtgcgccacggcgttggtcagcgcgtcggccaccttcttgccgtggcccttaacctgggcagagccgtggctcaggtcgaagtgcgggaagtaggtcttggtggtggggaaggacaggaacatcctgcggggagaagcagagtgaggggtggggtttgggtccggggccaggacggttgagggtggcctgtgggtccgggcgggcgaggagcccgggtcggagcaggggagggagcctcacctctccagggcctccgcaccatactcgccagcgtgcgcgccgaccttaccccaggcggccttgacgttggtcttgtcggcaggagacagcaccatggtgggttctctctgagtctgtggggaccagaagagtgccggcccgcgagcgcgccagggtttatgcttggggcgcgggggcacgcccggccgggcggcgctcattggctggcgcggagcccggggcgcggcctggaccgcaggggagtcccgggcggggcctgcgcggggccgggcggcgggctcggcctcccctgggggtgcacgcggggcgggggccaggacgtctccaccctccacccgccacccttttgccacctagctgtccaggggtgccttaaaatggcaaacaaggtttgttttcttttcctgttttcatgccttcctcttccatatccttgtttcatattaatacatgtgtatagatcctaaaaatctatacacatgtattaataaagcctgattctgccgcttctaggtatagaggccacctgcaagataaatatttgattcacaataactaatcattctatggcaattgataacaacaaatatatatatatatatatatacgtatatgtgtatatatatatatatatattcaggaaataatatattctagaatatgtcacattctgtctcaggcatccattttctttatgatgccgtttgaggtggagttttagtcaggtggtcagcttctccttttttttgccatctgccctgtaagcatcctgctggggacccagataggagtcatcactctaggctgagaacatctgggcacacaccctaagcctcagcatgactcatcatgactcagcattgctgtgcttgagccagaaggtttgcttagaaggttacacagaaccagaaggcgggggtggggcactgaccccgacaggggcctggccagaactgctcatgcttggactatgggaggtcactaatggagacacacagaaatgtaacaggaactaaggaaaaactgaagcttatttaatcagagatgaggatgctggaagggatagagggagctgagcttgtaaaaagtatagtaatcattcagcaaatggttttgaagcacctgctggatgctaaacactattttcagtgcttgaatcataaataagaataaaacatgtatcttattccccacaagagtccaagtaaaaaataacagttaattataatgtgctctgtcccccaggctggagtgcagtggcacgatctcagctcactgcaacctccgcctcccgggttcaagcaattctcctgcctcagccaccctaatagctgggattacaggtgcacaccaccatgccaggctaatttttgtactttttgtagaggcagggtatcaccatgttgtccaagatggtcttgaactcctgagctccaagcagtccacccacctcagcctcccaaagtgctgggattacaggtgtgagacaccatgcccagattttccatatttaatagaggtatttatgggatgggggaaaagaatgtttctctcactgtggattattttagagagtggagaatggtcaagatttttttaaaaattaagaaaacataagttggaccttgagaaatgaaaatttatttttttgttggaggatacccattctctatctcccatcagggcaagctgtaaggaactggctaagacacagtgagacagagtgacttagtcttagaggccccactggtacgacggtcaccaagctttcattaaaaaaagtctaaccagctgcattcgactttgactgcagcagctggttagaaggttctactggaggagggtcccagcccattgctaaattaacatcaggctctgagactggcagtatatctctaacagtggttgatgctatcttctggaacttgcctgctacattgagaccactgacccatacataggaagcccatagctctgtcctgaactgttaggccactggtccagagagtgtgcatctcctttgatcctcataataaccctatgagatagacacaattattactcttactttatagatgatgatcctgaaaacataggagtcaaggcacttgcccctagctgggggtataggggagcagtcccatgtagtagtagaatgaaaaatgctgctatgctgtgcctcccccacctttcccatgtctgccctctactcatggtctatctctcctggctcctgggagtcatggactccacccagcaccaccaacctgacctaaccacctatctgagcctgccagcctataacccatctgggccctgatagctggtggccagccctgaccccaccccaccctccctggaacctctgatagacacatctggcacaccagctcgcaaagtcaccgtgagggtcttgtgtttgctgagtcaaaattccttgaaatccaagtccttagagactcctgctcccaaatttacagtcatagacttcttcatggctgtctcctttatccacagaatgattcctttgcttcattgccccatccatctgatcctcctcatcagtgcagcacagggcccatgagcagtagctgcagagtctcacataggtctggcactgcctctgacatgtccgaccttaggcaaatgcttgactcttctgagctcagtcttgtcatggcaaaataaagataataatagtgtttttttatggagttagcgtgaggatggaaaacaatagcaaaattgattagactataaaaggtctcaacaaatagtagtagattttatcatccattaatccttccctctcctctcttactcatcccatcacgtatgcctcttaattttcccttacctataataagagttattcctcttattatattcttcttatagtgattctggatattaaagtgggaatgaggggcaggccactaacgaagaagatgtttctcaaagaagctaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgttggttttttgtgtgtgcattgtctgagtaggtgtcattctattctggggggtggggtggggcagcacagcaagggggaggattgggaagacaatagcaggcatgctggggatgcggtgggctctatggagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctctcactacttctggaatagctcagaggccgaggcggcctcggcctctgcataaataaaaaaaattagtcgaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggg

SEQ ID NO: 36 - DNA, 8221 bp, synthetic construct
  misc_feature 1..8221: note = Lentiviral vector
  source 1..8221: mol_type = other DNA
acgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatagcacctagatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacgggggggtcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgatctcgacacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagatgctgcccactcagactttattcaaagaccaggaagggccggtgcaaggaggggaggagggcccgttgggaggcccagcgggcaggaggaacggctaccgaggctccagctgtgctgctctgcggcgattaacggtatttggaggtcagcacggtgctcacagaagccaggaacttgtccagggaggcgtgcaccgcaggggtgaactcggcggggaggtgggcggccagggtcaccagcaggcagtggcttaggagctgtgcagagaagagggtcagtgcggcccaggcccgcagccgccgcctgcgctacacctcccgcaacccgcgtgatcctctgccctgagaggaaggcgccatctcgcccctcgacccagatcgctcccggcccgccgctcaccttgaagttgaccgggtccacccgaagcttgtgcgcgtgcaggtcgctcagggcggacagcgcgttgggcatgtcgtccacgtgcgccacggcgttggtcagcgcgtcggccaccttcttgccgtggcccttaacctgggcagagccgtggctcaggtcgaagtgcgggaagtaggtcttggtggtggggaaggacaggaacatcctgcggggagaagcagagtgaggggtggggtttgggtccggggccaggacggttgagggtggcctgtgggtccgggcgggcgaggagcccgggtcggagcaggggagggagcctcacctctccagggcctccgcaccatactcgccagcgtgcgcgccgaccttaccccaggcggccttgacgttggtcttgtcggcaggagacagcaccatggtgggttctctctgagtctgtggggaccagaagagtgccggcccgcgagcgcgccagggtttatgcttggggcgcgggggcacgcccggccgggcggcgctcattggctggcgcggagcccggggcgcggcctggaccgcaggggagtcccgggcggggcctgcgcggggccgggcggcgggctcggcctcccctgggggtgcacgcggggcgggggccaggacgtctccaccctccacccgccactacgtatatgtgtatatatatatatatattcaggaaataatatattctagaatatgtcacattctgtctcaggcatccattttctttatgatgccgtttgaggtggagttttagtcaggtggtcagcttctccttttttttgccatctgccctgtaagcatcctgctggggacccagataggagtcatcactctaggctgagaacatctgggcacacaccctaagcctcagcatgactcatcatgactcagcattgctgtgcttgagccagaaggtttgcttagaaggttacacagaaccagaaggcgggggtggggcactgaccccgacaggggcctggccagaactgctcatgcttggactatgggaggtcactaatggagacacacagaaatgtaacaggaactaaggaaaaactgaagctttgggggtataggggagcagtcccatgtagtagtagaatgaaaaatgctgctatgctgtgcctcccccacctttcccatgtctgccctctactcatggtctatctctcctggctcctgggagtcatggactccacccagcaccaccaacctgacctaaccacctatctgagcctgccagcctataacccatctgggccctgatagctggtggccagccctgaccccaccccaccctccctggaacctctgatagacacatctggcacaccagctcgcaaagtcaccgtgagggtcttgtgtttgctgagtcaaaattccttgaaatccaagtccttagagactcccaggcttggattcaaagctcctgactttctgtctagtgtatgtgcagtgagccccttttcctctaactgaaagaaggaaaaaaaaatggaacccaaaatattctacatagtttccatgtcacagccagggctgggcagtctcctgttatttcttttaaaataaatatatcatttaaatgcataaataagcaaaccctgctcgggaatgggagggagagtctctggagtccaccccttctcggccctggctctgcagatagtgctatcaaagccctgacagagccctgcccattgctgggccttggagtgagtcagcctagtagagaggcagggcaagccatctcatagctgctgagtgggagagagaaaagggctcattgtctataaactcaggtcatggctattcttattaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgttggttttttgtgtgtgcattgtctgagtaggtgtcattctattctggggggtggggtggggcagcacagcaagggggaggattgggaagacaatagcaggcatgctggggatgcggtgggctctatggagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctctcactacttctggaatagctcagaggccgaggcggcctcggcctctgcataaataaaaaaaattagtcgaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggg

SEQ ID NO: 37 - DNA, 8585 bp, synthetic construct; identical to SEQ ID NO: 27
  misc_feature 1..8585: note = Lentiviral vector
  source 1..8585: mol_type = other DNA

SEQ ID NO: 38 - DNA, 8017 bp, synthetic construct
  misc_feature 1..8017: note = Lentiviral vector
  source 1..8017: mol_type = other DNA
acgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatagcacctagatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacgggggggtcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgatctcgacacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagatgctgcccactcagactttattcaaagaccaggaagggccggtgcaaggaggggaggagggcccgttgggaggcccagcgggcaggaggaacggctaccgaggctccagctgtgctgctctgcggcgattaacggtatttggaggtcagcacggtgctcacagaagccaggaacttgtccagggaggcgtgcaccgcaggggtgaactcggcggggaggtgggcggccagggtcaccagcaggcagtggcttaggagcttgaagttgaccgggtccacccgaagcttgtgcgcgtgcaggtcgctcagggcggacagcgcgttgggcatgtcgtccacgtgcgccacggcgttggtcagcgcgtcggccaccttcttgccgtggcccttaacctgggcagagccgtggctcaggtcgaagtgcgggaagtaggtcttggtggtggggaaggacaggaacatcctctccagggcctccgcaccatactcgccagcgtgcgcgccgaccttaccccaggcggccttgacgttggtcttgtcggcaggagacagcaccatggtgggttctctctgagtctgtggggaccagaagagtaagcaatagatggctctgccctgacttttatgcccagccctggctcctgccctccctgctcctgggagtagattggccaaccctagggtgtggctccacagggtgaggtctaagtgatgacagccgtacctgtccttggctcttctggcactggcttaggagttggacttcaaaccctcagccctccctctaagatatatctcttggccccataccatcagtacaaattgctactaaaaacatcctcctttgcaagtgtatttactacgtatatgtgtatatatatatatatattcaggaaataatatattctagaatatgtcacattctgtctcaggcatccattttctttatgatgccgtttgaggtggagttttagtcaggtggtcagcttctccttttttttgccatctgccctgtaagcatcctgctggggacccagataggagtcatcactctaggctgagaacatctgggcacacaccctaagcctcagcatgactcatcatgactcagcattgctgtgcttgagccagaaggtttgcttagaaggttacacagaaccagaaggcgggggtggggcactgaccccgacaggggcctggccagaactgctcatgcttggactatgggaggtcactaatggagacacacagaaatgtaacaggaactaaggaaaaactgaagctttgggggtataggggagcagtcccatgtagtagtagaatgaaaaatgctgctatgctgtgcctcccccacctttcccatgtctgccctctactcatggtctatctctcctggctcctgggagtcatggactccacccagcaccaccaacctgacctaaccacctatctgagcctgccagcctataacccatctgggccctgatagctggtggccagccctgaccccaccccaccctccctggaacctctgatagacacatctggcacaccagctcgcaaagtcaccgtgagggtcttgtgtttgctgagtcaaaattccttgaaatccaagtccttagagactcccaggcttggattcaaagctcctgactttctgtctagtgtatgtgcagtgagccccttttcctctaactgaaagaaggaaaaaaaaatggaacccaaaatattctacatagtttccatgtcacagccagggctgggcagtctcctgttatttcttttaaaataaatatatcatttaaatgcataaataagcaaaccctgctcgggaatgggagggagagtctctggagtccaccccttctcggccctggctctgcagatagtgctatcaaagccctgacagagccctgcccattgctgggccttggagtgagtcagcctagtagagaggcagggcaagccatctcatagctgctgagtgggagagagaaaagggctcattgtctataaactcaggtcatggctattcttattaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgttggttttttgtgtgtgcattgtctgagtaggtgtcattctattctggggggtggggtggggcagcacagcaagggggaggattgggaagacaatagcaggcatgctggggatgcggtgggctctatggagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctctcactacttctggaatagctcagaggccgaggcggcctcggcctctgcataaataaaaaaaattagtcgaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggg

SEQ ID NO: 39 - DNA, 9560 bp, synthetic construct
  misc_feature 1..9560: note = Lentiviral vector
  source 1..9560: mol_type = other DNA
acgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatagcacctagatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacgggggggtcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgatctcgacacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagatgctgcccactcagactttattcaaagaccaggaagggccggtgcaaggaggggaggagggcccgttgggaggcccagcgggcaggaggaacggctaccgaggctccagcttaacggtatttggaggtcagcacggtgctcacagaagccaggaacttgtccagggaggcgtgcaccgcaggggtgaactcggcggggaggtgggcggccagggtcaccagcaggcagtggcttaggagcttgaagttgaccgggtccacccgaagcttgtgcgcgtgcaggtcgctcagggcggacagcgcgttgggcatgtcgtccacgtgcgccacggcgttggtcagcgcgtcggccaccttcttgccgtggcccttaacctgggcagagccgtggctcaggtcgaagtgcgggaagtaggtcttggtggtggggaaggacaggaacatcctgcggggagaagcagagtgaggggtggggtttgggtccggggccaggacggttgagggtggcctgtgggtccgggcgggcgaggagcccgggtcggagcaggggagggagcctcacctctccagggcctccgcaccatactcgccagcgtgcgcgccgaccttaccccaggcggccttgacgttggtcttgtcggcaggagacagcaccatggtgggttctctctgagtctgtggggaccagaagagtaagcaatagatggctctgccctgacttttatgcccagccctggctcctgccctccctgctcctgggagtagattggccaaccctagggtgtggctccacagggtgaggtctaagtgatgacagccgtacctgtccttggctcttctggcactggcttaggagttggacttcaaaccctcagccctccctctaagatatatctcttggccccataccatcagtacaaattgctactaaaaacatcctcctttgcaagtgtatttacccttttgccacctagctgtccaggggtgccttaaaatggcaaacaaggtttgttttcttttcctgttttcatgccttcctcttccatatccttgtttcatattaatacatgtgtatagatcctaaaaatctatacacatgtattaataaagcctgattctgccgcttctaggtatagaggccacctgcaagataaatatttgattcacaataactaatcattctatggcaattgataacaacaaatatatatatatatatatatacgtatatgtgtatatatatatatatatattcaggaaataatatattctagaatatgtcacattctgtctcaggcatccattttctttatgatgccgtttgaggtggagttttagtcaggtggtcagcttctccttttttttgccatctgccctgtaagcatcctgctggggacccagataggagtcatcactctaggctgagaacatctgggcacacaccctaagcctcagcatgactcatcatgactcagcattgctgtgcttgagccagaaggtttgcttagaaggttacacagaaccagaaggcgggggtggggcactgaccccgacaggggcctggccagaactgctcatgcttggactatgggaggtcactaatggagacacacagaaatgtaacaggaactaaggaaaaactgaagcttatttaatcagagatgaggatgctggaagggatagagggagctgagcttgtaaaaagtatagtaatcattcagcaaatggttttgaagcacctgctggatgctaaacactattttcagtgcttgaatcataaataagaataaaacatgtatcttattccccacaagagtccaagtaaaaaataacagttaattataatgtgctctgtcccccaggctggagtgcagtggcacgatctcagctcactgcaacctccgcctcccgggttcaagcaattctcctgcctcagccaccctaatagctgggattacaggtgcacaccaccatgccaggctaatttttgtactttttgtagaggcagggtatcaccatgttgtccaagatggtcttgaactcctgagctccaagcagtccacccacctcagcctcccaaagtgctgggattacaggtgtgagacaccatgcccagattttccatatttaatagaggtatttatgggatgggggaaaagaatgtttctctcactgtggattattttagagagtggagaatggtcaagatttttttaaaaattaagaaaacataagttggaccttgagaaatgaaaatttatttttttgttggaggatacccattctctatctcccatcagggcaagctgtaaggaactggctaagacacagtgagacagagtgacttagtcttagaggccccactggtacgacggtcaccaagctttcattaaaaaaagtctaaccagctgcattcgactttgactgcagcagctggttagaaggttctactggaggagggtcccagcccattgctaaattaacatcaggctctgagactggcagtatatctctaacagtggttgatgctatcttctggaacttgcctgctacattgagaccactgacccatacataggaagcccatagctctgtcctgaactgttaggccactggtccagagagtgtgcatctcctttgatcctcataataaccctatgagatagacacaattattactcttactttatagatgatgatcctgaaaacataggagtcaaggcacttgcccctagctgggggtataggggagcagtcccatgtagtagtagaatgaaaaatgctgctatgctgtgcctcccccacctttcccatgtctgccctctactcatggtctatctctcctggctcctgggagtcatggactccacccagcaccaccaacctgacctaaccacctatctgagcctgccagcctataacccatctgggccctgatagctggtggccagccctgaccccaccccaccctccctggaacctctgatagacacatctggcacaccagctcgcaaagtcaccgtgagggtcttgtgtttgctgagtcaaaattccttgaaatccaagtccttagagactcctgctcccaaatttacagtcatagacttcttcatggctgtctcctttatccacagaatgattcctttgcttcattgccccatccatctgatcctcctcatcagtgcagcacagggcccatgagcagtagctgcagagtctcacataggtctggcactgcctctgacatgtccgaccttaggcaaatgcttgactcttctgagctcagtcttgtcatggcaaaataaagataataatagtgtttttttatggagttagcgtgaggatggaaaacaatagcaaaattgattagactataaaaggtctcaacaaatagtagtagattttatcatccattaatccttccctctcctctcttactcatcccatcacgtatgcctcttaattttcccttacctataataagagttattcctcttattatattcttcttatagtgattctggatattaaagtgggaatgaggggcaggccactaacgaagaagatgtttctcaaagaagctaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgttggttttttgtgtgtgcattgtctgagtaggtgtcattctattctggggggtggggtggggcagcacagcaagggggaggattgggaagacaatagcaggcatgctggggatgcggtgggctctatggagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctctcactacttctggaatagctcagaggccgaggcggcctcggcctctgcataaataaaaaaaattagtcgaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggg

SEQ ID NO: 40 - DNA, 8116 bp, synthetic construct
  misc_feature 1..8116: note = Lentiviral vector
  source 1..8116: mol_type = other DNA
acgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatagcacctagatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacgggggggtcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgatctcgacacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagatgctgcccactcagactttattcaaagaccaggaagggccggtgcaaggaggggaggagggcccgttgggaggcccagcgggcaggaggaacggctaccgaggctccagcttaacggtatttggaggtcagcacggtgctcacagaagccaggaacttgtccagggaggcgtgcaccgcaggggtgaactcggcggggaggtgggcggccagggtcaccagcaggcagtggcttaggagcttgaagttgaccgggtccacccgaagcttgtgcgcgtgcaggtcgctcagggcggacagcgcgttgggcatgtcgtccacgtgcgccacggcgttggtcagcgcgtcggccaccttcttgccgtggcccttaacctgggcagagccgtggctcaggtcgaagtgcgggaagtaggtcttggtggtggggaaggacaggaacatcctgcggggagaagcagagtgaggggtggggtttgggtccggggccaggacggttgagggtggcctgtgggtccgggcgggcgaggagcccgggtcggagcaggggagggagcctcacctctccagggcctccgcaccatactcgccagcgtgcgcgccgaccttaccccaggcggccttgacgttggtcttgtcggcaggagacagcaccatggtgggttctctctgagtctgtggggaccagaagagtaagcaatagatggctctgccctgacttttatgcccagccctggctcctgccctccctgctcctgggagtagattggccaaccctagggtgtggctccacagggtgaggtctaagtgatgacagccgtacctgtccttggctcttctggcactggcttaggagttggacttcaaaccctcagccctccctctaagatatatctcttggccccataccatcagtacaaattgctactaaaaacatcctcctttgcaagtgtatttactacgtatatgtgtatatatatatatatattcaggaaataatatattctagaatatgtcacattctgtctcaggcatccattttctttatgatgccgtttgaggtggagttttagtcaggtggtcagcttctccttttttttgccatctgccctgtaagcatcctgctggggacccagataggagtcatcactctaggctgagaacatctgggcacacaccctaagcctcagcatgactcatcatgactcagcattgctgtgcttgagccagaaggtttgcttagaaggttacacagaaccagaaggcgggggtggggcactgaccccgacaggggcctggccagaactgctcatgcttggactatgggaggtcactaatggagacacacagaaatgtaacaggaactaaggaaaaactgaagctttgggggtataggggagcagtcccatgtagtagtagaatgaaaaatgctgctatgctgtgcctcccccacctttcccatgtctgccctctactcatggtctatctctcctggctcctgggagtcatggactccacccagcaccaccaacctgacctaaccacctatctgagcctgccagcctataacccatctgggccctgatagctggtggccagccctgaccccaccccaccctccctggaacctctgatagacacatctggcacaccagctcgcaaagtcaccgtgagggtcttgtgtttgctgagtcaaaattccttgaaatccaagtccttagagactcccaggcttggattcaaagctcctgactttctgtctagtgtatgtgcagtgagccccttttcctctaactgaaagaaggaaaaaaaaatggaacccaaaatattctacatagtttccatgtcacagccagggctgggcagtctcctgttatttcttttaaaataaatatatcatttaaatgcataaataagcaaaccctgctcgggaatgggagggagagtctctggagtccaccccttctcggccctggctctgcagatagtgctatcaaagccctgacagagccctgcccattgctgggccttggagtgagtcagcctagtagagaggcagggcaagccatctcatagctgctgagtgggagagagaaaagggctcattgtctataaactcaggtcatggctattcttattaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgttggttttttgtgtgtgcattgtctgagtaggtgtcattctattctggggggtggggtggggcagcacagcaagggggaggattgggaagacaatagcaggcatgctggggatgcggtgggctctatggagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctctcactacttctggaatagctcagaggccgaggcggcctcggcctctgcataaataaaaaaaattagtcgaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggg

SEQ ID NO: 41 - DNA, 9720 bp, synthetic construct
  misc_feature 1..9720: note = Lentiviral vector
  source 1..9720: mol_type = other DNA
acgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatagcacctagatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacgggggggtcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgatctcgacacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagatgctgcccactcagactttattcaaagaccaggaagggccggtgcaaggaggggaggagggcccgttgggaggcccagcgggcaggaggaacggctaccgaggctccagctgtgctgctctgcggcgattaacggtatttggaggtcagcacggtgctcacagaagccaggaacttgtccagggaggcgtgcaccgcaggggtgaactcggcggggaggtgggcggccagggtcaccagcaggcagtggcttaggagctgtgcagagaagagggtcagtgcggcccaggcccgcagccgccgcctgcgctacacctcccgcaacccgcgtgatcctctgccctgagaggaaggcgccatctcgcccctcgacccagatcgctcccggcccgccgctcaccttgaagttgaccgggtccacccgaagcttgtgcgcgtgcaggtcgctcagggcggacagcgcgttgggcatgtcgtccacgtgcgccacggcgttggtcagcgcgtcggccaccttcttgccgtggcccttaacctgggcagagccgtggctcaggtcgaagtgcgggaagtaggtcttggtggtggggaaggacaggaacatcctgcggggagaagcagagtgaggggtggggtttgggtccggggccaggacggttgagggtggcctgtgggtccgggcgggcgaggagcccgggtcggagcaggggagggagcctcacctctccagggcctccgcaccatactcgccagcgtgcgcgccgaccttaccccaggcggccttgacgttggtcttgtcggcaggagacagcaccatggtgggttctctctgagtctgtggggaccagaagagtaagcaatagatggctctgccctgacttttatgcccagccctggctcctgccctccctgctcctgggagtagattggccaaccctagggtgtggctccacagggtgaggtctaagtgatgacagccgtacctgtccttggctcttctggcactggcttaggagttggacttcaaaccctcagccctccctctaagatatatctcttggccccataccatcagtacaaattgctactaaaaacatcctcctttgcaagtgtatttacccttttgccacctagctgtccaggggtgccttaaaatggcaaacaaggtttgttttcttttcctgttttcatgccttcctcttccatatccttgtttcatattaatacatgtgtatagatcctaaaaatctatacacatgtattaataaagcctgattctgccgcttctaggtatagaggccacctgcaagataaatatttgattcacaataactaatcattctatggcaattgataacaacaaatatatatatatatatatatacgtatatgtgtatatatatatatatatattcaggaaataatatattctagaatatgtcacattctgtctcaggcatccattttctttatgatgccgtttgaggtggagttttagtcaggtggtcagcttctccttttttttgccatctgccctgtaagcatcctgctggggacccagataggagtcatcactctaggctgagaacatctgggcacacaccctaagcctcagcatgactcatcatgactcagcattgctgtgcttgagccagaaggtttgcttagaaggttacacagaaccagaaggcgggggtggggcactgaccccgacaggggcctggccagaactgctcatgcttggactatgggaggtcactaatggagacacacagaaatgtaacaggaactaaggaaaaactgaagcttatttaatcagagatgaggatgctggaagggatagagggagctgagcttgtaaaaagtatagtaatcattcagcaaatggttttgaagcacctgctggatgctaaacactattttcagtgcttgaatcataaataagaataaaacatgtatcttattccccacaagagtccaagtaaaaaataacagttaattataatgtgctctgtcccccaggctggagtgcagtggcacgatctcagctcactgcaacctccgcctcccgggttcaagcaattctcctgcctcagccaccctaatagctgggattacaggtgcacaccaccatgccaggctaatttttgtactttttgtagaggcagggtatcaccatgttgtccaagatggtcttgaactcctgagctccaagcagtccacccacctcagcctcccaaagtgctgggattacaggtgtgagacaccatgcccagattttccatatttaatagaggtatttatgggatgggggaaaagaatgtttctctcactgtggattattttagagagtggagaatggtcaagatttttttaaaaattaagaaaacataagttggaccttgagaaatgaaaatttatttttttgttggaggatacccattctctatctcccatcagggcaagctgtaaggaactggctaagacacagtgagacagagtgacttagtcttagaggccccactggtacgacggtcaccaagctttcattaaaaaaagtctaaccagctgcattcgactttgactgcagcagctggttagaaggttctactggaggagggtcccagcccattgctaaattaacatcaggctctgagactggcagtatatctctaacagtggttgatgctatcttctggaacttgcctgctacattgagaccactgacccatacataggaagcccatagctctgtcctgaactgttaggccactggtccagagagtgtgcatctcctttgatcctcataataaccctatgagatagacacaattattactcttactttatagatgatgatcctgaaaacataggagtcaaggcacttgcccctagctgggggtataggggagcagtcccatgtagtagtagaatgaaaaatgctgctatgctgtgcctcccccacctttcccatgtctgccctctactcatggtctatctctcctggctcctgggagtcatggactccacccagcaccaccaacctgacctaaccacctatctgagcctgccagcctataacccatctgggccctgatagctggtggccagccctgaccccaccccaccctccctggaacctctgatagacacatctggcacaccagctcgcaaagtcaccgtgagggtcttgtgtttgctgagtcaaaattccttgaaatccaagtccttagagactcctgctcccaaatttacagtcatagacttcttcatggctgtctcctttatccacagaatgattcctttgcttcattgccccatccatctgatcctcctcatcagtgcagcacagggcccatgagcagtagctgcagagtctcacataggtctggcactgcctctgacatgtccgaccttaggcaaatgcttgactcttctgagctcagtcttgtcatggcaaaataaagataataatagtgtttttttatggagttagcgtgaggatggaaaacaatagcaaaattgattagactataaaaggtctcaacaaatagtagtagattttatcatccattaatccttccctctcctctcttactcatcccatcacgtatgcctcttaattttcccttacctataataagagttattcctcttattatattcttcttatagtgattctggatattaaagtgggaatgaggggcaggccactaacgaagaagatgtttctcaaagaagctaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgttggttttttgtgtgtgcattgtctgagtaggtgtcattctattctggggggtggggtggggcagcacagcaagggggaggattgggaagacaatagcaggcatgctggggatgcggtgggctctatggagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctctcactacttctggaatagctcagaggccgaggcggcctcggcctctgcataaataaaaaaaattagtcgaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggg

SEQ ID NO: 42 - DNA, 8532 bp, synthetic construct
  misc_feature 1..8532: note = Lentiviral vector
  source 1..8532: mol_type = other DNA
acgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatagcacctagatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacgggggggtcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgatctcgacacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagatgctgcccactcagactttattcaaagaccaggaagggccggtgcaaggaggggaggagggcccgttgggaggcccagcgggcaggaggaacggctaccgaggctccagctgtgctgctctgcggcgattaacggtatttggaggtcagcacggtgctcacagaagccaggaacttgtccagggaggcgtgcaccgcaggggtgaactcggcggggaggtgggcggccagggtcaccagcaggcagtggcttaggagctgtgcagagaagagggtcagtgcggcccaggcccgcagccgccgcctgcgctacacctcccgcaacccgcgtgatcctctgccctgagaggaaggcgccatctcgcccctcgacccagatcgctcccggcccgccgctcaccttgaagttgaccgggtccacccgaagcttgtgcgcgtgcaggtcgctcagggcggacagcgcgttgggcatgtcgtccacgtgcgccacggcgttggtcagcgcgtcggccaccttcttgccgtggcccttaacctgggcagagccgtggctcaggtcgaagtgcgggaagtaggtcttggtggtggggaaggacaggaacatcctgcggggagaagcagagtgaggggtggggtttgggtccggggccaggacggttgagggtggcctgtgggtccgggcgggcgaggagcccgggtcggagcaggggagggagcctcacctctccagggcctccgcaccatactcgccagcgtgcgcgccgaccttaccccaggcggccttgacgttggtcttgtcggcaggagacagcaccatggtgggttctctctgagtctgtggggaccagaagagtaagcaatagatggctctgccctgacttttatgcccagccctggctcctgccctccctgctcctgggagtagattggccaaccctagggtgtggctccacagggtgaggtctaagtgatgacagccgtacctgtccttggctcttctggcactggcttaggagttggacttcaaaccctcagccctccctctaagatatatctcttggccccataccatcagtacaaattgctactaaaaacatcctcctttgcaagtgtatttacctctcagataaacaggagggggaggcagcaccagaggttgtaatcagcagtaccattcacccctttcccttgtcccacctccacccccacaggatgactcagcagtcctgttggcctccagaagcactgagtcatggttggcccagttatctgctccctcaagtgttttcacagacccccacagctgcatgcctccacccttggcttgggcagatgtgcttgcctggctgactgtggtccctgataggttccagagtacgtatatgtgtatatatatatatatattcaggaaataatatattctagaatatgtcacattctgtctcaggcatccattttctttatgatgccgtttgaggtggagttttagtcaggtggtcagcttctccttttttttgccatctgccctgtaagcatcctgctggggacccagataggagtcatcactctaggctgagaacatctgggcacacaccctaagcctcagcatgactcatcatgactcagcattgctgtgcttgagccagaaggtttgcttagaaggttacacagaaccagaaggcgggggtggggcactgaccccgacaggggcctggccagaactgctcatgcttggactatgggaggtcactaatggagacacacagaaatgtaacaggaactaaggaaaaactgaagctttgggggtataggggagcagtcccatgtagtagtagaatgaaaaatgctgctatgctgtgcctcccccacctttcccatgtctgccctctactcatggtctatctctcctggctcctgggagtcatggactccacccagcaccaccaacctgacctaaccacctatctgagcctgccagcctataacccatctgggccctgatagctggtggccagccctgaccccaccccaccctccctggaacctctgatagacacatctggcacaccagctcgcaaagtcaccgtgagggtcttgtgtttgctgagtcaaaattccttgaaatccaagtccttagagactcccaggcttggattcaaagctcctgactttctgtctagtgtatgtgcagtgagccccttttcctctaactgaaagaaggaaaaaaaaatggaacccaaaatattctacatagtttccatgtcacagccagggctgggcagtctcctgttatttcttttaaaataaatatatcatttaaatgcataaataagcaaaccctgctcgggaatgggagggagagtctctggagtccaccccttctcggccctggctctgcagatagtgctatcaaagccctgacagagccctgcccattgctgggccttggagtgagtcagcctagtagagaggcagggcaagccatctcatagctgctgagtgggagagagaaaagggctcattgtctataaactcaggtcatggctattcttattaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgttggttttttgtgtgtgcattgtctgagtaggtgtcattctattctggggggtggggtggggcagcacagcaagggggaggattgggaagacaatagcaggcatgctggggatgcggtgggctctatggagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctctcactacttctggaatagctcagaggccgaggcggcctcggcctctgcataaataaaaaaaattagtcgaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggg

SEQ ID NO: 43 - DNA, 8276 bp, synthetic construct
  misc_feature 1..8276: note = Lentiviral vector
  source 1..8276: mol_type = other DNA
acgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatagcacctagatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacgggggggtcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgatctcgacacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagatgctgcccactcagactttattcaaagaccaggaagggccggtgcaaggaggggaggagggcccgttgggaggcccagcgggcaggaggaacggctaccgaggctccagctgtgctgctctgcggcgattaacggtatttggaggtcagcacggtgctcacagaagccaggaacttgtccagggaggcgtgcaccgcaggggtgaactcggcggggaggtgggcggccagggtcaccagcaggcagtggcttaggagctgtgcagagaagagggtcagtgcggcccaggcccgcagccgccgcctgcgctacacctcccgcaacccgcgtgatcctctgccctgagaggaaggcgccatctcgcccctcgacccagatcgctcccggcccgccgctcaccttgaagttgaccgggtccacccgaagcttgtgcgcgtgcaggtcgctcagggcggacagcgcgttgggcatgtcgtccacgtgcgccacggcgttggtcagcgcgtcggccaccttcttgccgtggcccttaacctgggcagagccgtggctcaggtcgaagtgcgggaagtaggtcttggtggtggggaaggacaggaacatcctgcggggagaagcagagtgaggggtggggtttgggtccggggccaggacggttgagggtggcctgtgggtccgggcgggcgaggagcccgggtcggagcaggggagggagcctcacctctccagggcctccgcaccatactcgccagcgtgcgcgccgaccttaccccaggcggccttgacgttggtcttgtcggcaggagacagcaccatggtgggttctctctgagtctgtggggaccagaagagtaagcaatagatggctctgccctgacttttatgcccagccctggctcctgccctccctgctcctgggagtagattggccaaccctagggtgtggctccacagggtgaggtctaagtgatgacagccgtacctgtccttggctcttctggcactggcttaggagttggacttcaaaccctcagccctccctctaagatatatctcttggccccataccatcagtacaaattgctactaaaaacatcctcctttgcaagtgtatttactacgtatatgtgtatatatatatatatattcaggaaataatatattctagaatatgtcacattctgtctcaggcatccattttctttatgatgccgtttgaggtggagttttagtcaggtggtcagcttctccttttttttgccatctgccctgtaagcatcctgctggggacccagataggagtcatcactctaggctgagaacatctgggcacacaccctaagcctcagcatgactcatcatgactcagcattgctgtgcttgagccagaaggtttgcttagaaggttacacagaaccagaaggcgggggtggggcactgaccccgacaggggcctggccagaactgctcatgcttggactatgggaggtcactaatggagacacacagaaatgtaacaggaactaaggaaaaactgaagctttgggggtataggggagcagtcccatgtagtagtagaatgaaaaatgctgctatgctgtgcctcccccacctttcccatgtctgccctctactcatggtctatctctcctggctcctgggagtcatggactccacccagcaccaccaacctgacctaaccacctatctgagcctgccagcctataacccatctgggccctgatagctggtggccagccctgaccccaccccaccctccctggaacctctgatagacacatctggcacaccagctcgcaaagtcaccgtgagggtcttgtgtttgctgagtcaaaattccttgaaatccaagtccttagagactcccaggcttggattcaaagctcctgactttctgtctagtgtatgtgcagtgagccccttttcctctaactgaaagaaggaaaaaaaaatggaacccaaaatattctacatagtttccatgtcacagccagggctgggcagtctcctgttatttcttttaaaataaatatatcatttaaatgcataaataagcaaaccctgctcgggaatgggagggagagtctctggagtccaccccttctcggccctggctctgcagatagtgctatcaaagccctgacagagccctgcccattgctgggccttggagtgagtcagcctagtagagaggcagggcaagccatctcatagctgctgagtgggagagagaaaagggctcattgtctataaactcaggtcatggctattcttattaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgttggttttttgtgtgtgcattgtctgagtaggtgtcattctattctggggggtggggtggggcagcacagcaagggggaggattgggaagacaatagcaggcatgctggggatgcggtgggctctatggagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctctcactacttctggaatagctcagaggccgaggcggcctcggcctctgcataaataaaaaaaattagtcgaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggg

SEQ ID NO: 44 - DNA, 9840 bp, synthetic construct
  misc_feature 1..9840: note = Lentiviral vector
  source 1..9840: mol_type = other DNA
acgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatagcacctagatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacgggggggtcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgatctcgacacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagatcaaagaccaagaggtacaggtgcaagggagagaagaagggcatggccagaaggcaagccccgcagaaggcagctgtgctgctctgcggcgattaacggtacttggaggtcagcacggtgctcacagaggcaaggaatttgtccagagaggcatgcaccgcgggggtgaaatcggcagggtggtggctagccaaggtcaccagcaggcagtggctcaggagctgcggagacaaagtggacaccctgatgcctctgctccccttcctgggaccactatgttccctgccttgggcacgaggacccccaagcccttcctaggggtcccagatgccgcctgccaggtcccagcgcataccttgaagttgacgggatccacacgcagcttgtgggcatgcaggtcgctcagagcagacagggcaccgggcaggtcatcgaggtggcctgcagcattggccagagcatcggcgaccttcttgccgtgacccttgacctgggcagagccgtggcttacatcaaagtgagggaagtaggtcttggtggtggggaagctagcaaacatcctgggagaaggagagaagagaagttgaggtcacagaaaagcatagttagaagctgcccactgagcgagtgccaggtccatattggatcctgtgatccttacagatcaaggtcctgttctcacctttccagggcttcagctccatattcagcaccatggccaccaatcttcccccaggcagccttgatgttgcttttgtcttccccagagagcaccatggtttcttcctgagtctgtcagaatcagaagtgtcttggacctgcagcaagtagcacttatatgccctccaagggcgtgtcctccaggcagggtggacacgcccttggagcagttctcattggctggtttgtgcttgctctacccagtaaaacttgttagggcccctgagattctggggctgggttgtgcttccccaagcaatagatggctctgccctgacttttatgcccagccctggctcctgccctccctgctcctgggagtagattggccaaccctagggtgtggctccacagggtgaggtctaagtgatgacagccgtacctgtccttggctcttctggcactggcttaggagttggacttcaaaccctcagccctccctctaagatatatctcttggccccataccatcagtacaaattgctactaaaaacatcctcctttgcaagtgtatttacccttttgccacctagctgtccaggggtgccttaaaatggcaaacaaggtttgttttcttttcctgttttcatgccttcctcttccatatccttgtttcatattaatacatgtgtatagatcctaaaaatctatacacatgtattaataaagcctgattctgccgcttctaggtatagaggccacctgcaagataaatatttgattcacaataactaatcattctatggcaattgataacaacaaatatatatatatatatatatacgtatatgtgtatatatatatatatatattcaggaaataatatattctagaatatgtcacattctgtctcaggcatccattttctttatgatgccgtttgaggtggagttttagtcaggtggtcagcttctccttttttttgccatctgccctgtaagcatcctgctggggacccagataggagtcatcactctaggctgagaacatctgggcacacaccctaagcctcagcatgactcatcatgactcagcattgctgtgcttgagccagaaggtttgcttagaaggttacacagaaccagaaggcgggggtggggcactgaccccgacaggggcctggccagaactgctcatgcttggactatgggaggtcactaatggagacacacagaaatgtaacaggaactaaggaaaaactgaagcttatttaatcagagatgaggatgctggaagggatagagggagctgagcttgtaaaaagtatagtaatcattcagcaaatggttttgaagcacctgctggatgctaaacactattttcagtgcttgaatcataaataagaataaaacatgtatcttattccccacaagagtccaagtaaaaaataacagttaattataatgtgctctgtcccccaggctggagtgcagtggcacgatctcagctcactgcaacctccgcctcccgggttcaagcaattctcctgcctcagccaccctaatagctgggattacaggtgcacaccaccatgccaggctaatttttgtactttttgtagaggcagggtatcaccatgttgtccaagatggtcttgaactcctgagctccaagcagtccacccacctcagcctcccaaagtgctgggattacaggtgtgagacaccatgcccagattttccatatttaatagaggtatttatgggatgggggaaaagaatgtttctctcactgtggattattttagagagtggagaatggtcaagatttttttaaaaattaagaaaacataagttggaccttgagaaatgaaaatttatttttttgttggaggatacccattctctatctcccatcagggcaagctgtaaggaactggctaagacacagtgagacagagtgacttagtcttagaggccccactggtacgacggtcaccaagctttcattaaaaaaagtctaaccagctgcattcgactttgactgcagcagctggttagaaggttctactggaggagggtcccagcccattgctaaattaacatcaggctctgagactggcagtatatctctaacagtggttgatgctatcttctggaacttgcctgctacattgagaccactgacccatacataggaagcccatagctctgtcctgaactgttaggccactggtccagagagtgtgcatctcctttgatcctcataataaccctatgagatagacacaattattactcttactttatagatgatgatcctgaaaacataggagtcaaggcacttgcccctagctgggggtataggggagcagtcccatgtagtagtagaatgaaaaatgctgctatgctgtgcctcccccacctttcccatgtctgccctctactcatggtctatctctcctggctcctgggagtcatggactccacccagcaccaccaacctgacctaaccacctatctgagcctgccagcctataacccatctgggccctgatagctggtggccagccctgaccccaccccaccctccctggaacctctgatagacacatctggcacaccagctcgcaaagtcaccgtgagggtcttgtgtttgctgagtcaaaattccttgaaatccaagtccttagagactcctgctcccaaatttacagtcatagacttcttcatggctgtctcctttatccacagaatgattcctttgcttcattgccccatccatctgatcctcctcatcagtgcagcacagggcccatgagcagtagctgcagagtctcacataggtctggcactgcctctgacatgtccgaccttaggcaaatgcttgactcttctgagctcagtcttgtcatggcaaaataaagataataatagtgtttttttatggagttagcgtgaggatggaaaacaatagcaaaattgattagactataaaaggtctcaacaaatagtagtagattttatcatccattaatccttccctctcctctcttactcatcccatcacgtatgcctcttaattttcccttacctataataagagttattcctcttattatattcttcttatagtgattctggatattaaagtgggaatgaggggcaggccactaacgaagaagatgtttctcaaagaagctaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgttggttttttgtgtgtgcattgtctgagtaggtgtcattctattctggggggtggggtggggcagcacagcaagggggaggattgggaagacaatagcaggcatgctggggatgcggtgggctctatggagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctctcactacttctggaatagctcagaggccgaggcggcctcggcctctgcataaataaaaaaaattagtcgaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggg

SEQ ID NO: 45 - DNA, 8396 bp, synthetic construct
  misc_feature 1..8396: note = Lentiviral vector
  source 1..8396: mol_type = other DNA
acgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatagcacctagatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacgggggggtcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgatctcgacacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagatcaaagaccaagaggtacaggtgcaagggagagaagaagggcatggccagaaggcaagccccgcagaaggcagctgtgctgctctgcggcgattaacggtacttggaggtcagcacggtgctcacagaggcaaggaatttgtccagagaggcatgcaccgcgggggtgaaatcggcagggtggtggctagccaaggtcaccagcaggcagtggctcaggagctgcggagacaaagtggacaccctgatgcctctgctccccttcctgggaccactatgttccctgccttgggcacgaggacccccaagcccttcctaggggtcccagatgccgcctgccaggtcccagcgcataccttgaagttgacgggatccacacgcagcttgtgggcatgcaggtcgctcagagcagacagggcaccgggcaggtcatcgaggtggcctgcagcattggccagagcatcggcgaccttcttgccgtgacccttgacctgggcagagccgtggcttacatcaaagtgagggaagtaggtcttggtggtggggaagctagcaaacatcctgggagaaggagagaagagaagttgaggtcacagaaaagcatagttagaagctgcccactgagcgagtgccaggtccatattggatcctgtgatccttacagatcaaggtcctgttctcacctttccagggcttcagctccatattcagcaccatggccaccaatcttcccccaggcagccttgatgttgcttttgtcttccccagagagcaccatggtttcttcctgagtctgtcagaatcagaagtgtcttggacctgcagcaagtagcacttatatgccctccaagggcgtgtcctccaggcagggtggacacgcccttggagcagttctcattggctggtttgtgcttgctctacccagtaaaacttgttagggcccctgagattctggggctgggttgtgcttccccaagcaatagatggctctgccctgacttttatgcccagccctggctcctgccctccctgctcctgggagtagattggccaaccctagggtgtggctccacagggtgaggtctaagtgatgacagccgtacctgtccttggctcttctggcactggcttaggagttggacttcaaaccctcagccctccctctaagatatatctcttggccccataccatcagtacaaattgctactaaaaacatcctcctttgcaagtgtatttactacgtatatgtgtatatatatatatatattcaggaaataatatattctagaatatgtcacattctgtctcaggcatccattttctttatgatgccgtttgaggtggagttttagtcaggtggtcagcttctccttttttttgccatctgccctgtaagcatcctgctggggacccagataggagtcatcactctaggctgagaacatctgggcacacaccctaagcctcagcatgactcatcatgactcagcattgctgtgcttgagccagaaggtttgcttagaaggttacacagaaccagaaggcgggggtggggcactgaccccgacaggggcctggccagaactgctcatgcttggactatgggaggtcactaatggagacacacagaaatgtaacaggaactaaggaaaaactgaagctttgggggtataggggagcagtcccatgtagtagtagaatgaaaaatgctgctatgctgtgcctcccccacctttcccatgtctgccctctactcatggtctatctctcctggctcctgggagtcatggactccacccagcaccaccaacctgacctaaccacctatctgagcctgccagcctataacccatctgggccctgatagctggtggccagccctgaccccaccccaccctccctggaacctctgatagacacatctggcacaccagctcgcaaagtcaccgtgagggtcttgtgtttgctgagtcaaaattccttgaaatccaagtccttagagactcccaggcttggattcaaagctcctgactttctgtctagtgtatgtgcagtgagccccttttcctctaactgaaagaaggaaaaaaaaatggaacccaaaatattctacatagtttccatgtcacagccagggctgggcagtctcctgttatttcttttaaaataaatatatcatttaaatgcataaataagcaaaccctgctcgggaatgggagggagagtctctggagtccaccccttctcggccctggctctgcagatagtgctatcaaagccctgacagagccctgcccattgctgggccttggagtgagtcagcctagtagagaggcagggcaagccatctcatagctgctgagtgggagagagaaaagggctcattgtctataaactcaggtcatggctattcttattaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgttggttttttgtgtgtgcattgtctgagtaggtgtcattctattctggggggtggggtggggcagcacagcaagggggaggattgggaagacaatagcaggcatgctggggatgcggtgggctctatggagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctctcactacttctggaatagctcagaggccgaggcggcctcggcctctgcataaataaaaaaaattagtcgaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggg

(19) United States
(12) Patent Application Publication
(10) Pub. No.: US 2025/0381295 A1
(43) Pub. Date: Dec. 18, 2025

(54) LENTIVIRAL VECTORS EXPRESSING ALPHA-GLOBIN GENES FOR GENE THERAPY OF ALPHA THALASSEMIA

(71) Applicant: The Regents of the University of California, Oakland, CA (US)

(72) Inventors: Donald B. KOHN, Tarzana, CA (US); Roger HOLLIS, Los Angeles, CA (US); Eva ER SEGURA GENSLER, Los Angeles, CA (US)

(73) Assignee: The Regents of the University of California, Oakland, CA (US)

(21) Appl. No.: 18/878,842

(22) PCT Filed: Jun. 28, 2023
(86) PCT No.: PCT/US2023/026498

§ 371 Date: Dec. 24, 2024

Related U.S. Application Data
(60) Provisional application No. 63/356,936, filed on Jun. 29, 2022.

(51) Int. Cl. A61K 48/00; A61K 38/42; A61P 7/00; C12N 15/86

(52) U.S. Cl. CPC A61K 48/005; A61K 38/42; A61P 7/00; C12N 15/86; C07K 2319/20; C12N 2740/16022; C12N 2740/16043; C12N 2800/22

(57) ABSTRACT

This invention provides vectors (e.g., lentiviral vectors) and expression cassettes for incorporation into vectors for expressing an alpha-globin (α-globin) gene. In certain embodiments method of treatment using such vectors are provided.

### CROSS-REFERENCE TO RELATED APPLICATIONS

This application claims priority to and benefit of U.S. Ser. No. 63/356,936, filed on Jun. 29, 2022, which is incorporated herein by reference in its entirety for all purposes.

### STATEMENT OF GOVERNMENTAL SUPPORT

[Not Applicable]

### INCORPORATION BY REFERENCE OF SEQUENCE LISTING PROVIDED AS A TEXT FILE

[Not Applicable]

### BACKGROUND

Alpha Thalassemia (AT) and β-thalassemia (BT) are autosomal recessive disorders characterized by a reduced or absent synthesis of α- or β-globin peptide chains, which are both part of the hemoglobin hetero-tetramer. Hemoglobin is responsible for carrying oxygen in RBCs to deliver oxygen throughout the body, rendering its proper function critical for human health. Hemoglobin is an iron-(heme moiety)-containing hetero-tetrameric protein constituted of 2 α- and 2 β-globin chains that are both tightly transcriptionally regulated to maintain balanced α-globin to β-globin mRNA transcription and globin chain production needed for proper RBC development through effective erythropoiesis. In AT, the resulting imbalanced α/β mRNA and protein ratios, causes markedly decreased production of hemoglobin and the excess of free β-globin chains is toxic to developing erythroid cells. There is abnormal maturation of erythroblasts and their premature destruction in the bone marrow (ineffective erythropoiesis), which leads to anemia with decreased oxygen delivery and the clinical manifestations of AT.

There is a wide range of variability in the severity of anemia and other complications in AT, which are proportionate to the number of non-functional α-globin genes. While a typical genome contains four α-globin genes, a defect in 1 or 2 α-globin genes may be phenotypically silent or may be manifested with mild anemia. More severe forms of AT, with dysfunction of 3 or all 4 of the α-globin alleles, have severe and life-threatening disease.

A severe AT Diseases, designated HbH disease, arises from the loss or deficiency of 3 α-globin genes, HbH disease leads to a low a/Q mRNA ratio (˜0.3), and a 2- to 5-fold excess of β-globin chain production, resulting in the formation of nonfunctional and toxic β-globin tetramers, referred to as HbH. These HbH β-globin tetramers are not effective suppliers of oxygen as they have no Bohr effect, no heme-heme group interaction, and have a high affinity for oxygen, which prevents its release to tissues. Accumulation of HbH in RBCs eventually causes hemolytic anemia of variable severity, as well as splenomegaly, due to stress, and erythropoiesis occurring outside of the bone marrow (extramedullary hematopoiesis). Delayed growth in pediatric patients is also common.

Patients with Hemoglobin H-Constant Spring (HbH-CS) have 2 α-globin genes deleted and a third α-globin gene with the “Constant Spring” stop codon mutation that causes production of a non-functional, read-on α-globin chain. They have a severe AT phenotype that often requires chronic blood transfusions.

The most severe form of AT is known as alpha thalassemia major (ATM) or Hb Bart's Hydrops Fetalis and is most commonly caused by large deletions covering all four α-globin genes, yielding an α/β mRNA ratio of zero. ATM has historically been lethal in utero, although the advent of in utero blood transfusions has enabled safe full-term gestation, without generating any neurological abnormalities.

AT is one of the most common monogenic diseases in the world and is a major cause of morbidity and mortality. Yearly, ˜350,000 severely affected infants are born with AT due to high carrier prevalence affecting 300 million people, predominantly living in Asia, India, and the Middle East. Due to increasing migration of high-risk populations, AT has now become a clinical concern in the United States; there has been a recent increase in Asian immigration to the US, with more than half of this growth in the Western US. In California alone, where Asians make up 15% of the population, severe forms of AT, such as Hemoglobin H disease (HbH), have become the second most common hemoglobinopathy and have affected 1,594 newborns over a period of 10 years (2001-2011), detected by newborn screening data (NBS) programs. Thus, AT patients now represent a significant and increasing public health problem in California.

Patients suffering from severe HbH disease, as well as in utero transfusion-rescued ATM newborns, are deemed “transfusion-dependent” and require lifelong blood transfusions and iron chelation therapy. The need for frequent medical care episodes and continual close medical monitoring present a substantial burden to the affected patients and to healthcare systems.

A curative therapy for AT is allogeneic hematopoietic stem cell transplant (HSCT) for patients with an available matched donor. However, this option is limited by the scarce availability of matched donors, as well as an overall mortality rate of 5-10% due to complications such as graft-versus-host disease. Patients also face substantial risks from myeloablative regimens currently used to eradicate many or all HSC in the host's bone marrow to allow better engraftment of donor HSC.

Autologous transplantation of gene-modified hematopoietic stem cells (HSC) (HSC gene therapy) is an emergent and promising approach to treat inherited blood cell disorders. Early and pre-licensing clinical trials of gene therapy for primary immune deficiencies, hemoglobinopathies, lysosomal storage diseases, and other genetic blood cell diseases have employed ex vivo transduction of HSCs by LV. Using current methods, HSCs are collected from patients by mobilization into the peripheral blood and leukapheresis, processed for CD34⁺ cell selection, and then transduced with the LVs in a few days of cell culture. More than a dozen distinct genetic disorders have shown clinical benefits as good or better than with allogeneic HSCT, without any vector-related adverse effects. Unlike allogeneic HSCT, autologous transplants overcome the limits of identifying suitably matched donors as the patient's own cells are used.

This HSC gene therapy strategy has been safe and successful in ongoing clinical trials and has provided treatments to dozens of patients suffering from β-hemoglobinopathies, such as sickle cell disease (SCD) and β-thalassemia major (BTM) (two cases of leukemia post-GT in sickle cell disease patients were found not related to the LVs). LV gene therapy has been reported to provide significant clinical benefit, including freedom from ongoing transfusions in several BTM without adverse events up to six years. There are high similarities between pathogenesis and physiology of BT and AT, with severe deficiency of either globin chain leading to imbalances and ineffective erythropoiesis and anemia. Yet, all gene therapy and gene editing clinical trials for thalassemia to date have targeted BT major (BTM). Gene therapy and genome editing may be curative for ATM and severe HbH disease, but despite progress using these approaches for BTM, there are currently no analogous strategies for HSC gene therapy for patients with AT.

### SUMMARY

Various embodiments provided herein may include, but need not be limited to, one or more of the following:

Various embodiments provided herein may include, but need not be limited to, one or more of the following:

Embodiment 1: A recombinant lentiviral vector (LV) comprising: an expression cassette comprising a nucleic acid construct comprising:
an alpha-globin (α-globin) gene;
a 5′ UTR upstream from said α-globin gene and a 3′ UTR downstream from said α-globin gene where said 5′ and 3′ UTRs are from β-globin genes or from α-globin genes;
an α-globin promoter or a β globin promoter upstream from said 5′ UTR; and
a locus control region (LCR) upstream from said promoter, wherein said LCR is selected from the group consisting of core LCR UV (SEQ ID NO:11), GLOBE βLCR (A2-LCR) containing HS2 HS3 (SEQ ID NO:12), and GLOBE βLCR containing HS2 (SEQ ID NO:13), or an LCR having at least 95% or at least 98%, or at least 99% sequence identity to the foregoing LCRs.

Embodiment 2: The recombinant lentiviral vector of embodiment 1, wherein said expression cassette comprising a locus control region (LCR) selected from the group consisting of:
a core β-LCR comprising reduced length HS4, HS3, and HS2 DNase I hypersensitive sites; or
a Globe β-LCR comprising HS3 and HS2 DNase I hypersensitive sites; or
a Globe β-LCR comprising HS2 DNase I hypersensitive site and a β-LCR comprising reduced length HS4, and HS3, DNase I hypersensitive sites.

Embodiment 3: The recombinant lentiviral vector according to any one of embodiments 1-2, wherein said α-globin gene comprises a full-length α-globin gene including exon and introns.

Embodiment 4: The recombinant lentiviral vector according to any one of embodiments 1-2, wherein said α-globin gene comprises an HBA1 α-globin gene (SEQ ID NO:8).

Embodiment 5: The recombinant lentiviral vector according to any one of embodiments 1-2, wherein said α-globin gene comprises an HBA2 α-globin gene (SEQ ID NO:9).

Embodiment 6: The recombinant lentiviral vector according to any one of embodiments 1-2, wherein said α-globin gene comprises an alpha globin gene without intron 2.

Embodiment 7: The recombinant lentiviral vector according to any one of embodiments 1-2, wherein said α-globin gene comprises an alpha globin cDNA.

Embodiment 8: The recombinant lentiviral vector of embodiment 7, wherein said alpha globin cDNA is codon optimized.

Embodiment 9: The recombinant lentiviral vector according to any one of embodiments 3-8, wherein said alpha globin gene contains a tag.

Embodiment 10: The recombinant lentiviral vector of embodiment 9, wherein said tag comprises or consists of the sequence tgtgctgctctgcggcga (SEQ ID NO:18).

Embodiment 11: The recombinant lentiviral vector according to any one of embodiments 1-10, wherein said expression cassette comprises a 5′ UTR and a 3′UTR from a β-globin gene.

Embodiment 12: The recombinant lentiviral vector according to any one of embodiments 1-10 wherein said expression cassette comprises a 5′ UTR and a 3′UTR from an α-globin gene.

Embodiment 13: The recombinant lentiviral vector according to any one of embodiments 1-12, wherein said expression cassette comprises a full length β-globin promoter.

Embodiment 14: The recombinant lentiviral vector according to any one of embodiments 1-12, wherein said expression cassette comprises a shortened β-globin promoter (SEQ ID NO:1).

Embodiment 15: The recombinant lentiviral vector according to any one of embodiments 1-12, wherein said expression cassette comprises a full length α-globin promoter.

Embodiment 16: The recombinant lentiviral vector according to any one of embodiments 1-12 wherein said expression cassette comprises a shortened α-globin promoter (SEQ ID NO:14).

Embodiment 17: The recombinant lentiviral vector according to any one of embodiments 1-16, wherein said expression cassette comprises a core β-LCR comprising reduced length HS4 (SEQ ID NO:17), HS3 (SEQ ID NO:16), and HS2 (SEQ ID NO:15) DNase I hypersensitive sites.

Embodiment 18: The recombinant lentiviral vector according to any one of embodiments 1-16, wherein said expression cassette comprises a Globe 3-LCR comprising HS3 and HS2 DNase I hypersensitive sites (SEQ ID NO:12).

Embodiment 19: The recombinant lentiviral vector according to any one of embodiments 1-16, wherein said expression cassette comprises a Globe β-LCR comprising HS2 DNase I hypersensitive site (SEQ ID NO:13) and a β-LCR comprising reduced length HS4, and HS3, DNase I hypersensitive sites.

Embodiment 20: The recombinant lentiviral vector according to any one of embodiments 1-19, wherein said expression cassette comprises an α-globin regulatory element (HS40) (SEQ ID NO:10) between the LCR and the α-globin gene.

Embodiment 21: The recombinant lentiviral vector of embodiment 1, wherein said vector comprises the features of Vector I shown in FIG. 1, panel B.

Embodiment 22: The recombinant lentiviral vector of embodiment 21, wherein said vector comprises the features of Vector I shown in FIG. 4, panel A.

Embodiment 23: The recombinant lentiviral vector of embodiment 1, wherein said vector comprises the nucleic acid sequence of the expression cassette of vector I in SEQ ID NO:27.

Embodiment 24: The recombinant lentiviral vector of embodiment 23, wherein said vector comprises or consists of the nucleic acid sequence of SEQ ID NO:27.

Embodiment 25: The recombinant lentiviral vector of embodiment 1, wherein said vector comprises the features of Vector II shown in FIG. 1, panel B.

Embodiment 26: The recombinant lentiviral vector of embodiment 25, wherein said vector comprises the features of Vector II shown in FIG. 4, panel B.

Embodiment 27: The recombinant lentiviral vector of embodiment 1, wherein said vector comprises the nucleic acid sequence of the expression cassette of vector II in SEQ ID NO:28.

Embodiment 28: The recombinant lentiviral vector of embodiment 27, wherein said vector comprises or consists of the nucleic acid sequence of SEQ ID NO:28.

Embodiment 29: The recombinant lentiviral vector of embodiment 1, wherein said vector comprises the features of Vector III shown in FIG. 1, panel B.

Embodiment 30: The recombinant lentiviral vector of embodiment 29, wherein said vector comprises the features of Vector III shown in FIG. 4, panel C.

Embodiment 31: The recombinant lentiviral vector of embodiment 1, wherein said vector comprises the nucleic acid sequence of the expression cassette of vector III in SEQ ID NO:29.

Embodiment 32: The recombinant lentiviral vector of embodiment 31, wherein said vector comprises or consists of the nucleic acid sequence of SEQ ID NO:29.

Embodiment 33: The recombinant lentiviral vector of embodiment 1, wherein said vector comprises the features of Vector IV shown in FIG. 1, panel B.

Embodiment 34: The recombinant lentiviral vector of embodiment 33, wherein said vector comprises the features of Vector IV shown in FIG. 4, panel D.

Embodiment 35: The recombinant lentiviral vector of embodiment 1, wherein said vector comprises the nucleic acid sequence of the expression cassette of vector IV in SEQ ID NO:30.

Embodiment 36: The recombinant lentiviral vector of embodiment 35, wherein said vector comprises or consists of the nucleic acid sequence of SEQ ID NO:30.

Embodiment 37: The recombinant lentiviral vector of embodiment 1, wherein said vector comprises the features of Vector V shown in FIG. 1, panel B.

Embodiment 38: The recombinant lentiviral vector of embodiment 37, wherein said vector comprises the features of Vector V shown in FIG. 4, panel E.

Embodiment 39: The recombinant lentiviral vector of embodiment 1, wherein said vector comprises the nucleic acid sequence of the expression cassette of vector V in SEQ ID NO:31.

Embodiment 40: The recombinant lentiviral vector of embodiment 39, wherein said vector comprises or consists of the nucleic acid sequence of SEQ ID NO:31.

Embodiment 41: The recombinant lentiviral vector of embodiment 1, wherein said vector comprises the features of Vector VI shown in FIG. 1, panel B.

Embodiment 42: The recombinant lentiviral vector of embodiment 41, wherein said vector comprises the features of Vector VI shown in FIG. 4, panel F.

Embodiment 43: The recombinant lentiviral vector of embodiment 1, wherein said vector comprises the nucleic acid sequence of the expression cassette of vector VI in SEQ ID NO:32.

Embodiment 44: The recombinant lentiviral vector of embodiment 43, wherein said vector comprises or consists of the nucleic acid sequence of SEQ ID NO:32.

Embodiment 45: The recombinant lentiviral vector of embodiment 1, wherein said vector comprises the features of Vector VII shown in FIG. 1, panel B.

Embodiment 46: The recombinant lentiviral vector of embodiment 45, wherein said vector comprises the features of Vector VII shown in FIG. 4, panel G.

Embodiment 47: The recombinant lentiviral vector of embodiment 1, wherein said vector comprises the nucleic acid sequence of the expression cassette of vector VII in SEQ ID NO:33.

Embodiment 48: The recombinant lentiviral vector of embodiment 47, wherein said vector comprises or consists of the nucleic acid sequence of SEQ ID NO:33.

Embodiment 49: The recombinant lentiviral vector of embodiment 1, wherein said vector comprises the features of Vector VIII (A2-cDNA-UV) shown in FIG. 5.

Embodiment 50: The recombinant lentiviral vector of embodiment 1, wherein said vector comprises the nucleic acid sequence of the expression cassette of vector VIII (A2-cDNA-UV) in SEQ ID NO:38.

Embodiment 51: The recombinant lentiviral vector of embodiment 50, wherein said vector comprises or consists of the nucleic acid sequence of SEQ ID NO:38.

Embodiment 52: The recombinant lentiviral vector of embodiment 1, wherein said vector comprises the features of Vector IX (A2-ΔIVS2-UV) shown in FIG. 5.

Embodiment 53: The recombinant lentiviral vector of embodiment 1, wherein said vector comprises the nucleic acid sequence of the expression cassette of vector IX (A2-ΔIVS2-UV) in SEQ ID NO:40.

Embodiment 54: The recombinant lentiviral vector of embodiment 53, wherein said vector comprises or consists of the nucleic acid sequence of SEQ ID NO:40.

Embodiment 55: The recombinant lentiviral vector of embodiment 1, wherein said vector comprises the features of Vector X (A2-ΔIVS2-Globe) shown in FIG. 5.

Embodiment 56: The recombinant lentiviral vector of embodiment 1, wherein said vector comprises the nucleic acid sequence of the expression cassette of vector X (A2-ΔIVS2-Globe) in SEQ ID NO:39.

Embodiment 57: The recombinant lentiviral vector of embodiment 56, wherein said vector comprises or consists of the nucleic acid sequence of SEQ ID NO:39.

Embodiment 58: A host cell transduced with a vector according to any one of embodiments 1-57.

Embodiment 59: The host cell of embodiment 58, wherein the cell is a stem cell.

Embodiment 60: The host cell of embodiment 59, wherein said cell is a stem cell derived from bone marrow, and/or from umbilical cord blood, and/or from peripheral blood.

Embodiment 61: The host cell of embodiment 58, wherein the cell is a 293T cell.

Embodiment 62: The host cell of embodiment 58, wherein, wherein the cell is a human hematopoietic progenitor cell.

Embodiment 63: The host cell of embodiment 62, wherein the human hematopoietic progenitor cell is a CD34+ cell.

Embodiment 64: An expression cassette comprising a nucleic acid construct comprising:
an alpha-globin (α-globin) gene;
a 5′ UTR upstream from said α-globin gene and a 3′ UTR downstream from said α-globin gene where said 5′ and 3′ UTRs are from β-globin genes or from α-globin genes;
an α-globin promoter or a β globin promoter upstream from said 5′ UTR; and
a locus control region (LCR) upstream from said promoter, wherein said LCR is selected from the group consisting of core LCR UV (SEQ ID NO:11), GLOBE βLCR (A2-LCR) containing HS2 HS3 (SEQ ID NO:12), and GLOBE βLCR containing HS2 (SEQ ID NO:13), or an LCR having at least 95% or at least 98%, or at least 99% sequence identity to the foregoing LCRs.

Embodiment 65: The expression cassette of embodiment 64, wherein said expression cassette comprising a locus control region (LCR) selected from the group consisting of:
a core 3-LCR comprising reduced length HS4, HS3, and HS2 DNase I hypersensitive sites; or
a Globe 3-LCR comprising HS3 and HS2 DNase I hypersensitive sites; or
a Globe β-LCR comprising HS2 DNase I hypersensitive site and a β-LCR comprising reduced length HS4, and HS3, DNase I hypersensitive sites.

Embodiment 66: The expression cassette according to any one of embodiments 64-65, wherein said α-globin gene comprises a full-length α-globin gene including exon and introns.

Embodiment 67: The expression cassette according to any one of embodiments 64-65, wherein said α-globin gene comprises an HBA1 α-globin gene (SEQ ID NO:8).

Embodiment 68: The expression cassette according to any one of embodiments 64-65, wherein said α-globin gene comprises an HBA2 α-globin gene (SEQ ID NO:9).

Embodiment 69: The expression cassette according to any one of embodiments 64-65, wherein said α-globin gene comprises an alpha globin gene without intron 2.

Embodiment 70: The expression cassette according to any one of embodiments 64-65, wherein said α-globin gene comprises an alpha globin cDNA.

Embodiment 71: The expression cassette of embodiment 70, wherein said alpha globin cDNA is codon optimized.

Embodiment 72: The recombinant lentiviral vector according to any one of embodiments 66-71, wherein said alpha globin gene contains a tag.

Embodiment 73: The recombinant lentiviral vector of embodiment 72, wherein said tag comprises or consists of the sequence tgtgctgctctgcggcga (SEQ ID NO:18).

Embodiment 74: The expression cassette according to any one of embodiments 64-71, wherein said expression cassette comprises a 5′ UTR and a 3′UTR from a β-globin gene.

Embodiment 75: The expression cassette according to any one of embodiments 64-71, wherein said expression cassette comprises a 5′ UTR and a 3′UTR from an α-globin gene.

Embodiment 76: The expression cassette according to any one of embodiments 64-75, wherein said expression cassette comprises a full length β-globin promoter.

Embodiment 77: The expression cassette according to any one of embodiments 64-75, wherein said expression cassette comprises a shortened β-globin promoter (SEQ ID NO:1).

Embodiment 78: The expression cassette according to any one of embodiments 64-75, wherein said expression cassette comprises a full length α-globin promoter.

Embodiment 79: The expression cassette according to any one of embodiments 64-75, wherein said expression cassette comprises a shortened α-globin promoter (SEQ ID NO:14).

Embodiment 80: The expression cassette according to any one of embodiments 64-79, wherein said expression cassette comprises a core β-LCR comprising reduced length HS4 (SEQ ID NO:17), HS3 (SEQ ID NO:16), and HS2 (SEQ ID NO:15) DNase I hypersensitive sites.

Embodiment 81: The expression cassette according to any one of embodiments 64-79, wherein said expression cassette comprises a Globe β-LCR comprising HS3 and HS2 DNase I hypersensitive sites (SEQ ID NO:12).

Embodiment 82: The expression cassette according to any one of embodiments 64-79, wherein said expression cassette comprises a Globe β-LCR comprising HS2 DNase I hypersensitive site (SEQ ID NO:13) and a β-LCR comprising reduced length HS4, and HS3, DNase I hypersensitive sites.

Embodiment 83: The expression cassette according to any one of embodiments 64-82, wherein said expression cassette comprises an α-globin regulatory element (HS40) (SEQ ID NO:10) between the LCR and the α-globin gene.

Embodiment 84: The expression cassette of embodiment 64, wherein said expression cassette comprises or consists of the nucleic acid sequence of the expression cassette of vector I in SEQ ID NO:27.

Embodiment 85: The expression cassette of embodiment 64, wherein said expression cassette comprises or consists of the nucleic acid sequence of the expression cassette of vector II in SEQ ID NO:28.

Embodiment 86: The expression cassette of embodiment 64, wherein said expression cassette comprises or consists of the nucleic acid sequence of the expression cassette of vector III in SEQ ID NO:29.

Embodiment 87: The expression cassette of embodiment 64, wherein said expression cassette comprises or consists of the nucleic acid sequence of the expression cassette of vector IV in SEQ ID NO:30.

Embodiment 88: The expression cassette of embodiment 64, wherein said expression cassette comprises or consists of the nucleic acid sequence of expression cassette of vector V in SEQ ID NO:31.

Embodiment 89: The expression cassette of embodiment 64, wherein said expression cassette comprises or consists of the nucleic acid sequence of the expression cassette of vector VI in SEQ ID NO:32.

Embodiment 90: The expression cassette of embodiment 64, wherein said expression cassette comprises or consists of the nucleic acid sequence of the expression cassette of vector VII in SEQ ID NO:33.

Embodiment 91: The expression cassette of embodiment 64, wherein said expression cassette comprises or consists of the nucleic acid sequence of the expression cassette of vector VIII (A2-cDNA-UV) in SEQ ID NO:38.

Embodiment 92: The expression cassette of embodiment 64, wherein said expression cassette comprises or consists of the nucleic acid sequence of the expression cassette of vector IX (A2-ΔIVS2-UV) in SEQ ID NO:40.

Embodiment 93: The expression cassette of embodiment 64, wherein said expression cassette comprises or consists of the nucleic acid sequence of the expression cassette of vector X (A2-ΔIVS2-Globe) in SEQ ID NO:39.

Embodiment 94: A viral particle comprising the expression cassette recited in any one of embodiments 64-93.

Embodiment 95: The viral particle of embodiment 94, wherein said viral particle comprises a lentiviral particle.

Embodiment 96: A method of treating α-thalassemia in a subject, said method comprising:
transducing a stem cell and/or progenitor cell from said subject with a vector according to any one of embodiments 1-57; and
transplanting said transduced cell or cells derived therefrom into said subject where said cells or derivatives therefrom express an alpha globin.

Embodiment 97: The method of embodiment 96, wherein the cell is a stem cell.

Embodiment 98: The host cell of embodiment 96, wherein said cell is a stem cell derived from bone marrow.

Embodiment 99: The method of embodiment 96, wherein, wherein the cell is a human hematopoietic progenitor cell.

Embodiment 100: The method of embodiment 99, wherein the human hematopoietic progenitor cell is a CD34⁺ cell.

### Definitions

An “HS core sequence” as used herein refers to a reduced β-globin locus control region (LCR) hypersensitivity site (HS) sequence as defined herein (e.g., (HS2 (˜420 bp), HS3 (˜340 bp), and/or HS4 (˜410 bp)). Full-length HS sequences refers to LCR HS2, HS3, and HS4 as previously defined (e.g., HS2 (˜1.20 kb), HS3 (˜1.28 kb), and HS4 (˜1.1 kb)) (see, e.g., Forrester et al. (1986) Proc. Natl. Acad. Sci. USA, 83: 1359-1363).

“Recombinant” is used consistently with its usage in the art to refer to a nucleic acid sequence that comprises portions that do not naturally occur together as part of a single sequence or that have been rearranged relative to a naturally occurring sequence. A recombinant nucleic acid is created by a process that involves the hand of man and/or is generated from a nucleic acid that was created by hand of man (e.g., by one or more cycles of replication, amplification, transcription, etc.). A recombinant virus is one that comprises a recombinant nucleic acid. A recombinant cell is one that comprises a recombinant nucleic acid.

As used herein, the term “recombinant lentiviral vector” or “recombinant LV) refers to an artificially created polynucleotide vector assembled from an LV and a plurality of additional segments as a result of human intervention and manipulation.

By “globin nucleic acid molecule” is meant a nucleic acid molecule that encodes a globin polypeptide. In various embodiments the globin nucleic acid molecule may include regulatory sequences upstream and/or downstream of the coding sequence.

By “globin polypeptide” is meant a protein having at least 85%, or at least 90%, or at least 95%, or at least 98% amino acid sequence identity to a human alpha, beta or gamma globin.

The term “α-globin gene” (e.g., an HBA1 gene and/or an HBA2 gene) refers to a nucleotide sequence the expression of which leads to the expression of an α-globin protein. In certain embodiments α-globin gene may be a full length α-globin gene including introns as well as exons, or it may be an alpha globin gene excluding introns, or it may be a cDNA that encodes an alpha-globin. In certain embodiments the α-globin gene may be codon optimized. In certain embodiments the α-globin gene is effective to provide therapeutic benefits to an individual with a defective α-globin gene. The functional globin gene may encode a wild-type α-globin appropriate for a mammalian individual to be treated, or it may be a mutant form of α-globin, preferably one which provides for superior properties, for example superior oxygen transport properties.

By “an effective amount” is meant the amount of a required agent or composition comprising the agent to ameliorate or eliminate symptoms of a disease relative (e.g., alpha thalassemia) to an untreated patient. The effective amount of composition(s) used to practice the methods described herein for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

The term “sequence identity” refers to the extent to which two optimally aligned polynucleotide or polypeptide sequences are invariant throughout a window of alignment of components, e.g., nucleotides or amino acids. “Identity” can be readily calculated by known methods including, but not limited to, those described in: Computational Molecular Biology (Lesk, A. M., ed.) Oxford University Press, New York (1988); Biocomputing: Informatics and Genome Projects (Smith, D. W., ed.) Academic Press, New York (1993); Computer Analysis of Sequence Data, Part I (Griffin, A. M., and Griffin, H. G., eds.) Humana Press, New Jersey (1994); Sequence Analysis in Molecular Biology (von Heinje, G., ed.) Academic Press (1987); and Sequence Analysis Primer (Gribskov, M. and Devereux, J., eds.) Stockton Press, New York (1991); and the like.

An “identity fraction” for aligned segments of a test sequence and a reference sequence is the number of identical components which are shared by the two aligned sequences divided by the total number of components in the reference sequence segment, i.e., the entire reference sequence or a smaller defined part of the reference sequence. Percent sequence identity is typically represented as the identity fraction multiplied by 100. As used herein, the term “percent sequence identity” or “percent identity” refers to the percentage of identical nucleotides in a linear polynucleotide sequence of a reference (“query”) polynucleotide molecule (or its complementary strand) as compared to a test (“subject”) polynucleotide molecule (or its complementary strand) when the two sequences are optimally aligned (with appropriate nucleotide insertions, deletions, or gaps totaling less than 20 percent of the reference sequence over the window of comparison).

Optimal alignment of sequences for aligning a comparison window is well known to those skilled in the art and may be conducted by tools such as the local homology algorithm of Smith and Waterman, the homology alignment algorithm of Needleman and Wunsch, the search for similarity method of Pearson and Lipman, and optionally by computerized implementations of these algorithms such as GAP, BESTFIT, FASTA, and TFASTA available as part of the GCG® Wisconsin Package® (Accelrys Inc., Burlington, Mass.). The comparison of one or more polynucleotide sequences may be to a full-length polynucleotide sequence or a portion thereof, or to a longer polynucleotide sequence. In various embodiments “percent identity” may also be determined using BLASTX version 2.0 for translated nucleotide sequences and BLASTN version 2.0 for polynucleotide sequences.

The percent of sequence identity can be determined using the “Best Fit” or “Gap” program of the Sequence Analysis Software Package.TM. (Version 10; Genetics Computer Group, Inc., Madison, Wis.). “Gap” utilizes the algorithm of Needleman and Wunsch (1970) J. Mol. Biol. 48: 443-453, to find the alignment of two sequences that maximizes the number of matches and minimizes the number of gaps. “BestFit” performs an optimal alignment of the best segment of similarity between two sequences and inserts gaps to maximize the number of matches using the local homology algorithm of Smith and Waterman (1981) Adv. Appl. Math., 2: 482-489, Smith et al. (1983) Nucleic Acids Res. 11: 2205-2220. Useful methods for determining sequence identity are also disclosed in Guide to Huge Computers (Martin J. Bishop, ed., Academic Press, San Diego (1994)), and Carillo et al. (1988) Appl. Math, 48: 1073). Certain illustrative computer programs for determining sequence identity include, but are not limited to, the Basic Local Alignment Search Tool (BLAST) programs, that are publicly available from National Center Biotechnology Information (NCBI) at the National Library of Medicine, National Institute of Health, Bethesda, Md. 20894; see BLAST Manual, Altschul et al., NCBI, NLM, NIH; (Altschul et al., J. Mol. Biol. 215:403-410 (1990)); version 2.0 or higher of BLAST programs allows the introduction of gaps (deletions and insertions) into alignments; for peptide sequence, BLASTX can be used to determine sequence identity; and for polynucleotide sequence, BLASTN can be used to determine sequence identity.

### BRIEF DESCRIPTION OF THE DRAWINGS

FIG. 1, panels A-B, shows a schematic representation of (α-globin cassettes within lentiviral vectors. Panel A) Ultimate vector (UV) β-globin vector. Ultimate vector is the parental vector of α-globin lentiviral vectors. Grey boxes designate β-globin elements; β-LCR, containing core regions of DNase I hypersensitive site HS2, 3, and 4 (HS2, HS3, HS4); βp, representing short 265 bp β-globin promoter; Dark grey designate the β-globin gene (HBB) with its corresponding UTRs. Panel B) α-globin lentiviral vectors. Dark blue boxes designate HBA2 gene and lighter blue boxes represent HBA1 gene with their corresponding UTRs. To note, 5′ UTRs are identical in both HBA1 and HBA2 genes. Light blue box represents the 255 bp α-globin regulatory element core, denoted as HS240. αp denotes a 210 bp α-globin promoter core. In green boxes refer to element of LCR (2623 bp) from Globe vector.

FIG. 2, panels A-C, illustrates characterization of α-globin lentiviral vectors. Panel A) Globin lentiviral vector proviral length. The proviral length refers to the DNA integrated in the genome. Lenti-βAS3 and Globe-βAS3 LVs are clinical vectors for β-hemoglobinopathies. Ultimate vector is the parental vector of α-globin LVs, besides for LCR Globe and HS2 Globe vectors. Panel B) Lentiviral vector titer. Raw viruses were collected in biological duplicate and titer was measured in HT-29 cells in triplicate. Panel C) Alpha-globin expression per vector copy. Alpha-globin K/O HUDEP cells were transduced in triplicate with variable virus concentration yielding different VCN ranging from 0.45 to 3.5.

FIG. 3, panels A-D, illustrate hemoglobin analysis of α-globin lentiviral vectors done by HPLC. 1e6 HUDEP cells were collected on day 12 after transduction and differentiation for hemoglobin analysis. Peak at 12.4 min denoted with black arrow points to tetrameric adult hemoglobin (α₂β₂). Panel A) WT HUDEP cells serve as the positive control with a high peak at 12.4 min. Panel B) Alpha-globin knockout (α-globin K/O) HUDEP cells serve as the negative control with absence of peaks above background level illustrating absence of hemoglobin. Alpha-globin K/O HUDEP cells transduced with alpha2 globe vector (panel C) and LCR globe vectors (panel D) both display peak at 12.4 min representing successful hemoglobin production from α-globin lentiviral vector expression.

FIG. 4, panels A-G, illustrate show maps for vector I (panel A), vector II (panel B), vector III (panel C), vector IV (panel D), vector V (panel E), vector VI (panel F), and vector VII (panel G).

FIG. 5 shows a schematic representation of α-globin cassettes within lentiviral vectors with updated names and additional cassettes. Vector I beta globin 5′ and 3′ UTRs. Vector II HBA1 5′ and 3′ UTRs. Vectors III-X HBA2 5′ and 3′ UTRs

FIG. 6 schematically illustrates certain α-globin cassettes within lentiviral vectors used in various studies described herein.

FIG. 7 shows the raw titer of AGLV and expression of α/β-globin mRNA in a human erythroid cell line (HUDEP).

FIG. 8 illustrates the expression of an α-globin gene containing a Tag.

FIG. 9 illustrates the raw titer, VCN, and α/β mRNA ratio per vector copy number for parental Tag, an codon-optimized α-globin genes.

FIG. 10 illustrates hemoglobin production with tagged vectors.

FIG. 11 shows a preliminary assessment of the functionality of vectors described herein (Globe-bAS3, A2-Globe, A2-HS40-UV, A2-UV, A2-αₚ-UV, and A2-cDNA-UV). Studies were done in a human erythroid cell line enabled to be differentiated into red blood cells and engineered with homologous deletion of α-globin loci. Alpha globin lentiviral vectors enable production of adult hemoglobin (HbA). For the titer assay vectors were transduced into HT-29 cells. The cells were collected three days post transduction. As illustrated, alpha globin vectors yielded greater titer than GLOBE-bAS3.

FIG. 12 illustrates results of vector assessment, in particular with respect to in vitro erythroid Differentiation of healthy donor hematopoietic stem and progenitor cells.

FIG. 13 shows the evaluation of Alpha Thalassemia Major Hematopoietic Stem and Progenitor Cells.

FIG. 14 illustrates α-globin and β-globin expression in alpha thalassemia major cells.

FIG. 15 illustrates hemoglobin restoration in alpha thalassemia major hematopoietic stem and progenitor cells.

FIG. 16 schematically illustrates candidate vectors for in vivo studies.

FIG. 17 shows raw titer and vector copy number using a murine vector.

FIG. 18 shows the alpha/beta globin mRNA ratio and this ratio normalized to VCN for two murine vectors.

FIG. 19 shows the results of murine vector analysis in MEL cells.

### DETAILED DESCRIPTION

In various embodiments expression cassettes comprising an alpha-globin (α-globin gene) suitable for incorporation into gene therapy vectors, e.g., lentiviral vectors (LV) are provided herein. Additionally, methods of treating alpha Thalassemia (AT) using such vectors are provided.

We believe integration of functional copies of α-globin genes into hematopoietic stem cell genome by vector-mediated (e.g., LV-mediated transduction) can normalize the globin chain imbalance and restore hemoglobin function in the context of severe AT. This proposed stem cell/gene therapy approach highly resembles the therapy for β-thalassemia (BT) and sickle cell disease (SCD), and utilizes similar, but optimized vectors, harboring an α-globin gene cassette. Upon successful engraftment, the patient's α-globin gene-corrected hematopoietic system gives rise to red blood cells (RBCs) expressing α-globin, thus producing functional hemoglobin, supporting effective erythropoiesis, and ideally eliminating the need for blood transfusion.

Without being bound to a particular theory, it is believed this approach can provide a potential cure for patients with AT as a one-time autologous treatment for patients that currently require lifelong blood transfusions and chelation therapy. In this regard, it is believed that LV-mediated delivery of the α-globin gene to HSC from AT patients will increase α-globin expression to restore functional hemoglobin production. We have an extensive track record developing LVs for treatment of hemoglobinopathies. These vectors have not only been shown to restore sufficient β-globin levels to reverse the SCD phenotype, but also harbor erythroid-specific regulatory regions surrounding the transgene that have been demonstrated to restrict expression to the erythroid lineage, regardless of the genomic integration site in multipotent HSCs.

The goal for effective transduction of long-term engrafting stem cells is to have the maximal percentage of HSC transduced by the vector, but at the minimal vector copy per cell needed to effectively ameliorate disease—i.e., a broad distribution at relatively narrow range of vector copy number (VCN). Each lentiviral vector integrant has a finite risk of triggering an insertional event, albeit quite low. General regulatory expectations are that LV VCN will be limited to a target below a limit, such as 3-5. Thus, a vector desirably provides sufficient expression potency such that 3-5 copies/cell confer a 6-8 gm/dl increase in hemoglobin concentration to confer transfusion independence from the severe forms of AT.

### Design of α-Globin Cassettes

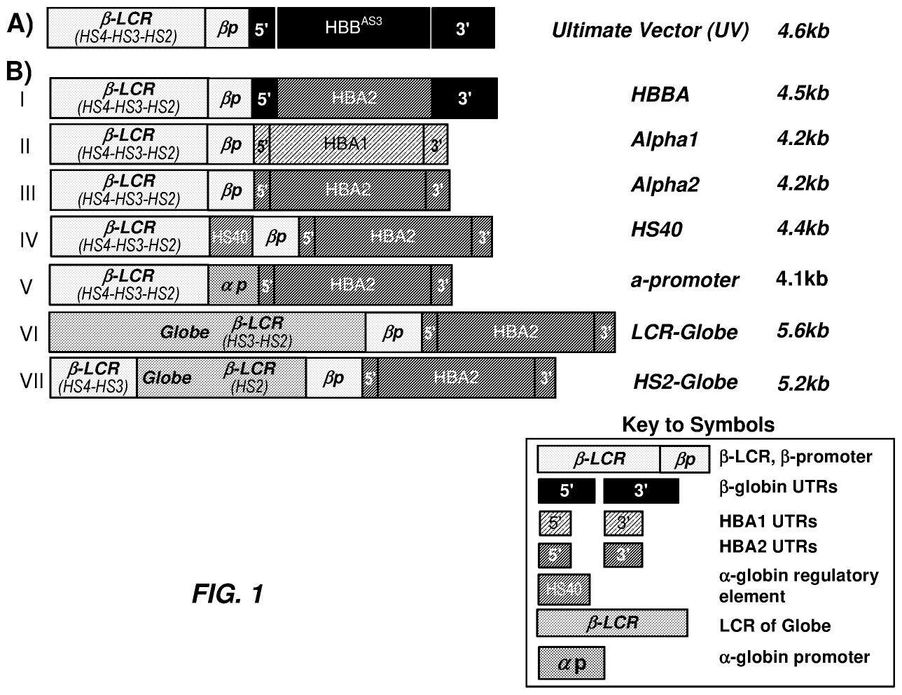

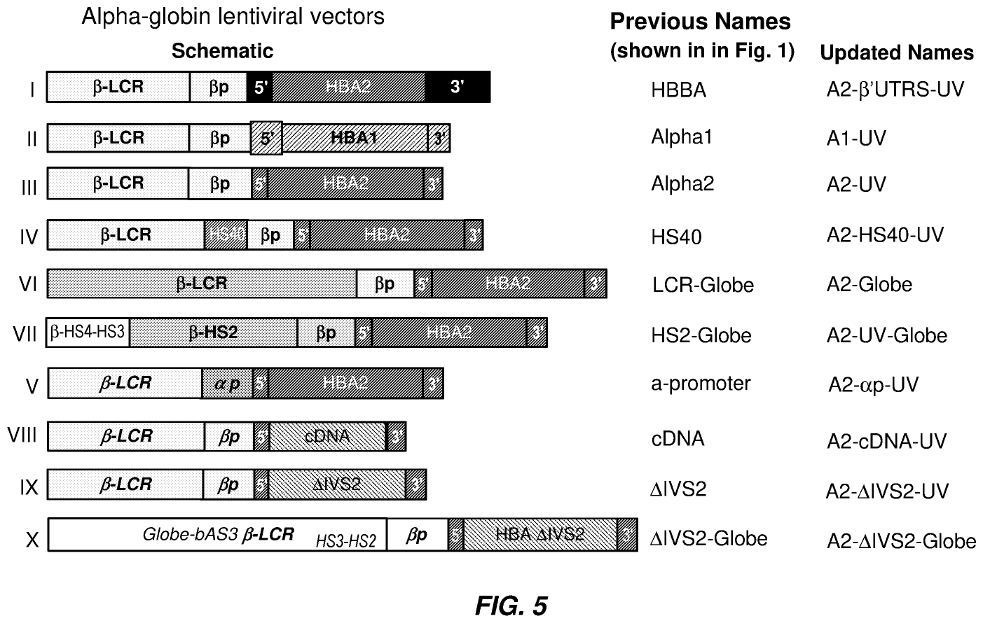

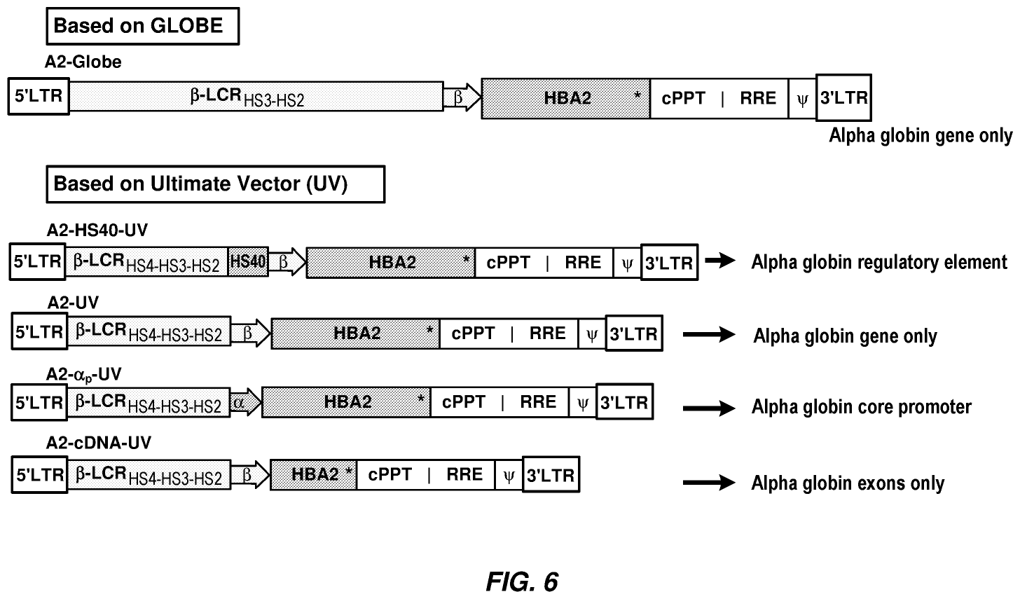

The LV designs commonly used to express β-globin for gene therapy of BTM or SCD are typically near the upper size limit for LV packaging (e.g., 9.0 kb), and thus suffer from low titers and poor CD34⁺ cell transduction. The use of transduction enhancers moderately improves transduction, but large amounts of vector preps are typically needed per patient dose. Therefore, we developed refined vectors of significantly improved titer and CD34⁺ cell transduction using ENCODE data to define the active core regions of the β-globin locus control region (LCR) DNAse hypersensitive sites (HS). The best of these newly developed vectors, named Ultimate Vector (UV), is 4.7 kb in proviral length and has a 5-10-fold higher titer than the full-length vectors and transduces human CD34⁺ cells 5-10 times more effectively at equivalent multiplicity of infection (MOI) due to a significantly higher percentage of complete full-length virion RNA genomes. The result is higher net amounts of β-globin expression (mRNA by RT-ddPCR and protein by HPLC) than from the larger vectors used at the same vector concentration and MOI following in vitro erythroid differentiation. Hence, we used this UV vector and replacing the β-globin gene cassette with different variations of α-globin gene cassettes (see, e.g., FIGS. 1, 5, and 6). The α-globin LVs (AGLV) are evaluated for their relative titer, CD34⁺ cell infectivity, and erythroid-specific expression for application to gene therapy of ATM. This approach has potential to treat and potentially cure AT by 1) increasing the amount of α-globin to produce functional hemoglobin, and 2) decreasing the formation of toxic HbH tetramers from free excess (3-globin chains, reducing cell damage and improving effective erythropoiesis.

To develop a gene addition stem cell therapy to treat AT in patient stem cells-derived RBCs, we first designed an alpha-globin lentiviral vector (AGLV) based on the UV backbone elements and incorporated various combinations of α-globin genes (HBA1 or HBA2) and regulatory elements. These AGLV retain the optimized β-globin LCR regulatory elements from UV and have replaced the β-globin coding sequences with the entire ˜700 bp human α-globin structural gene (exon and introns), with 5′ and 3′ UTRs coming from either the β-globin (FIG. 1, panel A) or α-globin genes (FIG. 1, panel B, and FIG. 5, vectors I, and II (SEQ ID NOs:27 and 28, respectively).

These AGLV retain the optimized β-globin LCR regulatory elements from UV, and have replaced the β^{AS3}-globin coding sequences with the entire ˜700 bp human α-globin structural gene (exon and introns), with 5′ and 3′ UTRs coming from either β-globin (FIG. 1, panel B, or FIG. 5 vector I) or α-globin genes (FIG. 1, panel B, or FIG. 5 vectors II, and III, SEQ ID NOs:28 and 29, respectively). HBA1 (SEQ ID NO:8) and HBA2 (SEQ ID NO:9) have identical exons and 5′UTR, but differ slightly in intron 2, and more extensively in their 3′UTRs. Another vector incorporates the HS40 position-dependent α-globin regulatory element (SEQ ID NO:10) adjacent to the β-globin LCR, both of which contain enhancer activities (FIG. 1, panel B, or FIG. 5 vector IV, SEQ ID NO:30). Regulation of the α-globin gene by β-globin regulatory elements has previously been demonstrated in transgenic mice, however, it is possible that interactions among additional α-globin elements may augment α-globin gene expression. Therefore, we designed a vector replacing the β-globin promoter with the α-globin promoter (FIG. 1, panel B, or FIG. 5 vector V, SEQ ID NO:31).

Additional larger vectors have been developed to enhance gene expression by replacing the core β-LCR of UV (1.1 kb) with the larger β-LCR from the GLOBE vector (2.6 kb) (Miccio et al. (2008) Proc. Natl. Acad. Sci. USA, 105: 10547-10552) which is currently being used in a clinical trial for β-thalassemia (FIG. 1, panel B, or FIG. 5 vector VI, SEQ ID NO:32) (Marktel, et al. (2019) Nat. Med. 25: 234-241). We have used the GLOBE backbone in our Lenti/GLOBE-Beta-AS3 vector in a trial for sickle cell disease (CIRM DR3-06945; NCT02247843). Incorporating a longer β-LCR region has been shown to increase β-globin expression, and thus is expected to enhance α-globin expression as well. To optimize aspects of higher titer and increased gene expression, an additional vector was created by coalescing the β-LCR from UV and GLOBE-βAS3 vectors. The large 1.4 kb HS2 element from GLOBE (the LCR element with highest enhancer-like activity) has replaced the 0.43 kb HS2 from UV, while retaining core regions of UV-HS3 and HS4 (FIG. 1, panel B, or FIG. 5, vector VII, SEQ ID NO:33). This newly formed β-LCR is 2.1 kb in size, compared to the parental β-LCR of 2.6 kb and 1.2 kb from GLOBE-bAS3 and UV vectors, respectively. We hypothesized that these last two vectors would yield higher gene expression/VCN compared to all previously described α-globin vectors, while retaining an adequate titer.

Additional expression cassettes/LV vectors schematically illustrated in FIG. 5 include A2-cDNA-UV which contains the alpha globin gene without its 2 introns (see, e.g., SEQ ID NO:38), A2-deltaIVS2-UVwhich contains the alpha globin gene without its second intron (removal of intron2) (see, e.g., SEQ ID NO:40), and A2-deltaIVS2-GLOBE which contains the alpha globin gene without its second intron (removal of intron2), with regulation by the larger LCR (see, e.g., SEQ ID NO:39).

The sequences of the various features comprising these vectors are shown in Table 1, and nucleotide sequences for vectors I-VII (see e.g., are shown in SEQ ID NOs:27-33, respectively. The nucleic acid sequences of vectors VIII, IX, and X (see, e.g., FIG. 5) are shown in SEQ ID NOs:38, 40, and 39, respectively.

TABLE 1
Illustrative features comprising vectors I-X.

| | | SEQ |
|---|---|---|
| | | ID |
| Feature | Nucleotide Sequence | NO |
| β-promoter | aagcaatagatggctctgccctgacttttatgcccagccctggctcctgccctccctgctcc | 1 |
| | tgggagtagattggccaaccctagggtgtggctccacagggtgaggtctaagtgatgaca | |
| | gccgtacctgtccttggctcttctggcactggcttaggagttggacttcaaaccctcagccc | |
| | tccctctaagatatatctcttggccccataccatcagtacaaattgctactaaaaacatcctcc | |
| | tttgcaagtgtatttac | |
| β-globin 5′UTR | tgtttgaggttgctagtgaacacagttgtgtcagaagcaaatgt | 2 |
| B-globin 3′UTR | catgaggacagctaaaacaataagtaatgtaaaatacagcatagcaaaactttaacctcca | 3 |
| | aatcaagcctctacttgaatccttttctgagggatgaataaggcataggcatcaggggctgt | |
| | tgccaatgtgcattagctgtttgcagcctcaccttctttcatggagtttaagatatagtgtatttt | |
| | cccaaggtttgaactagctcttcatttctttatgttttaaatgcactgacctcccacattccctttt | |
| | tagtaaaatattcagaaataatttaaatacatcattgcaatgaaaataaatgttttttattaggca | |
| | gaatccagatgctcaaggcccttcataatatcccccagtttagtagttggacttagggaaca | |
| | aaggaacctttaatagaaattggacagcaagaaagcgagc | |
| HBA1 5′UTR | GGTGGGTTCTCTCTGAGTCTGTGGGGACCAGAAGAGT | 4 |
| HBA1 3′UTR | TGCCGCCCACTCAGACTTTATTCAAAGACCACGGGGG | 5 |
| | TACGGGTGCAGGAAGGGGAGGAGGGGCTGGGGGGAG | |
| | GCCCAAGGGGCAAGAAGCATGGCCACCGAGGCTCCA | |
| | GC | |
| HBA2 5′UTR | GGTGGGTTCTCTCTGAGTCTGTGGGGACCAGAAGAGT | 6 |
| (note-5′ UTR | | |
| of HBA1 and | | |
| HBA2 is | | |
| identical) | | |
| HBA2 3′UTR | TGCTGCCCACTCAGACTTTATTCAAAGACCAGGAAGG | 7 |
| | GCCGGTGCAAGGAGGGGAGGAGGGCCCGTTGGGAGG | |
| | CCCAGCGGGCAGGAGGAACGGCTACCGAGGCTCCAG | |
| | C | |
| HBA1 | TTAACGGTATTTGGAGGTCAGCACGGTGCTCACAGAA | 8 |
| (showing | GCCAGGAACTTGTCCAGGGAGGCGTGCACCGCAGGGG | |
| changes in | TGAACTCGGCGGGGAGGTGGGCGGCCAGGGTCACCA | |
| intron 2) | GCAGGCAGTGGCTTAGGAGCTGTGCAGAGAAGAGGG | |
| Bold/underlined | TCAGTGgggcCGaggGCCCAGGCCCGCAGCCGCCGCCTG | |
| represent | CGCTACACCTCCCGCAACCCGCGTGATCCTCTGCCCTG | |
| sequence in | cGAGGAAGGCGCCATCTCGCCCCTCGACCCAGATCGC | |
| HBA1 not | TCCCGGCCCGCCGCTCACCTTGAAGTTGACCGGGTCC | |
| found in HBA2) | ACCCGAAGCTTGTGCGCGTGCAGGTCGCTCAGGGCGG | |
| | ACAGCGCGTTGGGCATGTCGTCCACGTGCGCCACGGC | |
| | GTTGGTCAGCGCGTCGGCCACCTTCTTGCCGTGGCCCT | |
| | TAACCTGGGCAGAGCCGTGGCTCAGGTCGAAGTGCGG | |
| | GAAGTAGGTCTTGGTGGTGGGGAAGGACAGGAACATC | |
| | CTGCGGGGAGAAGCAGAGTGAGGGGTGGGGTTTGGG | |
| | TCCGGGGCCAGGACGGTTGAGGGTGGCCTGTGGGTCC | |
| | GGGCGGGCGAGGAGCCCGGGTCGGAGCAGGGGAGGG | |
| | AGCCTCACCTCTCCAGGGCCTCCGCACCATACTCGCC | |
| | AGCGTGCGCGCCGACCTTACCCCAGGCGGCCTTGACG | |
| | TTGGTCTTGTCGGCAGGAGACAGCACCAT | |
| HBA2 | TTAACGGTATTTGGAGGTCAGCACGGTGCTCACAGAA | 9 |
| (showing | GCCAGGAACTTGTCCAGGGAGGCGTGCACCGCAGGGG | |
| changes in | TGAACTCGGCGGGGAGGTGGGCGGCCAGGGTCACCA | |
| intron 2) | GCAGGCAGTGGCTTAGGAGCTGTGCAGAGAAGAGGG | |
| (lower case “a” | TCAGTGCGGCCCAGGCCCGCAGCCGCCGCCTGCGCTA | |
| A -> C (HBA2 -> | CACCTCCCGCAACCCGCGTGATCCTCTGCCCTGaGAGG | |
| HBA1)) | AAGGCGCCATCTCGCCCCTCGACCCAGATCGCTCCCG | |
| Represents | GCCCGCCGCTCACCTTGAAGTTGACCGGGTCCACCCG | |
| nucleotide | AAGCTTGTGCGCGTGCAGGTCGCTCAGGGCGGACAGC | |
| change between | GCGTTGGGCATGTCGTCCACGTGCGCCACGGCGTTGG | |
| HBA1 and | TCAGCGCGTCGGCCACCTTCTTGCCGTGGCCCTTAACC | |
| HBA2 | TGGGCAGAGCCGTGGCTCAGGTCGAAGTGCGGGAAGT | |
| | AGGTCTTGGTGGTGGGGAAGGACAGGAACATCCTGCG | |
| | GGGAGAAGCAGAGTGAGGGGTGGGGTTTGGGTCCGG | |
| | GGCCAGGACGGTTGAGGGTGGCCTGTGGGTCCGGGCG | |
| | GGCGAGGAGCCCGGGTCGGAGCAGGGGAGGGAGCCT | |
| | CACCTCTCCAGGGCCTCCGCACCATACTCGCCAGCGT | |
| | GCGCGCCGACCTTACCCCAGGCGGCCTTGACGTTGGT | |
| | CTTGTCGGCAGGAGACAGCACCAT | |
| HS40 α-globin | CTCTCAGATAAACAGGAGGGGGAGGCAGCACCAGAG | 10 |
| regulatory | GTTGTAATCAGCAGTACCATTCACCCCTTTCCCTTGTC | |
| element | CCACCTCCACCCCCACAGGATGACTCAGCAGTCCTGT | |
| | TGGCCTCCAGAAGCACTGAGTCATGGTTGGCCCAGTT | |
| | ATCTGCTCCCTCAAGTGTTTTCACAGACCCCCACAGCT | |
| | GCATGCCTCCACCCTTGGCTTGGGCAGATGTGCTTGCC | |
| | TGGCTGACTGTGGTCCCTGATAGGTTCCAGAG | |
| CORE LCR | TACGTaTATGTGTATATATATATATATATTCAGGAAAT | 11 |
| (UV) | AATATATTCTAGAATATGTCACATTCTGTCTCAGGCAT | |
| This is made up | CCATTTTCTTTATGATGCCGTTTGAGGTGGAGTTTTAG | |
| of the core | TCAGGTGGTCAGCTTCTCCTTTTTTTTGCCATCTGCCCT | |
| regions of the | GTAAGCATCCTGCTGGGGACCCAGATAGGAGTCATCA | |
| β-LCR of Globe | CTCTAGGCTGAGAACATCTGGGCACACACCCTAAGCC | |
| | TCAGCATGACTCATCATGACTCAGCATTGCTGTGCTTG | |
| | AGCCAGAAGGTTTGCTTAGAAGGTTACACAGAACCAG | |
| | AAGGCGGGGGTGGGGCACTGACCCCGACAGGGGCCT | |
| | GGCCAGAACTGCTCATGCTTGGACTATGGGAGGTCAC | |
| | TAATGGAGACACACAGAAATGTAACAGGAACTAAGG | |
| | AAAAACTGAAGCTTtgggggtataggggagcagtcccatgtagtagtagaa | |
| | tgaaaaatgctgctatgctgtgcctcccccacctttcccatgtctgccctctactcatggtcta | |
| | tctctcctggctcctgggagtcatggactccacccagcaccaccaacctgacctaaccac | |
| | ctatctgagcctgccagcctataacccatctgggccctgatagctggtggccagccctga | |
| | ccccaccccaccctccctggaacctctgatagacacatctggcacaccagctcgcaaagt | |
| | caccgtgagggtcttgtgtttgctgagtcaaaattccttgaaatccaagtccttagagactcc | |
| | caggcttggattcaaagctcctgactttctgtctagtgtatgtgcagtgagccccttttcctct | |
| | aactgaaagaaggaaaaaaaaatggaacccaaaatattctacatagtttccatgtcacagc | |
| | cagggctgggcagtctcctgttatttcttttaaaataaatatatcatttaaatgcataaataagc | |
| | aaaccctgctcgggaatgggagggagagtctctggagtccaccccttctcggccctggct | |
| | ctgcagatagtgctatcaaagccctgacagagccctgcccattgctgggccttggagtga | |
| | gtcagcctagtagagaggcagggcaagccatctcatagctgctgagtgggagagagaa | |
| | aagggctcattgtctataaactcaggtcatggctattcttat | |
| GLOBE βLCR | ccttttgccacctagctgtccaggggtgccttaaaatggcaaacaaggtttgttttcttttcct | 12 |
| containing HS2 | gttttcatgccttcctcttccatatccttgtttcatattaatacatgtgtatagatcctaaaaatcta | |
| HS3 | tacacatgtattaataaagcctgattctgccgcttctaggtatagaggccacctgcaagata | |
| (sequence is | aatatttgattcacaataactaatcattctatggcaattgataacaacaaatatatatatatatat | |
| the same as A2- | atatacgtatatgtgtatatatatatatatatattcaggaaataatatattctagaatatgtcacat | |
| LCR. LCR in | tctgtctcaggcatccattttctttatgatgccgtttgaggtggagttttagtcaggtggtcagc | |
| A2-Globe | ttctccttttttttgccatctgccctgtaagcatcctgctggggacccagataggagtcatcac | |
| vectors or any | tctaggctgagaacatctgggcacacaccctaagcctcagcatgactcatcatgactcag | |
| of the -Globe | cattgctgtgcttgagccagaaggtttgcttagaaggttacacagaaccagaaggcgggg | |
| based vectors | gtggggcactgaccccgacaggggcctggccagaactgctcatgcttggactatggga | |
| are the same.) | ggtcactaatggagacacacagaaatgtaacaggaactaaggaaaaactgaagcttattt | |
| | aatcagagatgaggatgctggaagggatagagggagctgagcttgtaaaaagtatagta | |
| | atcattcagcaaatggttttgaagcacctgctggatgctaaacactattttcagtgcttgaatc | |
| | ataaataagaataaaacatgtatcttattccccacaagagtccaagtaaaaaataacagtta | |
| | attataatgtgctctgtcccccaggctggagtgcagtggcacgatctcagctcactgcaac | |
| | ctccgcctcccgggttcaagcaattctcctgcctcagccaccctaatagctgggattacag | |
| | gtgcacaccaccatgccaggctaatttttgtactttttgtagaggcagggtatcaccatgttg | |
| | tccaagatggtcttgaactcctgagctccaagcagtccacccacctcagcctcccaaagtg | |
| | ctgggattacaggtgtgagacaccatgcccagattttccatatttaatagaggtatttatggg | |
| | atgggggaaaagaatgtttctctcactgtggattattttagagagtggagaatggtcaagatt | |
| | tttttaaaaattaagaaaacataagttggaccttgagaaatgaaaatttatttttttgttggagg | |
| | atacccattctctatctcccatcagggcaagctgtaaggaactggctaagacacagtgaga | |
| | cagagtgacttagtcttagaggccccactggtacgacggtcaccaagctttcattaaaaaa | |
| | agtctaaccagctgcattcgactttgactgcagcagctggttagaaggttctactggagga | |
| | gggtcccagcccattgctaaattaacatcaggctctgagactggcagtatatctctaacagt | |
| | ggttgatgctatcttctggaacttgcctgctacattgagaccactgacccatacataggaag | |
| | cccatagctctgtcctgaactgttaggccactggtccagagagtgtgcatctcctttgatcct | |
| | cataataaccctatgagatagacacaattattactcttactttatagatgatgatcctgaaaac | |
| | ataggagtcaaggcacttgcccctagctgggggtataggggagcagtcccatgtagtagt | |
| | agaatgaaaaatgctgctatgctgtgcctcccccacctttcccatgtctgccctctactcatg | |
| | gtctatctctcctggctcctgggagtcatggactccacccagcaccaccaacctgacctaa | |
| | ccacctatctgagcctgccagcctataacccatctgggccctgatagctggtggccagcc | |
| | ctgaccccaccccaccctccctggaacctctgatagacacatctggcacaccagctcgca | |
| | aagtcaccgtgagggtcttgtgtttgctgagtcaaaattccttgaaatccaagtccttagaga | |
| | ctcctgctcccaaatttacagtcatagacttcttcatggctgtctcctttatccacagaatgatt | |
| | cctttgcttcattgccccatccatctgatcctcctcatcagtgcagcacagggcccatgagc | |
| | agtagctgcagagtctcacataggtctggcactgcctctgacatgtccgaccttaggcaaa | |
| | tgcttgactcttctgagctcagtcttgtcatggcaaaataaagataataatagtgtttttttatgg | |
| | agttagcgtgaggatggaaaacaatagcaaaattgattagactataaaaggtctcaacaaa | |
| | tagtagtagattttatcatccattaatccttccctctcctctcttactcatcccatcacgtatgcct | |
| | cttaattttcccttacctataataagagttattcctcttattatattcttcttatagtgattctggata | |
| | ttaaagtgggaatgaggggcaggccactaacgaagaagatgtttctcaaagaagc | |
| GLOBE βLCR | ccttttgccacctagctgtccaggggtgccttaaaatggcaaacaaggtttgttttcttttcct | 13 |
| containing HS2 | gttttcatgccttcctcttccatatccttgtttcatattaatacatgtgtatagatcctaaaaatcta | |
| | tacacatgtattaataaagcctgattctgccgcttctaggtatagaggccacctgcaagata | |
| | aatatttgattcacaataactaatcattctatggcaattgataacaacaaatatatatatatatat | |
| | atatacgtatatgtgtatatatatatatatatattcaggaaataatatattctagaatatgtcacat | |
| | tctgtctcaggcatccattttctttatgatgccgtttgaggtggagttttagtcaggtggtcagc | |
| | ttctccttttttttgccatctgccctgtaagcatcctgctggggacccagataggagtcatcac | |
| | tctaggctgagaacatctgggcacacaccctaagcctcagcatgactcatcatgactcag | |
| | cattgctgtgcttgagccagaaggtttgcttagaaggttacacagaaccagaaggcgggg | |
| | gtggggcactgaccccgacaggggcctggccagaactgctcatgcttggactatggga | |
| | ggtcactaalggagacacacagaaatgtaacaggaactaaggaaaaactgaagcttattt | |
| | aatcagagatgaggatgctggaagggatagagggagctgagcttgtaaaaagtatagta | |
| | alcaticagcaaatgglittgaagcaccigciggatgctaaacactattttcagigcttgaatc | |
| | ataaataagaataaaacatgtatcttattccccacaagagtccaagtaaaaaataacagtta | |
| | attataatgtgctctgtcccccaggctggagtgcagtggcacgatctcagctcactgcaac | |
| | ctccgcctccegggttcaagcaattctcctgcctcagccaccctaatagctgggattacag | |
| | gtgcacaccaccatgccaggctaatttttgtactttttgtagaggcagggtatcaccatgttg | |
| | tccaagatggtcttgaactcctgagctccaagcagtccacccacctcagcctcccaaagtg | |
| | ctgggattacaggtgtgagacaccatgcccagattttccatatttaatagaggtatttatggg | |
| | atgggggaaaagaatgtttctctcactgtggattattttagagagtggagaatggtcaagatt | |
| | tttttaaaaattaagaaaacataagttggaccttgagaaatgaaaatttatttttttgttggagg | |
| | atacccattctctatctcccatcagggcaagctgtaaggaactggctaagacacagtgaga | |
| | cagagtgacttagtcttagaggccccactggtacgacggtcacctgggggtatagggga | |
| | gcagtcccatgtagtagtagaatgaaaaatgctgctatgctgtgcctcccccacctttccca | |
| | tgtctgccctctactcatggtctatctctcctggctcctgggagtcatggactccacccagca | |
| | ccaccaacctgacctaaccacctatctgagcctgccagcctataacccatctgggccctg | |
| | atagctggtggccagccctgaccccaccccaccctccctggaacctctgatagacacatc | |
| | lggcacaccagctcgcaaagicaccgigagggicligigitigcigagtcaaaattccttga | |
| | aatccaagtccttagagactcccaggcttggattcaaagctcctgactttctgtctagtgtat | |
| | gtgcagtgagccccttttcctctaactgaaagaaggaaaaaaaaatggaacccaaaatatt | |
| | ctacatagtttccatgtcacagccagggctgggcagtctcctgttatttcttttaaaataaatat | |
| | atcatttaaatgcataaataagcaaaccctgctcgggaatgggagggagagtctctggagt | |
| | ccaccccttctcggccctggctctgcagatagtgctatcaaagccctgacagagccctgc | |
| | ccattgctgggccttggagtgagtcagcctagtagagaggcagggcaagccatctcata | |
| | gctgctgagtgggagagagaaaagggctcattgtctataaactcaggtcatggctattctta | |
| | t | |
| Core α-globin | GCCGGCCCGCGAGCGCGCCAGGGTTTATGCTTGGGGC | 14 |
| promoter | GCGGGGGCACGCCCGGCCGGGCGGCGCTCATTGGCTG | |
| 3′ to 5′ | GCGCGGAGCCCGGGGCGCGGCCTGGACCGCAGGGGA | |
| | GTCCCGGGCGGGGCCTGCGCGGGGCCGGGCGGCGGG | |
| | CTCGGCCTCCCCTGGGGGTGCACGCGGGGCGGGGGCC | |
| | AGGACGTCTCCACCCTCCACCCGCCAC | |
| HS2 (EC2) | TACGTaTATGTGTATATATATATATATATTCAGGAAAT | 15 |
| | AATATATTCTAGAATATGTCACATTCTGTCTCAGGCAT | |
| | CCATTTTCTTTATGATGCCGTTTGAGGTGGAGTTTTAG | |
| | TCAGGTGGTCAGCTTCTCCTTTTTTTTGCCATCTGCCCT | |
| | GTAAGCATCCTGCTGGGGACCCAGATAGGAGTCATCA | |
| | CTCTAGGCTGAGAACATCTGGGCACACACCCTAAGCC | |
| | TCAGCATGACTCATCATGACTCAGCATTGCTGTGCTTG | |
| | AGCCAGAAGGTTTGCTTAGAAGGTTACACAGAACCAG | |
| | AAGGCGGGGGTGGGGCACTGACCCCGACAGGGGCCT | |
| | GGCCAGAACTGCTCATGCTTGGACTATGGGAGGTCAC | |
| | TAATGGAGACACACAGAAATGTAACAGGAACTAAGG | |
| | AAAAACTGAAGCTT | |
| HS3 (EC3) | tgggggtataggggagcagtcccatgtagtagtagaatgaaaaatgctgctatgctgtgc | 16 |
| | ctcccccacctttcccatgtctgccctctactcatggtctatctctcctggctcctgggagtca | |
| | tggactccacccagcaccaccaacctgacctaaccacctatctgagcctgccagcctata | |
| | acccatctgggccctgatagctggtggccagccctgaccccaccccaccctccctggaa | |
| | cctctgatagacacatctggcacaccagctcgcaaagtcaccgtgagggtcttgtgtttgct | |
| | gagtcaaaattccutgaaatccaagtccttagagactcc | |
| HS4 (EC4) | caggcttggattcaaagctcctgactttctgtctagtgtatgtgcagtgagccccttttcctct | 17 |
| | aactgaaagaaggaaaaaaaaatggaacccaaaatattctacatagtttccatgtcacagc | |
| | cagggctgggcagtctcctgttatttcttttaaaataaatatatcatttaaatgcataaataagc | |
| | aaaccctgctcgggaatgggagggagagtctctggagtccaccccttctcggccctggct | |
| | ctgcagatagtgctatcaaagccctgacagagccctgcccattgctgggccttggagtga | |
| | gtcagcctagtagagaggcagggcaagccatctcatagctgctgagtgggagagagaa | |
| | aagggctcattgtctataaactcaggtcatggctattcttat | |
| Tag | tgtgctgctctgcggcga | 18 |
| α-globin gene | TGCTGCCCACTCAGACTTTATTCAAAGACCAGGAAGG | 19 |
| with Tag (3′ to | GCCGGTGCAAGGAGGGGAGGAGGGCCCGTTGGGAGG | |
| 5′) | CCCAGCGGGCAGGAGGAACGGCTACCGAGGCTCCAG | |
| | CtgtgctgctctgcggcgaTTAACGGTATTTGGAGGTCAGCACG | |
| bold/lower- | GTGCTCACAGAAGCCAGGAACTTGTCCAGGGAGGCGT | |
| case = tag | GCACCGCAGGGGTGAACTCGGCGGGGAGGTGGGCGG | |
| | CCAGGGTCACCAGCAGGCAGTGGCTTAGGAGCTGTGC | |
| | AGAGAAGAGGGTCAGTGCGGCCCAGGCCCGCAGCCG | |
| | CCGCCTGCGCTACACCTCCCGCAACCCGCGTGATCCTC | |
| | TGCCCTGAGAGGAAGGCGCCATCTCGCCCCTCGACCC | |
| | AGATCGCTCCCGGCCCGCCGCTCACCTTGAAGTTGAC | |
| | CGGGTCCACCCGAAGCTTGTGCGCGTGCAGGTCGCTC | |
| | AGGGCGGACAGCGCGTTGGGCATGTCGTCCACGTGCG | |
| | CCACGGCGTTGGTCAGCGCGTCGGCCACCTTCTTGCCG | |
| | TGGCCCTTAACCTGGGCAGAGCCGTGGCTCAGGTCGA | |
| | AGTGCGGGAAGTAGGTCTTGGTGGTGGGGAAGGACA | |
| | GGAACATCCTGCGGGGAGAAGCAGAGTGAGGGGTGG | |
| | GGTTTGGGTCCGGGGCCAGGACGGTTGAGGGTGGCCT | |
| | GTGGGTCCGGGCGGGCGAGGAGCCCGGGTCGGAGCA | |
| | GGGGAGGGAGCCTCACCTCTCCAGGGCCTCCGCACCA | |
| | TACTCGCCAGCGTGCGCGCCGACCTTACCCCAGGCGG | |
| | CCTTGACGTTGGTCTTGTCGGCAGGAGACAGCACCAT | |
| | GGTGGGTTCTCTCTGAGTCTGTGGGGACCAGAAGAGT | |
| cDNA with | TGCTGCCCACTCAGACTTTATTCAAAGACCAGGAAGG | 20 |
| TAG (3′ TO 5′ | GCCGGTGCAAGGAGGGGAGGAGGGCCCGTTGGGAGG | |
| UTR) | CCCAGCGGGCAGGAGGAACGGCTACCGAGGCTCCAG | |
| bold/lower- | CtgtgctgctctgcggcgaTTAACGGTATTTGGAGGTCAGCACG | |
| case = tag | GTGCTCACAGAAGCCAGGAACTTGTCCAGGGAGGCGT | |
| | GCACCGCAGGGGTGAACTCGGCGGGGAGGTGGGCGG | |
| | CCAGGGTCACCAGCAGGCAGTGGCTTAGGAGCTTGAA | |
| | GTTGACCGGGTCCACCCGAAGCTTGTGCGCGTGCAGG | |
| | TCGCTCAGGGCGGACAGCGCGTTGGGCATGTCGTCCA | |
| | CGTGCGCCACGGCGTTGGTCAGCGCGTCGGCCACCTT | |
| | CTTGCCGTGGCCCTTAACCTGGGCAGAGCCGTGGCTC | |
| | AGGTCGAAGTGCGGGAAGTAGGTCTTGGTGGTGGGGA | |
| | AGGACAGGAACATCCTCTCCAGGGCCTCCGCACCATA | |
| | CTCGCCAGCGTGCGCGCCGACCTTACCCCAGGCGGCC | |
| | TTGACGTTGGTCTTGTCGGCAGGAGACAGCACCATGG | |
| | TGGGTTCTCTCTGAGTCTGTGGGGACCAGAAGAGT | |
| Hba-a1 gene | TCAAAGACCAAGAGGTACAGGTGCAAGGGAGAGAAG | 21 |
| with TAG (3′ | AAGGGCATGGCCAGAAGGCAAGCCCCGCAGAAGGCA | |
| TO 5′ UTR) | GCtgtgctgctctgcggcgaTTAACGGTACTTGGAGGTCAGCAC | |
| bold/lower- | GGTGCTCACAGAGGCAAGGAATTTGTCCAGAGAGGCA | |
| case = tag | TGCACCGCGGGGGTGAAATCGGCAGGGTGGTGGCTAG | |
| Lower | CCAAGGTCACCAGCAGGCAGTGGCTCAGGAGctgcggaga | |
| case = introns | caaagtggacaccctgatgcctctgctccccttcctgggaccactatgttccctgccttggg | |
| | cacgaggacccccaagcccttcctaggggtcccagatgccgcctgccaggtcccagcg | |
| | catacCTTGAAGTTGACGGGATCCACACGCAGCTTGTGG | |
| | GCATGCAGGTCGCTCAGAGCAGACAGGGCACCGGGC | |
| | AGGTCATCGAGGTGGCCTGCAGCATTGGCCAGAGCAT | |
| | CGGCGACCTTCTTGCCGTGACCCTTGACCTGGGCAGA | |
| | GCCGTGGCTTACATCAAAGTGAGGGAAGTAGGTCTTG | |
| | GTGGTGGGGAAGCTAGCAAACATCctgggagaaggagagaaga | |
| | gaagttgaggtcacagaaaagcatagttagaagctgcccactgagcgagtgccaggtcc | |
| | atattggatcctgtgatccttacagatcaaggtcCtgttctcacCTTTCCAGGGCT | |
| | TCAGCTCCATATTCAGCACCATGGCCACCAATCTTCCC | |
| | CCAGGCAGCCTTGATGTTGCTTTTGTCTTCCCCAGAGA | |
| | GCACCATGGTTTCTTCCTGAGTCTGTCAGAATCAGAAG | |
| | TGTCTTGGACCTGCAGCAAGTAGCACTTATATGCCCTC | |
| | CAAGGGCGTGTCCTCCAGGCAGGGTGGACACGCCCTT | |
| | GGAGCAGTTCTCATTGGCTGGTTTGTGCTTGCTCTACC | |
| | CAGTAAAACTTGTTAGGGCCCCTGAGATTCTGGGGCT | |
| | GGGTTGTGCTTCCCC | |
| cPPT | aaaagaaaagggggg | 22 |
| RRE | agtagcacccaccaaggcaaagagaagagtggtgcagagagaaaaaagaGCAGT | 23 |
| | GGGAATaggagctttgttccttgggttcttgggagcagcaggaagcactatgggcgc | |
| | agcgtcaatgacgctgacggtacaggccagacaattattgtctggtatagtgcagcagc | |
| | agaacaatttgctgagggctattgaggcgcaacagcatctgttgcaactcacagtctgg | |
| | ggcatcaagcagctccaggcaagaatcctggctgtggaaa | |
| Psi | tcgacgcaggactcggcttgctgaagcgcgcacggcaagaggcgaggggcggcgact | 24 |
| | ggtgagtacgccaaaaattttgactagcggaggctagaaggagagagatgggtgcga | |
| | gagcgtcagtattaagcgggggag | |
| 5′ LTR | GGGTCTCTCTGGTTAGACCAGATCTGAGCCTGGGAGC | 25 |
| | TCTCTGGCTAACTAGGGAACCCACTGCTTAAGCCTCA | |
| | ATAAAGCTTGCCTTGAGTGCTTCAAGTAGTGTGTGCCC | |
| | GTCTGTTGTGTGACTCTGGTAACTAGAGATCCCTCAGA | |
| | CCCTTTTAGTCAGTGTGGAAAATCTCTAGC | |
| 3′ LTR | tggaagggctaattcactcccaacgaagacaagatctgctttttgcttgtactGGGTCT | 26 |
| | CTCTGGTTAGACCAGATCTGAGCCTGGGAGCTCTCTG | |
| | GCTAACTAGGGAACCCACTGCTTAAGCCTCAATAAAG | |
| | CTTGCCTTGAGTGCTT | |

Note:
The LCR in the A2-Globe vectors or any of the -Globe based vectors are the same.

In view of the foregoing, in various embodiments an expression cassette for expression of an α-globin gene is provided where the expression cassette is designed to be contained within a vector, e.g., a lentiviral vector. In certain embodiments the expression cassette comprises a nucleic acid construct comprising an alpha-globin (α-globin) gene; a 5′ UTR upstream from said α-globin gene and a 3′ UTR downstream from said α-globin gene where said 5′ and 3′ UTRs are from β-globin genes or from α-globin genes; an α-globin promoter or a β globin promoter upstream from said 5′ UTR; and a locus control region (LCR) upstream from said promoter, wherein said LCR is selected from the group consisting of core LCR UV (SEQ ID NO: 11), GLOBE βLCR containing HS2 HS3 (same as A2-LCR) (SEQ ID NO: 12), and GLOBE βLCR containing HS2 (SEQ ID NO: 13), or an LCR having at least 95% or at least 98%, or at least 99% sequence identity to the foregoing LCRs. In certain embodiments the expression cassette comprises a nucleic acid construct comprising an alpha-globin (α-globin) gene; a 5′ UTR upstream from said α-globin gene and a 3′ UTR downstream from said α-globin gene where said 5′ and 5′ UTRs are from β-globin genes or from α-globin genes; an α-globin promoter or a β globin promoter upstream from said 5′ UTR; and a locus control region (LCR) comprising: a core β-LCR comprising reduced length HS4, HS3, and HS2 DNase I hypersensitive sites; or a Globe β-LCR comprising HS3 and HS2 DNase I hypersensitive sites; or a Globe β-LCR comprising HS2 DNase I hypersensitive site and a β-LCR comprising reduced length HS4, and HS3, DNase I hypersensitive sites. In certain embodiments the α-globin gene comprises a full-length α-globin gene including exon and introns. In certain embodiments the α-globin gene comprises an HBA1 α-globin gene (SEQ ID NO:8) or an HBA2 α-globin gene (SEQ ID NO:9). In certain embodiments the expression cassette comprises a 5′ UTR and a 3′UTR from a β-globin gene or a 5′ UTR and a 3′UTR from an α-globin gene. In certain embodiments the expression cassette comprises a full length β-globin promoter. In certain embodiments the expression cassette comprises a shortened β-globin promoter (SEQ ID NO:1). In certain embodiments the expression cassette comprises a full length β-globin promoter. In certain embodiments the expression cassette comprises a shortened α-globin promoter (SEQ ID NO:14). In certain embodiments the expression cassette comprises a core 3-LCR comprising reduced length HS4 (SEQ ID NO:17), HS3 (SEQ ID NO:16), and HS2 (SEQ ID NO:15) DNase I hypersensitive sites. In certain embodiments the expression cassette comprises a Globe β-LCR comprising HS3 and HS2 DNase I hypersensitive sites (SEQ ID NO:12). In certain embodiments the expression cassette comprises a Globe β-LCR comprising HS2 DNase I hypersensitive site (SEQ ID NO:13) and a β-LCR comprising reduced length HS4, and HS3, DNase I hypersensitive sites. In certain embodiments the expression cassette comprises an α-globin regulatory element (HS40) (SEQ ID NO:10) between the LCR and the α-globin gene.

In certain embodiments the expression cassette comprises the features of the expression cassette of Vector I shown in FIG. 1, panel B. In certain embodiments the expression cassette comprises the nucleic acid sequence of the expression cassette in SEQ ID NO:27. In certain embodiments the expression cassette comprises the features of the expression cassette of Vector II shown in FIG. 1, panel B. In certain embodiments the expression cassette comprises the nucleic acid sequence of the expression cassette in SEQ ID NO:28. In certain embodiments the expression cassette comprises the features of the expression cassette of Vector III shown in FIG. 1, panel B. In certain embodiments the expression cassette comprises the nucleic acid sequence of the expression cassette in SEQ ID NO:29. In certain embodiments the expression cassette comprises the features of the expression cassette of Vector IV shown in FIG. 1, panel B. In certain embodiments the expression cassette comprises the nucleic acid sequence of the expression cassette in SEQ ID NO:30. In certain embodiments the expression cassette comprises the features of the expression cassette of Vector V shown in FIG. 1, panel B. In certain embodiments the expression cassette comprises the nucleic acid sequence of the expression cassette in SEQ ID NO:31. In certain embodiments the expression cassette comprises the features of the expression cassette of Vector VI shown in FIG. 1, panel B. In certain embodiments the expression cassette comprises the nucleic acid sequence of the expression cassette in SEQ ID NO:32. In certain embodiments the expression cassette comprises the features of the expression cassette of Vector VII shown in FIG. 1, panel B. In certain embodiments the expression cassette comprises the nucleic acid sequence of the expression cassette in SEQ ID NO:33.

Also provided are vectors (e.g., lentiviral vectors) comprises the expression cassette(s) described herein. Accordingly, in certain embodiments, a recombinant lentiviral vector (LV) is provided where the vector comprise an expression cassette as described herein. In certain embodiments the vector comprises the nucleic acid sequence of the expression cassette of vector I in SEQ ID NO:27. In certain embodiments the vector comprises the features of Vector I shown in FIG. 1, panel B. In certain embodiments the vector comprises the nucleic acid sequence of SEQ ID NO:27. In certain embodiments the vector comprises the nucleic acid sequence of the expression cassette of vector II in SEQ ID NO:28. In certain embodiments the vector comprises the features of Vector II shown in FIG. 1, panel B. In certain embodiments the vector comprises the nucleic acid sequence of SEQ ID NO:28. In certain embodiments the vector comprises the nucleic acid sequence of the expression cassette of vector III in SEQ ID NO:29. In certain embodiments the vector comprises the features of Vector III shown in FIG. 1, panel B. In certain embodiments the vector comprises the nucleic acid sequence of SEQ ID NO:29. In certain embodiments the vector comprises the nucleic acid sequence of the expression cassette of vector IV in SEQ ID NO:30. In certain embodiments the vector comprises the features of Vector IV shown in FIG. 1, panel B. In certain embodiments the vector comprises the nucleic acid sequence of SEQ ID NO:30. In certain embodiments the vector comprises the nucleic acid sequence of the expression cassette of vector V in SEQ ID NO:31. In certain embodiments the vector comprises the features of Vector V shown in FIG. 1, panel B. In certain embodiments the vector comprises the nucleic acid sequence of SEQ ID NO:31. In certain embodiments the vector comprises the nucleic acid sequence of the expression cassette of vector VI in SEQ ID NO:32. In certain embodiments the vector comprises the features of Vector VI shown in FIG. 1, panel B. In certain embodiments the vector comprises the nucleic acid sequence of SEQ ID NO:32. In certain embodiments the vector comprises the nucleic acid sequence of the expression cassette of vector VII in SEQ ID NO:33. In certain embodiments the vector comprises the features of Vector VII shown in FIG. 1, panel B. In certain embodiments the vector comprises the nucleic acid sequence of SEQ ID NO:33.

Additional, in certain embodiments, host cells transduced with the vectors described herein are provided. In certain embodiments the host cell is a stem cell. In certain embodiments the host cell is a stem cell derived from bone marrow, and/or from umbilical cord blood, and/or from peripheral blood. In certain embodiments the cell is a 293T cell. In certain embodiments the cell is a human hematopoietic progenitor cell. In certain embodiments the cell is a CD34+ human hematopoietic progenitor cell is a CD34+ cell.

Also provided are packaging cells transduced with a vector described herein.

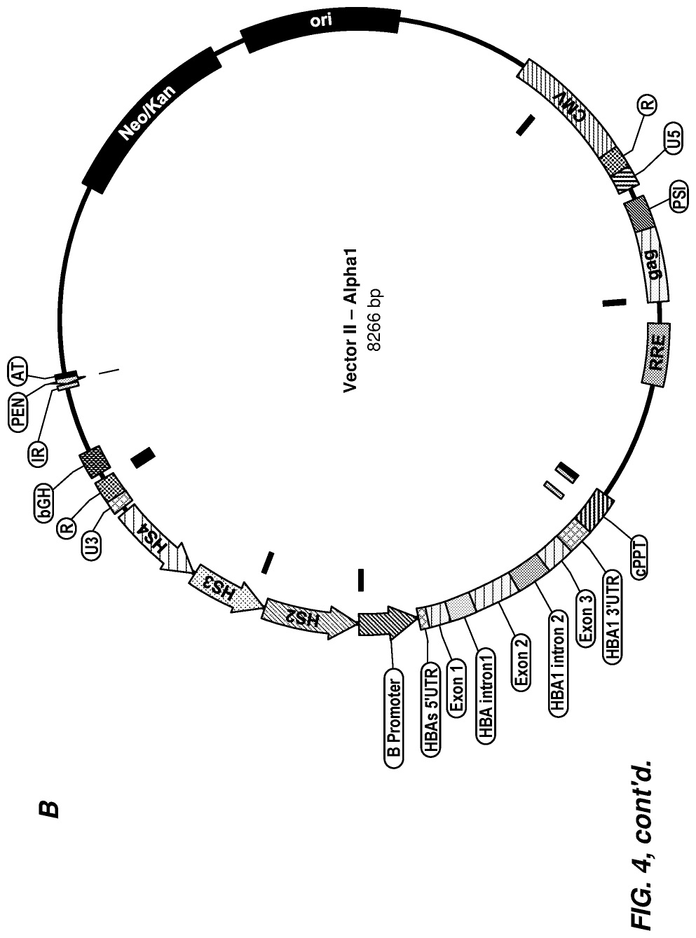

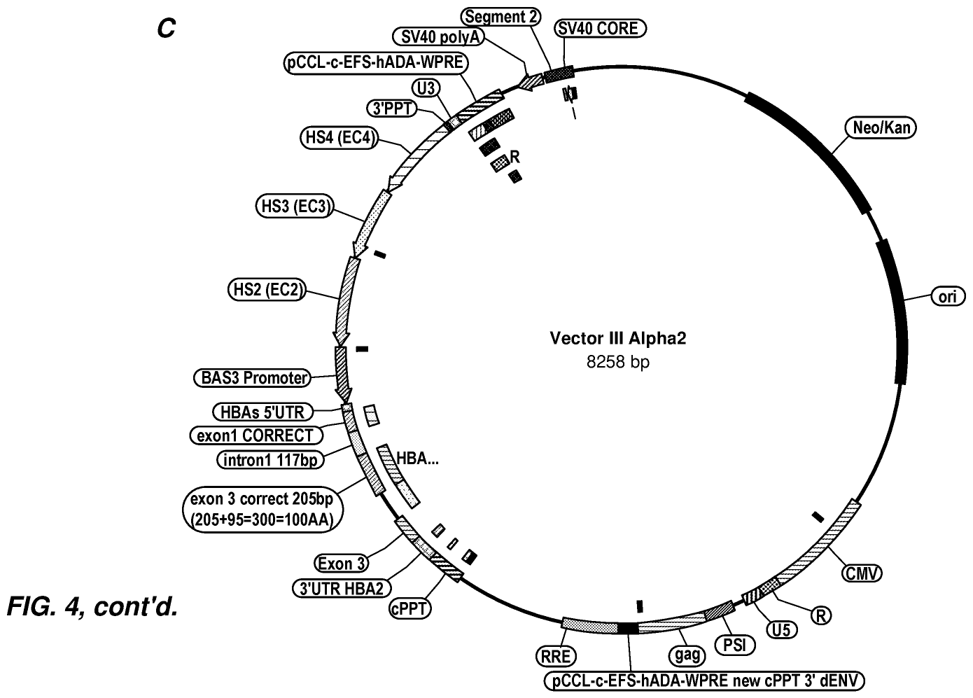

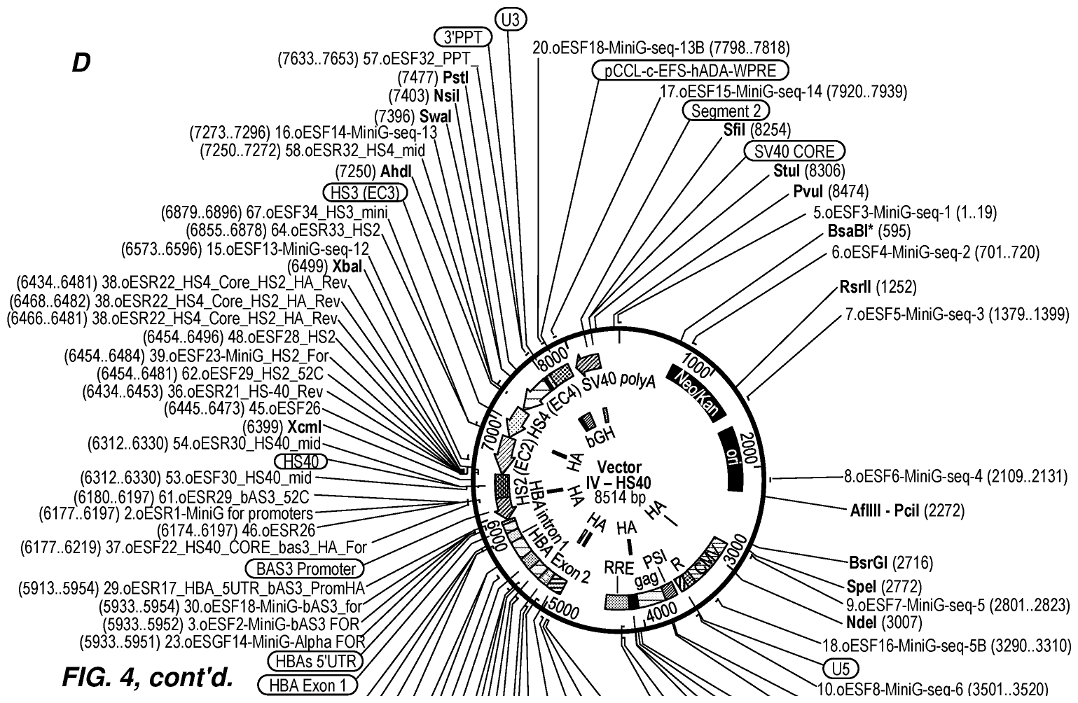

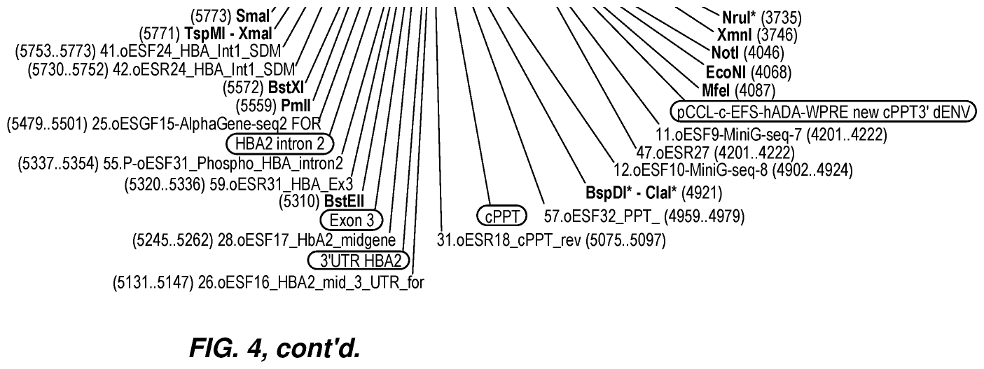

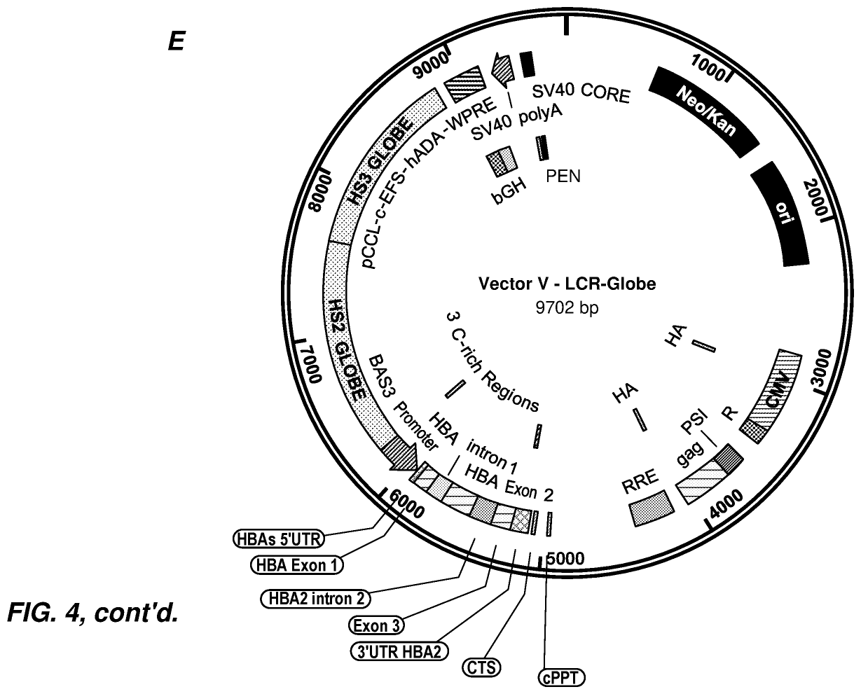

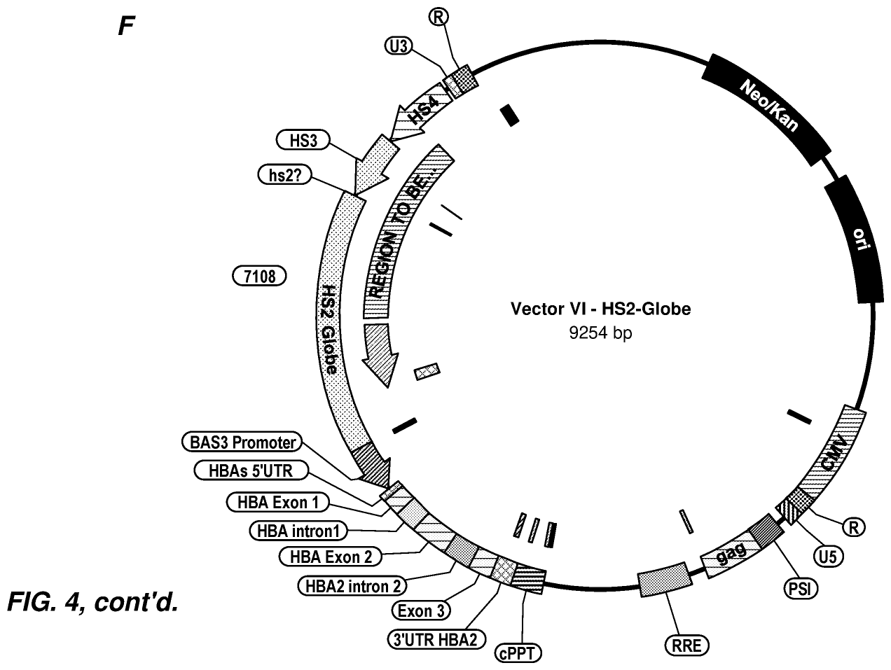

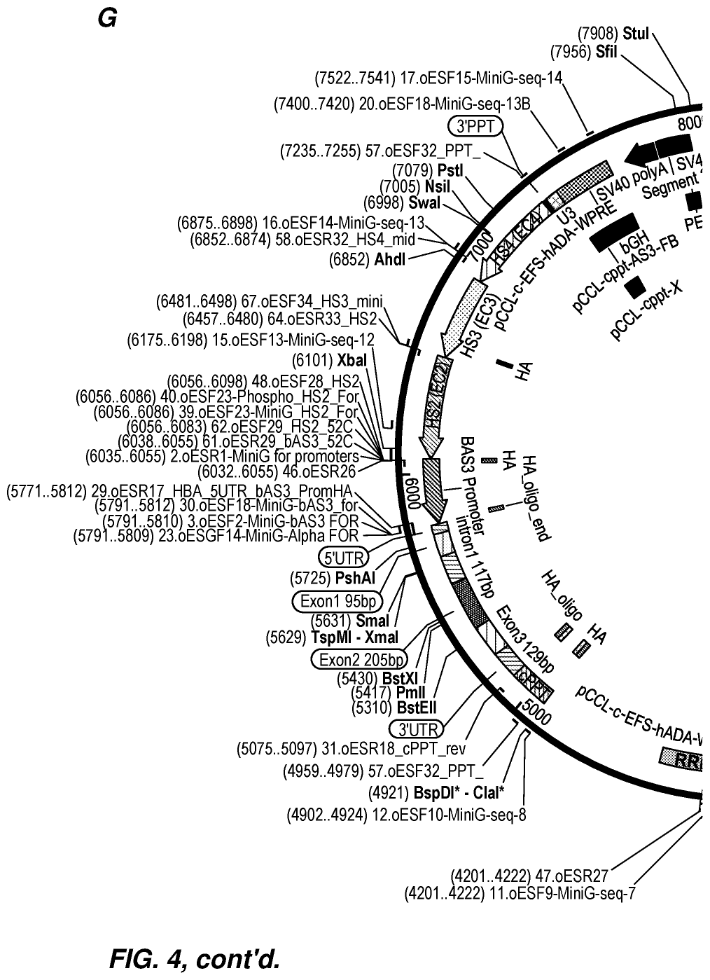

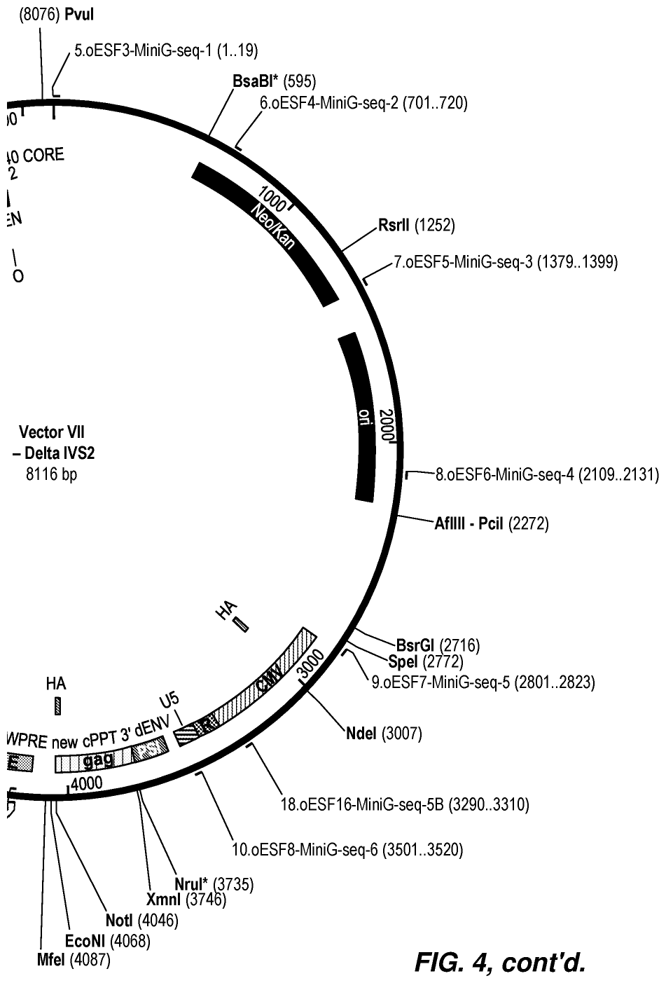

It is noted that full sequences for vectors T-VII, shown in FIG. 1, panel B, are provided in the Sequence Listing and maps of vectors I-VII are shown in FIG. 4, panels A-F, respectively.

The foregoing expression cassettes and vectors (e.g., LVs) are illustrative and non-limiting. Using the teaching provided herein numerous other alpha-globin expression cassettes and vectors containing such cassettes will be available to one of skill in the alt.

### TAT-Independent and Self Inactivating Lentiviral Vectors.

As noted above, in various embodiments the LVs described herein can comprise various safety features. For example, the HIV LTR has been substituted with a CMV promoter to yield higher titer vector without the inclusion of the HIV TAT protein during packaging. In certain embodiments an insulator (e.g., the FB insulator) is introduced into the 3′LTR for safety. The LVs are also constructed to provide efficient transduction and high titer.

To further improve safety, in various embodiments, the lentiviral vectors described herein comprise a TAT-independent, self-inactivating (SIN) configuration. Thus, in various embodiments it is desirable to employ in the LVs described herein an LTR region that has reduced promoter activity relative to wild-type LTR. Such constructs can be provided that are effectively “self-inactivating” (SIN) which provides a biosafety feature. SIN vectors are ones in which the production of full-length vector RNA in transduced cells is greatly reduced or abolished altogether. This feature minimizes the risk that replication-competent recombinants (RCRs) will emerge. Furthermore, it reduces the risk that that cellular coding sequences located adjacent to the vector integration site will be aberrantly expressed.

Furthermore, a SIN design reduces the possibility of interference between the LTR and the promoter that is driving the expression of the transgene. SIN LVs can often permit full activity of the internal promoter.

The SIN design increases the biosafety of the LVs. The majority of the HIV LTR is comprised of the U3 sequences. The U3 region contains the enhancer and promoter elements that modulate basal and induced expression of the HIV genome in infected cells and in response to cell activation. Several of these promoter elements are essential for viral replication. Some of the enhancer elements are highly conserved among viral isolates and have been implicated as critical virulence factors in viral pathogenesis. The enhancer elements may act to influence replication rates in the different cellular target of the virus

As viral transcription starts at the 3′ end of the U3 region of the 5′ LTR, those sequences are not part of the viral mRNA and a copy thereof from the 3′ LTR acts as template for the generation of both LTR's in the integrated provirus. If the 3′ copy of the U3 region is altered in a retroviral vector construct, the vector RNA is still produced from the intact 5′ LTR in producer cells but cannot be regenerated in target cells. Transduction of such a vector results in the inactivation of both LTR's in the progeny virus. Thus, the retrovirus is self-inactivating (SIN) and those vectors are known as SIN transfer vectors.

In certain embodiments self-inactivation is achieved through the introduction of a deletion in the U3 region of the 3′ LTR of the vector DNA, i.e., the DNA used to produce the vector RNA. During RT, this deletion is transferred to the 5′ LTR of the proviral DNA. Typically, it is desirable to eliminate enough of the U3 sequence to greatly diminish or abolish altogether the transcriptional activity of the LTR, thereby greatly diminishing or abolishing the production of full-length vector RNA in transduced cells. However, it is generally desirable to retain those elements of the LTR that are involved in polyadenylation of the viral RNA, a function typically spread out over U3, R and U5. Accordingly, in certain embodiments, it is desirable to eliminate as many of the transcriptionally important motifs from the LTR as possible while sparing the polyadenylation determinants.

The SIN design is described in detail in Zufferey et al. (1998) J Virol. 72(12): 9873-9880, and in U.S. Pat. No. 5,994,136. As described therein, there are, however, limits to the extent of the deletion at the 3′ LTR. First, the 5′ end of the U3 region serves another essential function in vector transfer, being required for integration (terminal dinucleotide+att sequence). Thus, the terminal dinucleotide and the att sequence may represent the 5′ boundary of the U3 sequences which can be deleted. In addition, some loosely defined regions may influence the activity of the downstream polyadenylation site in the R region. Excessive deletion of U3 sequence from the 3′LTR may decrease polyadenylation of vector transcripts with adverse consequences both on the titer of the vector in producer cells and the transgene expression in target cells.

Additional SIN designs are described in U.S. Patent Publication No: 2003/0039636. As described therein, in certain embodiments, the lentiviral sequences removed from the LTRs are replaced with comparable sequences from a non-lentiviral retrovirus, thereby forming hybrid LTRs. In particular, the lentiviral R region within the LTR can be replaced in whole or in part by the R region from a non-lentiviral retrovirus. In certain embodiments, the lentiviral TAR sequence, a sequence which interacts with TAT protein to enhance viral replication, is removed, preferably in whole, from the R region. The TAR sequence is then replaced with a comparable portion of the R region from a non-lentiviral retrovirus, thereby forming a hybrid R region. The LTRs can be further modified to remove and/or replace with non-lentiviral sequences all or a portion of the lentiviral U3 and U5 regions.

Accordingly, in certain embodiments, the SIN configuration provides a retroviral LTR comprising a hybrid lentiviral R region that lacks all or a portion of its TAR sequence, thereby eliminating any possible activation by TAT, wherein the TAR sequence or portion thereof is replaced by a comparable portion of the R region from a non-lentiviral retrovirus, thereby forming a hybrid R region. In a particular embodiment, the retroviral LTR comprises a hybrid R region, wherein the hybrid R region comprises a portion of the HIV R region (e.g., a portion comprising or consisting of the nucleotide sequence shown in SEQ ID NO:10 in US 2003/0039636) lacking the TAR sequence, and a portion of the MoMSV R region (e.g., a portion comprising or consisting of the nucleotide sequence shown in SEQ ID NO:9 in 2003/0039636) comparable to the TAR sequence lacking from the HIV R region. In another particular embodiment, the entire hybrid R region comprises or consists of the nucleotide sequence shown in SEQ ID NO:11 in 2003/0039636.

Suitable lentiviruses from which the R region can be derived include, for example, HIV (HIV-1 and HIV-2), EIV, SIV and FIV. Suitable retroviruses from which non-lentiviral sequences can be derived include, for example, MoMSV, MoMLV, Friend, MSCV, RSV and Spumaviruses. In one illustrative embodiment, the lentivirus is HIV and the non-lentiviral retrovirus is MoMSV.

In another embodiment described in US 2003/0039636, the LTR comprising a hybrid R region is a left (5′) LTR and further comprises a promoter sequence upstream from the hybrid R region. Preferred promoters are non-lentiviral in origin and include, for example, the U3 region from a non-lentiviral retrovirus (e.g., the MoMSV U3 region). In one particular embodiment, the U3 region comprises the nucleotide sequence shown in SEQ ID NO:12 in US 2003/0039636. In another embodiment, the left (5′) LTR further comprises a lentiviral U5 region downstream from the hybrid R region. In one embodiment, the U5 region is the HIV U5 region including the HIV att site necessary for genomic integration. In another embodiment, the U5 region comprises the nucleotide sequence shown in SEQ ID NO:13 in US 2003/0039636. In yet another embodiment, the entire left (5′) hybrid LTR comprises the nucleotide sequence shown in SEQ ID NO:1 in US 2003/0039636.

In another illustrative embodiment, the LTR comprising a hybrid R region is a right (3′) LTR and further comprises a modified (e.g., truncated) lentiviral U3 region upstream from the hybrid R region. The modified lentiviral U3 region can include the att sequence, but lack any sequences having promoter activity, thereby causing the vector to be SIN in that viral transcription cannot go beyond the first round of replication following chromosomal integration. In a particular embodiment, the modified lentiviral U3 region upstream from the hybrid R region consists of the 3′ end of a lentiviral (e.g., HIV) U3 region up to and including the lentiviral U3 att site. In one embodiment, the U3 region comprises the nucleotide sequence shown in SEQ ID NO:15 in US 2003/0039636. In another embodiment, the right (3′) LTR further comprises a polyadenylation sequence downstream from the hybrid R region. In another embodiment, the polyadenylation sequence comprises the nucleotide sequence shown in SEQ ID NO:16 in US 2003/0039636. In yet another embodiment, the entire right (5′) LTR comprises the nucleotide sequence shown in SEQ ID NO:2 or 17 of US 2003/0039636.

Thus, in the case of HIV based LV, it has been discovered that such vectors tolerate significant U3 deletions, including the removal of the LTR TATA box (e.g., deletions from −418 to −18), without significant reductions in vector titers. These deletions render the LTR region substantially transcriptionally inactive in that the transcriptional ability of the LTR in reduced to about 90% or lower.

It has also been demonstrated that the trans-acting function of Tat becomes dispensable if part of the upstream LTR in the transfer vector construct is replaced by constitutively active promoter sequences (see, e.g., Dull et al. (1998) J Virol. 72(11): 8463-8471. Furthermore, we show that the expression of rev in trans allows the production of high-titer HIV-derived vector stocks from a packaging construct which contains only gag and pol. This design makes the expression of the packaging functions conditional on complementation available only in producer cells. The resulting gene delivery system, conserves only three of the nine genes of HIV-1 and relies on four separate transcriptional units for the production of transducing particles.

The foregoing SIN configurations are illustrative and non-limiting. Numerous SIN configurations are known to those of skill in the art. As indicated above, in certain embodiments, the LTR transcription is reduced by about 95% to about 99%. In certain embodiments LTR may be rendered at least about 90%, at least about 91%, at least about 92%, at least about 93%, at least about 94%, at least about 95% at least about 96%, at least about 97%, at least about 98%, or at least about 99% transcriptionally inactive.

### Insulator Element

In certain embodiments, to further enhance biosafety, insulators are inserted into the lentiviral vectors described herein. Insulators are DNA sequence elements present throughout the genome. They bind proteins that modify chromatin and alter regional gene expression. The placement of insulators in the vectors described herein offer various potential benefits including, inter alia: 1) Shielding of the vector from positional effect variegation of expression by flanking chromosomes (i.e., barrier activity); and 2) Shielding flanking chromosomes from insertional trans-activation of gene expression by the vector (enhancer blocking). Thus, insulators can help to preserve the independent function of genes or transcription units embedded in a genome or genetic context in which their expression may otherwise be influenced by regulatory signals within the genome or genetic context (see, e.g., Burgess-Beusse et al. (2002) Proc. Natl. Acad. Sci. USA, 99: 16433; and Zhan et al. (2001) Hum. Genet., 109: 471). In the present context insulators may contribute to protecting lentivirus-expressed sequences from integration site effects, which may be mediated by cis-acting elements present in genomic DNA and lead to deregulated expression of transferred sequences. In various embodiments LVs are provided in which an insulator sequence is inserted into one or both LTRs or elsewhere in the region of the vector that integrates into the cellular genome.

The first and best characterized vertebrate chromatin insulator is located within the chicken β-globin locus control region. This element, which contains a DNase-I hypersensitive site-4 (cHS4), appears to constitute the 5′ boundary of the chicken β-globin locus (Prioleau et al. (1999) EMBO J. 18: 4035-4048). A 1.2-kb fragment containing the cHS4 element displays classic insulator activities, including the ability to block the interaction of globin gene promoters and enhancers in cell lines (Chung et al. (1993) Cell, 74: 505-514), and the ability to protect expression cassettes in Drosophila (Id.), transformed cell lines (Pikaart et al. (1998) Genes Dev. 12: 2852-2862), and transgenic mammals (Wang et al. (1997) Nat. Biotechnol., 15: 239-243; Taboit-Dameron et al. (1999) Transgenic Res., 8: 223-235) from position effects. Much of this activity is contained in a 250-bp fragment. Within this stretch is a 49-bp cHS4 core (Chung et al. (1997) Proc. Natl. Acad. Sci., USA, 94: 575-580) that interacts with the zinc finger DNA binding protein CTCF implicated in enhancer-blocking assays (Bell et al. (1999) Cell, 98: 387-396).

One illustrative and suitable insulator is FB (FII/BEAD-A), a 77 bp insulator element, that contains the minimal CTCF binding site enhancer-blocking components of the chicken β-globin 5′ HS4 insulators and a homologous region from the human T-cell receptor alpha/delta blocking element alpha/delta I (BEAD-I) insulator described by Ramezani et al. (2008) Stem Cell 26: 3257-3266. The FB “synthetic” insulator has full enhancer blocking activity. This insulator is illustrative and non-limiting. Other suitable insulators may be used including, for example, the full-length chicken beta-globin HS4 or insulator sub-fragments thereof, the ankyrin gene insulator, and other synthetic insulator elements.

### Packaging Signal.

In various embodiments the vectors described herein further comprise a packaging signal. A “packaging signal,” “packaging sequence,” or “psi sequence” is any nucleic acid sequence sufficient to direct packaging of a nucleic acid whose sequence comprises the packaging signal into a retroviral particle. The term includes naturally occurring packaging sequences and also engineered variants thereof. Packaging signals of a number of different retroviruses, including lentiviruses, are known in the art.

### Rev Responsive Element (RRE).

In certain embodiments the lentiviral vectors described herein comprise a Rev response element (RRE) to enhance nuclear export of unspliced RNA. RREs are well known to those of skill in the art. Illustrative RREs include but are not limited to RREs such as that located at positions 7622-8459 in the HIV NL4-3 genome (Genbank accession number AF003887) as well as RREs from other strains of HIV or other retroviruses. Such sequences are readily available from Genbank or from the database with URL hiv-web.lanl.gov/content/index. One illustrative, but non-limiting RRE is provided by the sequence of SEQ ID NO:23.

Central PolyPurine Tract (cPPT).

In various embodiments the lentiviral vectors described herein further include a central polypurine tract. Insertion of a fragment containing the central polypurine tract (cPPT) in lentiviral (e.g., HIV-1) vector constructs is known to enhance transduction efficiency drastically, reportedly by facilitating the nuclear import of viral cDNA through a central DNA flap.

### Expression-Stimulating Posttranscriptional Regulatory Element (PRE)

In certain embodiments the lentiviral vectors (LVs) described herein may comprise any of a variety of posttranscriptional regulatory elements (PREs) whose presence within a transcript increases expression of the heterologous nucleic acid (e.g., βAS3) at the protein level. PREs may be particularly useful in certain embodiments, especially those that involve lentiviral constructs with modest promoters.

One type of PRE is an intron positioned within the expression cassette, which can stimulate gene expression. However, introns can be spliced out during the life cycle events of a lentivirus. Hence, if introns are used as PRE's they are typically placed in an opposite orientation to the vector genomic transcript.

Posttranscriptional regulatory elements that do not rely on splicing events offer the advantage of not being removed during the viral life cycle. Some examples are the posttranscriptional processing element of herpes simplex virus, the posttranscriptional regulatory element of the hepatitis B virus (HPRE) and the woodchuck hepatitis virus (WPRE). Of these the WPRE is typically preferred as it contains an additional cis-acting element not found in the HPRE. This regulatory element is typically positioned within the vector so as to be included in the RNA transcript of the transgene, but outside of stop codon of the transgene translational unit.

The WPRE is characterized and described in U.S. Pat. No. 6,136,597. As described therein, the WPRE is an RNA export element that mediates efficient transport of RNA from the nucleus to the cytoplasm. It enhances the expression of transgenes by insertion of a cis-acting nucleic acid sequence, such that the element and the transgene are contained within a single transcript. Presence of the WPRE in the sense orientation was shown to increase transgene expression by up to 7- to 10-fold. Retroviral vectors transfer sequences in the form of cDNAs instead of complete intron-containing genes as introns are generally spliced out during the sequence of events leading to the formation of the retroviral particle. Introns mediate the interaction of primary transcripts with the splicing machinery. Because the processing of RNAs by the splicing machinery facilitates their cytoplasmic export, due to a coupling between the splicing and transport machineries, cDNAs are often inefficiently expressed. Thus, the inclusion of the WPRE in a vector can result in enhanced expression of transgenes.

### Transduced Host Cells and Methods of Cell Transduction.

The recombinant lentiviral vectors (LV) and resulting virus described herein are capable of transferring a heterologous nucleic acid (e.g., a nucleic acid encoding an α-globin gene) sequence into a mammalian cell. In various embodiments, for delivery to cells, vectors described herein are preferably used in conjunction with a suitable packaging cell line or co-transfected into cells in vitro along with other vector plasmids containing the necessary retroviral genes (e.g., gag and pol) to form replication incompetent virions capable of packaging the vectors of the present invention and infecting cells.

The recombinant LVs and resulting virus described herein are capable of transferring a nucleic acid (e.g., a nucleic acid encoding an α-globin or other sequence) into a mammalian cell. For delivery to cells, various vectors described herein are preferably used in conjunction with a suitable packaging cell line or co-transfected into cells in vitro along with other vector plasmids containing the necessary retroviral genes (e.g., gag and pol) to form replication incompetent virions capable of packaging the vectors of the present invention and infecting cells.

In certain embodiments the vectors are introduced via transfection into the packaging cell line. The packaging cell line produces viral particles that contain the vector genome. Methods for transfection are well known by those of skill in the art. After cotransfection of the packaging vectors and the transfer vector to the packaging cell line, the recombinant virus is recovered from the culture media and titered by standard methods used by those of skill in the art. Thus, the packaging constructs can be introduced into human cell lines by calcium phosphate transfection, lipofection or electroporation, generally together with or without a dominant selectable marker, such as neomycin, DHFR, Glutamine synthetase, followed by selection in the presence of the appropriate drug and isolation of clones. In certain embodiments the selectable marker gene can be linked physically to the packaging genes in the construct.

Stable cell lines wherein the packaging functions are configured to be expressed by a suitable packaging cell are known (see, e.g., U.S. Pat. No. 5,686,279, which describes packaging cells). In general, for the production of virus particles, one may employ any cell that is compatible with the expression of lentiviral Gag and Pol genes, or any cell that can be engineered to support such expression. For example, producer cells such as 293T cells and HT1080 cells may be used.

The packaging cells with a lentiviral vector incorporated therein form producer cells. Producer cells are thus cells or cell-lines that can produce or release packaged infectious viral particles carrying the therapeutic gene of interest (e.g., an α-globin gene). These cells can further be anchorage dependent which means that these cells will grow, survive, or maintain function optimally when attached to a surface such as glass or plastic. Some examples of anchorage dependent cell lines used as lentiviral vector packaging cell lines when the vector is replication competent are HeLa or 293 cells and PERC.6 cells.

Accordingly, in certain embodiments, methods are provided of delivering a gene to a cell which is then integrated into the genome of the cell, comprising contacting the cell with a virion containing a lentiviral vector described herein. The cell (e.g., in the form of tissue or an organ) can be contacted (e.g., infected) with the virion ex vivo and then delivered to a subject (e.g., a mammal, animal or human) in which the gene (e.g., an α-globin gene) will be expressed. In various embodiments the cell can be autologous to the subject (i.e., from the subject) or it can be non-autologous (i.e., allogeneic or xenogenic) to the subject. Moreover, because the vectors described herein are capable of being delivered to both dividing and non-dividing cells, the cells can be from a wide variety including, for example, bone marrow cells, mesenchymal stem cells (e.g., obtained from adipose tissue), and other primary cells derived from human and animal sources. Alternatively, the virion can be directly administered in vivo to a subject or a localized area of a subject (e.g., bone marrow).

Of course, as noted above, the lentivectors described herein will be particularly useful in the transduction of human hematopoietic progenitor cells or a hematopoietic stem cells, obtained either from the bone marrow, the peripheral blood or the umbilical cord blood, as well as in the transduction of a CD4⁺ T cell, a peripheral blood B or T lymphocyte cell, and the like. In certain embodiments particularly preferred targets are CD34⁺ hematopoietic stem and progenitor cells.

### Gene Therapy.

In still other embodiments, methods are provided for transducing a human hematopoietic stem cell. In certain embodiments the methods involve contacting a population of human cells that include hematopoietic stem cells with one of the foregoing lentivectors under conditions to affect the transduction of a human hematopoietic progenitor cell in said population by the vector. The stem cells may be transduced in vivo or in vitro, depending on the ultimate application. Even in the context of human gene therapy, such as gene therapy of human stem cells, one may transduce the stem cell in vivo or, alternatively, transduce in vitro followed by infusion of the transduced stem cell into a human subject. In one aspect of this embodiment, the human stem cell can be removed from a human, e.g., a human patient, using methods well known to those of skill in the art and transduced as noted above. The transduced stem cells are then reintroduced into the same or a different human.

As an illustrative, but non-limiting example of a method of treatment of alpha thalassemia (AT) in a subject or reducing the risk of developing alpha thalassemia (AT), the method comprises administering to the subject a lentiviral composition as described herein that expresses an α-globin gene. In one embodiment, the method further comprises identifying a subject as having α-thalassemia, or as at risk of developing alpha thalassemia prior to administering a treatment as described herein.

### Stem Cell/Progenitor Cell Gene Therapy.

In various embodiments the lentivectors described herein are particularly useful for the transduction of human hematopoietic progenitor cells or hematopoietic stem cells (HSCs), obtained either from the bone marrow, the peripheral blood or the umbilical cord blood, as well as in the transduction of a CD4⁺ T cell, a peripheral blood B or T lymphocyte cell, and the like. In certain embodiments particularly preferred targets are CD34⁺ hematopoietic stem and progenitor cells.

When cells, for instance CD34⁺ cells, dendritic cells, peripheral blood cells or tumor cells are transduced ex vivo, the vector particles are incubated with the cells using a dose generally in the order of between 1 to 50 multiplicities of infection (MOI) which also corresponds to 1×10⁵ to 50×10⁵ transducing units of the viral vector per 10⁵ cells. This can include amounts of vector corresponding to 1, 2, 3, 4, 5, 6, 7, 8, 9, 10, 15, 20, 25, 30, 35, 40, 45, and 50 MOI. Typically, the amount of vector may be expressed in terms of HT-29 transducing units (TU).

In certain embodiments cell-based therapies involve providing stem cells and/or hematopoietic precursors, transduce the cells with the lentivirus encoding, e.g., an α-globin gene, and then introduce the transformed cells into a subject in need thereof (e.g., a subject with the sickle cell mutation).

In certain embodiments the methods involve isolating population of cells, e.g., stem cells from a subject, optionally expand the cells in tissue culture, and administer the lentiviral vector whose presence within a cell(s) results in production of an α-globin in the cells in vitro. The cells are then returned to the subject, where, for example, they may provide a population of red blood cells that produce the α-globin.

In some illustrative, but non-limiting, embodiments, a population of cells, which may be cells from a cell line or from an individual other than the subject, can be used. Methods of isolating stem cells, immune system cells, etc., from a subject and returning them to the subject are well known in the art. Such methods are used, e.g., for bone marrow transplant, peripheral blood stem cell transplant, etc., in patients undergoing chemotherapy.

Where stem cells are to be used, it will be recognized that such cells can be derived from a number of sources including bone marrow (BM), cord blood (CB), mobilized peripheral blood stem cells (mPBSC), and the like. In certain embodiments the use of induced pluripotent stem cells (IPSCs) is contemplated. Methods of isolating hematopoietic stem cells (HSCs), transducing such cells and introducing them into a mammalian subject are well known to those of skill in the art.

In certain embodiments a lentiviral vector described herein (see, e.g., FIG. 1, panel B) is used in stem cell gene therapy for AT by introducing the βAS3 α-globin gene into the bone marrow stem cells of patients with alpha thalassemia followed by autologous transplantation.

### Direct Introduction of Vector.

In certain embodiments direct treatment of a subject by direct introduction of the vector(s) described herein is contemplated. The lentiviral compositions may be formulated for delivery by any available route including, but not limited to parenteral (e.g., intravenous), intradermal, subcutaneous, oral (e.g., inhalation), transdermal (topical), transmucosal, rectal, and vaginal. Commonly used routes of delivery include inhalation, parenteral, and transmucosal.

In various embodiments pharmaceutical compositions can include an LV in combination with a pharmaceutically acceptable carrier. As used herein the language “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into the compositions.

In some embodiments, active agents, i.e., a lentiviral described herein and/or other agents to be administered together the vector, are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such compositions will be apparent to those skilled in the art. Suitable materials can also be obtained commercially from Alza Corporation and Nova Pharmaceuticals, Inc. Liposomes can also be used as pharmaceutically acceptable carriers. These can be prepared according to methods known to those skilled in the art, for example, as described in U.S. Pat. No. 4,522,811. In some embodiments the composition is targeted to particular cell types or to cells that are infected by a virus. For example, compositions can be targeted using monoclonal antibodies to cell surface markers, e.g., endogenous markers or viral antigens expressed on the surface of infected cells.

It is advantageous to formulate compositions in dosage unit form for ease of administration and uniformity of dosage. Dosage unit form as used herein refers to physically discrete units suited as unitary dosages for the subject to be treated; each unit comprising a predetermined quantity of a LV calculated to produce the desired therapeutic effect in association with a pharmaceutical carrier.

A unit dose need not be administered as a single injection but may comprise continuous infusion over a set period of time. Unit dose of the LV described herein may conveniently be described in terms of transducing units (T.U.) of lentivector, as defined by titering the vector on a cell line such as HeLa or 293. In certain embodiments unit doses can range from 10³, 10⁴, 10⁵, 10⁶, 10⁷, 10⁸, 10⁹, 10¹⁰, 10¹¹, 10¹², 10¹³ T.U. and higher.

Pharmaceutical compositions can be administered at various intervals and over different periods of time as required, e.g., one time per week for between about 1 to about 10 weeks; between about 2 to about 8 weeks; between about 3 to about 7 weeks; about 4 weeks; about 5 weeks; about 6 weeks, etc. It may be necessary to administer the therapeutic composition on an indefinite basis. The skilled artisan will appreciate that certain factors can influence the dosage and timing required to effectively treat a subject, including but not limited to the severity of the disease or disorder, previous treatments, the general health and/or age of the subject, and other diseases present. Treatment of a subject with a LV can include a single treatment or, in many cases, can include a series of treatments.

Illustrative, but non-limiting, doses for administration of gene therapy vectors and methods for determining suitable doses are known in the art. It is furthermore understood that appropriate doses of a LV may depend upon the particular recipient and the mode of administration. The appropriate dose level for any particular subject may depend upon a variety of factors including the age, body weight, general health, gender, and diet of the subject, the time of administration, the route of administration, the rate: of excretion, other administered therapeutic agents, and the like.

In certain embodiments lentiviral gene therapy vectors described herein can be delivered to a subject by, for example, intravenous injection, local administration, or by stereotactic injection (see, e.g., Chen et al. (1994) Proc. Natl. Acad. Sci. USA, 91: 3054). In certain embodiments vectors may be delivered orally or via inhalation and may be encapsulated or otherwise manipulated to protect them from degradation, enhance uptake into tissues or cells, etc. Pharmaceutical preparations can include a LV in an acceptable diluent, or can comprise a slow release matrix in which a LV is imbedded. Alternatively or additionally, where a vector can be produced intact from recombinant cells, as is the case for retroviral or lentiviral vectors as described herein, a pharmaceutical preparation can include one or more cells which produce vectors. Pharmaceutical compositions comprising a LV described herein can be included in a container, pack, or dispenser, optionally together with instructions for administration.

The foregoing compositions, methods and uses are intended to be illustrative and not limiting. Using the teachings provided herein other variations on the compositions, methods and uses will be readily available to one of skill in the art.

The approach to generate reduced length enhance regions is superior to previous strategies for generating tissue-specific enhancers for, among other reasons: 1) The cost of goods is decreased due to a low number of outputs required to be tested, 2) Strength of synthetic enhancers may be superior to those produced with current methods, or they may be less active but more suitable for LV-mediated delivery, and 3). Enhancers can be of minimal length.

Additionally, without being bound to a particular theory, it is believed the enhancer mapping strategy described herein can be modified to generate genome-wide enhancer maps using a similar cloning strategy and sonicated human genomic DNA and that the mapping strategies can be used to generate synthetic enhancers responsive to an array of distinct cellular perturbations.

### Kits.

In certain embodiments, kits are provided for use of the α-globin expression cassettes or vectors comprising the α-globin expression cassettes described herein. In certain embodiments the kits comprise a container containing an expression cassette as described herein that expresses an α-globin gene as described herein. In certain embodiments the kits comprise a container containing a viral vector (e.g., a lentiviral vector) comprising the α-globin expression cassette as described herein. In certain embodiments the kits optionally further include cells e.g., viral packaging cells, and the like.

In addition, the kits optionally include labeling and/or instructional materials providing directions (i.e., protocols) for the of the expression cassette and/or the vectors and/or viral particles described herein.

While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

### EXAMPLES

The following examples are offered to illustrate, but not to limit the claimed invention.

### Example 1

### Preliminary Studies of α-Globin Lentiviral Vectors (AGLV)

Once cloned, the seven vectors (I-VII in FIG. 1, panel B) were packaged into replication-defective lentiviruses using the HEK293T PKR knockout cell line we developed. Following virus collection, HT-29 cells were transduced, and collected for DNA extraction three days post-transduction (dpt) to determine viral titer in transduction units per milliliters (TU/mL)—a measurement reflecting concentration of transducing particles. Titer is measured by the product of cell number and VCN, in which the latter is detected through droplet digital PCR (ddPCR) utilizing primers and a probe binding to the PSI (Ψ) region of provirus DNA incorporated in the genome.

### Analysis of AGLV

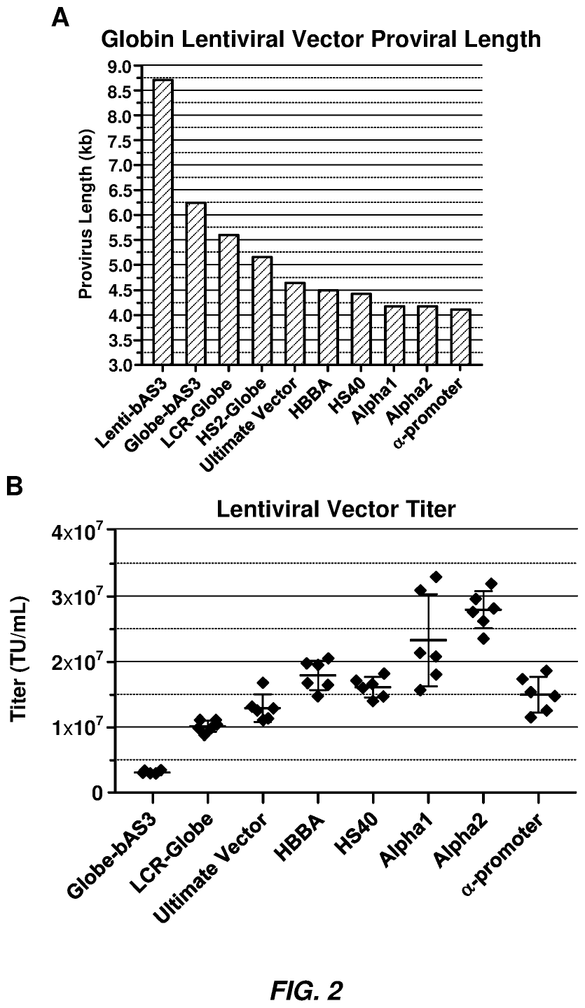

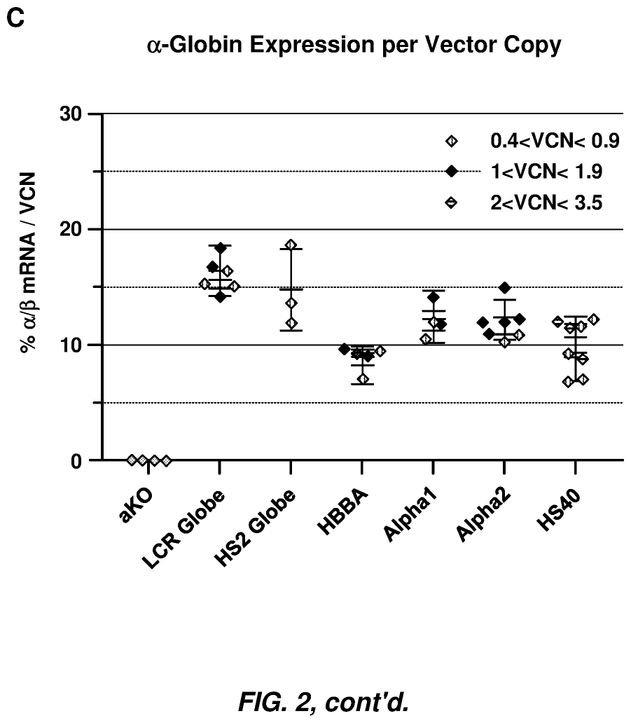

As shown in FIG. 2, panel A, all seven vectors are ˜2-4 kb shorter than (3-globin vectors employed in clinical trials for BT (i.e., our Lenti-βAS3 and GLOBE-βAS3). They display a high titer reaching up to 2.7e7 TU/mL, compared to the GLOBE-βAS3 LV with log-lower titer of 3e6 TU/mL (FIG. 2, panel B). Higher titers are advantageous for clinical scale vector production and cost per patient dose.

Thereafter, gene expression assessments were conducted in Human Umbilical Cord Blood-Derived Erythroid Progenitor (HUDEP-2) cells containing a ˜20 kb homologous deletion of all 4 α-globin genes (produced with Cas9), similar to genotypes of ATM patients with the SEA deletion (α-globin K/O HUDEP).

HUDEP is a unique erythroid cell line exhibiting adult hemoglobin expression following a two-week differentiation process; most other human erythroleukemia cell lines only produce fetal globins.

α-globin K/O HUDEP cells were first transduced at different virus concentrations to target an optimal VCN range of 0.5 to 3.5. Extraction of DNA and RNA (reverse transcribed)—is conducted for VCN and mRNA analyses, respectively. Similar to VCN, the α/β mRNA ratio is determined by ddPCR using different sets of primer and probes binding to α- and β-globin cDNA transcripts. Translation of the α-globin chains followed by their assembly with endogenous β-globin chains forms hemoglobin tetramers, identified by high-performance liquid chromatography (HPLC).

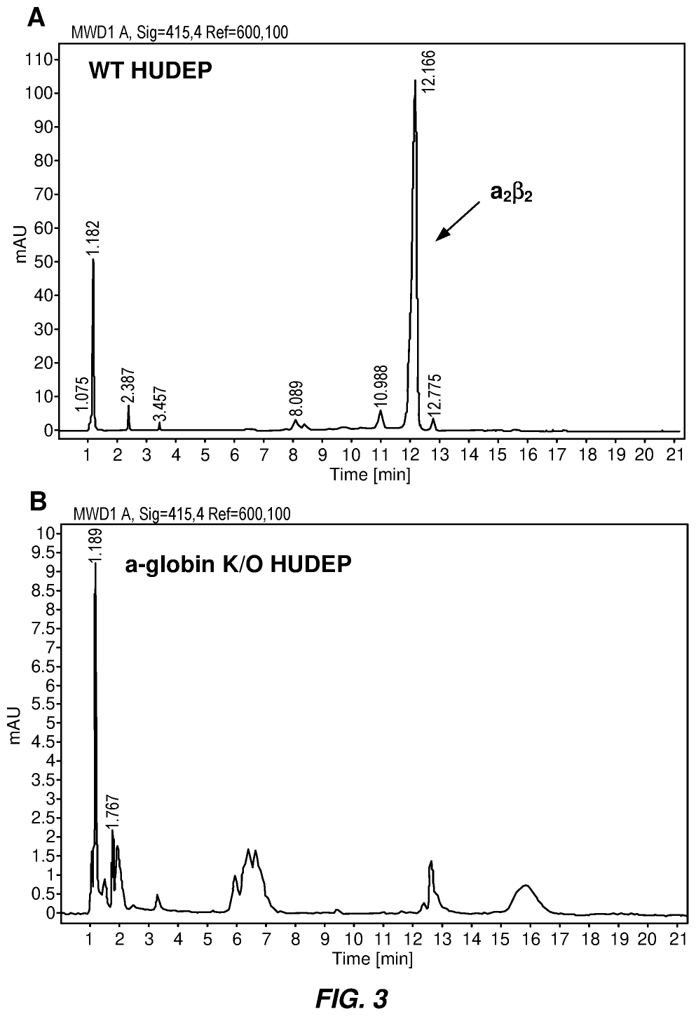

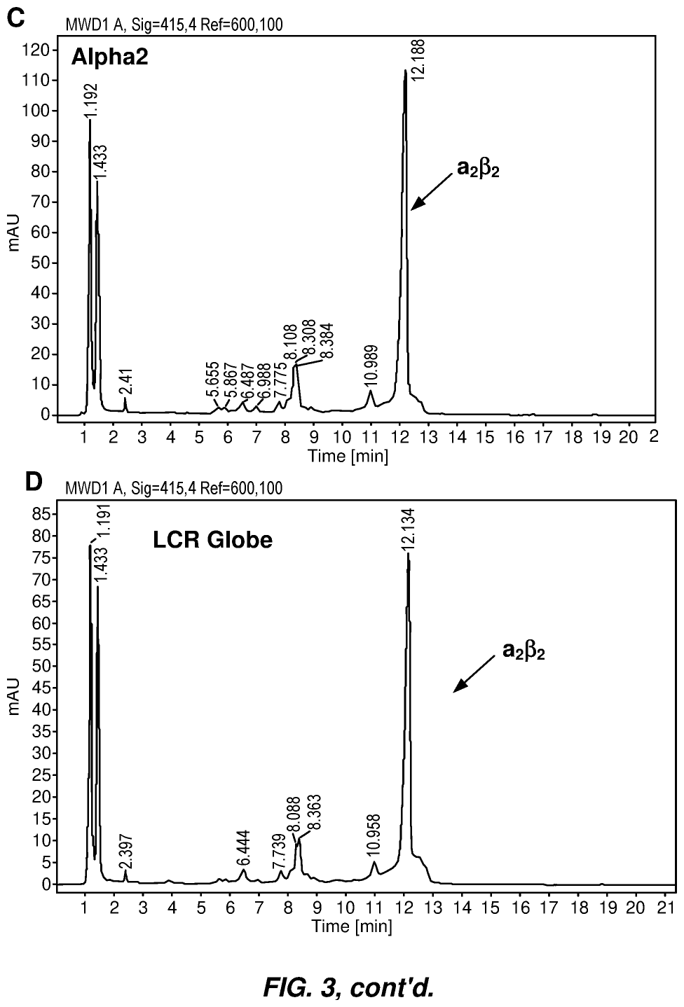

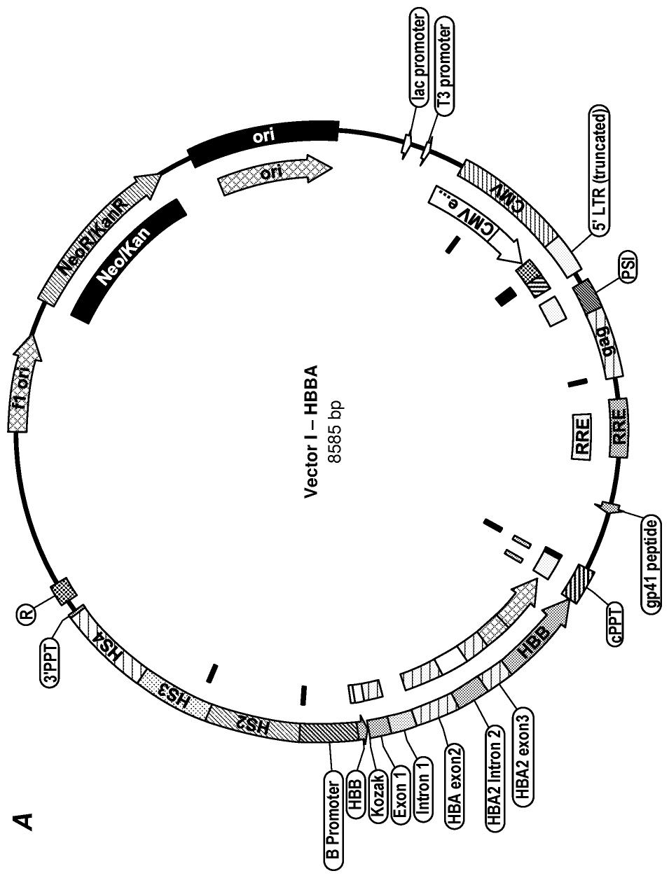

All of the AGLV constructs were functional and properly expressed α-globin mRNA and protein in differentiated erythroid-like cells (FIG. 2, panel C; FIG. 3). [The 4 endogenous α-globin alleles express equivalently to the 2 β-globin alleles, and thus a single α-globin allele expresses ˜25% as much as the total β-globin mRNA].

The LCR-Globe and HS2-Globe vectors produced the highest gene expression per vector copy (˜15%/total β-globin/VCN). The other LVs based in the UV backbone, with the smaller β-LCR, produced lower levels of α-globin gene expression (˜10%), demonstrating the stronger enhancer activity of the larger β-LCR.

We have analyzed the protein expression from the transduced and differentiated HUDEP cells by HPLC. As shown in FIG. 3, panel A, wild-type HUDEP cells present a major peak at elution time 12.4 minutes, representing the expression of adult hemoglobin (α₂β₂). As expected, non-transduced α-globin K/O HUDEP cells lack hemoglobin expression and serve as a negative control (FIG. 3, panel B). FIG. 3, panels C and D illustrate protein analysis from α-globin K/O HUDEP cells transduced with the Alpha2 and LCR-Globe vectors, respectively. The high peak at 12.4 minutes confirms the presence and high concentration of adult hemoglobin formation arising from α-globin transgene expression, and further establishes the ability of our α-globin LVs to produce adult hemoglobin in differentiated erythrocytes. Although not shown, all our α-globin LVs have also demonstrated proper hemoglobin production.

Thus far, Alpha2 and LCR-Globe vectors are the preliminary candidate vectors to move forward for in vitro studies as they have demonstrated the highest titers (Alpha2) and gene expression (LCR-Globe), respectively, and have both successfully generated adult hemoglobin. Further HUDEP assays (LV transduction and differentiation) are necessary to support and confirm our preliminary data in order to definitively identify lead candidate vectors for in vitro and in vivo studies.

Overall, these preliminary data illustrate the ability of these AGLV to transduce and integrate into the genomes of erythroid progenitors and properly express the α-globin gene—mRNA and protein—by regulation from β-globin regulatory elements—in differentiated erythrocytes.

### Example 2

### Further Studies of α-Globin Lentiviral Vectors (AGLV)

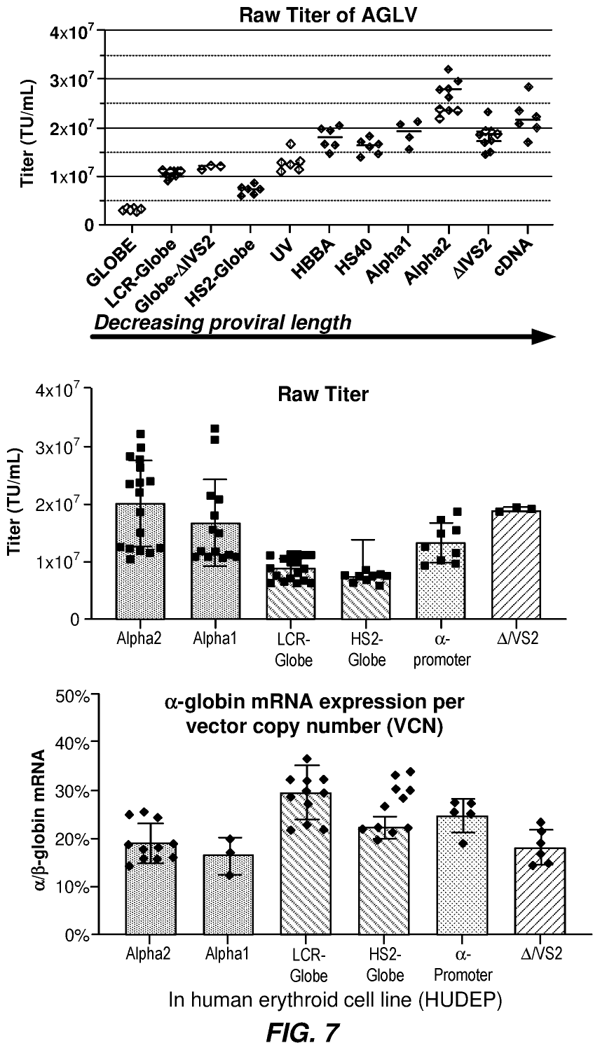

The first panel of FIG. 7 shows the raw titer of AGLV, illustrated in a decreasing proviral length. These vectors were packaged and titered at different times and all data are combined. The second panel shows the titer of the vectors from which gene expression in HUDEP are represented below. The third panel demonstrates the expression of α/β-globin mRNA in a human erythroid cell line (HUDEP) whose alpha globin loci have been removed. HUDEP cells can be transduced and differentiated into red blood cells. The cells were first plated, and transduced (3e5 TU/mL) by raw lentiviral vectors 24 hours later. 24 hours post-transduction, HUDEP cells were collected, spin and resuspend in differentiating media. Cells were under erythroid differentiation conditions for 10 days. Day 7 of the HUDEP differentiation, cells were collected and analyzed for VCN (reflecting gene transfer efficiency) and for mRNA gene expression levels. The gene expression is evaluated by quantifying alpha globin transcript from the transgene (there is no endogenous alpha globin mRNA in this ax-globin KO HUDEP cell line). As illustrated vector Alpha 1 and Alpha 2 appeared to produce the highest titer, while α-globin mRNA expression per VCN appeared to be higher using the LCR-Globe vector, although the differences may not be significantly significant.

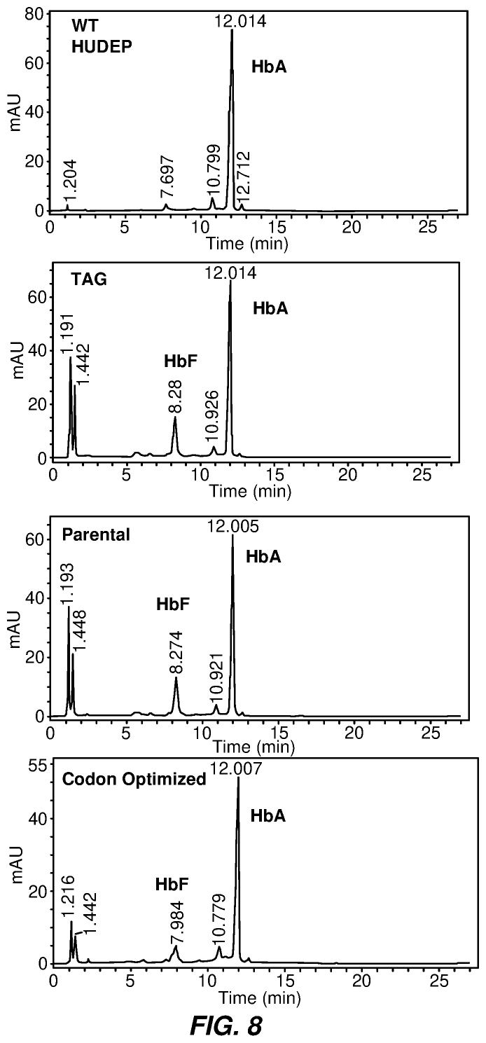

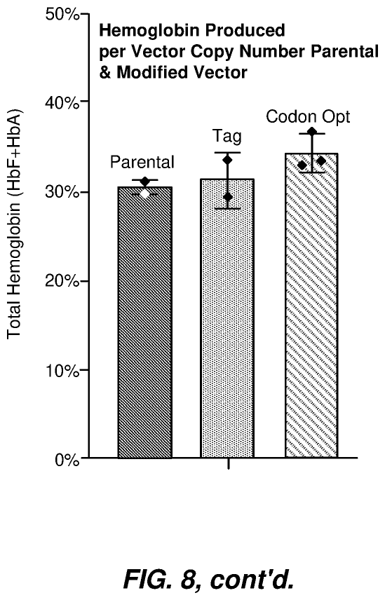

FIG. 8 illustrates the result of an experiment validating a Tag inserted in the alpha-globin gene. A comparison of the hemoglobin produced by the parental cells, the cells transformed with an LV containing a Tag and the cells containing a codon-optimized alpha globin cDNA did not show any significant difference.

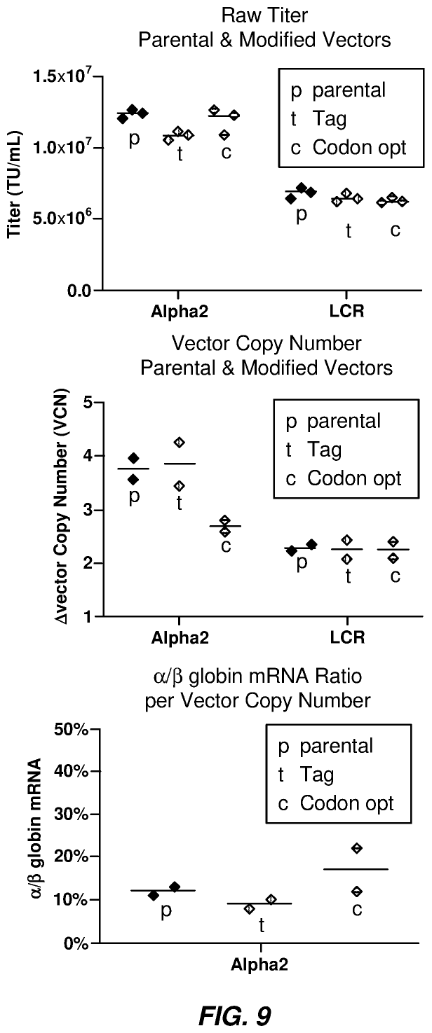

FIG. 9 illustrates the raw titer, VCN, and α/β mRNA ratio per vector copy number for parental Tag, an codon-optimized α-globin genes in Alpha 2 (later renamed A2-UV) and LCR (later renamed A2-Globe) vectors. Because endogenous alpha globin transcripts are identical to alpha globin transcripts from the transgene, we developed two tagging strategies to identify and quantify vector-derived transcripts. These vectors are “labeled/tagged” at the transcriptional level through two strategies: 1) inserting a ‘tag’ sequence downstream of the STOP codon. This tag sequence was invented in this lab and is an 18 nucleotide base pair sequence. Vectors containing this modification are known as their name with the word ‘-TAG’ attached to it. Example A2-UV containing tag sequence: A2-UV-tag. 2) codon optimizing a small region of the gene, in this case the last 8 codons of exon 3. Both strategies allow us to identify the vector-derived transcripts through these regions, not found in the endogenous alpha globin transcripts. FIG. 9 shows confirms that introducing the tag or codon optimized region does not alter vector efficiency, such as the titer, the gene transfer efficiency, and the gene expression levels. From these assays, the vectors continuing into further studies, in primary cells and in vivo, will contain the tag region.

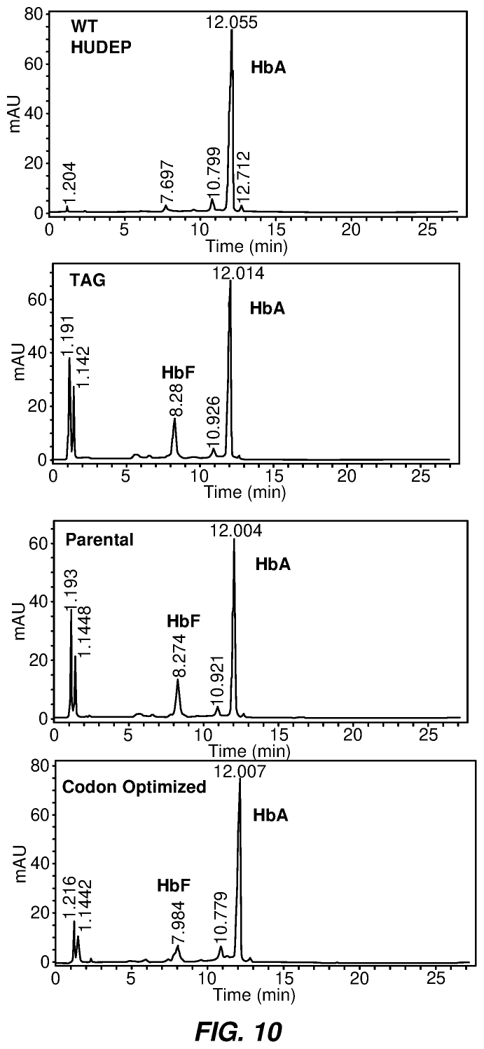

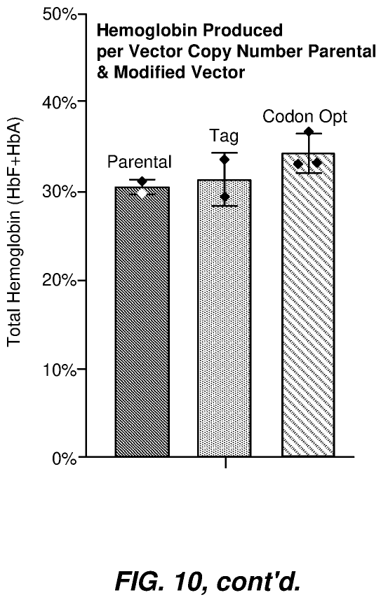

FIG. 10 illustrates hemoglobin production with tagged vectors. This figure demonstrates that the tag does not alter the structure of the protein.

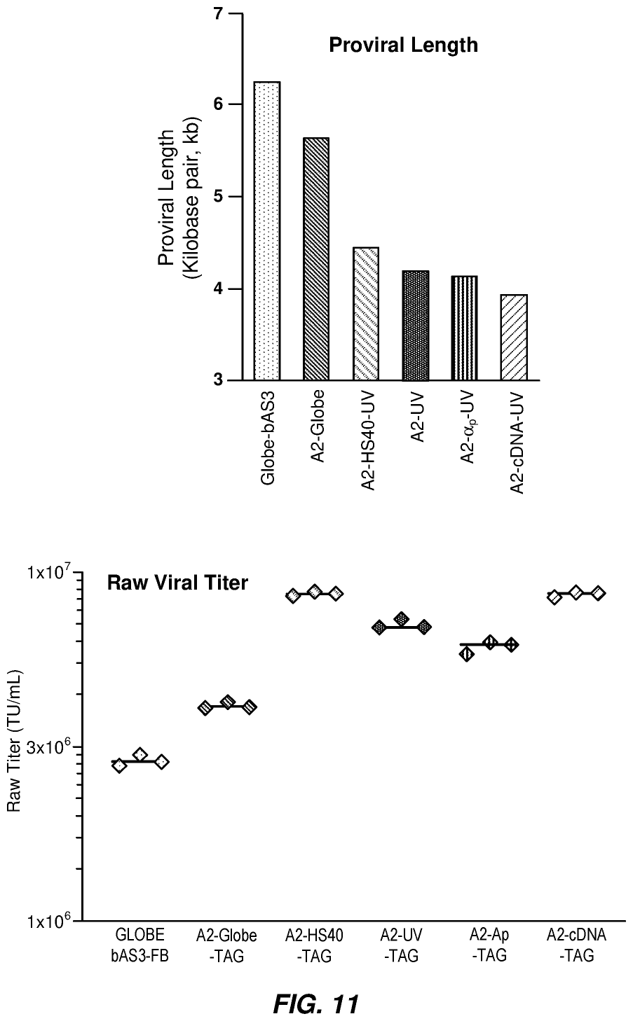

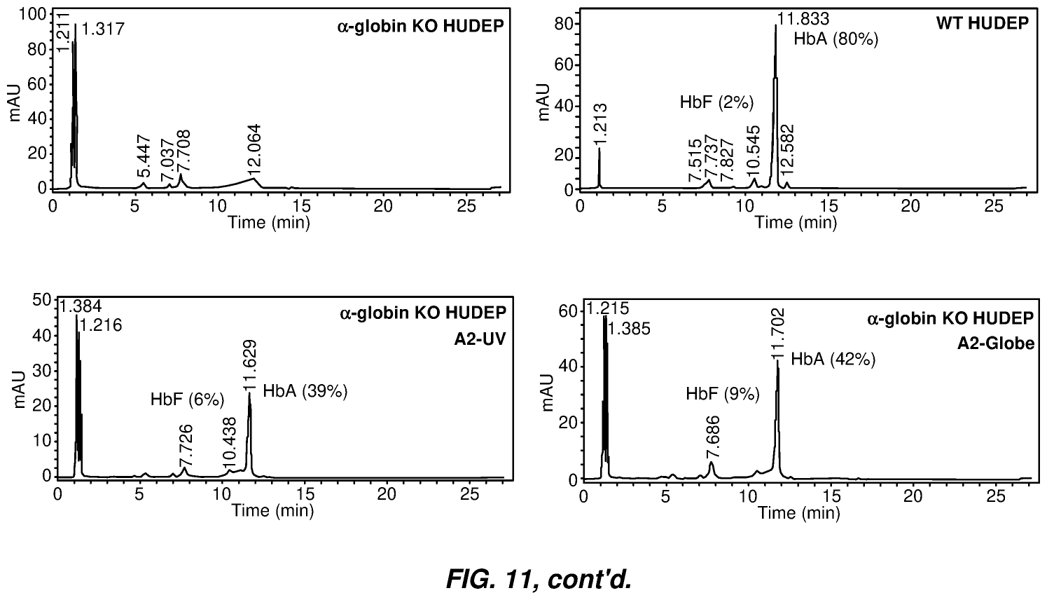

FIG. 11 shows a preliminary assessment of the functionality of vectors described herein (Globe-bAS3, A2-Globe, A2-HS40-UV, A2-UV, A2-αₚ-UV, and A2-cDNA-UV). Raw viral titer and hemoglobin production (HbA, HbF) was determined for the different vectors.

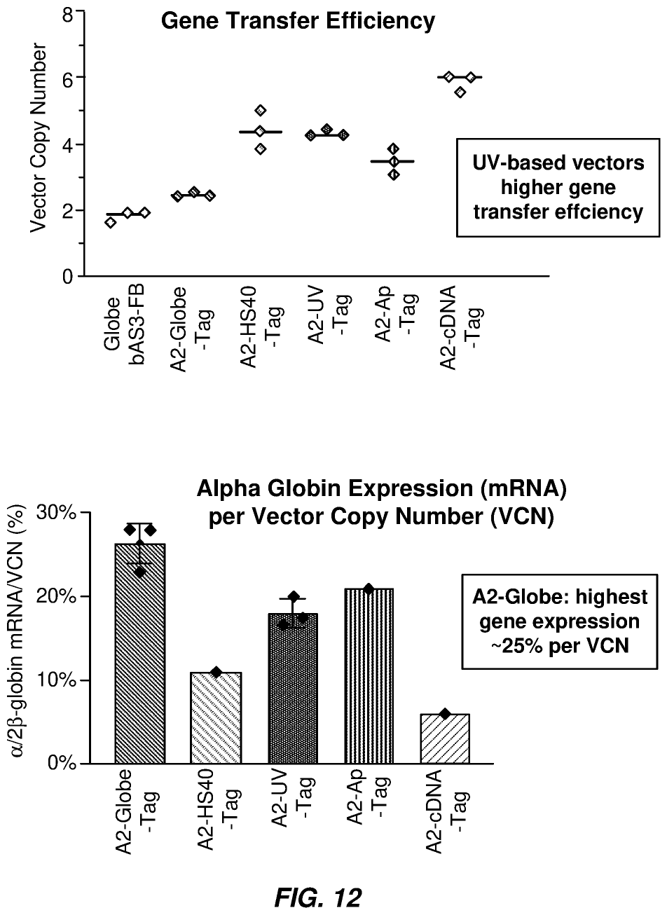

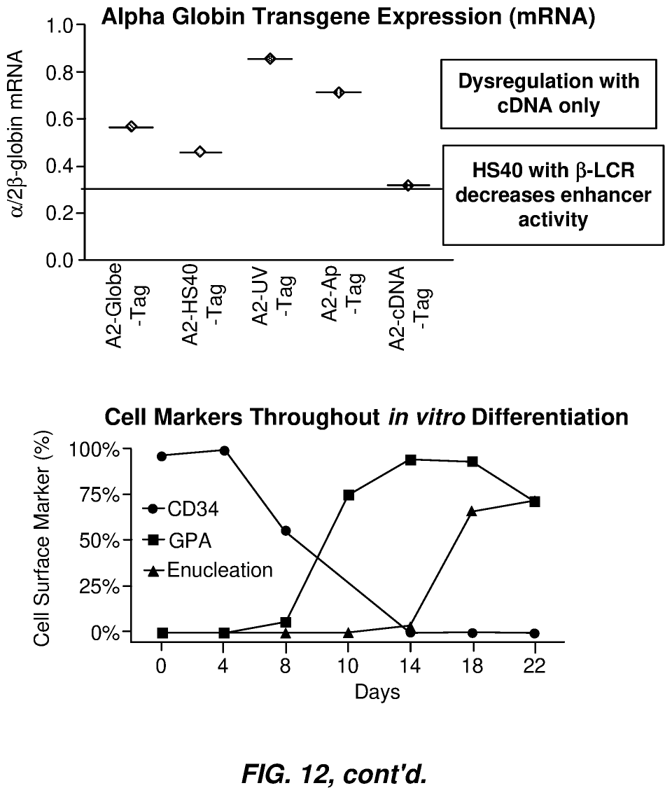

FIG. 12 illustrates results of vector assessment, in particular with respect to in vitro erythroid Differentiation of healthy donor hematopoietic stem and progenitor cells. AS shown, UV-based vectors had a higher gene transfer efficiency. The A2-Globe vector showed the highest gene expression (˜25% per VCN). Incorporation of HS40 with β-LCR appeared to decrease enhancer activity.

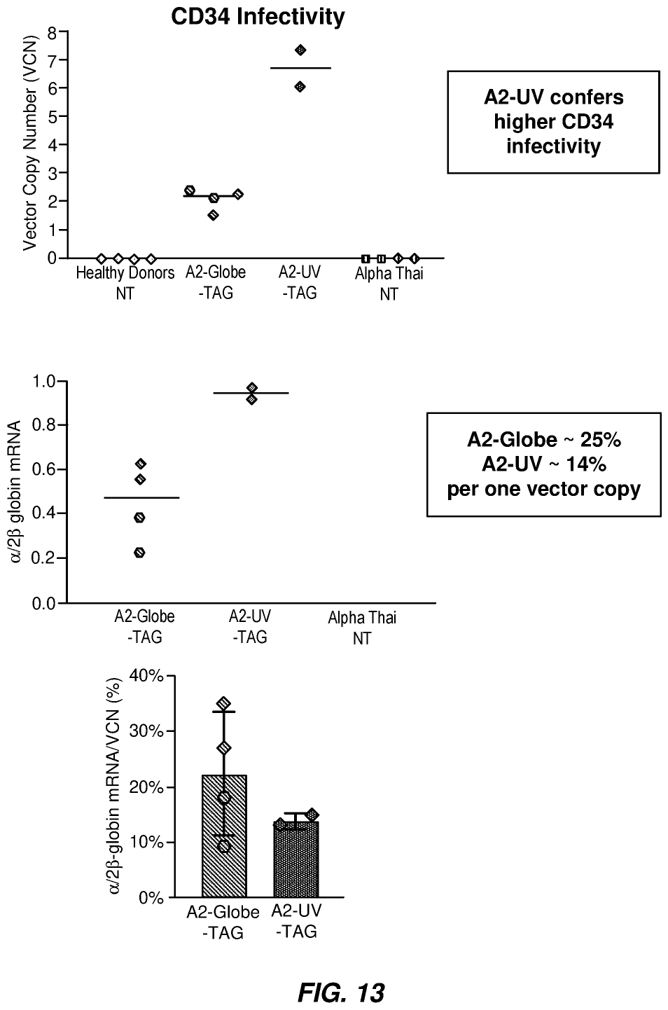

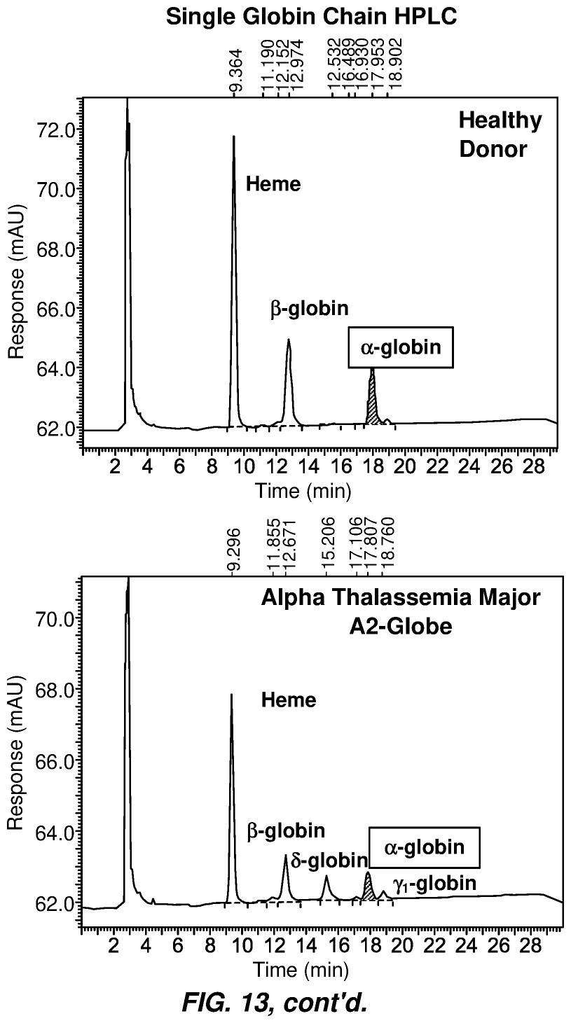

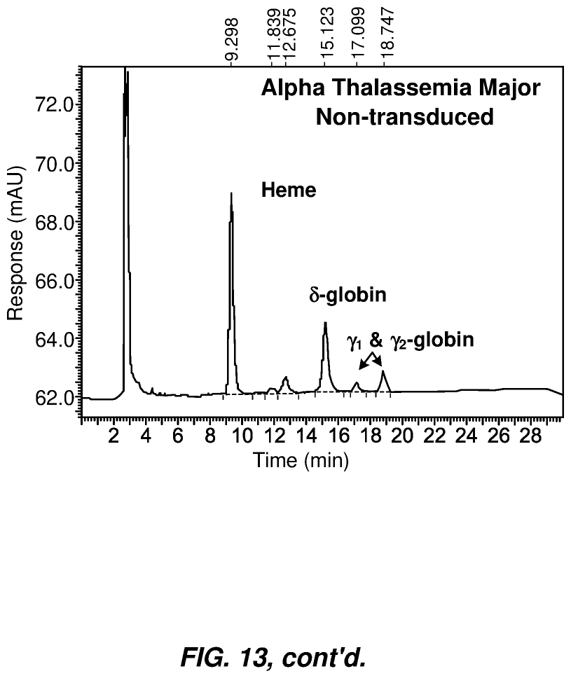

FIG. 13 shows the evaluation of alpha thalassemia major hematopoietic stem and progenitor cells. A2-UV appeared to confer higher CD34 infectivity. The A2 globe showed approximately 25% α/2β-globin mRNA per one vector copy while A2-UV showed about 14%. The A2-Globe-Tag vector appeared to show higher α/2β-globin mRNA/VCN than the A2-UV-Tag vector, although the difference may not be statistically significant. The chromatograms in this figure represents single globin chain HPLC of HSPCs differentiated into red blood cells following our in lab erythroid differentiation protocol. The top panel shows single globin chain from healthy donor, with the alpha globin chain highlighted in bleu. The chromatogram below represents red blood cells obtained after transduction and differentiation of alpha thalassemia major patient cells. As shown, alpha globin chain, highlighted in blue, is present and dominant, compared to the non-transduced patient cells. These data demonstrate alpha globin chain synthesis and thereby the correction of the alpha to beta globin chain imbalance in alpha thalassemia major.

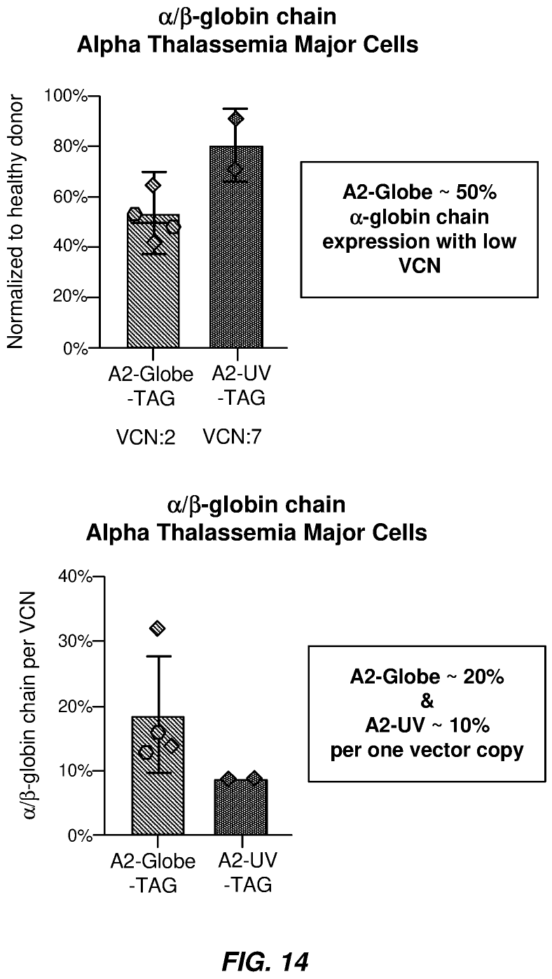

FIG. 14 illustrates α-globin and β-globin expression in alpha thalassemia major cells. A2-Globe vector showed about 50% of the endogenous α-globin chain expression in healthy donors with low VCN. When normalized to one vector copy A2-Globe showed about 20% expression, while A2-UV showed about 10% expression.

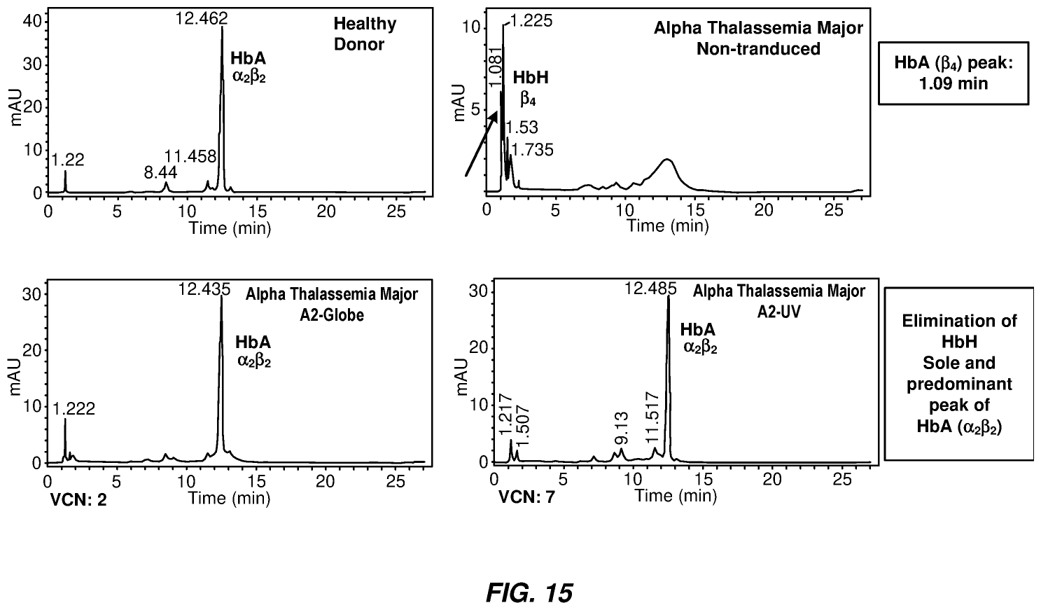

FIG. 15 illustrates hemoglobin restoration in alpha thalassemia major hematopoietic stem and progenitor cells. Hemoglobin expression was restored to significantly levels.

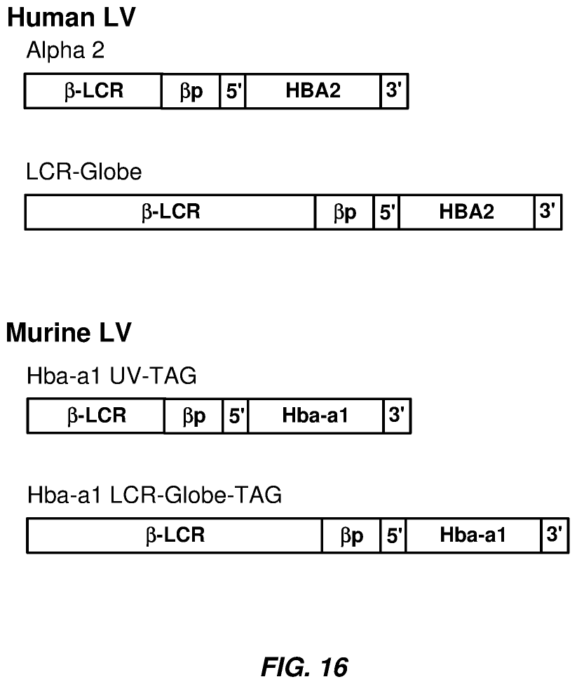

FIG. 16 schematically illustrates candidate human and murine vectors used in certain in vivo studies.

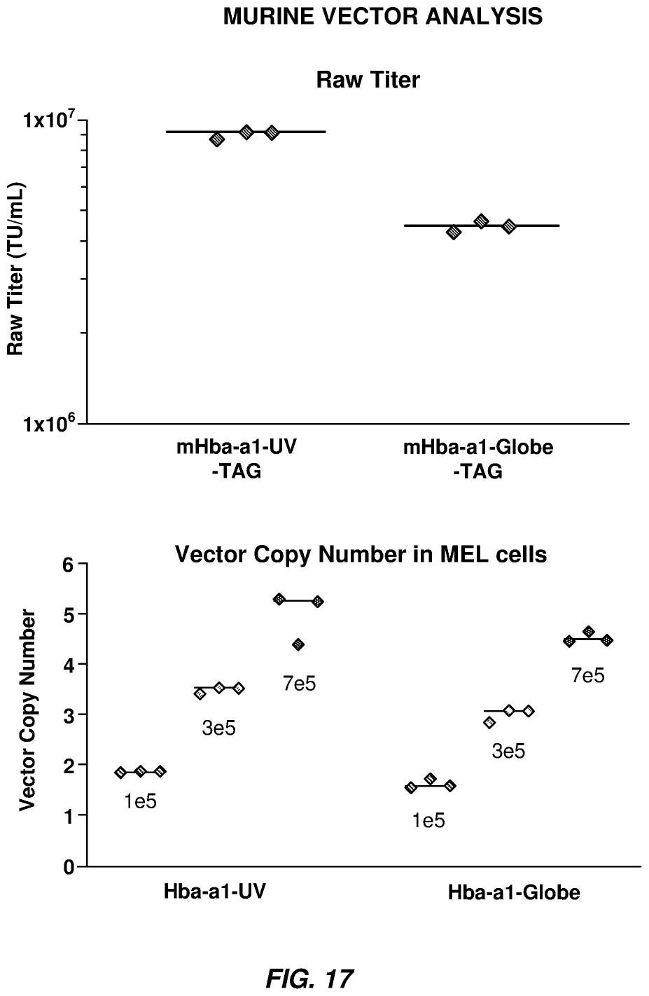

FIG. 17 shows raw titer produced by murine vectors and vector copy number produced in MEL cells.

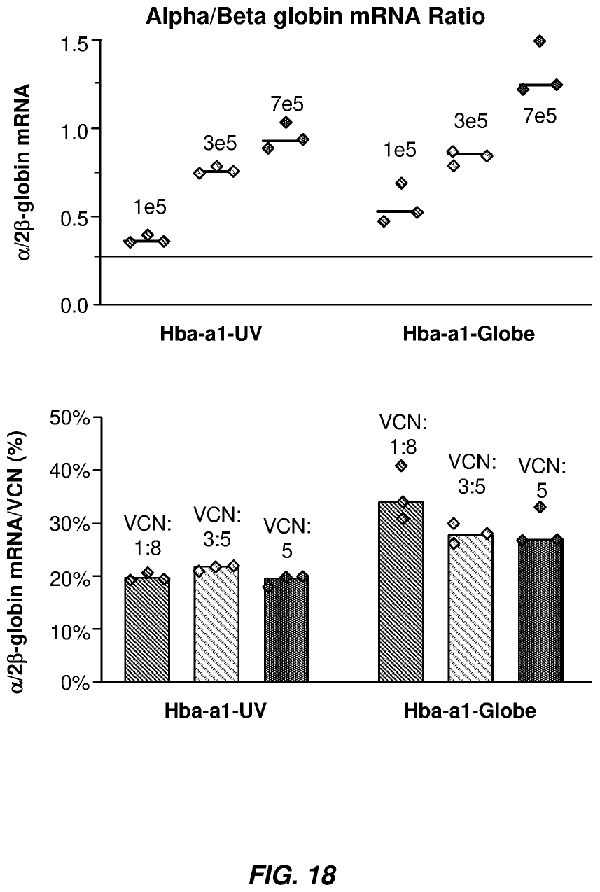

FIG. 18 shows the alpha/beta globin mRNA ration and this ratio normalized to VCN for two murine vectors.

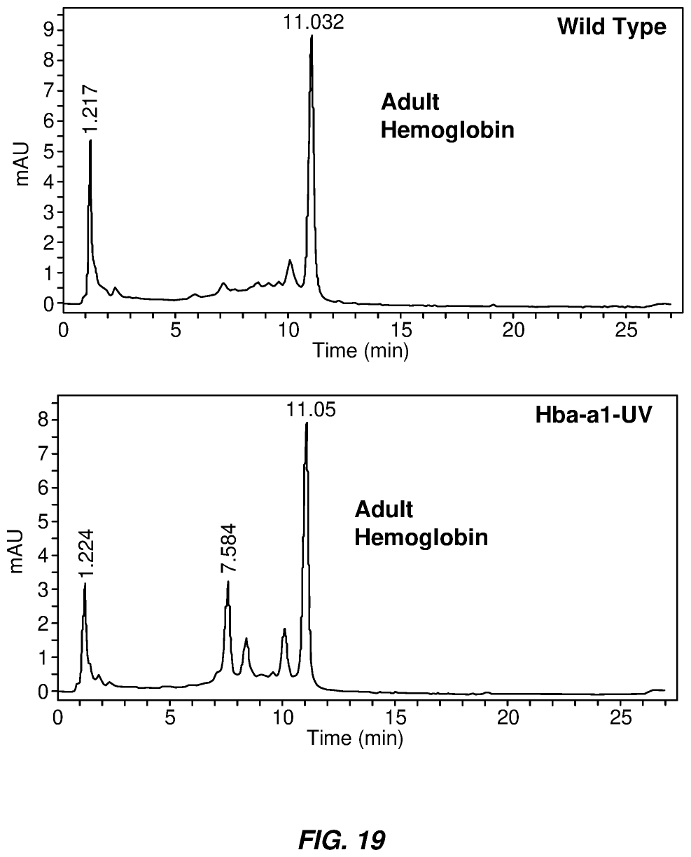

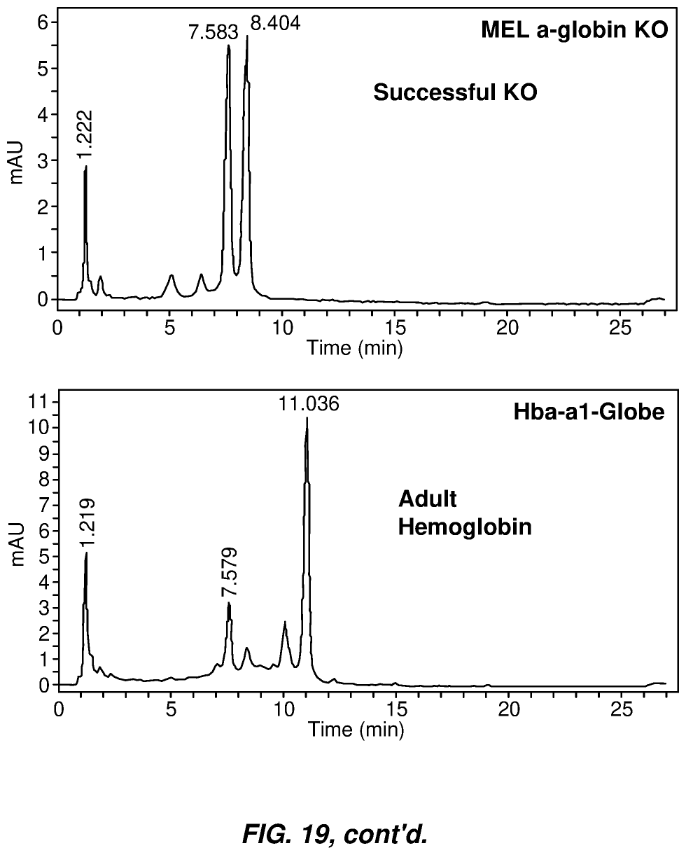

FIG. 19 shows the results of murine vector analysis in MEL cells.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

SEQUENCE LISTING
Vector I (FIG. 1, panel B).
Sequence ID No: 27
acgcgccctgtagcggcgcattaagcgcggcgggtgtggtggttacgcgcagcgtgaccgct
acacttgccagcgccctagcgcccgctcctttcgctttcttcccttcctttctcgccacgtt
cgccggctttccccgtcaagctctaaatcgggggctccctttagggttccgatttagtgctt
tacggcacctcgaccccaaaaaacttgattagggtgatggttcacgtagtgggccatcgccc
tgatagacggtttttcgccctttgacgttggagtccacgttctttaatagtggactcttgtt
ccaaactggaacaacactcaaccctatctcggtctattcttttgatttataagggattttgc
cgatttcggcctattggttaaaaaatgagctgatttaacaaaaatttaacgcgaattttaac
aaaatattaacgcttacaatttaggtggcacttttcggggaaatgtgcgcggaacccctatt
tgtttatttttctaaatacattcaaatatgtatccgctcatgagacaataaccctgataaat
gcttcaataatagcacctagatcaagagacaggatgaggatcgtttcgcatgattgaacaag
atggattgcacgcaggttctccggccgcttgggtggagaggctattcggctatgactgggca
caacagacaatcggctgctctgatgccgccgtgttccggctgtcagcgcaggggcgcccggt
tctttttgtcaagaccgacctgtccggtgccctgaatgaactgcaagacgaggcagcgcggc
tatcgtggctggccacgacgggcgttccttgcgcagctgtgctcgacgttgtcactgaagcg
ggaagggactggctgctattgggcgaagtgccggggcaggatctcctgtcatctcaccttgc
tcctgccgagaaagtatccatcatggctgatgcaatgcggcggctgcatacgcttgatccgg
ctacctgcccattcgaccaccaagcgaaacatcgcatcgagcgagcacgtactcggatggaa
gccggtcttgtcgatcaggatgatctggacgaagagcatcaggggctcgcgccagccgaact
gttcgccaggctcaaggcgagcatgcccgacggcgaggatctcgtcgtgacccatggcgatg
cctgcttgccgaatatcatggtggaaaatggccgcttttctggattcatcgactgtggccgg
ctgggtgtggcggaccgctatcaggacatagcgttggctacccgtgatattgctgaagagct
tggcggcgaatgggctgaccgcttcctcgtgctttacggtatcgccgctcccgattcgcagc
gcatcgccttctatcgccttcttgacgagttcttctgaattattaacgcttacaatttcctg
atgcggtattttctccttacgcatctgtgcggtatttcacaccgcatcaggtggcacttttc
ggggaaatgtgcgcggaacccctatttgtttatttttctaaatacattcaaatatgtatccg
ctcatgaccaaaatcccttaacgtgagttttcgttccactgagcgtcagaccccgtagaaaa
gatcaaaggatcttcttgagatcctttttttctgcgcgtaatctgctgcttgcaaacaaaaa
aaccaccgctaccagcggtggtttgtttgccggatcaagagctaccaactctttttccgaag
gtaactggcttcagcagagcgcagataccaaatactgttcttctagtgtagccgtagttagg
ccaccacttcaagaactctgtagcaccgcctacatacctcgctctgctaatcctgttaccag
tggctgctgccagtggcgataagtcgtgtcttaccgggttggactcaagacgatagttaccg
gataaggcgcagcggtcgggctgaacgggggggtcgtgcacacagcccagcttggagcgaac
gacctacaccgaactgagatacctacagcgtgagctatgagaaagcgccacgcttcccgaag
ggagaaaggcggacaggtatccggtaagcggcagggtcggaacaggagagcgcacgagggag
cttccagggggaaacgcctggtatctttatagtcctgtcgggtttcgccacctctgacttga
gcgtcgatttttgtgatgctcgtcaggggggcggagcctatggaaaaacgccagcaacgcgg
cctttttacggttcctggccttttgctggccttttgctcacatgttctttcctgcgttatcc
cctgattctgtggataaccgtattaccgcctttgagtgagctgataccgctcgccgcagccg
aacgaccgagcgcagcgagtcagtgagcgaggaagcggaagagcgcccaatacgcaaaccgc
ctctccccgcgcgttggccgattcattaatgcagctggcacgacaggtttcccgactggaaa
gcgggcagtgagcgcaacgcaattaatgtgagttagctcactcattaggcaccccaggcttt
acactttatgcttccggctcgtatgttgtgtggaattgtgagcggataacaatttcacacag
gaaacagctatgaccatgattacgccaagcgcgcaattaaccctcactaaagggaacaaaag
ctggagctgcaagcttggccattgcatacgttgtatccatatcataatatgtacatttatat
tggctcatgtccaacattaccgccatgttgacattgattattgactagttattaatagtaat
caattacggggtcattagttcatagcccatatatggagttccgcgttacataacttacggta
aatggcccgcctggctgaccgcccaacgacccccgcccattgacgtcaataatgacgtatgt
tcccatagtaacgccaatagggactttccattgacgtcaatgggtggagtatttacggtaaa
ctgcccacttggcagtacatcaagtgtatcatatgccaagtacgccccctattgacgtcaat
gacggtaaatggcccgcctggcattatgcccagtacatgaccttatgggactttcctacttg
gcagtacatctacgtattagtcatcgctattaccatggtgatgcggttttggcagtacatca
atgggcgtggatagcggtttgactcacggggatttccaagtctccaccccattgacgtcaat
gggagtttgttttggcaccaaaatcaacgggactttccaaaatgtcgtaacaactccgcccc
attgacgcaaatgggcggtaggcgtgtacggtgggaggtctatataagcagagctcgtttag
tgaaccgGGGTCTCTCTGGTTAGACCAGATCTGAGCCTGGGAGCTCTCTGGCTAACTAGGGA
ACCCACTGCTTAAGCCTCAATAAAGCTTGCCTTGAGTGCTTCAAGTAGTGTGTGCCCGTCTG
TTGTGTGACTCTGGTAACTAGAGATCCCTCAGACCCTTTTAGTCAGTGTGGAAAATCTCTAG
CagtggcgcccgaacagggacttgaaagcgaaagggaaaccagaggagctctcTCGACGCAG
GACTCGGCTTGCTGAAGCGCGCACGGCAAGAGGCGAGGGGCGGCGACTGGTGAGTACGCCAA
AAATTTTGACTAGCGGAGGCTAGAAGGAGAGAGATGGGTGCGAGAGCGTCAGTATTAAGCGG
GGGAGaattagatcgcgatgggaaaaaattcggttaaggccagggggaaagaaaaaatataa
attaaaacatatagtatgggcaagcagggagctagaacgattcgcagttaatcctggcctgt
tagaaacatcagaaggctgtagacaaatactgggacagctacaaccatcccttcagacagga
tcagaagaacttagatcattatataatacagtagcaaccctctattgtgtgcatcaaaggat
agagataaaagacaccaaggaagctttagacaagatagaggaagagcaaaacaaaagtaaga
ccaccgcacagcaagcGGCCGCTgatcttcagacctggaggaggagatatgagggacaattg
gagaagtgaattatataaatataaagtagtaaaaattgaaccattaggagtagcacccacca
aggcaaagagaagagtggtgcagagagaaaaaagaGCAGTGGGAATaggagctttgttcctt
gggttcttgggagcagcaggaagcactatgggcgcagcgtcaatgacgctgacggtacaggc
cagacaattattgtctggtatagtgcagcagcagaacaatttgctgagggctattgaggcgc
aacagcatctgttgcaactcacagtctggggcatcaagcagctccaggcaagaatcctggct
gtggaaagatacctaaaggatcaacagctcctggggatttggggttgctctggaaaactcat
ttgcaccactgctgtgccttggaatgctagttggagtaataaatctctggaacagatttgga
atcacacgacctggatggagtgggacagagaaattaacaattacacaagcttaatacactcc
ttaattgaagaatcgcaaaaccagcaagaaaagaatgaacaagaattattggaattagataa
atgggcaagtttgtggaattggtttaacataacaaattggctgtggtatataaaattattca
taatgatagtaggaggcttggtaggtttaagaatagtttttgctgtactttctatagtgaat
agagttaggcagggatattcaccattatcgtttcagacccacctcccaaccccgaggggacc
cgacaggcccgaaggaatagaagaagaaggtggagagagagacagagacagatccattcgat
tagtgaacggatCTCGACGGTATCGATCTCGAcacaaatggcagtattcatccacaatttta
aaagaaaaggggggattggggggtacagtgcaggggaaagaatagtagacataatagcaaca
gacatacaaactaaagaattacaaaaacaaattacaaaaattcaaaattttcgggtttatta
cagggacagcagacatgaggacagctaaaacaataagtaatgtaaaatacagcatagcaaaa
ctttaacctccaaatcaagcctctacttgaatccttttctgagggatgaataaggcataggc
atcaggggctgttgccaatgtgcattagctgtttgcagcctcaccttctttcatggagttta
agatatagtgtattttcccaaggtttgaactagctcttcatttctttatgttttaaatgcac
tgacctcccacattccctttttagtaaaatattcagaaataatttaaatacatcattgcaat
gaaaataaatgttttttattaggcagaatccagatgctcaaggcccttcataatatccccca
gtttagtagttggacttagggaacaaaggaacctttaatagaaattggacagcaagaaagcg
agcTTAACGGTATTTGGAGGTCAGCACGGTGCTCACAGAAGCCAGGAACTTGTCCAGGGAGG
CGTGCACCGCAGGGGTGAACTCGGCGGGGAGGTGGGCGGCCAGGGTCACCAGCAGGCAGTGG
CTTAGGAGCTGTGCAGAGAAGAGGGTCAGTGCGGCCCAGGCCCGCAGCCGCCGCCTGCGCTA
CACCTCCCGCAACCCGCGTGATCCTCTGCCCTGAGAGGAAGGCGCCATCTCGCCCCTCGACC
CAGATCGCTCCCGGCCCGCCGCTCACCTTGAAGTTGACCGGGTCCACCCGAAGCTTGTGCGC
GTGCAGGTCGCTCAGGGCGGACAGCGCGTTGGGCATGTCGTCCACGTGCGCCACGGCGTTGG
TCAGCGCGTCGGCCACCTTCTTGCCGTGGCCCTTAACCTGGGCAGAGCCGTGGCTCAGGTCG
AAGTGCGGGAAGTAGGTCTTGGTGGTGGGGAAGGACAGGAACATCCTGCGGGGAGAAGCAGA
GTGAGGGGTGGGGTTTGGGTCCGGGGCCAGGACGGTTGAGGGTGGCCTGTGGGTCCGGGCGG
GCGAGGAGCCCGGGTCGGAGCAGGGGAGGGAGCCTCACCTCTCCAGGGCCTCCGCACCATAC
TCGCCAGCGTGCGCGCCGACCTTACCCCAGGCGGCCTTGACGTTGGTCTTGTCGGCAGGAGA
CAGCACCATggtggctgtttgaggttgctagtgaacacagttgtgtcagaagcaaatgtaag
caatagatggctctgccctgacttttatgcccagccctggctcctgccctccctgctcctgg
gagtagattggccaaccctagggtgtggctccacagggtgaggtctaagtgatgacagccgt
acctgtccttggctcttctggcactggcttaggagttggacttcaaaccctcagccctccct
ctaagatatatctcttggccccataccatcagtacaaattgctactaaaaacatcctccttt
gcaagtgtatttacTACGTaTATGTGTATATATATATATATATTCAGGAAATAATATATTCT
AGAATATGTCACATTCTGTCTCAGGCATCCATTTTCTTTATGATGCCGTTTGAGGTGGAGTT
TTAGTCAGGTGGTCAGCTTCTCCTTTTTTTTGCCATCTGCCCTGTAAGCATCCTGCTGGGGA
CCCAGATAGGAGTCATCACTCTAGGCTGAGAACATCTGGGCACACACCCTAAGCCTCAGCAT
GACTCATCATGACTCAGCATTGCTGTGCTTGAGCCAGAAGGTTTGCTTAGAAGGTTACACAG
AACCAGAAGGCGGGGGTGGGGCACTGACCCCGACAGGGGCCTGGCCAGAACTGCTCATGCTT
GGACTATGGGAGGTCACTAATGGAGACACACAGAAATGTAACAGGAACTAAGGAAAAACTGA
AGCTTtgggggtataggggagcagtcccatgtagtagtagaatgaaaaatgctgctatgctg
tgcctcccccacctttcccatgtctgccctctactcatggtctatctctcctggctcctggg
agtcatggactccacccagcaccaccaacctgacctaaccacctatctgagcctgccagcct
ataacccatctgggccctgatagctggtggccagccctgaccccaccccaccctccctggaa
cctctgatagacacatctggcacaccagctcgcaaagtcaccgtgagggtcttgtgtttgct
gagtcaaaattccttgaaatccaagtccttagagactcccaggcttggattcaaagctcctg
actttctgtctagtgtatgtgcagtgagccccttttcctctaactgaaagaaggaaaaaaaa
atggaacccaaaatattctacatagtttccatgtcacagccagggctgggcagtctcctgtt
atttcttttaaaataaatatatcatttaaatgcataaataagcaaaccctgctcgggaatgg
gagggagagtctctggagtccaccccttctcggccctggctctgcagatagtgctatcaaag
ccctgacagagccctgcccattgctgggccttggagtgagtcagcctagtagagaggcaggg
caagccatctcatagctgctgagtgggagagagaaaagggctcattgtctataaactcaggt
catggctattcttattaaaagaaaaggggggactggaagggctaattcactcccaacgaaga
caagatctgctttttgcttgtactGGGTCTCTCTGGTTAGACCAGATCTGAGCCTGGGAGCT
CTCTGGCTAACTAGGGAACCCACTGCTTAAGCCTCAATAAAGCTTGCCTTGAGTGCTTCAAG
TAGTGTGTTGGTTTTTTGTGTGTGCATTGTCTGAGTAGGTGTCATTCTATTCTGGGGGGTGG
GGTGGGGCAGCACAGCAAGGGGGAGGATTGGGAAGACAATAGCAGGCATGCTGGGGATGCGG
TGGGCTCTATGGagtagtagttcatgtcatcttattattcagtatttataacttgcaaagaa
atgaatatcagagagtgagaggaacttgtttattgcagcttataatggttacaaataaagca
atagcatcacaaatttcacaaataaagcatttttttcactgcattctagttgtggtttgtcc
aaactcatcaatgtatcttatcatgtctggctctagctatcccgcccctaactccgcccATC
CCGCCCCTAACTCCGCCCAGTTCCGCCCATTCTCCGCCCCATGGCTCTCACTACTTCTGGAA
TAGCTCAGAGGCCGAGGCGGCCTCGGCCTCTGCATAAATAAAAAAAATTAGTCGAGGCTTTT
TTGGAGGCCTAGGgacgtacccaattcgccctatagtgagtcgtattacgcgcgctcactgg
ccgtcgttttacaacgtcgtgactgggaaaaccctggcgttacccaacttaatcgccttgca
gcacatccccctttcgccagctggcgtaatagcgaagaggcccgcaccgatcgcccttccca
acagttgcgcagcctgaatggcgaatggg
Vector II (FIG. 1, panel B).
Sequence ID No: 28
acgcgccctgtagcggcgcattaagcgcggcgggtgtggtggttacgcgcagcgtgaccgct
acacttgccagcgccctagcgcccgctcctttcgctttcttcccttcctttctcgccacgtt
cgccggctttccccgtcaagctctaaatcgggggctccctttagggttccgatttagtgctt
tacggcacctcgaccccaaaaaacttgattagggtgatggttcacgtagtgggccatcgccc
tgatagacggtttttcgccctttgacgttggagtccacgttctttaatagtggactcttgtt
ccaaactggaacaacactcaaccctatctcggtctattcttttgatttataagggattttgc
cgatttcggcctattggttaaaaaatgagctgatttaacaaaaatttaacgcgaattttaac
aaaatattaacgcttacaatttaggtggcacttttcggggaaatgtgcgcggaacccctatt
tgtttatttttctaaatacattcaaatatgtatccgctcatgagacaataaccctgataaat
gcttcaataatagcacctagatcaagagacaggatgaggatcgtttcgcatgattgaacaag
atggattgcacgcaggttctccggccgcttgggtggagaggctattcggctatgactgggca
caacagacaatcggctgctctgatgccgccgtgttccggctgtcagcgcaggggcgcccggt
tctttttgtcaagaccgacctgtccggtgccctgaatgaactgcaagacgaggcagcgcggc
tatcgtggctggccacgacgggcgttccttgcgcagctgtgctcgacgttgtcactgaagcg
ggaagggactggctgctattgggcgaagtgccggggcaggatctcctgtcatctcaccttgc
tcctgccgagaaagtatccatcatggctgatgcaatgcggcggctgcatacgcttgatccgg
ctacctgcccattcgaccaccaagcgaaacatcgcatcgagcgagcacgtactcggatggaa
gccggtcttgtcgatcaggatgatctggacgaagagcatcaggggctcgcgccagccgaact
gttcgccaggctcaaggcgagcatgcccgacggcgaggatctcgtcgtgacccatggcgatg
cctgcttgccgaatatcatggtggaaaatggccgcttttctggattcatcgactgtggccgg
ctgggtgtggcggaccgctatcaggacatagcgttggctacccgtgatattgctgaagagct
tggcggcgaatgggctgaccgcttcctcgtgctttacggtatcgccgctcccgattcgcagc
gcatcgccttctatcgccttcttgacgagttcttctgaattattaacgcttacaatttcctg
atgcggtattttctccttacgcatctgtgcggtatttcacaccgcatcaggtggcacttttc
ggggaaatgtgcgcggaacccctatttgtttatttttctaaatacattcaaatatgtatccg
ctcatgaccaaaatcccttaacgtgagttttcgttccactgagcgtcagaccccgtagaaaa
gatcaaaggatcttcttgagatcctttttttctgcgcgtaatctgctgcttgcaaacaaaaa
aaccaccgctaccagcggtggtttgtttgccggatcaagagctaccaactctttttccgaag
gtaactggcttcagcagagcgcagataccaaatactgttcttctagtgtagccgtagttagg
ccaccacttcaagaactctgtagcaccgcctacatacctcgctctgctaatcctgttaccag
tggctgctgccagtggcgataagtcgtgtcttaccgggttggactcaagacgatagttaccg
gataaggcgcagcggtcgggctgaacgggggggtcgtgcacacagcccagcttggagcgaac
gacctacaccgaactgagatacctacagcgtgagctatgagaaagcgccacgcttcccgaag
ggagaaaggcggacaggtatccggtaagcggcagggtcggaacaggagagcgcacgagggag
cttccagggggaaacgcctggtatctttatagtcctgtcgggtttcgccacctctgacttga
gcgtcgatttttgtgatgctcgtcaggggggcggagcctatggaaaaacgccagcaacgcgg
cctttttacggttcctggccttttgctggccttttgctcacatgttctttcctgcgttatcc
cctgattctgtggataaccgtattaccgcctttgagtgagctgataccgctcgccgcagccg
aacgaccgagcgcagcgagtcagtgagcgaggaagcggaagagcgcccaatacgcaaaccgc
ctctccccgcgcgttggccgattcattaatgcagctggcacgacaggtttcccgactggaaa
gcgggcagtgagcgcaacgcaattaatgtgagttagctcactcattaggcaccccaggcttt
acactttatgcttccggctcgtatgttgtgtggaattgtgagcggataacaatttcacacag
gaaacagctatgaccatgattacgccaagcgcgcaattaaccctcactaaagggaacaaaag
ctggagctgcaagcttggccattgcatacgttgtatccatatcataatatgtacatttatat
tggctcatgtccaacattaccgccatgttgacattgattattgactagttattaatagtaat
caattacggggtcattagttcatagcccatatatggagttccgcgttacataacttacggta
aatggcccgcctggctgaccgcccaacgacccccgcccattgacgtcaataatgacgtatgt
tcccatagtaacgccaatagggactttccattgacgtcaatgggtggagtatttacggtaaa
ctgcccacttggcagtacatcaagtgtatcatatgccaagtacgccccctattgacgtcaat
gacggtaaatggcccgcctggcattatgcccagtacatgaccttatgggactttcctacttg
gcagtacatctacgtattagtcatcgctattaccatggtgatgcggttttggcagtacatca
atgggcgtggatagcggtttgactcacggggatttccaagtctccaccccattgacgtcaat
gggagtttgttttggcaccaaaatcaacgggactttccaaaatgtcgtaacaactccgcccc
attgacgcaaatgggcggtaggcgtgtacggtgggaggtctatataagcagagctcgtttag
tgaaccgGGGTCTCTCTGGTTAGACCAGATCTGAGCCTGGGAGCTCTCTGGCTAACTAGGGA
ACCCACTGCTTAAGCCTCAATAAAGCTTGCCTTGAGTGCTTCAAGTAGTGTGTGCCCGTCTG
TTGTGTGACTCTGGTAACTAGAGATCCCTCAGACCCTTTTAGTCAGTGTGGAAAATCTCTAG
CagtggcgcccgaacagggacttgaaagcgaaagggaaaccagaggagctctcTCGACGCAG
GACTCGGCTTGCTGAAGCGCGCACGGCAAGAGGCGAGGGGCGGCGACTGGTGAGTACGCCAA
AAATTTTGACTAGCGGAGGCTAGAAGGAGAGAGATGGGTGCGAGAGCGTCAGTATTAAGCGG
GGGAGaattagatcgcgatgggaaaaaattcggttaaggccagggggaaagaaaaaatataa
attaaaacatatagtatgggcaagcagggagctagaacgattcgcagttaatcctggcctgt
tagaaacatcagaaggctgtagacaaatactgggacagctacaaccatcccttcagacagga
tcagaagaacttagatcattatataatacagtagcaaccctctattgtgtgcatcaaaggat
agagataaaagacaccaaggaagctttagacaagatagaggaagagcaaaacaaaagtaaga
ccaccgcacagcaagcGGCCGCTgatcttcagacctggaggaggagatatgagggacaattg
gagaagtgaattatataaatataaagtagtaaaaattgaaccattaggagtagcacccacca
aggcaaagagaagagtggtgcagagagaaaaaagaGCAGTGGGAATaggagctttgttcctt
gggttcttgggagcagcaggaagcactatgggcgcagcgtcaatgacgctgacggtacaggc
cagacaattattgtctggtatagtgcagcagcagaacaatttgctgagggctattgaggcgc
aacagcatctgttgcaactcacagtctggggcatcaagcagctccaggcaagaatcctggct
gtggaaagatacctaaaggatcaacagctcctggggatttggggttgctctggaaaactcat
ttgcaccactgctgtgccttggaatgctagttggagtaataaatctctggaacagatttgga
atcacacgacctggatggagtgggacagagaaattaacaattacacaagcttaatacactcc
ttaattgaagaatcgcaaaaccagcaagaaaagaatgaacaagaattattggaattagataa
atgggcaagtttgtggaattggtttaacataacaaattggctgtggtatataaaattattca
taatgatagtaggaggcttggtaggtttaagaatagtttttgctgtactttctatagtgaat
agagttaggcagggatattcaccattatcgtttcagacccacctcccaaccccgaggggacc
cgacaggcccgaaggaatagaagaagaaggtggagagagagacagagacagatccattcgat
tagtgaacggatCTCGACGGTATCGATCTCGAcacaaatggcagtattcatccacaatttta
aaagaaaaggggggattggggggtacagtgcaggggaaagaatagtagacataatagcaaca
gacatacaaactaaagaattacaaaaacaaattacaaaaattcaaaattttcgggtttatta
cagggacagcagaTGCCGCCCACTCAGACTTTATTCAAAGACCACGGGGGTACGGGTGCAGG
AAGGGGAGGAGGGGCTGGGGGGAGGCCCAAGGGGCAAGAAGCATGGCCACCGAGGCTCCAGC
TTAACGGTATTTGGAGGTCAGCACGGTGCTCACAGAAGCCAGGAACTTGTCCAGGGAGGCGT
GCACCGCAGGGGTGAACTCGGCGGGGAGGTGGGCGGCCAGGGTCACCAGCAGGCAGTGGCTT
AGGAGCTGTGCAGAGAAGAGGGTCAGTGGGGCCGAGGGCCCAGGCCCGCAGCCGCCGCCTGC
GCTACACCTCCCGCAACCCGCGTGATCCTCTGCCCTGCGAGGAAGGCGCCATCTCGCCCCTC
GACCCAGATCGCTCCCGGCCCGCCGCTCACCTTGAAGTTGACCGGGTCCACCCGAAGCTTGT
GCGCGTGCAGGTCGCTCAGGGCGGACAGCGCGTTGGGCATGTCGTCCACGTGCGCCACGGCG
TTGGTCAGCGCGTCGGCCACCTTCTTGCCGTGGCCCTTAACCTGGGCAGAGCCGTGGCTCAG
GTCGAAGTGCGGGAAGTAGGTCTTGGTGGTGGGGAAGGACAGGAACATCCTGCGGGGAGAAG
CAGAGTGAGGGGTGGGGTTTGGGTCCGGGGCCAGGACGGTTGAGGGTGGCCTGTGGGTCCGG
GCGGGCGAGGAGCCCGGGTCGGAGCAGGGGAGGGAGCCTCACCTCTCCAGGGCCTCCGCACC
ATACTCGCCAGCGTGCGCGCCGACCTTACCCCAGGCGGCCTTGACGTTGGTCTTGTCGGCAG
GAGACAGCACCATGGTGGGTTCTCTCTGAGTCTGTGGGGACCAGAAGAGTaagcaatagatg
gctctgccctgacttttatgcccagccctggctcctgccctccctgctcctgggagtagatt
ggccaaccctagggtgtggctccacagggtgaggtctaagtgatgacagccgtacctgtcct
tggctcttctggcactggcttaggagttggacttcaaaccctcagccctccctctaagatat
atctcttggccccataccatcagtacaaattgctactaaaaacatcctcctttgcaagtgta
tttacTACGTaTATGTGTATATATATATATATATTCAGGAAATAATATATTCTAGAATATGT
CACATTCTGTCTCAGGCATCCATTTTCTTTATGATGCCGTTTGAGGTGGAGTTTTAGTCAGG
TGGTCAGCTTCTCCTTTTTTTTGCCATCTGCCCTGTAAGCATCCTGCTGGGGACCCAGATAG
GAGTCATCACTCTAGGCTGAGAACATCTGGGCACACACCCTAAGCCTCAGCATGACTCATCA
TGACTCAGCATTGCTGTGCTTGAGCCAGAAGGTTTGCTTAGAAGGTTACACAGAACCAGAAG
GCGGGGGTGGGGCACTGACCCCGACAGGGGCCTGGCCAGAACTGCTCATGCTTGGACTATGG
GAGGTCACTAATGGAGACACACAGAAATGTAACAGGAACTAAGGAAAAACTGAAGCTTtggg
ggtataggggagcagtcccatgtagtagtagaatgaaaaatgctgctatgctgtgcctcccc
cacctttcccatgtctgccctctactcatggtctatctctcctggctcctgggagtcatgga
ctccacccagcaccaccaacctgacctaaccacctatctgagcctgccagcctataacccat
ctgggccctgatagctggtggccagccctgaccccaccccaccctccctggaacctctgata
gacacatctggcacaccagctcgcaaagtcaccgtgagggtcttgtgtttgctgagtcaaaa
ttccttgaaatccaagtccttagagactcccaggcttggattcaaagctcctgactttctgt
ctagtgtatgtgcagtgagccccttttcctctaactgaaagaaggaaaaaaaaatggaaccc
aaaatattctacatagtttccatgtcacagccagggctgggcagtctcctgttatttctttt
aaaataaatatatcatttaaatgcataaataagcaaaccctgctcgggaatgggagggagag
tctctggagtccaccccttctcggccctggctctgcagatagtgctatcaaagccctgacag
agccctgcccattgctgggccttggagtgagtcagcctagtagagaggcagggcaagccatc
tcatagctgctgagtgggagagagaaaagggctcattgtctataaactcaggtcatggctat
tcttattaaaagaaaaggggggactggaagggctaattcactcccaacgaagacaagatctg
ctttttgcttgtactGGGTCTCTCTGGTTAGACCAGATCTGAGCCTGGGAGCTCTCTGGCTA
ACTAGGGAACCCACTGCTTAAGCCTCAATAAAGCTTGCCTTGAGTGCTTCAAGTAGTGTGTT
GGTTTTTTGTGTGTGCATTGTCTGAGTAGGTGTCATTCTATTCTGGGGGGTGGGGTGGGGCA
GCACAGCAAGGGGGAGGATTGGGAAGACAATAGCAGGCATGCTGGGGATGCGGTGGGCTCTA
TGGagtagtagttcatgtcatcttattattcagtatttataacttgcaaagaaatgaatatc
agagagtgagaggaacttgtttattgcagcttataatggttacaaataaagcaatagcatca
caaatttcacaaataaagcatttttttcactgcattctagttgtggtttgtccaaactcatc
aatgtatcttatcatgtctggctctagctatcccgcccctaactccgcccATCCCGCCCCTA
ACTCCGCCCAGTTCCGCCCATTCTCCGCCCCATGGCTCTCACTACTTCTGGAATAGCTCAGA
GGCCGAGGCGGCCTCGGCCTCTGCATAAATAAAAAAAATTAGTCGAGGCTTTTTTGGAGGCC
TAGGgacgtacccaattcgccctatagtgagtcgtattacgcgcgctcactggccgtcgttt
tacaacgtcgtgactgggaaaaccctggcgttacccaacttaatcgccttgcagcacatccc
cctttcgccagctggcgtaatagcgaagaggcccgcaccgatcgcccttcccaacagttgcg
cagcctgaatggcgaatggg
Vector III (FIG. 1, panel B).
Sequence ID No: 29
acgcgccctgtagcggcgcattaagcgcggcgggtgtggtggttacgcgcagcgtgaccgct
acacttgccagcgccctagcgcccgctcctttcgctttcttcccttcctttctcgccacgtt
cgccggctttccccgtcaagctctaaatcgggggctccctttagggttccgatttagtgctt
tacggcacctcgaccccaaaaaacttgattagggtgatggttcacgtagtgggccatcgccc
tgatagacggtttttcgccctttgacgttggagtccacgttctttaatagtggactcttgtt
ccaaactggaacaacactcaaccctatctcggtctattcttttgatttataagggattttgc
cgatttcggcctattggttaaaaaatgagctgatttaacaaaaatttaacgcgaattttaac
aaaatattaacgcttacaatttaggtggcacttttcggggaaatgtgcgcggaacccctatt
tgtttatttttctaaatacattcaaatatgtatccgctcatgagacaataaccctgataaat
gcttcaataatagcacctagatcaagagacaggatgaggatcgtttcgcatgattgaacaag
atggattgcacgcaggttctccggccgcttgggtggagaggctattcggctatgactgggca
caacagacaatcggctgctctgatgccgccgtgttccggctgtcagcgcaggggcgcccggt
tctttttgtcaagaccgacctgtccggtgccctgaatgaactgcaagacgaggcagcgcggc
tatcgtggctggccacgacgggcgttccttgcgcagctgtgctcgacgttgtcactgaagcg
ggaagggactggctgctattgggcgaagtgccggggcaggatctcctgtcatctcaccttgc
tcctgccgagaaagtatccatcatggctgatgcaatgcggcggctgcatacgcttgatccgg
ctacctgcccattcgaccaccaagcgaaacatcgcatcgagcgagcacgtactcggatggaa
gccggtcttgtcgatcaggatgatctggacgaagagcatcaggggctcgcgccagccgaact
gttcgccaggctcaaggcgagcatgcccgacggcgaggatctcgtcgtgacccatggcgatg
cctgcttgccgaatatcatggtggaaaatggccgcttttctggattcatcgactgtggccgg
ctgggtgtggcggaccgctatcaggacatagcgttggctacccgtgatattgctgaagagct
tggcggcgaatgggctgaccgcttcctcgtgctttacggtatcgccgctcccgattcgcagc
gcatcgccttctatcgccttcttgacgagttcttctgaattattaacgcttacaatttcctg
atgcggtattttctccttacgcatctgtgcggtatttcacaccgcatcaggtggcacttttc
ggggaaatgtgcgcggaacccctatttgtttatttttctaaatacattcaaatatgtatccg
ctcatgaccaaaatcccttaacgtgagttttcgttccactgagcgtcagaccccgtagaaaa
gatcaaaggatcttcttgagatcctttttttctgcgcgtaatctgctgcttgcaaacaaaaa
aaccaccgctaccagcggtggtttgtttgccggatcaagagctaccaactctttttccgaag
gtaactggcttcagcagagcgcagataccaaatactgttcttctagtgtagccgtagttagg
ccaccacttcaagaactctgtagcaccgcctacatacctcgctctgctaatcctgttaccag
tggctgctgccagtggcgataagtcgtgtcttaccgggttggactcaagacgatagttaccg
gataaggcgcagcggtcgggctgaacgggggggtcgtgcacacagcccagcttggagcgaac
gacctacaccgaactgagatacctacagcgtgagctatgagaaagcgccacgcttcccgaag
ggagaaaggcggacaggtatccggtaagcggcagggtcggaacaggagagcgcacgagggag
cttccagggggaaacgcctggtatctttatagtcctgtcgggtttcgccacctctgacttga
gcgtcgatttttgtgatgctcgtcaggggggcggagcctatggaaaaacgccagcaacgcgg
cctttttacggttcctggccttttgctggccttttgctcacatgttctttcctgcgttatcc
cctgattctgtggataaccgtattaccgcctttgagtgagctgataccgctcgccgcagccg
aacgaccgagcgcagcgagtcagtgagcgaggaagcggaagagcgcccaatacgcaaaccgc
ctctccccgcgcgttggccgattcattaatgcagctggcacgacaggtttcccgactggaaa
gcgggcagtgagcgcaacgcaattaatgtgagttagctcactcattaggcaccccaggcttt
acactttatgcttccggctcgtatgttgtgtggaattgtgagcggataacaatttcacacag
gaaacagctatgaccatgattacgccaagcgcgcaattaaccctcactaaagggaacaaaag
ctggagctgcaagcttggccattgcatacgttgtatccatatcataatatgtacatttatat
tggctcatgtccaacattaccgccatgttgacattgattattgactagttattaatagtaat
caattacggggtcattagttcatagcccatatatggagttccgcgttacataacttacggta
aatggcccgcctggctgaccgcccaacgacccccgcccattgacgtcaataatgacgtatgt
tcccatagtaacgccaatagggactttccattgacgtcaatgggtggagtatttacggtaaa
ctgcccacttggcagtacatcaagtgtatcatatgccaagtacgccccctattgacgtcaat
gacggtaaatggcccgcctggcattatgcccagtacatgaccttatgggactttcctacttg
gcagtacatctacgtattagtcatcgctattaccatggtgatgcggttttggcagtacatca
atgggcgtggatagcggtttgactcacggggatttccaagtctccaccccattgacgtcaat
gggagtttgttttggcaccaaaatcaacgggactttccaaaatgtcgtaacaactccgcccc
attgacgcaaatgggcggtaggcgtgtacggtgggaggtctatataagcagagctcgtttag
tgaaccgGGGTCTCTCTGGTTAGACCAGATCTGAGCCTGGGAGCTCTCTGGCTAACTAGGGA
ACCCACTGCTTAAGCCTCAATAAAGCTTGCCTTGAGTGCTTCAAGTAGTGTGTGCCCGTCTG
TTGTGTGACTCTGGTAACTAGAGATCCCTCAGACCCTTTTAGTCAGTGTGGAAAATCTCTAG
CagtggcgcccgaacagggacttgaaagcgaaagggaaaccagaggagctctcTCGACGCAG
GACTCGGCTTGCTGAAGCGCGCACGGCAAGAGGCGAGGGGCGGCGACTGGTGAGTACGCCAA
AAATTTTGACTAGCGGAGGCTAGAAGGAGAGAGATGGGTGCGAGAGCGTCAGTATTAAGCGG
GGGAGaattagatcgcgatgggaaaaaattcggttaaggccagggggaaagaaaaaatataa
attaaaacatatagtatgggcaagcagggagctagaacgattcgcagttaatcctggcctgt
tagaaacatcagaaggctgtagacaaatactgggacagctacaaccatcccttcagacagga
tcagaagaacttagatcattatataatacagtagcaaccctctattgtgtgcatcaaaggat
agagataaaagacaccaaggaagctttagacaagatagaggaagagcaaaacaaaagtaaga
ccaccgcacagcaagcGGCCGCTgatcttcagacctggaggaggagatatgagggacaattg
gagaagtgaattatataaatataaagtagtaaaaattgaaccattaggagtagcacccacca
aggcaaagagaagagtggtgcagagagaaaaaagaGCAGTGGGAATaggagctttgttcctt
gggttcttgggagcagcaggaagcactatgggcgcagcgtcaatgacgctgacggtacaggc
cagacaattattgtctggtatagtgcagcagcagaacaatttgctgagggctattgaggcgc
aacagcatctgttgcaactcacagtctggggcatcaagcagctccaggcaagaatcctggct
gtggaaagatacctaaaggatcaacagctcctggggatttggggttgctctggaaaactcat
ttgcaccactgctgtgccttggaatgctagttggagtaataaatctctggaacagatttgga
atcacacgacctggatggagtgggacagagaaattaacaattacacaagcttaatacactcc
ttaattgaagaatcgcaaaaccagcaagaaaagaatgaacaagaattattggaattagataa
atgggcaagtttgtggaattggtttaacataacaaattggctgtggtatataaaattattca
taatgatagtaggaggcttggtaggtttaagaatagtttttgctgtactttctatagtgaat
agagttaggcagggatattcaccattatcgtttcagacccacctcccaaccccgaggggacc
cgacaggcccgaaggaatagaagaagaaggtggagagagagacagagacagatccattcgat
tagtgaacggatCTCGACGGTATCGATCTCGAcacaaatggcagtattcatccacaatttta
aaagaaaaggggggattggggggtacagtgcaggggaaagaatagtagacataatagcaaca
gacatacaaactaaagaattacaaaaacaaattacaaaaattcaaaattttcgggtttatta
cagggacagcagaTGCTGCCCACTCAGACTTTATTCAAAGACCAGGAAGGGCCGGTGCAAGG
AGGGGAGGAGGGCCCGTTGGGAGGCCCAGCGGGCAGGAGGAACGGCTACCGAGGCTCCAGCT
TAACGGTATTTGGAGGTCAGCACGGTGCTCACAGAAGCCAGGAACTTGTCCAGGGAGGCGTG
CACCGCAGGGGTGAACTCGGCGGGGAGGTGGGCGGCCAGGGTCACCAGCAGGCAGTGGCTTA
GGAGCTGTGCAGAGAAGAGGGTCAGTGCGGCCCAGGCCCGCAGCCGCCGCCTGCGCTACACC
TCCCGCAACCCGCGTGATCCTCTGCCCTGAGAGGAAGGCGCCATCTCGCCCCTCGACCCAGA
TCGCTCCCGGCCCGCCGCTCACCTTGAAGTTGACCGGGTCCACCCGAAGCTTGTGCGCGTGC
AGGTCGCTCAGGGCGGACAGCGCGTTGGGCATGTCGTCCACGTGCGCCACGGCGTTGGTCAG
CGCGTCGGCCACCTTCTTGCCGTGGCCCTTAACCTGGGCAGAGCCGTGGCTCAGGTCGAAGT
GCGGGAAGTAGGTCTTGGTGGTGGGGAAGGACAGGAACATCCTGCGGGGAGAAGCAGAGTGA
GGGGTGGGGTTTGGGTCCGGGGCCAGGACGGTTGAGGGTGGCCTGTGGGTCCGGGCGGGCGA
GGAGCCCGGGTCGGAGCAGGGGAGGGAGCCTCACCTCTCCAGGGCCTCCGCACCATACTCGC
CAGCGTGCGCGCCGACCTTACCCCAGGCGGCCTTGACGTTGGTCTTGTCGGCAGGAGACAGC
ACCATGGTGGGTTCTCTCTGAGTCTGTGGGGACCAGAAGAGTaagcaatagatggctctgcc
ctgacttttatgcccagccctggctcctgccctccctgctcctgggagtagattggccaacc
ctagggtgtggctccacagggtgaggtctaagtgatgacagccgtacctgtccttggctctt
ctggcactggcttaggagttggacttcaaaccctcagccctccctctaagatatatctcttg
gccccataccatcagtacaaattgctactaaaaacatcctcctttgcaagtgtatttacTAC
GTaTATGTGTATATATATATATATATTCAGGAAATAATATATTCTAGAATATGTCACATTCT
GTCTCAGGCATCCATTTTCTTTATGATGCCGTTTGAGGTGGAGTTTTAGTCAGGTGGTCAGC
TTCTCCTTTTTTTTGCCATCTGCCCTGTAAGCATCCTGCTGGGGACCCAGATAGGAGTCATC
ACTCTAGGCTGAGAACATCTGGGCACACACCCTAAGCCTCAGCATGACTCATCATGACTCAG
CATTGCTGTGCTTGAGCCAGAAGGTTTGCTTAGAAGGTTACACAGAACCAGAAGGCGGGGGT
GGGGCACTGACCCCGACAGGGGCCTGGCCAGAACTGCTCATGCTTGGACTATGGGAGGTCAC
TAATGGAGACACACAGAAATGTAACAGGAACTAAGGAAAAACTGAAGCTTtgggggtatagg
ggagcagtcccatgtagtagtagaatgaaaaatgctgctatgctgtgcctcccccacctttc
ccatgtctgccctctactcatggtctatctctcctggctcctgggagtcatggactccaccc
agcaccaccaacctgacctaaccacctatctgagcctgccagcctataacccatctgggccc
tgatagctggtggccagccctgaccccaccccaccctccctggaacctctgatagacacatc
tggcacaccagctcgcaaagtcaccgtgagggtcttgtgtttgctgagtcaaaattccttga
aatccaagtccttagagactcccaggcttggattcaaagctcctgactttctgtctagtgta
tgtgcagtgagccccttttcctctaactgaaagaaggaaaaaaaaatggaacccaaaatatt
ctacatagtttccatgtcacagccagggctgggcagtctcctgttatttcttttaaaataaa
tatatcatttaaatgcataaataagcaaaccctgctcgggaatgggagggagagtctctgga
gtccaccccttctcggccctggctctgcagatagtgctatcaaagccctgacagagccctgc
ccattgctgggccttggagtgagtcagcctagtagagaggcagggcaagccatctcatagct
gctgagtgggagagagaaaagggctcattgtctataaactcaggtcatggctattcttatta
aaagaaaaggggggactggaagggctaattcactcccaacgaagacaagatctgctttttgc
ttgtactGGGTCTCTCTGGTTAGACCAGATCTGAGCCTGGGAGCTCTCTGGCTAACTAGGGA
ACCCACTGCTTAAGCCTCAATAAAGCTTGCCTTGAGTGCTTCAAGTAGTGTGTTGGTTTTTT
GTGTGTGCATTGTCTGAGTAGGTGTCATTCTATTCTGGGGGGTGGGGTGGGGCAGCACAGCA
AGGGGGAGGATTGGGAAGACAATAGCAGGCATGCTGGGGATGCGGTGGGCTCTATGGagtag
tagttcatgtcatcttattattcagtatttataacttgcaaagaaatgaatatcagagagtg
agaggaacttgtttattgcagcttataatggttacaaataaagcaatagcatcacaaatttc
acaaataaagcatttttttcactgcattctagttgtggtttgtccaaactcatcaatgtatc
ttatcatgtctggctctagctatcccgcccctaactccgcccATCCCGCCCCTAACTCCGCC
CAGTTCCGCCCATTCTCCGCCCCATGGCTCTCACTACTTCTGGAATAGCTCAGAGGCCGAGG
CGGCCTCGGCCTCTGCATAAATAAAAAAAATTAGTCGAGGCTTTTTTGGAGGCCTAGGgacg
tacccaattcgccctatagtgagtcgtattacgcgcgctcactggccgtcgttttacaacgt
cgtgactgggaaaaccctggcgttacccaacttaatcgccttgcagcacatccccctttcgc
cagctggcgtaatagcgaagaggcccgcaccgatcgcccttcccaacagttgcgcagcctga
atggcgaatggg
Vector IV (FIG. 1, panel B).
Sequence ID No: 30
acgcgccctgtagcggcgcattaagcgcggcgggtgtggtggttacgcgcagcgtgaccgct
acacttgccagcgccctagcgcccgctcctttcgctttcttcccttcctttctcgccacgtt
cgccggctttccccgtcaagctctaaatcgggggctccctttagggttccgatttagtgctt
tacggcacctcgaccccaaaaaacttgattagggtgatggttcacgtagtgggccatcgccc
tgatagacggtttttcgccctttgacgttggagtccacgttctttaatagtggactcttgtt
ccaaactggaacaacactcaaccctatctcggtctattcttttgatttataagggattttgc
cgatttcggcctattggttaaaaaatgagctgatttaacaaaaatttaacgcgaattttaac
aaaatattaacgcttacaatttaggtggcacttttcggggaaatgtgcgcggaacccctatt
tgtttatttttctaaatacattcaaatatgtatccgctcatgagacaataaccctgataaat
gcttcaataatagcacctagatcaagagacaggatgaggatcgtttcgcatgattgaacaag
atggattgcacgcaggttctccggccgcttgggtggagaggctattcggctatgactgggca
caacagacaatcggctgctctgatgccgccgtgttccggctgtcagcgcaggggcgcccggt
tctttttgtcaagaccgacctgtccggtgccctgaatgaactgcaagacgaggcagcgcggc
tatcgtggctggccacgacgggcgttccttgcgcagctgtgctcgacgttgtcactgaagcg
ggaagggactggctgctattgggcgaagtgccggggcaggatctcctgtcatctcaccttgc
tcctgccgagaaagtatccatcatggctgatgcaatgcggcggctgcatacgcttgatccgg
ctacctgcccattcgaccaccaagcgaaacatcgcatcgagcgagcacgtactcggatggaa
gccggtcttgtcgatcaggatgatctggacgaagagcatcaggggctcgcgccagccgaact
gttcgccaggctcaaggcgagcatgcccgacggcgaggatctcgtcgtgacccatggcgatg
cctgcttgccgaatatcatggtggaaaatggccgcttttctggattcatcgactgtggccgg
ctgggtgtggcggaccgctatcaggacatagcgttggctacccgtgatattgctgaagagct
tggcggcgaatgggctgaccgcttcctcgtgctttacggtatcgccgctcccgattcgcagc
gcatcgccttctatcgccttcttgacgagttcttctgaattattaacgcttacaatttcctg
atgcggtattttctccttacgcatctgtgcggtatttcacaccgcatcaggtggcacttttc
ggggaaatgtgcgcggaacccctatttgtttatttttctaaatacattcaaatatgtatccg
ctcatgaccaaaatcccttaacgtgagttttcgttccactgagcgtcagaccccgtagaaaa
gatcaaaggatcttcttgagatcctttttttctgcgcgtaatctgctgcttgcaaacaaaaa
aaccaccgctaccagcggtggtttgtttgccggatcaagagctaccaactctttttccgaag
gtaactggcttcagcagagcgcagataccaaatactgttcttctagtgtagccgtagttagg
ccaccacttcaagaactctgtagcaccgcctacatacctcgctctgctaatcctgttaccag
tggctgctgccagtggcgataagtcgtgtcttaccgggttggactcaagacgatagttaccg
gataaggcgcagcggtcgggctgaacgggggggtcgtgcacacagcccagcttggagcgaac
gacctacaccgaactgagatacctacagcgtgagctatgagaaagcgccacgcttcccgaag
ggagaaaggcggacaggtatccggtaagcggcagggtcggaacaggagagcgcacgagggag
cttccagggggaaacgcctggtatctttatagtcctgtcgggtttcgccacctctgacttga
gcgtcgatttttgtgatgctcgtcaggggggcggagcctatggaaaaacgccagcaacgcgg
cctttttacggttcctggccttttgctggccttttgctcacatgttctttcctgcgttatcc
cctgattctgtggataaccgtattaccgcctttgagtgagctgataccgctcgccgcagccg
aacgaccgagcgcagcgagtcagtgagcgaggaagcggaagagcgcccaatacgcaaaccgc
ctctccccgcgcgttggccgattcattaatgcagctggcacgacaggtttcccgactggaaa
gcgggcagtgagcgcaacgcaattaatgtgagttagctcactcattaggcaccccaggcttt
acactttatgcttccggctcgtatgttgtgtggaattgtgagcggataacaatttcacacag
gaaacagctatgaccatgattacgccaagcgcgcaattaaccctcactaaagggaacaaaag
ctggagctgcaagcttggccattgcatacgttgtatccatatcataatatgtacatttatat
tggctcatgtccaacattaccgccatgttgacattgattattgactagttattaatagtaat
caattacggggtcattagttcatagcccatatatggagttccgcgttacataacttacggta
aatggcccgcctggctgaccgcccaacgacccccgcccattgacgtcaataatgacgtatgt
tcccatagtaacgccaatagggactttccattgacgtcaatgggtggagtatttacggtaaa
ctgcccacttggcagtacatcaagtgtatcatatgccaagtacgccccctattgacgtcaat
gacggtaaatggcccgcctggcattatgcccagtacatgaccttatgggactttcctacttg
gcagtacatctacgtattagtcatcgctattaccatggtgatgcggttttggcagtacatca
atgggcgtggatagcggtttgactcacggggatttccaagtctccaccccattgacgtcaat
gggagtttgttttggcaccaaaatcaacgggactttccaaaatgtcgtaacaactccgcccc
attgacgcaaatgggcggtaggcgtgtacggtgggaggtctatataagcagagctcgtttag
tgaaccgGGGTCTCTCTGGTTAGACCAGATCTGAGCCTGGGAGCTCTCTGGCTAACTAGGGA
ACCCACTGCTTAAGCCTCAATAAAGCTTGCCTTGAGTGCTTCAAGTAGTGTGTGCCCGTCTG
TTGTGTGACTCTGGTAACTAGAGATCCCTCAGACCCTTTTAGTCAGTGTGGAAAATCTCTAG
CagtggcgcccgaacagggacttgaaagcgaaagggaaaccagaggagctctcTCGACGCAG
GACTCGGCTTGCTGAAGCGCGCACGGCAAGAGGCGAGGGGCGGCGACTGGTGAGTACGCCAA
AAATTTTGACTAGCGGAGGCTAGAAGGAGAGAGATGGGTGCGAGAGCGTCAGTATTAAGCGG
GGGAGaattagatcgcgatgggaaaaaattcggttaaggccagggggaaagaaaaaatataa
attaaaacatatagtatgggcaagcagggagctagaacgattcgcagttaatcctggcctgt
tagaaacatcagaaggctgtagacaaatactgggacagctacaaccatcccttcagacagga
tcagaagaacttagatcattatataatacagtagcaaccctctattgtgtgcatcaaaggat
agagataaaagacaccaaggaagctttagacaagatagaggaagagcaaaacaaaagtaaga
ccaccgcacagcaagcGGCCGCTgatcttcagacctggaggaggagatatgagggacaattg
gagaagtgaattatataaatataaagtagtaaaaattgaaccattaggagtagcacccacca
aggcaaagagaagagtggtgcagagagaaaaaagaGCAGTGGGAATaggagctttgttcctt
gggttcttgggagcagcaggaagcactatgggcgcagcgtcaatgacgctgacggtacaggc
cagacaattattgtctggtatagtgcagcagcagaacaatttgctgagggctattgaggcgc
aacagcatctgttgcaactcacagtctggggcatcaagcagctccaggcaagaatcctggct
gtggaaagatacctaaaggatcaacagctcctggggatttggggttgctctggaaaactcat
ttgcaccactgctgtgccttggaatgctagttggagtaataaatctctggaacagatttgga
atcacacgacctggatggagtgggacagagaaattaacaattacacaagcttaatacactcc
ttaattgaagaatcgcaaaaccagcaagaaaagaatgaacaagaattattggaattagataa
atgggcaagtttgtggaattggtttaacataacaaattggctgtggtatataaaattattca
taatgatagtaggaggcttggtaggtttaagaatagtttttgctgtactttctatagtgaat
agagttaggcagggatattcaccattatcgtttcagacccacctcccaaccccgaggggacc
cgacaggcccgaaggaatagaagaagaaggtggagagagagacagagacagatccattcgat
tagtgaacggatCTCGACGGTATCGATCTCGAcacaaatggcagtattcatccacaatttta
aaagaaaaggggggattggggggtacagtgcaggggaaagaatagtagacataatagcaaca
gacatacaaactaaagaattacaaaaacaaattacaaaaattcaaaattttcgggtttatta
cagggacagcagaTGCTGCCCACTCAGACTTTATTCAAAGACCAGGAAGGGCCGGTGCAAGG
AGGGGAGGAGGGCCCGTTGGGAGGCCCAGCGGGCAGGAGGAACGGCTACCGAGGCTCCAGCT
TAACGGTATTTGGAGGTCAGCACGGTGCTCACAGAAGCCAGGAACTTGTCCAGGGAGGCGTG
CACCGCAGGGGTGAACTCGGCGGGGAGGTGGGCGGCCAGGGTCACCAGCAGGCAGTGGCTTA
GGAGCTGTGCAGAGAAGAGGGTCAGTGCGGCCCAGGCCCGCAGCCGCCGCCTGCGCTACACC
TCCCGCAACCCGCGTGATCCTCTGCCCTGAGAGGAAGGCGCCATCTCGCCCCTCGACCCAGA
TCGCTCCCGGCCCGCCGCTCACCTTGAAGTTGACCGGGTCCACCCGAAGCTTGTGCGCGTGC
AGGTCGCTCAGGGCGGACAGCGCGTTGGGCATGTCGTCCACGTGCGCCACGGCGTTGGTCAG
CGCGTCGGCCACCTTCTTGCCGTGGCCCTTAACCTGGGCAGAGCCGTGGCTCAGGTCGAAGT
GCGGGAAGTAGGTCTTGGTGGTGGGGAAGGACAGGAACATCCTGCGGGGAGAAGCAGAGTGA
GGGGTGGGGTTTGGGTCCGGGGCCAGGACGGTTGAGGGTGGCCTGTGGGTCCGGGCGGGCGA
GGAGCCCGGGTCGGAGCAGGGGAGGGAGCCTCACCTCTCCAGGGCCTCCGCACCATACTCGC
CAGCGTGCGCGCCGACCTTACCCCAGGCGGCCTTGACGTTGGTCTTGTCGGCAGGAGACAGC
ACCATGGTGGGTTCTCTCTGAGTCTGTGGGGACCAGAAGAGTaagcaatagatggctctgcc
ctgacttttatgcccagccctggctcctgccctccctgctcctgggagtagattggccaacc
ctagggtgtggctccacagggtgaggtctaagtgatgacagccgtacctgtccttggctctt
ctggcactggcttaggagttggacttcaaaccctcagccctccctctaagatatatctcttg
gccccataccatcagtacaaattgctactaaaaacatcctcctttgcaagtgtatttacCTC
TCAGATAAACAGGAGGGGGAGGCAGCACCAGAGGTTGTAATCAGCAGTACCATTCACCCCTT
TCCCTTGTCCCACCTCCACCCCCACAGGATGACTCAGCAGTCCTGTTGGCCTCCAGAAGCAC
TGAGTCATGGTTGGCCCAGTTATCTGCTCCCTCAAGTGTTTTCACAGACCCCCACAGCTGCA
TGCCTCCACCCTTGGCTTGGGCAGATGTGCTTGCCTGGCTGACTGTGGTCCCTGATAGGTTC
CAGAGTACGTaTATGTGTATATATATATATATATTCAGGAAATAATATATTCTAGAATATGT
CACATTCTGTCTCAGGCATCCATTTTCTTTATGATGCCGTTTGAGGTGGAGTTTTAGTCAGG
TGGTCAGCTTCTCCTTTTTTTTGCCATCTGCCCTGTAAGCATCCTGCTGGGGACCCAGATAG
GAGTCATCACTCTAGGCTGAGAACATCTGGGCACACACCCTAAGCCTCAGCATGACTCATCA
TGACTCAGCATTGCTGTGCTTGAGCCAGAAGGTTTGCTTAGAAGGTTACACAGAACCAGAAG
GCGGGGGTGGGGCACTGACCCCGACAGGGGCCTGGCCAGAACTGCTCATGCTTGGACTATGG
GAGGTCACTAATGGAGACACACAGAAATGTAACAGGAACTAAGGAAAAACTGAAGCTTtggg
ggtataggggagcagtcccatgtagtagtagaatgaaaaatgctgctatgctgtgcctcccc
cacctttcccatgtctgccctctactcatggtctatctctcctggctcctgggagtcatgga
ctccacccagcaccaccaacctgacctaaccacctatctgagcctgccagcctataacccat
ctgggccctgatagctggtggccagccctgaccccaccccaccctccctggaacctctgata
gacacatctggcacaccagctcgcaaagtcaccgtgagggtcttgtgtttgctgagtcaaaa
ttccttgaaatccaagtccttagagactcccaggcttggattcaaagctcctgactttctgt
ctagtgtatgtgcagtgagccccttttcctctaactgaaagaaggaaaaaaaaatggaaccc
aaaatattctacatagtttccatgtcacagccagggctgggcagtctcctgttatttctttt
aaaataaatatatcatttaaatgcataaataagcaaaccctgctcgggaatgggagggagag
tctctggagtccaccccttctcggccctggctctgcagatagtgctatcaaagccctgacag
agccctgcccattgctgggccttggagtgagtcagcctagtagagaggcagggcaagccatc
tcatagctgctgagtgggagagagaaaagggctcattgtctataaactcaggtcatggctat
tcttattaaaagaaaaggggggactggaagggctaattcactcccaacgaagacaagatctg
ctttttgcttgtactGGGTCTCTCTGGTTAGACCAGATCTGAGCCTGGGAGCTCTCTGGCTA
ACTAGGGAACCCACTGCTTAAGCCTCAATAAAGCTTGCCTTGAGTGCTTCAAGTAGTGTGTT
GGTTTTTTGTGTGTGCATTGTCTGAGTAGGTGTCATTCTATTCTGGGGGGTGGGGTGGGGCA
GCACAGCAAGGGGGAGGATTGGGAAGACAATAGCAGGCATGCTGGGGATGCGGTGGGCTCTA
TGGagtagtagttcatgtcatcttattattcagtatttataacttgcaaagaaatgaatatc
agagagtgagaggaacttgtttattgcagcttataatggttacaaataaagcaatagcatca
caaatttcacaaataaagcatttttttcactgcattctagttgtggtttgtccaaactcatc
aatgtatcttatcatgtctggctctagctatcccgcccctaactccgcccATCCCGCCCCTA
ACTCCGCCCAGTTCCGCCCATTCTCCGCCCCATGGCTCTCACTACTTCTGGAATAGCTCAGA
GGCCGAGGCGGCCTCGGCCTCTGCATAAATAAAAAAAATTAGTCGAGGCTTTTTTGGAGGCC
TAGGgacgtacccaattcgccctatagtgagtcgtattacgcgcgctcactggccgtcgttt
tacaacgtcgtgactgggaaaaccctggcgttacccaacttaatcgccttgcagcacatccc
cctttcgccagctggcgtaatagcgaagaggcccgcaccgatcgcccttcccaacagttgcg
cagcctgaatggcgaatggg
Vector V (FIG. 1, panel B).
Sequence ID No: 31
acgcgccctgtagcggcgcattaagcgcggcgggtgtggtggttacgcgcagcgtgaccgct
acacttgccagcgccctagcgcccgctcctttcgctttcttcccttcctttctcgccacgtt
cgccggctttccccgtcaagctctaaatcgggggctccctttagggttccgatttagtgctt
tacggcacctcgaccccaaaaaacttgattagggtgatggttcacgtagtgggccatcgccc
tgatagacggtttttcgccctttgacgttggagtccacgttctttaatagtggactcttgtt
ccaaactggaacaacactcaaccctatctcggtctattcttttgatttataagggattttgc
cgatttcggcctattggttaaaaaatgagctgatttaacaaaaatttaacgcgaattttaac
aaaatattaacgcttacaatttaggtggcacttttcggggaaatgtgcgcggaacccctatt
tgtttatttttctaaatacattcaaatatgtatccgctcatgagacaataaccctgataaat
gcttcaataatagcacctagatcaagagacaggatgaggatcgtttcgcatgattgaacaag
atggattgcacgcaggttctccggccgcttgggtggagaggctattcggctatgactgggca
caacagacaatcggctgctctgatgccgccgtgttccggctgtcagcgcaggggcgcccggt
tctttttgtcaagaccgacctgtccggtgccctgaatgaactgcaagacgaggcagcgcggc
tatcgtggctggccacgacgggcgttccttgcgcagctgtgctcgacgttgtcactgaagcg
ggaagggactggctgctattgggcgaagtgccggggcaggatctcctgtcatctcaccttgc
tcctgccgagaaagtatccatcatggctgatgcaatgcggcggctgcatacgcttgatccgg
ctacctgcccattcgaccaccaagcgaaacatcgcatcgagcgagcacgtactcggatggaa
gccggtcttgtcgatcaggatgatctggacgaagagcatcaggggctcgcgccagccgaact
gttcgccaggctcaaggcgagcatgcccgacggcgaggatctcgtcgtgacccatggcgatg
cctgcttgccgaatatcatggtggaaaatggccgcttttctggattcatcgactgtggccgg
ctgggtgtggcggaccgctatcaggacatagcgttggctacccgtgatattgctgaagagct
tggcggcgaatgggctgaccgcttcctcgtgctttacggtatcgccgctcccgattcgcagc
gcatcgccttctatcgccttcttgacgagttcttctgaattattaacgcttacaatttcctg
atgcggtattttctccttacgcatctgtgcggtatttcacaccgcatcaggtggcacttttc
ggggaaatgtgcgcggaacccctatttgtttatttttctaaatacattcaaatatgtatccg
ctcatgaccaaaatcccttaacgtgagttttcgttccactgagcgtcagaccccgtagaaaa
gatcaaaggatcttcttgagatcctttttttctgcgcgtaatctgctgcttgcaaacaaaaa
aaccaccgctaccagcggtggtttgtttgccggatcaagagctaccaactctttttccgaag
gtaactggcttcagcagagcgcagataccaaatactgttcttctagtgtagccgtagttagg
ccaccacttcaagaactctgtagcaccgcctacatacctcgctctgctaatcctgttaccag
tggctgctgccagtggcgataagtcgtgtcttaccgggttggactcaagacgatagttaccg
gataaggcgcagcggtcgggctgaacgggggggtcgtgcacacagcccagcttggagcgaac
gacctacaccgaactgagatacctacagcgtgagctatgagaaagcgccacgcttcccgaag
ggagaaaggcggacaggtatccggtaagcggcagggtcggaacaggagagcgcacgagggag
cttccagggggaaacgcctggtatctttatagtcctgtcgggtttcgccacctctgacttga
gcgtcgatttttgtgatgctcgtcaggggggcggagcctatggaaaaacgccagcaacgcgg
cctttttacggttcctggccttttgctggccttttgctcacatgttctttcctgcgttatcc
cctgattctgtggataaccgtattaccgcctttgagtgagctgataccgctcgccgcagccg
aacgaccgagcgcagcgagtcagtgagcgaggaagcggaagagcgcccaatacgcaaaccgc
ctctccccgcgcgttggccgattcattaatgcagctggcacgacaggtttcccgactggaaa
gcgggcagtgagcgcaacgcaattaatgtgagttagctcactcattaggcaccccaggcttt
acactttatgcttccggctcgtatgttgtgtggaattgtgagcggataacaatttcacacag
gaaacagctatgaccatgattacgccaagcgcgcaattaaccctcactaaagggaacaaaag
ctggagctgcaagcttggccattgcatacgttgtatccatatcataatatgtacatttatat
tggctcatgtccaacattaccgccatgttgacattgattattgactagttattaatagtaat
caattacggggtcattagttcatagcccatatatggagttccgcgttacataacttacggta
aatggcccgcctggctgaccgcccaacgacccccgcccattgacgtcaataatgacgtatgt
tcccatagtaacgccaatagggactttccattgacgtcaatgggtggagtatttacggtaaa
ctgcccacttggcagtacatcaagtgtatcatatgccaagtacgccccctattgacgtcaat
gacggtaaatggcccgcctggcattatgcccagtacatgaccttatgggactttcctacttg
gcagtacatctacgtattagtcatcgctattaccatggtgatgcggttttggcagtacatca
atgggcgtggatagcggtttgactcacggggatttccaagtctccaccccattgacgtcaat
gggagtttgttttggcaccaaaatcaacgggactttccaaaatgtcgtaacaactccgcccc
attgacgcaaatgggcggtaggcgtgtacggtgggaggtctatataagcagagctcgtttag
tgaaccgGGGTCTCTCTGGTTAGACCAGATCTGAGCCTGGGAGCTCTCTGGCTAACTAGGGA
ACCCACTGCTTAAGCCTCAATAAAGCTTGCCTTGAGTGCTTCAAGTAGTGTGTGCCCGTCTG
TTGTGTGACTCTGGTAACTAGAGATCCCTCAGACCCTTTTAGTCAGTGTGGAAAATCTCTAG
CagtggcgcccgaacagggacttgaaagcgaaagggaaaccagaggagctctcTCGACGCAG
GACTCGGCTTGCTGAAGCGCGCACGGCAAGAGGCGAGGGGCGGCGACTGGTGAGTACGCCAA
AAATTTTGACTAGCGGAGGCTAGAAGGAGAGAGATGGGTGCGAGAGCGTCAGTATTAAGCGG
GGGAGaattagatcgcgatgggaaaaaattcggttaaggccagggggaaagaaaaaatataa
attaaaacatatagtatgggcaagcagggagctagaacgattcgcagttaatcctggcctgt
tagaaacatcagaaggctgtagacaaatactgggacagctacaaccatcccttcagacagga
tcagaagaacttagatcattatataatacagtagcaaccctctattgtgtgcatcaaaggat
agagataaaagacaccaaggaagctttagacaagatagaggaagagcaaaacaaaagtaaga
ccaccgcacagcaagcGGCCGCTgatcttcagacctggaggaggagatatgagggacaattg
gagaagtgaattatataaatataaagtagtaaaaattgaaccattaggagtagcacccacca
aggcaaagagaagagtggtgcagagagaaaaaagaGCAGTGGGAATaggagctttgttcctt
gggttcttgggagcagcaggaagcactatgggcgcagcgtcaatgacgctgacggtacaggc
cagacaattattgtctggtatagtgcagcagcagaacaatttgctgagggctattgaggcgc
aacagcatctgttgcaactcacagtctggggcatcaagcagctccaggcaagaatcctggct
gtggaaagatacctaaaggatcaacagctcctggggatttggggttgctctggaaaactcat
ttgcaccactgctgtgccttggaatgctagttggagtaataaatctctggaacagatttgga
atcacacgacctggatggagtgggacagagaaattaacaattacacaagcttaatacactcc
ttaattgaagaatcgcaaaaccagcaagaaaagaatgaacaagaattattggaattagataa
atgggcaagtttgtggaattggtttaacataacaaattggctgtggtatataaaattattca
taatgatagtaggaggcttggtaggtttaagaatagtttttgctgtactttctatagtgaat
agagttaggcagggatattcaccattatcgtttcagacccacctcccaaccccgaggggacc
cgacaggcccgaaggaatagaagaagaaggtggagagagagacagagacagatccattcgat
tagtgaacggatCTCGACGGTATCGATCTCGAcacaaatggcagtattcatccacaatttta
aaagaaaaggggggattggggggtacagtgcaggggaaagaatagtagacataatagcaaca
gacatacaaactaaagaattacaaaaacaaattacaaaaattcaaaattttcgggtttatta
cagggacagcagaTGCTGCCCACTCAGACTTTATTCAAAGACCAGGAAGGGCCGGTGCAAGG
AGGGGAGGAGGGCCCGTTGGGAGGCCCAGCGGGCAGGAGGAACGGCTACCGAGGCTCCAGCT
TAACGGTATTTGGAGGTCAGCACGGTGCTCACAGAAGCCAGGAACTTGTCCAGGGAGGCGTG
CACCGCAGGGGTGAACTCGGCGGGGAGGTGGGCGGCCAGGGTCACCAGCAGGCAGTGGCTTA
GGAGCTGTGCAGAGAAGAGGGTCAGTGCGGCCCAGGCCCGCAGCCGCCGCCTGCGCTACACC
TCCCGCAACCCGCGTGATCCTCTGCCCTGAGAGGAAGGCGCCATCTCGCCCCTCGACCCAGA
TCGCTCCCGGCCCGCCGCTCACCTTGAAGTTGACCGGGTCCACCCGAAGCTTGTGCGCGTGC
AGGTCGCTCAGGGCGGACAGCGCGTTGGGCATGTCGTCCACGTGCGCCACGGCGTTGGTCAG
CGCGTCGGCCACCTTCTTGCCGTGGCCCTTAACCTGGGCAGAGCCGTGGCTCAGGTCGAAGT
GCGGGAAGTAGGTCTTGGTGGTGGGGAAGGACAGGAACATCCTGCGGGGAGAAGCAGAGTGA
GGGGTGGGGTTTGGGTCCGGGGCCAGGACGGTTGAGGGTGGCCTGTGGGTCCGGGCGGGCGA
GGAGCCCGGGTCGGAGCAGGGGAGGGAGCCTCACCTCTCCAGGGCCTCCGCACCATACTCGC
CAGCGTGCGCGCCGACCTTACCCCAGGCGGCCTTGACGTTGGTCTTGTCGGCAGGAGACAGC
ACCATGGTGGGTTCTCTCTGAGTCTGTGGGGACCAGAAGAGTaagcaatagatggctctgcc
ctgacttttatgcccagccctggctcctgccctccctgctcctgggagtagattggccaacc
ctagggtgtggctccacagggtgaggtctaagtgatgacagccgtacctgtccttggctctt
ctggcactggcttaggagttggacttcaaaccctcagccctccctctaagatatatctcttg
gccccataccatcagtacaaattgctactaaaaacatcctcctttgcaagtgtatttaccct
tttgccacctagctgtccaggggtgccttaaaatggcaaacaaggtttgttttcttttcctg
ttttcatgccttcctcttccatatccttgtttcatattaatacatgtgtatagatcctaaaa
atctatacacatgtattaataaagcctgattctgccgcttctaggtatagaggccacctgca
agataaatatttgattcacaataactaatcattctatggcaattgataacaacaaatatata
tatatatatatatacgtatatgtgtatatatatatatatatattcaggaaataatatattct
agaatatgtcacattctgtctcaggcatccattttctttatgatgccgtttgaggtggagtt
ttagtcaggtggtcagcttctccttttttttgccatctgccctgtaagcatcctgctgggga
cccagataggagtcatcactctaggctgagaacatctgggcacacaccctaagcctcagcat
gactcatcatgactcagcattgctgtgcttgagccagaaggtttgcttagaaggttacacag
aaccagaaggcgggggtggggcactgaccccgacaggggcctggccagaactgctcatgctt
ggactatgggaggtcactaatggagacacacagaaatgtaacaggaactaaggaaaaactga
agcttatttaatcagagatgaggatgctggaagggatagagggagctgagcttgtaaaaagt
atagtaatcattcagcaaatggttttgaagcacctgctggatgctaaacactattttcagtg
cttgaatcataaataagaataaaacatgtatcttattccccacaagagtccaagtaaaaaat
aacagttaattataatgtgctctgtcccccaggctggagtgcagtggcacgatctcagctca
ctgcaacctccgcctcccgggttcaagcaattctcctgcctcagccaccctaatagctggga
ttacaggtgcacaccaccatgccaggctaatttttgtactttttgtagaggcagggtatcac
catgttgtccaagatggtcttgaactcctgagctccaagcagtccacccacctcagcctccc
aaagtgctgggattacaggtgtgagacaccatgcccagattttccatatttaatagaggtat
ttatgggatgggggaaaagaatgtttctctcactgtggattattttagagagtggagaatgg
tcaagatttttttaaaaattaagaaaacataagttggaccttgagaaatgaaaatttatttt
tttgttggaggatacccattctctatctcccatcagggcaagctgtaaggaactggctaaga
cacagtgagacagagtgacttagtcttagaggccccactggtacgacggtcaccaagctttc
attaaaaaaagtctaaccagctgcattcgactttgactgcagcagctggttagaaggttcta
ctggaggagggtcccagcccattgctaaattaacatcaggctctgagactggcagtatatct
ctaacagtggttgatgctatcttctggaacttgcctgctacattgagaccactgacccatac
ataggaagcccatagctctgtcctgaactgttaggccactggtccagagagtgtgcatctcc
tttgatcctcataataaccctatgagatagacacaattattactcttactttatagatgatg
atcctgaaaacataggagtcaaggcacttgcccctagctgggggtataggggagcagtccca
tgtagtagtagaatgaaaaatgctgctatgctgtgcctcccccacctttcccatgtctgccc
tctactcatggtctatctctcctggctcctgggagtcatggactccacccagcaccaccaac
ctgacctaaccacctatctgagcctgccagcctataacccatctgggccctgatagctggtg
gccagccctgaccccaccccaccctccctggaacctctgatagacacatctggcacaccagc
tcgcaaagtcaccgtgagggtcttgtgtttgctgagtcaaaattccttgaaatccaagtcct
tagagactcctgctcccaaatttacagtcatagacttcttcatggctgtctcctttatccac
agaatgattcctttgcttcattgccccatccatctgatcctcctcatcagtgcagcacaggg
cccatgagcagtagctgcagagtctcacataggtctggcactgcctctgacatgtccgacct
taggcaaatgcttgactcttctgagctcagtcttgtcatggcaaaataaagataataatagt
gtttttttatggagttagcgtgaggatggaaaacaatagcaaaattgattagactataaaag
gtctcaacaaatagtagtagattttatcatccattaatccttccctctcctctcttactcat
cccatcacgtatgcctcttaattttcccttacctataataagagttattcctcttattatat
tcttcttatagtgattctggatattaaagtgggaatgaggggcaggccactaacgaagaaga
tgtttctcaaagaagctaaaagaaaaggggggactggaagggctaattcactcccaacgaag
acaagatctgctttttgcttgtactGGGTCTCTCTGGTTAGACCAGATCTGAGCCTGGGAGC
TCTCTGGCTAACTAGGGAACCCACTGCTTAAGCCTCAATAAAGCTTGCCTTGAGTGCTTCAA
GTAGTGTGTTGGTTTTTTGTGTGTGCATTGTCTGAGTAGGTGTCATTCTATTCTGGGGGGTG
GGGTGGGGCAGCACAGCAAGGGGGAGGATTGGGAAGACAATAGCAGGCATGCTGGGGATGCG
GTGGGCTCTATGGagtagtagttcatgtcatcttattattcagtatttataacttgcaaaga
aatgaatatcagagagtgagaggaacttgtttattgcagcttataatggttacaaataaagc
aatagcatcacaaatttcacaaataaagcatttttttcactgcattctagttgtggtttgtc
caaactcatcaatgtatcttatcatgtctggctctagctatcccgcccctaactccgcccAT
CCCGCCCCTAACTCCGCCCAGTTCCGCCCATTCTCCGCCCCATGGCTCTCACTACTTCTGGA
ATAGCTCAGAGGCCGAGGCGGCCTCGGCCTCTGCATAAATAAAAAAAATTAGTCGAGGCTTT
TTTGGAGGCCTAGGgacgtacccaattcgccctatagtgagtcgtattacgcgcgctcactg
gccgtcgttttacaacgtcgtgactgggaaaaccctggcgttacccaacttaatcgccttgc
agcacatccccctttcgccagctggcgtaatagcgaagaggcccgcaccgatcgcccttccc
aacagttgcgcagcctgaatggcgaatggg
Vector VI (FIG. 1, panel B).
Sequence ID No: 32
acgcgccctgtagcggcgcattaagcgcggcgggtgtggtggttacgcgcagcgtgaccgct
acacttgccagcgccctagcgcccgctcctttcgctttcttcccttcctttctcgccacgtt
cgccggctttccccgtcaagctctaaatcgggggctccctttagggttccgatttagtgctt
tacggcacctcgaccccaaaaaacttgattagggtgatggttcacgtagtgggccatcgccc
tgatagacggtttttcgccctttgacgttggagtccacgttctttaatagtggactcttgtt
ccaaactggaacaacactcaaccctatctcggtctattcttttgatttataagggattttgc
cgatttcggcctattggttaaaaaatgagctgatttaacaaaaatttaacgcgaattttaac
aaaatattaacgcttacaatttaggtggcacttttcggggaaatgtgcgcggaacccctatt
tgtttatttttctaaatacattcaaatatgtatccgctcatgagacaataaccctgataaat
gcttcaataatagcacctagatcaagagacaggatgaggatcgtttcgcatgattgaacaag
atggattgcacgcaggttctccggccgcttgggtggagaggctattcggctatgactgggca
caacagacaatcggctgctctgatgccgccgtgttccggctgtcagcgcaggggcgcccggt
tctttttgtcaagaccgacctgtccggtgccctgaatgaactgcaagacgaggcagcgcggc
tatcgtggctggccacgacgggcgttccttgcgcagctgtgctcgacgttgtcactgaagcg
ggaagggactggctgctattgggcgaagtgccggggcaggatctcctgtcatctcaccttgc
tcctgccgagaaagtatccatcatggctgatgcaatgcggcggctgcatacgcttgatccgg
ctacctgcccattcgaccaccaagcgaaacatcgcatcgagcgagcacgtactcggatggaa
gccggtcttgtcgatcaggatgatctggacgaagagcatcaggggctcgcgccagccgaact
gttcgccaggctcaaggcgagcatgcccgacggcgaggatctcgtcgtgacccatggcgatg
cctgcttgccgaatatcatggtggaaaatggccgcttttctggattcatcgactgtggccgg
ctgggtgtggcggaccgctatcaggacatagcgttggctacccgtgatattgctgaagagct
tggcggcgaatgggctgaccgcttcctcgtgctttacggtatcgccgctcccgattcgcagc
gcatcgccttctatcgccttcttgacgagttcttctgaattattaacgcttacaatttcctg
atgcggtattttctccttacgcatctgtgcggtatttcacaccgcatcaggtggcacttttc
ggggaaatgtgcgcggaacccctatttgtttatttttctaaatacattcaaatatgtatccg
ctcatgaccaaaatcccttaacgtgagttttcgttccactgagcgtcagaccccgtagaaaa
gatcaaaggatcttcttgagatcctttttttctgcgcgtaatctgctgcttgcaaacaaaaa
aaccaccgctaccagcggtggtttgtttgccggatcaagagctaccaactctttttccgaag
gtaactggcttcagcagagcgcagataccaaatactgttcttctagtgtagccgtagttagg
ccaccacttcaagaactctgtagcaccgcctacatacctcgctctgctaatcctgttaccag
tggctgctgccagtggcgataagtcgtgtcttaccgggttggactcaagacgatagttaccg
gataaggcgcagcggtcgggctgaacgggggggtcgtgcacacagcccagcttggagcgaac
gacctacaccgaactgagatacctacagcgtgagctatgagaaagcgccacgcttcccgaag
ggagaaaggcggacaggtatccggtaagcggcagggtcggaacaggagagcgcacgagggag
cttccagggggaaacgcctggtatctttatagtcctgtcgggtttcgccacctctgacttga
gcgtcgatttttgtgatgctcgtcaggggggcggagcctatggaaaaacgccagcaacgcgg
cctttttacggttcctggccttttgctggccttttgctcacatgttctttcctgcgttatcc
cctgattctgtggataaccgtattaccgcctttgagtgagctgataccgctcgccgcagccg
aacgaccgagcgcagcgagtcagtgagcgaggaagcggaagagcgcccaatacgcaaaccgc
ctctccccgcgcgttggccgattcattaatgcagctggcacgacaggtttcccgactggaaa
gcgggcagtgagcgcaacgcaattaatgtgagttagctcactcattaggcaccccaggcttt
acactttatgcttccggctcgtatgttgtgtggaattgtgagcggataacaatttcacacag
gaaacagctatgaccatgattacgccaagcgcgcaattaaccctcactaaagggaacaaaag
ctggagctgcaagcttggccattgcatacgttgtatccatatcataatatgtacatttatat
tggctcatgtccaacattaccgccatgttgacattgattattgactagttattaatagtaat
caattacggggtcattagttcatagcccatatatggagttccgcgttacataacttacggta
aatggcccgcctggctgaccgcccaacgacccccgcccattgacgtcaataatgacgtatgt
tcccatagtaacgccaatagggactttccattgacgtcaatgggtggagtatttacggtaaa
ctgcccacttggcagtacatcaagtgtatcatatgccaagtacgccccctattgacgtcaat
gacggtaaatggcccgcctggcattatgcccagtacatgaccttatgggactttcctacttg
gcagtacatctacgtattagtcatcgctattaccatggtgatgcggttttggcagtacatca
atgggcgtggatagcggtttgactcacggggatttccaagtctccaccccattgacgtcaat
gggagtttgttttggcaccaaaatcaacgggactttccaaaatgtcgtaacaactccgcccc
attgacgcaaatgggcggtaggcgtgtacggtgggaggtctatataagcagagctcgtttag
tgaaccgGGGTCTCTCTGGTTAGACCAGATCTGAGCCTGGGAGCTCTCTGGCTAACTAGGGA
ACCCACTGCTTAAGCCTCAATAAAGCTTGCCTTGAGTGCTTCAAGTAGTGTGTGCCCGTCTG
TTGTGTGACTCTGGTAACTAGAGATCCCTCAGACCCTTTTAGTCAGTGTGGAAAATCTCTAG
CagtggcgcccgaacagggacttgaaagcgaaagggaaaccagaggagctctcTCGACGCAG
GACTCGGCTTGCTGAAGCGCGCACGGCAAGAGGCGAGGGGCGGCGACTGGTGAGTACGCCAA
AAATTTTGACTAGCGGAGGCTAGAAGGAGAGAGATGGGTGCGAGAGCGTCAGTATTAAGCGG
GGGAGaattagatcgcgatgggaaaaaattcggttaaggccagggggaaagaaaaaatataa
attaaaacatatagtatgggcaagcagggagctagaacgattcgcagttaatcctggcctgt
tagaaacatcagaaggctgtagacaaatactgggacagctacaaccatcccttcagacagga
tcagaagaacttagatcattatataatacagtagcaaccctctattgtgtgcatcaaaggat
agagataaaagacaccaaggaagctttagacaagatagaggaagagcaaaacaaaagtaaga
ccaccgcacagcaagcGGCCGCTgatcttcagacctggaggaggagatatgagggacaattg
gagaagtgaattatataaatataaagtagtaaaaattgaaccattaggagtagcacccacca
aggcaaagagaagagtggtgcagagagaaaaaagaGCAGTGGGAATaggagctttgttcctt
gggttcttgggagcagcaggaagcactatgggcgcagcgtcaatgacgctgacggtacaggc
cagacaattattgtctggtatagtgcagcagcagaacaatttgctgagggctattgaggcgc
aacagcatctgttgcaactcacagtctggggcatcaagcagctccaggcaagaatcctggct
gtggaaagatacctaaaggatcaacagctcctggggatttggggttgctctggaaaactcat
ttgcaccactgctgtgccttggaatgctagttggagtaataaatctctggaacagatttgga
atcacacgacctggatggagtgggacagagaaattaacaattacacaagcttaatacactcc
ttaattgaagaatcgcaaaaccagcaagaaaagaatgaacaagaattattggaattagataa
atgggcaagtttgtggaattggtttaacataacaaattggctgtggtatataaaattattca
taatgatagtaggaggcttggtaggtttaagaatagtttttgctgtactttctatagtgaat
agagttaggcagggatattcaccattatcgtttcagacccacctcccaaccccgaggggacc
cgacaggcccgaaggaatagaagaagaaggtggagagagagacagagacagatccattcgat
tagtgaacggatCTCGACGGTATCGATCTCGAcacaaatggcagtattcatccacaatttta
aaagaaaaggggggattggggggtacagtgcaggggaaagaatagtagacataatagcaaca
gacatacaaactaaagaattacaaaaacaaattacaaaaattcaaaattttcgggtttatta
cagggacagcagaTGCTGCCCACTCAGACTTTATTCAAAGACCAGGAAGGGCCGGTGCAAGG
AGGGGAGGAGGGCCCGTTGGGAGGCCCAGCGGGCAGGAGGAACGGCTACCGAGGCTCCAGCT
TAACGGTATTTGGAGGTCAGCACGGTGCTCACAGAAGCCAGGAACTTGTCCAGGGAGGCGTG
CACCGCAGGGGTGAACTCGGCGGGGAGGTGGGCGGCCAGGGTCACCAGCAGGCAGTGGCTTA
GGAGCTGTGCAGAGAAGAGGGTCAGTGCGGCCCAGGCCCGCAGCCGCCGCCTGCGCTACACC
TCCCGCAACCCGCGTGATCCTCTGCCCTGAGAGGAAGGCGCCATCTCGCCCCTCGACCCAGA
TCGCTCCCGGCCCGCCGCTCACCTTGAAGTTGACCGGGTCCACCCGAAGCTTGTGCGCGTGC
AGGTCGCTCAGGGCGGACAGCGCGTTGGGCATGTCGTCCACGTGCGCCACGGCGTTGGTCAG
CGCGTCGGCCACCTTCTTGCCGTGGCCCTTAACCTGGGCAGAGCCGTGGCTCAGGTCGAAGT
GCGGGAAGTAGGTCTTGGTGGTGGGGAAGGACAGGAACATCCTGCGGGGAGAAGCAGAGTGA
GGGGTGGGGTTTGGGTCCGGGGCCAGGACGGTTGAGGGTGGCCTGTGGGTCCGGGCGGGCGA
GGAGCCCGGGTCGGAGCAGGGGAGGGAGCCTCACCTCTCCAGGGCCTCCGCACCATACTCGC
CAGCGTGCGCGCCGACCTTACCCCAGGCGGCCTTGACGTTGGTCTTGTCGGCAGGAGACAGC
ACCATGGTGGGTTCTCTCTGAGTCTGTGGGGACCAGAAGAGTaagcaatagatggctctgcc
ctgacttttatgcccagccctggctcctgccctccctgctcctgggagtagattggccaacc
ctagggtgtggctccacagggtgaggtctaagtgatgacagccgtacctgtccttggctctt
ctggcactggcttaggagttggacttcaaaccctcagccctccctctaagatatatctcttg
gccccataccatcagtacaaattgctactaaaaacatcctcctttgcaagtgtatttaccct
tttgccacctagctgtccaggggtgccttaaaatggcaaacaaggtttgttttcttttcctg
ttttcatgccttcctcttccatatccttgtttcatattaatacatgtgtatagatcctaaaa
atctatacacatgtattaataaagcctgattctgccgcttctaggtatagaggccacctgca
agataaatatttgattcacaataactaatcattctatggcaattgataacaacaaatatata
tatatatatatatacgtatatgtgtatatatatatatatatattcaggaaataatatattct
agaatatgtcacattctgtctcaggcatccattttctttatgatgccgtttgaggtggagtt
ttagtcaggtggtcagcttctccttttttttgccatctgccctgtaagcatcctgctgggga
cccagataggagtcatcactctaggctgagaacatctgggcacacaccctaagcctcagcat
gactcatcatgactcagcattgctgtgcttgagccagaaggtttgcttagaaggttacacag
aaccagaaggcgggggtggggcactgaccccgacaggggcctggccagaactgctcatgctt
ggactatgggaggtcactaatggagacacacagaaatgtaacaggaactaaggaaaaactga
agcttatttaatcagagatgaggatgctggaagggatagagggagctgagcttgtaaaaagt
atagtaatcattcagcaaatggttttgaagcacctgctggatgctaaacactattttcagtg
cttgaatcataaataagaataaaacatgtatcttattccccacaagagtccaagtaaaaaat
aacagttaattataatgtgctctgtcccccaggctggagtgcagtggcacgatctcagctca
ctgcaacctccgcctcccgggttcaagcaattctcctgcctcagccaccctaatagctggga
ttacaggtgcacaccaccatgccaggctaatttttgtactttttgtagaggcagggtatcac
catgttgtccaagatggtcttgaactcctgagctccaagcagtccacccacctcagcctccc
aaagtgctgggattacaggtgtgagacaccatgcccagattttccatatttaatagaggtat
ttatgggatgggggaaaagaatgtttctctcactgtggattattttagagagtggagaatgg
tcaagatttttttaaaaattaagaaaacataagttggaccttgagaaatgaaaatttatttt
tttgttggaggatacccattctctatctcccatcagggcaagctgtaaggaactggctaaga
cacagtgagacagagtgacttagtcttagaggccccactggtacgacggtcacctgggggta
taggggagcagtcccatgtagtagtagaatgaaaaatgctgctatgctgtgcctcccccacc
tttcccatgtctgccctctactcatggtctatctctcctggctcctgggagtcatggactcc
acccagcaccaccaacctgacctaaccacctatctgagcctgccagcctataacccatctgg
gccctgatagctggtggccagccctgaccccaccccaccctccctggaacctctgatagaca
catctggcacaccagctcgcaaagtcaccgtgagggtcttgtgtttgctgagtcaaaattcc
ttgaaatccaagtccttagagactcccaggcttggattcaaagctcctgactttctgtctag
tgtatgtgcagtgagccccttttcctctaactgaaagaaggaaaaaaaaatggaacccaaaa
tattctacatagtttccatgtcacagccagggctgggcagtctcctgttatttcttttaaaa
taaatatatcatttaaatgcataaataagcaaaccctgctcgggaatgggagggagagtctc
tggagtccaccccttctcggccctggctctgcagatagtgctatcaaagccctgacagagcc
ctgcccattgctgggccttggagtgagtcagcctagtagagaggcagggcaagccatctcat
agctgctgagtgggagagagaaaagggctcattgtctataaactcaggtcatggctattctt
attaaaagaaaaggggggactggaagggctaattcactcccaacgaagacaagatctgcttt
ttgcttgtactGGGTCTCTCTGGTTAGACCAGATCTGAGCCTGGGAGCTCTCTGGCTAACTA
GGGAACCCACTGCTTAAGCCTCAATAAAGCTTGCCTTGAGTGCTTCAAGTAGTGTGTTGGTT
TTTTGTGTGTGCATTGTCTGAGTAGGTGTCATTCTATTCTGGGGGGTGGGGTGGGGCAGCAC
AGCAAGGGGGAGGATTGGGAAGACAATAGCAGGCATGCTGGGGATGCGGTGGGCTCTATGGa
gtagtagttcatgtcatcttattattcagtatttataacttgcaaagaaatgaatatcagag
agtgagaggaacttgtttattgcagcttataatggttacaaataaagcaatagcatcacaaa
tttcacaaataaagcatttttttcactgcattctagttgtggtttgtccaaactcatcaatg
tatcttatcatgtctggctctagctatcccgcccctaactccgcccATCCCGCCCCTAACTC
CGCCCAGTTCCGCCCATTCTCCGCCCCATGGCTCTCACTACTTCTGGAATAGCTCAGAGGCC
GAGGCGGCCTCGGCCTCTGCATAAATAAAAAAAATTAGTCGAGGCTTTTTTGGAGGCCTAGG
gacgtacccaattcgccctatagtgagtcgtattacgcgcgctcactggccgtcgttttaca
acgtcgtgactgggaaaaccctggcgttacccaacttaatcgccttgcagcacatccccctt
tcgccagctggcgtaatagcgaagaggcccgcaccgatcgcccttcccaacagttgcgcagc
ctgaatggcgaatggg
Vector VII (FIG. 1, panel B).
Sequence ID No: 33
acgcgccctgtagcggcgcattaagcgcggcgggtgtggtggttacgcgcagcgtgaccgct
acacttgccagcgccctagcgcccgctcctttcgctttcttcccttcctttctcgccacgtt
cgccggctttccccgtcaagctctaaatcgggggctccctttagggttccgatttagtgctt
tacggcacctcgaccccaaaaaacttgattagggtgatggttcacgtagtgggccatcgccc
tgatagacggtttttcgccctttgacgttggagtccacgttctttaatagtggactcttgtt
ccaaactggaacaacactcaaccctatctcggtctattcttttgatttataagggattttgc
cgatttcggcctattggttaaaaaatgagctgatttaacaaaaatttaacgcgaattttaac
aaaatattaacgcttacaatttaggtggcacttttcggggaaatgtgcgcggaacccctatt
tgtttatttttctaaatacattcaaatatgtatccgctcatgagacaataaccctgataaat
gcttcaataatagcacctagatcaagagacaggatgaggatcgtttcgcatgattgaacaag
atggattgcacgcaggttctccggccgcttgggtggagaggctattcggctatgactgggca
caacagacaatcggctgctctgatgccgccgtgttccggctgtcagcgcaggggcgcccggt
tctttttgtcaagaccgacctgtccggtgccctgaatgaactgcaagacgaggcagcgcggc
tatcgtggctggccacgacgggcgttccttgcgcagctgtgctcgacgttgtcactgaagcg
ggaagggactggctgctattgggcgaagtgccggggcaggatctcctgtcatctcaccttgc
tcctgccgagaaagtatccatcatggctgatgcaatgcggcggctgcatacgcttgatccgg
ctacctgcccattcgaccaccaagcgaaacatcgcatcgagcgagcacgtactcggatggaa
gccggtcttgtcgatcaggatgatctggacgaagagcatcaggggctcgcgccagccgaact
gttcgccaggctcaaggcgagcatgcccgacggcgaggatctcgtcgtgacccatggcgatg
cctgcttgccgaatatcatggtggaaaatggccgcttttctggattcatcgactgtggccgg
ctgggtgtggcggaccgctatcaggacatagcgttggctacccgtgatattgctgaagagct
tggcggcgaatgggctgaccgcttcctcgtgctttacggtatcgccgctcccgattcgcagc
gcatcgccttctatcgccttcttgacgagttcttctgaattattaacgcttacaatttcctg
atgcggtattttctccttacgcatctgtgcggtatttcacaccgcatcaggtggcacttttc
ggggaaatgtgcgcggaacccctatttgtttatttttctaaatacattcaaatatgtatccg
ctcatgaccaaaatcccttaacgtgagttttcgttccactgagcgtcagaccccgtagaaaa
gatcaaaggatcttcttgagatcctttttttctgcgcgtaatctgctgcttgcaaacaaaaa
aaccaccgctaccagcggtggtttgtttgccggatcaagagctaccaactctttttccgaag
gtaactggcttcagcagagcgcagataccaaatactgttcttctagtgtagccgtagttagg
ccaccacttcaagaactctgtagcaccgcctacatacctcgctctgctaatcctgttaccag
tggctgctgccagtggcgataagtcgtgtcttaccgggttggactcaagacgatagttaccg
gataaggcgcagcggtcgggctgaacgggggggtcgtgcacacagcccagcttggagcgaac
gacctacaccgaactgagatacctacagcgtgagctatgagaaagcgccacgcttcccgaag
ggagaaaggcggacaggtatccggtaagcggcagggtcggaacaggagagcgcacgagggag
cttccagggggaaacgcctggtatctttatagtcctgtcgggtttcgccacctctgacttga
gcgtcgatttttgtgatgctcgtcaggggggcggagcctatggaaaaacgccagcaacgcgg
cctttttacggttcctggccttttgctggccttttgctcacatgttctttcctgcgttatcc
cctgattctgtggataaccgtattaccgcctttgagtgagctgataccgctcgccgcagccg
aacgaccgagcgcagcgagtcagtgagcgaggaagcggaagagcgcccaatacgcaaaccgc
ctctccccgcgcgttggccgattcattaatgcagctggcacgacaggtttcccgactggaaa
gcgggcagtgagcgcaacgcaattaatgtgagttagctcactcattaggcaccccaggcttt
acactttatgcttccggctcgtatgttgtgtggaattgtgagcggataacaatttcacacag
gaaacagctatgaccatgattacgccaagcgcgcaattaaccctcactaaagggaacaaaag
ctggagctgcaagcttggccattgcatacgttgtatccatatcataatatgtacatttatat
tggctcatgtccaacattaccgccatgttgacattgattattgactagttattaatagtaat
caattacggggtcattagttcatagcccatatatggagttccgcgttacataacttacggta
aatggcccgcctggctgaccgcccaacgacccccgcccattgacgtcaataatgacgtatgt
tcccatagtaacgccaatagggactttccattgacgtcaatgggtggagtatttacggtaaa
ctgcccacttggcagtacatcaagtgtatcatatgccaagtacgccccctattgacgtcaat
gacggtaaatggcccgcctggcattatgcccagtacatgaccttatgggactttcctacttg
gcagtacatctacgtattagtcatcgctattaccatggtgatgcggttttggcagtacatca
atgggcgtggatagcggtttgactcacggggatttccaagtctccaccccattgacgtcaat
gggagtttgttttggcaccaaaatcaacgggactttccaaaatgtcgtaacaactccgcccc
attgacgcaaatgggcggtaggcgtgtacggtgggaggtctatataagcagagctcgtttag
tgaaccgGGGTCTCTCTGGTTAGACCAGATCTGAGCCTGGGAGCTCTCTGGCTAACTAGGGA
ACCCACTGCTTAAGCCTCAATAAAGCTTGCCTTGAGTGCTTCAAGTAGTGTGTGCCCGTCTG
TTGTGTGACTCTGGTAACTAGAGATCCCTCAGACCCTTTTAGTCAGTGTGGAAAATCTCTAG
CagtggcgcccgaacagggacttgaaagcgaaagggaaaccagaggagctctcTCGACGCAG
GACTCGGCTTGCTGAAGCGCGCACGGCAAGAGGCGAGGGGCGGCGACTGGTGAGTACGCCAA
AAATTTTGACTAGCGGAGGCTAGAAGGAGAGAGATGGGTGCGAGAGCGTCAGTATTAAGCGG
GGGAGaattagatcgcgatgggaaaaaattcggttaaggccagggggaaagaaaaaatataa
attaaaacatatagtatgggcaagcagggagctagaacgattcgcagttaatcctggcctgt
tagaaacatcagaaggctgtagacaaatactgggacagctacaaccatcccttcagacagga
tcagaagaacttagatcattatataatacagtagcaaccctctattgtgtgcatcaaaggat
agagataaaagacaccaaggaagctttagacaagatagaggaagagcaaaacaaaagtaaga
ccaccgcacagcaagcGGCCGCTgatcttcagacctggaggaggagatatgagggacaattg
gagaagtgaattatataaatataaagtagtaaaaattgaaccattaggagtagcacccacca
aggcaaagagaagagtggtgcagagagaaaaaagaGCAGTGGGAATaggagctttgttcctt
gggttcttgggagcagcaggaagcactatgggcgcagcgtcaatgacgctgacggtacaggc
cagacaattattgtctggtatagtgcagcagcagaacaatttgctgagggctattgaggcgc
aacagcatctgttgcaactcacagtctggggcatcaagcagctccaggcaagaatcctggct
gtggaaagatacctaaaggatcaacagctcctggggatttggggttgctctggaaaactcat
ttgcaccactgctgtgccttggaatgctagttggagtaataaatctctggaacagatttgga
atcacacgacctggatggagtgggacagagaaattaacaattacacaagcttaatacactcc
ttaattgaagaatcgcaaaaccagcaagaaaagaatgaacaagaattattggaattagataa
atgggcaagtttgtggaattggtttaacataacaaattggctgtggtatataaaattattca
taatgatagtaggaggcttggtaggtttaagaatagtttttgctgtactttctatagtgaat
agagttaggcagggatattcaccattatcgtttcagacccacctcccaaccccgaggggacc
cgacaggcccgaaggaatagaagaagaaggtggagagagagacagagacagatccattcgat
tagtgaacggatCTCGACGGTATCGATCTCGAcacaaatggcagtattcatccacaatttta
aaagaaaaggggggattggggggtacagtgcaggggaaagaatagtagacataatagcaaca
gacatacaaactaaagaattacaaaaacaaattacaaaaattcaaaattttcgggtttatta
cagggacagcagaTGCTGCCCACTCAGACTTTATTCAAAGACCAGGAAGGGCCGGTGCAAGG
AGGGGAGGAGGGCCCGTTGGGAGGCCCAGCGGGCAGGAGGAACGGCTACCGAGGCTCCAGCT
TAACGGTATTTGGAGGTCAGCACGGTGCTCACAGAAGCCAGGAACTTGTCCAGGGAGGCGTG
CACCGCAGGGGTGAACTCGGCGGGGAGGTGGGCGGCCAGGGTCACCAGCAGGCAGTGGCTTA
GGAGCTGTGCAGAGAAGAGGGTCAGTGCGGCCCAGGCCCGCAGCCGCCGCCTGCGCTACACC
TCCCGCAACCCGCGTGATCCTCTGCCCTGAGAGGAAGGCGCCATCTCGCCCCTCGACCCAGA
TCGCTCCCGGCCCGCCGCTCACCTTGAAGTTGACCGGGTCCACCCGAAGCTTGTGCGCGTGC
AGGTCGCTCAGGGCGGACAGCGCGTTGGGCATGTCGTCCACGTGCGCCACGGCGTTGGTCAG
CGCGTCGGCCACCTTCTTGCCGTGGCCCTTAACCTGGGCAGAGCCGTGGCTCAGGTCGAAGT
GCGGGAAGTAGGTCTTGGTGGTGGGGAAGGACAGGAACATCCTGCGGGGAGAAGCAGAGTGA
GGGGTGGGGTTTGGGTCCGGGGCCAGGACGGTTGAGGGTGGCCTGTGGGTCCGGGCGGGCGA
GGAGCCCGGGTCGGAGCAGGGGAGGGAGCCTCACCTCTCCAGGGCCTCCGCACCATACTCGC
CAGCGTGCGCGCCGACCTTACCCCAGGCGGCCTTGACGTTGGTCTTGTCGGCAGGAGACAGC
ACCATGGTGGGTTCTCTCTGAGTCTGTGGGGACCAGAAGAGTGCCGGCCCGCGAGCGCGCCA
GGGTTTATGCTTGGGGCGCGGGGGCACGCCCGGCCGGGCGGCGCTCATTGGCTGGCGCGGAG
CCCGGGGCGCGGCCTGGACCGCAGGGGAGTCCCGGGGGGGGCCTGCGCGGGGCCGGGCGGCG
GGCTCGGCCTCCCCTGGGGGTGCACGCGGGGCGGGGGCCAGGACGTCTCCACCCTCCACCCG
CCACTACGTaTATGTGTATATATATATATATATTCAGGAAATAATATATTCTAGAATATGTC
ACATTCTGTCTCAGGCATCCATTTTCTTTATGATGCCGTTTGAGGTGGAGTTTTAGTCAGGT
GGTCAGCTTCTCCTTTTTTTTGCCATCTGCCCTGTAAGCATCCTGCTGGGGACCCAGATAGG
AGTCATCACTCTAGGCTGAGAACATCTGGGCACACACCCTAAGCCTCAGCATGACTCATCAT
GACTCAGCATTGCTGTGCTTGAGCCAGAAGGTTTGCTTAGAAGGTTACACAGAACCAGAAGG
CGGGGGTGGGGCACTGACCCCGACAGGGGCCTGGCCAGAACTGCTCATGCTTGGACTATGGG
AGGTCACTAATGGAGACACACAGAAATGTAACAGGAACTAAGGAAAAACTGAAGCTTtgggg
gtataggggagcagtcccatgtagtagtagaatgaaaaatgctgctatgctgtgcctccccc
acctttcccatgtctgccctctactcatggtctatctctcctggctcctgggagtcatggac
tccacccagcaccaccaacctgacctaaccacctatctgagcctgccagcctataacccatc
tgggccctgatagctggtggccagccctgaccccaccccaccctccctggaacctctgatag
acacatctggcacaccagctcgcaaagtcaccgtgagggtcttgtgtttgctgagtcaaaat
tccttgaaatccaagtccttagagactcccaggcttggattcaaagctcctgactttctgtc
tagtgtatgtgcagtgagccccttttcctctaactgaaagaaggaaaaaaaaatggaaccca
aaatattctacatagtttccatgtcacagccagggctgggcagtctcctgttatttctttta
aaataaatatatcatttaaatgcataaataagcaaaccctgctcgggaatgggagggagagt
ctctggagtccaccccttctcggccctggctctgcagatagtgctatcaaagccctgacaga
gccctgcccattgctgggccttggagtgagtcagcctagtagagaggcagggcaagccatct
catagctgctgagtgggagagagaaaagggctcattgtctataaactcaggtcatggctatt
cttattaaaagaaaaggggggactggaagggctaattcactcccaacgaagacaagatctgc
tttttgcttgtactGGGTCTCTCTGGTTAGACCAGATCTGAGCCTGGGAGCTCTCTGGCTAA
CTAGGGAACCCACTGCTTAAGCCTCAATAAAGCTTGCCTTGAGTGCTTCAAGTAGTGTGTTG
GTTTTTTGTGTGTGCATTGTCTGAGTAGGTGTCATTCTATTCTGGGGGGTGGGGTGGGGCAG
CACAGCAAGGGGGAGGATTGGGAAGACAATAGCAGGCATGCTGGGGATGCGGTGGGCTCTAT
GGagtagtagttcatgtcatcttattattcagtatttataacttgcaaagaaatgaatatca
gagagtgagaggaacttgtttattgcagcttataatggttacaaataaagcaatagcatcac
aaatttcacaaataaagcatttttttcactgcattctagttgtggtttgtccaaactcatca
atgtatcttatcatgtctggctctagctatcccgcccctaactccgcccATCCCGCCCCTAA
CTCCGCCCAGTTCCGCCCATTCTCCGCCCCATGGCTCTCACTACTTCTGGAATAGCTCAGAG
GCCGAGGCGGCCTCGGCCTCTGCATAAATAAAAAAAATTAGTCGAGGCTTTTTTGGAGGCCT
AGGgacgtacccaattcgccctatagtgagtcgtattacgcgcgctcactggccgtcgtttt
acaacgtcgtgactgggaaaaccctggcgttacccaacttaatcgccttgcagcacatcccc
ctttcgccagctggcgtaatagcgaagaggcccgcaccgatcgcccttcccaacagttgcgc
agcctgaatggcgaatggg
A1-UV
Sequence ID No: 34
acgcgccctgtagcggcgcattaagcgcggcgggtgtggtggttacgcgcagcgtgaccgctacacttgccagcgccctagcgccc
gctcctttcgctttcttcccttcctttctcgccacgttcgccggctttccccgtcaagctctaaatcgggggctccctttagggttccgattta
gtgctttacggcacctcgaccccaaaaaacttgattagggtgatggttcacgtagtgggccatcgccctgatagacggtttttcgcccttt
gacgttggagtccacgttctttaatagtggactcttgttccaaactggaacaacactcaaccctatctcggtctattcttttgatttataaggg
attttgccgatttcggcctattggttaaaaaatgagctgatttaacaaaaatttaacgcgaattttaacaaaatattaacgcttacaatttaggt
ggcacttttcggggaaatgtgcgcggaacccctatttgtttatttttctaaatacattcaaatatgtatccgctcatgagacaataaccctgat
aaatgcttcaataatagcacctagatcaagagacaggatgaggatcgtttcgcatgattgaacaagatggattgcacgcaggttctccg
gccgcttgggtggagaggctattcggctatgactgggcacaacagacaatcggctgctctgatgccgccgtgttccggctgtcagcgc
aggggcgcccggttctttttgtcaagaccgacctgtccggtgccctgaatgaactgcaagacgaggcagcgcggctatcgtggctgg
ccacgacgggcgttccttgcgcagctgtgctcgacgttgtcactgaagcgggaagggactggctgctattgggcgaagtgccgggg
caggatctcctgtcatctcaccttgctcctgccgagaaagtatccatcatggctgatgcaatgcggcggctgcatacgcttgatccggct
acctgcccattcgaccaccaagcgaaacatcgcatcgagcgagcacgtactcggatggaagccggtcttgtcgatcaggatgatctg
gacgaagagcatcaggggctcgcgccagccgaactgttcgccaggctcaaggcgagcatgcccgacggcgaggatctcgtcgtga
cccatggcgatgcctgcttgccgaatatcatggtggaaaatggccgcttttctggattcatcgactgtggccggctgggtgtggcggac
cgctatcaggacatagcgttggctacccgtgatattgctgaagagcttggcggcgaatgggctgaccgcttcctcgtgctttacggtatc
gccgctcccgattcgcagcgcatcgccttctatcgccttcttgacgagttcttctgaattattaacgcttacaatttcctgatgcggtattttct
ccttacgcatctgtgcggtatttcacaccgcatcaggtggcacttttcggggaaatgtgcgcggaacccctatttgtttatttttctaaatac
attcaaatatgtatccgctcatgaccaaaatcccttaacgtgagttttcgttccactgagcgtcagaccccgtagaaaagatcaaaggatc
ttcttgagatcctttttttctgcgcgtaatctgctgcttgcaaacaaaaaaaccaccgctaccagcggtggtttgtttgccggatcaagagct
accaactctttttccgaaggtaactggcttcagcagagcgcagataccaaatactgttcttctagtgtagccgtagttaggccaccacttc
aagaactctgtagcaccgcctacatacctcgctctgctaatcctgttaccagtggctgctgccagtggcgataagtcgtgtcttaccggg
ttggactcaagacgatagttaccggataaggcgcagcggtcgggctgaacgggggggtcgtgcacacagcccagcttggagcgaa
cgacctacaccgaactgagatacctacagcgtgagctatgagaaagcgccacgcttcccgaagggagaaaggcggacaggtatcc
ggtaagcggcagggtcggaacaggagagcgcacgagggagcttccagggggaaacgcctggtatctttatagtcctgtcgggtttc
gccacctctgacttgagcgtcgatttttgtgatgctcgtcaggggggcggagcctatggaaaaacgccagcaacgcggcctttttacgg
ttcctggccttttgctggccttttgctcacatgttctttcctgcgttatcccctgattctgtggataaccgtattaccgcctttgagtgagctgat
accgctcgccgcagccgaacgaccgagcgcagcgagtcagtgagcgaggaagcggaagagcgcccaatacgcaaaccgcctct
ccccgcgcgttggccgattcattaatgcagctggcacgacaggtttcccgactggaaagcgggcagtgagcgcaacgcaattaatgt
gagttagctcactcattaggcaccccaggctttacactttatgcttccggctcgtatgttgtgtggaattgtgagcggataacaatttcaca
caggaaacagctatgaccatgattacgccaagcgcgcaattaaccctcactaaagggaacaaaagctggagctgcaagcttggccat
tgcatacgttgtatccatatcataatatgtacatttatattggctcatgtccaacattaccgccatgttgacattgattattgactagttattaata
gtaatcaattacggggtcattagttcatagcccatatatggagttccgcgttacataacttacggtaaatggcccgcctggctgaccgccc
aacgacccccgcccattgacgtcaataatgacgtatgttcccatagtaacgccaatagggactttccattgacgtcaatgggggagtat
ttacggtaaactgcccacttggcagtacatcaagtgtatcatatgccaagtacgccccctattgacgtcaatgacggtaaatggcccgcc
tggcattatgcccagtacatgaccttatgggactttcctacttggcagtacatctacgtattagtcatcgctattaccatggtgatgcggtttt
ggcagtacatcaatgggcgtggatagcggtttgactcacggggatttccaagtctccaccccattgacgtcaatgggagtttgttttggc
accaaaatcaacgggactttccaaaatgtcgtaacaactccgccccattgacgcaaatgggcggtaggcgtgtacggtgggaggtcta
tataagcagagctcgtttagtgaaccgGGGTCTCTCTGGTTAGACCAGATCTGAGCCTGGGAGCT
CTCTGGCTAACTAGGGAACCCACTGCTTAAGCCTCAATAAAGCTTGCCTTGAGTG
CTTCAAGTAGTGTGTGCCCGTCTGTTGTGTGACTCTGGTAACTAGAGATCCCTCA
GACCCTTTTAGTCAGTGTGGAAAATCTCTAGCagtggcgcccgaacagggacttgaaagcgaaaggg
aaaccagaggagctctcTCGACGCAGGACTCGGCTTGCTGAAGCGCGCACGGCAAGAGGC
GAGGGGCGGCGACTGGTGAGTACGCCAAAAATTTTGACTAGCGGAGGCTAGAAG
GAGAGAGATGGGTGCGAGAGCGTCAGTATTAAGCGGGGGAGaattagatcgcgatgggaaa
aaattcggttaaggccagggggaaagaaaaaatataaattaaaacatatagtatgggcaagcagggagctagaacgattcgcagttaa
tcctggcctgttagaaacatcagaaggctgtagacaaatactgggacagctacaaccatcccttcagacaggatcagaagaacttagat
cattatataatacagtagcaaccctctattgtgtgcatcaaaggatagagataaaagacaccaaggaagctttagacaagatagaggaa
gagcaaaacaaaagtaagaccaccgcacagcaagcGGCCGCTgatcttcagacctggaggaggagatatgagggacaattg
gagaagtgaattatataaatataaagtagtaaaaattgaaccattaggagtagcacccaccaaggcaaagagaagagtggtgcagag
agaaaaaagaGCAGTGGGAATaggagctttgttcctttgggutcutgggagcagcaggaagcactatgggcgcagcgtcaat
gacgctgacggtacaggccagacaattattgtctggtatagtgcagcagcagaacaatttgctgagggctattgaggcgcaacagcat
ctgttgcaactcacagtctggggcatcaagcagctccaggcaagaatcctggctgtggaaagatacctaaaggatcaacagctcctgg
ggatttggggttgctctggaaaactcatttgcaccactgctgtgccttggaatgctagttggagtaataaatctctggaacagatttggaat
cacacgacctggatggagtgggacagagaaattaacaattacacaagcttaatacactccttaattgaagaatcgcaaaaccagcaag
aaaagaatgaacaagaattattggaattagataaatgggcaagtttgtggaattggtttaacataacaaattggctgtggtatataaaattat
tcataatgatagtaggaggcttggtaggtttaagaatagtttttgctgtactttctatagtgaatagagttaggcagggatattcaccattatc
gtttcagacccacctcccaaccccgaggggacccgacaggcccgaaggaatagaagaagaaggtggagagagagacagagaca
gatccattcgattagtgaacggatCTCGACGGTATCGATCTCGAcacaaatggcagtattcatccacaattttaaaag
aaaaggggggattggggggtacagtgcaggggaaagaatagtagacataatagcaacagacatacaaactaaagaattacaaaaac
aaattacaaaaattcaaaattttcgggtttattacagggacagcagaTGCCGCCCACTCAGACTTTATTCAAAG
ACCACGGGGGTACGGGTGCAGGAAGGGGAGGAGGGGCTGGGGGGAGGCCCAAG
GGGCAAGAAGCATGGCCACCGAGGCTCCAGCTTAACGGTATTTGGAGGTCAGCA
CGGTGCTCACAGAAGCCAGGAACTTGTCCAGGGAGGCGTGCACCGCAGGGGTGA
ACTCGGCGGGGAGGTGGGCGGCCAGGGTCACCAGCAGGCAGTGGCTTAGGAGCT
GTGCAGAGAAGAGGGTCAGTGGGGCCGAGGGCCCAGGCCCGCAGCCGCCGCCTG
CGCTACACCTCCCGCAACCCGCGTGATCCTCTGCCCTGCGAGGAAGGCGCCATCT
CGCCCCTCGACCCAGATCGCTCCCGGCCCGCCGCTCACCTTGAAGTTGACCGGGT
CCACCCGAAGCTTGTGCGCGTGCAGGTCGCTCAGGGCGGACAGCGCGTTGGGCA
TGTCGTCCACGTGCGCCACGGCGTTGGTCAGCGCGTCGGCCACCTTCTTGCCGTG
GCCCTTAACCTGGGCAGAGCCGTGGCTCAGGTCGAAGTGCGGGAAGTAGGTCTT
GGTGGTGGGGAAGGACAGGAACATCCTGCGGGGAGAAGCAGAGTGAGGGGTGG
GGTTTGGGTCCGGGGCCAGGACGGTTGAGGGTGGCCTGTGGGTCCGGGGGGCG
AGGAGCCCGGGTCGGAGCAGGGGAGGGAGCCTCACCTCTCCAGGGCCTCCGCAC
CATACTCGCCAGCGTGCGCGCCGACCTTACCCCAGGCGGCCTTGACGTTGGTCTT
GTCGGCAGGAGACAGCACCATGGTGGGTTCTCTCTGAGTCTGTGGGGACCAGAA
GAGTaagcaatagatggctctgccctgacttttatgcccagccctggctcctgccctccctgctcctgggagtagattggccaaccc
tagggtgtggctccacagggtgaggtctaagtgatgacagccgtacctgtccttggctcttctggcactggcttaggagttggacttcaa
accctcagccctccctctaagatatatctcttggccccataccatcagtacaaattgctactaaaaacatcctcctttgcaagtgtatttacT
ACGTaTATGTGTATATATATATATATATTCAGGAAATAATATATTCTAGAATATGT
CACATTCTGTCTCAGGCATCCATTTTCTTTATGATGCCGTTTGAGGTGGAGTTTTA
GTCAGGTGGTCAGCTTCTCCTTTTTTTTGCCATCTGCCCTGTAAGCATCCTGCTGG
GGACCCAGATAGGAGTCATCACTCTAGGCTGAGAACATCTGGGCACACACCCTA
AGCCTCAGCATGACTCATCATGACTCAGCATTGCTGTGCTTGAGCCAGAAGGTTT
GCTTAGAAGGTTACACAGAACCAGAAGGCGGGGGTGGGGCACTGACCCCGACAG
GGGCCTGGCCAGAACTGCTCATGCTTGGACTATGGGAGGTCACTAATGGAGACA
CACAGAAATGTAACAGGAACTAAGGAAAAACTGAAGCTTtgggggtataggggagcagtccc
atgtagtagtagaatgaaaaatgctgctatgctgtgcctcccccacctttcccatgtctgccctctactcatggtctatctctcctggctcct
gggagtcatggactccacccagcaccaccaacctgacctaaccacctatctgagcctgccagcctataacccatctgggccctgatag
ctggtggccagccctgaccccaccccaccctccctggaacctctgatagacacatctggcacaccagctcgcaaagtcaccgtgagg
gtcttgtgtttgctgagtcaaaattccttgaaatccaagtccttagagactcccaggcttggattcaaagctcctgactttctgtctagtgtat
gtgcagtgagccccttttcctctaactgaaagaaggaaaaaaaaatggaacccaaaatattctacatagtttccatgtcacagccagggc
tgggcagtctcctgttatttcttttaaaataaatatatcatttaaatgcataaataagcaaaccctgctcgggaatgggagggagagtctctg
gagtccaccccttctcggccctggctctgcagatagtgctatcaaagccctgacagagccctgcccattgctgggccttggagtgagtc
agcctagtagagaggcagggcaagccatctcatagctgctgagtgggagagagaaaagggctcattgtctataaactcaggtcatgg
ctattcttattaaaagaaaaggggggactggaagggctaattcactcccaacgaagacaagatctgctttttgcttgtactGGGTCT
CTCTGGTTAGACCAGATCTGAGCCTGGGAGCTCTCTGGCTAACTAGGGAACCCAC
TGCTTAAGCCTCAATAAAGCTTGCCTTGAGTGCTTCAAGTAGTGTGTTGGTTTTTT
GTGTGTGCATTGTCTGAGTAGGTGTCATTCTATTCTGGGGGGTGGGGGGGGCAG
CACAGCAAGGGGGAGGATTGGGAAGACAATAGCAGGCATGCTGGGGATGCGGT
GGGCTCTATGGagtagtagttcatgtcatcttattattcagtatttataacttgcaaagaaatgaatatcagagagtgagaggaa
cttgtttattgcagcttataatggttacaaataaagcaatagcatcacaaatttcacaaataaagcatttttttcactgcattctagttgtggttt
gtccaaactcatcaatgtatcttatcatgtctggctctagctatcccgcccctaactccgcccATCCCGCCCCTAACTCCG
CCCAGTTCCGCCCATTCTCCGCCCCATGGCTCTCACTACTTCTGGAATAGCTCAGA
GGCCGAGGCGGCCTCGGCCTCTGCATAAATAAAAAAAATTAGTCGAGGCTTTTTT
GGAGGCCTAGGgacgtacccaattcgccctatagtgagtcgtattacgcgcgctcactggccgtcgttttacaacgtcgtgac
tgggaaaaccctggcgttacccaacttaatcgccttgcagcacatccccctttcgccagctggcgtaatagcgaagaggcccgcaccg
atcgcccttcccaacagttgcgcagcctgaatggcgaatggg
A2-αP-GLOBE
Sequence ID No: 35
acgcgccctgtagcggcgcattaagcgcggcgggtgtggtggttacgcgcagcgtgaccgctacacttgccagcgccctagcgccc
gctcctttcgctttcttcccttcctttctcgccacgttcgccggctttccccgtcaagctctaaatcgggggctccctttagggttccgattta
gtgcttttacggcacctcgaccccaaaaaacttgattagggtgatggtttcacgtagtgggccatcgccctgatagacggtttttcgcccttt
gacgttggagtccacgttctttaatagtggactcttgttccaaactggaacaacactcaaccctatctcggtctattcttttgatttataaggg
attttgccgatttcggcctattggttaaaaaatgagctgatttaacaaaaatttaacgcgaattttaacaaaatattaacgcttacaatttaggt
ggcacttttcggggaaatgtgcgcggaacccctatttgtttatttttctaaatacattcaaatatgtatccgctcatgagacaataaccctgat
aaatgcttcaataatagcacctagatcaagagacaggatgaggatcgtttcgcatgattgaacaagatggattgcacgcaggttctccg
gccgcttgggtggagaggctattcggctatgactgggcacaacagacaatcggctgctctgatgccgccgtgttccggctgtcagcgc
aggggcgcccggttctttttgtcaagaccgacctgtccggtgccctgaatgaactgcaagacgaggcagcgcggctatcgtggctgg
ccacgacgggcgttccttgcgcagctgtgctcgacgttgtcactgaagcgggaagggactggctgctattgggcgaagtgccgggg
caggatctcctgtcatctcaccttgctcctgccgagaaagtatccatcatggctgatgcaatgcggcggctgcatacgcttgatccggct
acctgcccattcgaccaccaagcgaaacatcgcatcgagcgagcacgtactcggatggaagccggtcttgtcgatcaggatgatctg
gacgaagagcatcaggggctcgcgccagccgaactgttcgccaggctcaaggcgagcatgcccgacggcgaggatctcgtcgtga
cccatggcgatgcctgcttgccgaatatcatggtggaaaatggccgcttttctggattcatcgactgtggccggctgggtgtggcggac
cgctatcaggacatagcgttggctacccgtgatattgctgaagagcttggcggcgaatgggctgaccgcttcctcgtgctttacggtatc
gccgctcccgattcgcagcgcatcgccttctatcgccttcttgacgagttcttctgaattattaacgcttacaatttcctgatgcggtattttct
ccttacgcatctgtgcggtatttcacaccgcatcaggtggcacttttcggggaaatgtgcgcggaacccctatttgtttatttttctaaatac
attcaaatatgtatccgctcatgaccaaaatcccttaacgtgagttttcgttccactgagcgtcagaccccgtagaaaagatcaaaggatc
ttcttgagatcctttttttctgcgcgtaatctgctgcttgcaaacaaaaaaaccaccgctaccagcggtggtttgtttgccggatcaagagct
accaactctttttccgaaggtaactggcttcagcagagcgcagataccaaatactgttcttctagtgtagccgtagttaggccaccacttc
aagaactctgtagcaccgcctacatacctcgctctgctaatcctgttaccagtggctgctgccagtggcgataagtcgtgtcttaccggg
ttggactcaagacgatagttaccggataaggcgcagcggtcgggctgaacgggggggtcgtgcacacagcccagcttggagcgaa
cgacctacaccgaactgagatacctacagcgtgagctatgagaaagcgccacgcttcccgaagggagaaaggcggacaggtatcc
ggtaagcggcagggtcggaacaggagagcgcacgagggagcttccagggggaaacgcctggtatctttatagtcctgtcgggtttc
gccacctctgacttgagcgtcgatttttgtgatgctcgtcaggggggcggagcctatggaaaaacgccagcaacgcggcctttttacgg
ttcctggccttttgctggccttttgctcacatgttctttcctgcgttatcccctgattctgtggataaccgtattaccgcctttgagtgagctgat
accgctcgccgcagccgaacgaccgagcgcagcgagtcagtgagcgaggaagcggaagagcgcccaatacgcaaaccgcctct
ccccgcgcgttggccgattcattaatgcagctggcacgacaggtttcccgactggaaagcgggcagtgagcgcaacgcaattaatgt
gagttagctcactcattaggcaccccaggctttacactttatgcttccggctcgtatgttgtgtggaattgtgagcggataacaatttcaca
caggaaacagctatgaccatgattacgccaagcgcgcaattaaccctcactaaagggaacaaaagctggagctgcaagcttggccat
tgcatacgttgtatccatatcataatatgtacatttatattggctcatgtccaacattaccgccatgttgacattgattattgactagttattaata
gtaatcaattacggggtcattagttcatagcccatatatggagttccgcgttacataacttacggtaaatggcccgcctggctgaccgccc
aacgacccccgcccattgacgtcaataatgacgtatgttcccatagtaacgccaatagggactttccattgacgtcaatgggtggagtat
ttacggtaaactgcccacttggcagtacatcaagtgtatcatatgccaagtacgccccctattgacgtcaatgacggtaaatggcccgcc
tggcattatgcccagtacatgaccttatgggactttcctacttggcagtacatctacgtattagtcatcgctattaccatggtgatgcggtttt
ggcagtacatcaatgggcgtggatagcggtttgactcacggggatttccaagtctccaccccattgacgtcaatgggagtttgttttggc
accaaaatcaacgggactttccaaaatgtcgtaacaactccgccccattgacgcaaatgggcggtaggcgtgtacggtgggaggtcta
tataagcagagctcgtttagtgaaccgGGGTCTCTCTGGTTAGACCAGATCTGAGCCTGGGAGCT
CTCTGGCTAACTAGGGAACCCACTGCTTAAGCCTCAATAAAGCTTGCCTTGAGTG
CTTCAAGTAGTGTGTGCCCGTCTGTTGTGTGACTCTGGTAACTAGAGATCCCTCA
GACCCTTTTAGTCAGTGTGGAAAATCTCTAGCagtggcgcccgaacagggacttgaaagcgaaaggg
aaaccagaggagctctcTCGACGCAGGACTCGGCTTGCTGAAGCGCGCACGGCAAGAGGC
GAGGGGCGGCGACTGGTGAGTACGCCAAAAATTTTGACTAGCGGAGGCTAGAAG
GAGAGAGATGGGTGCGAGAGCGTCAGTATTAAGCGGGGGAGaattagatcgcgatgggaaa
aaattcggttaaggccagggggaaagaaaaaatataaattaaaacatatagtatgggcaagcagggagctagaacgattcgcagttaa
tcctggcctgttagaaacatcagaaggctgtagacaaatactgggacagctacaaccatcccttcagacaggatcagaagaacttagat
cattatataatacagtagcaaccctctattgtgtgcatcaaaggatagagataaaagacaccaaggaagctttagacaagatagaggaa
gagcaaaacaaaagtaagaccaccgcacagcaagcGGCCGCTgatcttcagacctggaggaggagatatgagggacaattg
gagaagtgaattatataaatataaagtagtaaaaattgaaccattaggagtagcacccaccaaggcaaagagaagagtggtgcagag
agaaaaaagaGCAGTGGGAATaggagctttgttccttgggttcttgggagcagcaggaagcactatgggcgcagcgtcaat
gacgctgacggtacaggccagacaattattgtctggtatagtgcagcagcagaacaatttgctgagggctattgaggcgcaacagcat
ctgttgcaactcacagtctggggcatcaagcagctccaggcaagaatcctggctgtggaaagatacctaaaggatcaacagctcctgg
ggatttggggttgctctggaaaactcatttgcaccactgctgtgccttggaatgctagttggagtaataaatctctggaacagatttggaat
cacacgacctggatggagtgggacagagaaattaacaattacacaagcttaatacactccttaattgaagaatcgcaaaaccagcaag
aaaagaatgaacaagaattattggaattagataaatgggcaagtttgtggaattggtttaacataacaaattggctgtggtatataaaattat
tcataatgatagtaggaggcttggtaggtttaagaatagtttttgctgtactttctatagtgaatagagttaggcagggatattcaccattatc
gtttcagacccacctcccaaccccgaggggacccgacaggcccgaaggaatagaagaagaaggtggagagagagacagagaca
gatccattcgattagtgaacggatCTCGACGGTATCGATCTCGAcacaaatggcagtattcatccacaattttaaaag
aaaaggggggattggggggtacagtgcaggggaaagaatagtagacataatagcaacagacatacaaactaaagaattacaaaaac
aaattacaaaaattcaaaattttcgggtttattacagggacagcagaTGCTGCCCACTCAGACTTTATTCAAAG
ACCAGGAAGGGCCGGTGCAAGGAGGGGAGGAGGGCCCGTTGGGAGGCCCAGCG
GGCAGGAGGAACGGCTACCGAGGCTCCAGCTTAtCtaTAcTTGctcGTgAGtACaGTGC
TCACAGAAGCCAGGAACTTGTCCAGGGAGGCGTGCACCGCAGGGGTGAACTCGG
CGGGGAGGTGGGCGGCCAGGGTCACCAGCAGGCAGTGGCTTAGGAGCTGTGCAG
AGAAGAGGGTCAGTGCGGCCCAGGCCCGCAGCCGCCGCCTGCGCTACACCTCCC
GCAACCCGCGTGATCCTCTGCCCTGAGAGGAAGGCGCCATCTCGCCCCTCGACCC
AGATCGCTCCCGGCCCGCCGCTCACCTTGAAGTTGACCGGGTCCACCCGAAGCTT
GTGCGCGTGCAGGTCGCTCAGGGCGGACAGCGCGTTGGGCATGTCGTCCACGTG
CGCCACGGCGTTGGTCAGCGCGTCGGCCACCTTCTTGCCGTGGCCCTTAACCTGG
GCAGAGCCGTGGCTCAGGTCGAAGTGCGGGAAGTAGGTCTTGGTGGTGGGGAAG
GACAGGAACATCCTGCGGGGAGAAGCAGAGTGAGGGGTGGGGTTTGGGTCCGG
GGCCAGGACGGTTGAGGGTGGCCTGTGGGTCCGGGCGGGCGAGGAGCCCGGGTC
GGAGCAGGGGAGGGAGCCTCACCTCTCCAGGGCCTCCGCACCATACTCGCCAGC
GTGCGCGCCGACCTTACCCCAGGCGGCCTTGACGTTGGTCTTGTCGGCAGGAGAC
AGCACCATGGTGGGTTCTCTCTGAGTCTGTGGGGACCAGAAGAGTGCCGGCCCG
CGAGCGCGCCAGGGTTTATGCTTGGGGCGCGGGGGCACGCCCGGCCGGGCGGCG
CTCATTGGCTGGCGCGGAGCCCGGGGCGCGGCCTGGACCGCAGGGGAGTCCCGG
GCGGGGCCTGCGCGGGGCCGGGCGGCGGGCTCGGCCTCCCCTGGGGGTGCACGC
GGGGCGGGGGCCAGGACGTCTCCACCCTCCACCCGCCACccttttgccacctagctgtccagggg
tgccttaaaatggcaaacaaggtttgttttcttttcctgttttcatgccttcctcttccatatccttgtttcatattaatacatgtgtatagatcctaa
aaatctatacacatgtattaataaagcctgattctgccgcttctaggtatagaggccacctgcaagataaatatttgattcacaataactaat
cattctatggcaattgataacaacaaatatatatatatatatatatacgtatatgtgtatatatatatatatatattcaggaaataatatattctag
aatatgtcacattctgtctcaggcatccattttctttatgatgccgtttgaggtggagttttagtcaggtggtcagcttctccttttttttgccatc
tgccctgtaagcatcctgctggggacccagataggagtcatcactctaggctgagaacatctgggcacacaccctaagcctcagcatg
actcatcatgactcagcattgctgtgcttgagccagaaggtttgcttagaaggttacacagaaccagaaggcgggggtggggcactga
ccccgacaggggcctggccagaactgctcatgcttggactatgggaggtcactaatggagacacacagaaatgtaacaggaactaa
ggaaaaactgaagcttatttaatcagagatgaggatgctggaagggatagagggagctgagcttgtaaaaagtatagtaatcattcagc
aaatggttttgaagcacctgctggatgctaaacactattttcagtgcttgaatcataaataagaataaaacatgtatcttattccccacaaga
gtccaagtaaaaaataacagttaattataatgtgctctgtcccccaggctggagtgcagtggcacgatctcagctcactgcaacctccgc
ctcccgggttcaagcaattctcctgcctcagccaccctaatagctgggattacaggtgcacaccaccatgccaggctaatttttgtactttt
tgtagaggcagggtatcaccatgttgtccaagatggtcttgaactcctgagctccaagcagtccacccacctcagcctcccaaagtgct
gggattacaggtgtgagacaccatgcccagattttccatatttaatagaggtatttatgggatgggggaaaagaatgtttctctcactgtg
gattattttagagagtggagaatggtcaagatttttttaaaaattaagaaaacataagttggaccttgagaaatgaaaatttatttttttgttgg
aggatacccattctctatctcccatcagggcaagctgtaaggaactggctaagacacagtgagacagagtgacttagtcttagaggccc
cactggtacgacggtcaccaagctttcattaaaaaaagtctaaccagctgcattcgactttgactgcagcagctggttagaaggttctact
ggaggagggtcccagcccattgctaaattaacatcaggctctgagactggcagtatatctctaacagtggttgatgctatcttctggaact
tgcctgctacattgagaccactgacccatacataggaagcccatagctctgtcctgaactgttaggccactggtccagagagtgtgcat
ctcctttgatcctcataataaccctatgagatagacacaattattactcttactttatagatgatgatcctgaaaacataggagtcaaggcac
ttgcccctagctgggggtataggggagcagtcccatgtagtagtagaatgaaaaatgctgctatgctgtgcctcccccacctttcccatg
tctgccctctactcatggtctatctctcctggctcctgggagtcatggactccacccagcaccaccaacctgacctaaccacctatctgag
cctgccagcctataacccatctgggccctgatagctggtggccagccctgaccccaccccaccctccctggaacctctgatagacaca
tctggcacaccagctcgcaaagtcaccgtgagggtcttgtgtttgctgagtcaaaattccttgaaatccaagtccttagagactcctgctc
ccaaatttacagtcatagacttcttcatggctgtctcctttatccacagaatgattcctttgcttcattgccccatccatctgatcctcctcatca
gtgcagcacagggcccatgagcagtagctgcagagtctcacataggtctggcactgcctctgacatgtccgaccttaggcaaatgctt
gactcttctgagctcagtcttgtcatggcaaaataaagataataatagtgtttttttatggagttagcgtgaggatggaaaacaatagcaaa
attgattagactataaaaggtctcaacaaatagtagtagattttatcatccattaatccttccctctcctctcttactcatcccatcacgtatgc
ctcttaattttcccttacctataataagagttattcctcttattatattcttcttatagtgattctggatattaaagtgggaatgaggggcaggcc
actaacgaagaagatgtttctcaaagaagctaaaagaaaaggggggactggaagggctaattcactcccaacgaagacaagatctgc
tttttgcttgtactGGGTCTCTCTGGTTAGACCAGATCTGAGCCTGGGAGCTCTCTGGCTAA
CTAGGGAACCCACTGCTTAAGCCTCAATAAAGCTTGCCTTGAGTGCTTCAAGTAG
TGTGTTGGTTTTTTGTGTGTGCATTGTCTGAGTAGGTGTCATTCTATTCTGGGGGG
TGGGGTGGGGCAGCACAGCAAGGGGGAGGATTGGGAAGACAATAGCAGGCATG
CTGGGGATGCGGTGGGCTCTATGGagtagtagttcatgtcatcttattattcagtatttataacttgcaaagaaatga
atatcagagagtgagaggaacttgtttattgcagcttataatggttacaaataaagcaatagcatcacaaatttcacaaataaagcattttttt
cactgcattctagttgtggtttgtccaaactcatcaatgtatcttatcatgtctggctctagctatcccgcccctaactccgcccATCCC
GCCCCTAACTCCGCCCAGTTCCGCCCATTCTCCGCCCCATGGCTCTCACTACTTCT
GGAATAGCTCAGAGGCCGAGGCGGCCTCGGCCTCTGCATAAATAAAAAAAATTA
GTCGAGGCTTTTTTGGAGGCCTAGGgacgtacccaattcgccctatagtgagtcgtattacgcgcgctcactgg
ccgtcgttttacaacgtcgtgactgggaaaaccctggcgttacccaacttaatcgccttgcagcacatccccctttcgccagctggcgta
atagcgaagaggcccgcaccgatcgcccttcccaacagttgcgcagcctgaatggcgaatggg
A2-αP-UV
Sequence ID No: 36
acgcgccctgtagcggcgcattaagcgcggcgggtgtggtggttacgcgcagcgtgaccgctacacttgccagcgccctagcgccc
gctcctttcgctttcttcccttcctttctcgccacgttcgccggctttccccgtcaagctctaaatcgggggctccctttagggttccgattta
gtgctttacggcacctcgaccccaaaaaacttgattagggtgatggttcacgtagtgggccatcgccctgatagacggtttttcgcccttt
gacgttggagtccacgttctttaatagtggactcttgttccaaactggaacaacactcaaccctatctcggtctattcttttgatttataaggg
attttgccgatttcggcctattggttaaaaaatgagctgatttaacaaaaatttaacgcgaattttaacaaaatattaacgcttacaatttaggt
ggcacttttcggggaaatgtgcgcggaacccctatttgtttatttttctaaatacattcaaatatgtatccgctcatgagacaataaccctgat
aaatgcttcaataatagcacctagatcaagagacaggatgaggatcgtttcgcatgattgaacaagatggattgcacgcaggttctccg
gccgcttgggtggagaggctattcggctatgactgggcacaacagacaatcggctgctctgatgccgccgtgttccggctgtcagcgc
aggggcgcccggttctttttgtcaagaccgacctgtccggtgccctgaatgaactgcaagacgaggcagcgcggctatcgtggctgg
ccacgacgggcgttccttgcgcagctgtgctcgacgttgtcactgaagcgggaagggactggctgctattgggcgaagtgccgggg
caggatctcctgtcatctcaccttgctcctgccgagaaagtatccatcatggctgatgcaatgcggcggctgcatacgcttgatccggct
acctgcccattcgaccaccaagcgaaacatcgcatgagcgagcacgtactcggatggaagccggtcttgtcgatcaggatgatctg
gacgaagagcatcaggggctcgcgccagccgaactgttcgccaggctcaaggcgagcatgcccgacggcgaggatctcgtcgtga
cccatggcgatgcctgcttgccgaatatcatggtggaaaatggccgcttttctggattcatcgactgtggccggctgggtgtggcggac
cgctatcaggacatagcgttggctacccgtgatattgctgaagagcttggcggcgaatgggctgaccgcttcctcgtgctttacggtatc
gccgctcccgattcgcagcgcatcgccttctatcgccttcttgacgagttcttctgaattattaacgcttacaatttcctgatgcggtattttct
ccttacgcatctgtgcggtatttcacaccgcatcaggtggcacttttcggggaaatgtgcgcggaacccctatttgtttatttttctaaatac
attcaaatatgtatccgctcatgaccaaaatcccttaacgtgagttttcgttccactgagcgtcagaccccgtagaaaagatcaaaggatc
ttcttgagatcctttttttctgcgcgtaatctgctgcttgcaaacaaaaaaaccaccgctaccagcggtggtttgtttgccggatcaagagct
accaactctttttccgaaggtaactggcttcagcagagcgcagataccaaatactgttcttctagtgtagccgtagttaggccaccacttc
aagaactctgtagcaccgcctacatacctcgctctgctaatcctgttaccagtggctgctgccagtggcgataagtcgtgtcttaccggg
ttggactcaagacgatagttaccggataaggcgcagcggtcgggctgaacgggggggtcgtgcacacagcccagcttggagcgaa
cgacctacaccgaactgagatacctacagcgtgagctatgagaaagcgccacgcttcccgaagggagaaaggcggacaggtatcc
ggtaagcggcagggtcggaacaggagagcgcacgagggagcttccagggggaaacgcctggtatctttatagtcctgtcgggtttc
gccacctctgacttgagcgtcgatttttgtgatgctcgtcaggggggcggagcctatggaaaaacgccagcaacgcggcctttttacgg
ttcctggccttttgctggccttttgctcacatgttctttcctgcgttatcccctgattctgtggataaccgtattaccgcctttgagtgagctgat
accgctcgccgcagccgaacgaccgagcgcagcgagtcagtgagcgaggaagcggaagagcgcccaatacgcaaaccgcctct
ccccgcgcgttggccgattcattaatgcagctggcacgacaggtttcccgactggaaagcgggcagtgagcgcaacgcaattaatgt
gagttagctcactcattaggcaccccaggctttacactttatgcttccggctcgtatgttgtgtggaattgtgagcggataacaatttcaca
caggaaacagctatgaccatgattacgccaagcgcgcaattaaccctcactaaagggaacaaaagctggagctgcaagcttggccat
tgcatacgttgtatccatatcataatatgtacatttatattggctcatgtccaacattaccgccatgttgacattgattattgactagttattaata
gtaatcaattacggggtcattagttcatagcccatatatggagttccgcgttacataacttacggtaaatggcccgcctggctgaccgccc
aacgacccccgcccattgacgtcaataatgacgtatgttcccatagtaacgccaatagggactttccattgacgtcaatgggtggagtat
ttacggtaaactgcccacttggcagtacatcaagtgtatcatatgccaagtacgccccctattgacgtcaatgacggtaaatggcccgcc
tggcattatgcccagtacatgaccttatgggactttcctacttggcagtacatctacgtattagtcatcgctattaccatggtgatgcggtttt
ggcagtacatcaatgggcgtggatagcggtttgactcacggggatttccaagtctccaccccattgacgtcaatgggagtttgttttggc
accaaaatcaacgggactttccaaaatgtcgtaacaactccgccccattgacgcaaatgggcggtaggcgtgtacggtgggaggtcta
tataagcagagctcgtttagtgaaccgGGGTCTCTCTGGTTAGACCAGATCTGAGCCTGGGAGCT
CTCTGGCTAACTAGGGAACCCACTGCTTAAGCCTCAATAAAGCTTGCCTTGAGTG
CTTCAAGTAGTGTGTGCCCGTCTGTTGTGTGACTCTGGTAACTAGAGATCCCTCA
GACCCTTTTAGTCAGTGTGGAAAATCTCTAGCagtggcgcccgaacagggacttgaaagcgaaaggg
aaaccagaggagctctcTCGACGCAGGACTCGGCTTGCTGAAGCGCGCACGGCAAGAGGC
GAGGGGCGGCGACTGGTGAGTACGCCAAAAATTTTGACTAGCGGAGGCTAGAAG
GAGAGAGATGGGTGCGAGAGCGTCAGTATTAAGCGGGGGAGaattagatcgcgatgggaaa
aaattcggttaaggccagggggaaagaaaaaatataaattaaaacatatagtatgggcaagcagggagctagaacgattcgcagttaa
tcctggcctgttagaaacatcagaaggctgtagacaaatactgggacagctacaaccatcccttcagacaggatcagaagaacttagat
cattatataatacagtagcaaccctctattgtgtgcatcaaaggatagagataaaagacaccaaggaagctttagacaagatagaggaa
gagcaaaacaaaagtaagaccaccgcacagcaagcGGCCGCTgatcttcagacctggaggaggagatatgagggacaattg
gagaagtgaattatataaatataaagtagtaaaaattgaaccattaggagtagcacccaccaaggcaaagagaagagtggtgcagag
agaaaaaagaGCAGTGGGAATaggagctttgttccttgggttcttgggagcagcaggaagcactatgggcgcagcgtcaat
gacgctgacggtacaggccagacaattattgtctggtatagtgcagcagcagaacaatttgctgagggctattgaggcgcaacagcat
ctgttgcaactcacagtctggggcatcaagcagctccaggcaagaatcctggctgtggaaagatacctaaaggatcaacagctcctgg
ggatttggggttgctctggaaaactcatttgcaccactgctgtgccttggaatgctagttggagtaataaatctctggaacagatttggaat
cacacgacctggatggagtgggacagagaaattaacaattacacaagcttaatacactccttaattgaagaatcgcaaaaccagcaag
aaaagaatgaacaagaattattggaattagataaatgggcaagtttgtggaattggtttaacataacaaattggctgtggtatataaaattat
tcataatgatagtaggaggcttggtaggtttaagaatagtttttgctgtactttctatagtgaatagagttaggcagggatattcaccattatc
gtttcagacccacctcccaaccccgaggggacccgacaggcccgaaggaatagaagaagaaggtggagagagagacagagaca
gatccattcgattagtgaacggatCTCGACGGTATCGATCTCGAcacaaatggcagtattcatccacaattttaaaag
aaaaggggggattggggggtacagtgcaggggaaagaatagtagacataatagcaacagacatacaaactaaagaattacaaaaac
aaattacaaaaattcaaaattttcgggtttattacagggacagcagaTGCTGCCCACTCAGACTTTATTCAAAG
ACCAGGAAGGGCCGGTGCAAGGAGGGGAGGAGGGCCCGTTGGGAGGCCCAGCG
GGCAGGAGGAACGGCTACCGAGGCTCCAGCtgtgctgctctgcggcgaTTAACGGTATTTG
GAGGTCAGCACGGTGCTCACAGAAGCCAGGAACTTGTCCAGGGAGGCGTGCACC
GCAGGGGTGAACTCGGCGGGGAGGTGGGCGGCCAGGGTCACCAGCAGGCAGTG
GCTTAGGAGCTGTGCAGAGAAGAGGGTCAGTGCGGCCCAGGCCCGCAGCCGCCG
CCTGCGCTACACCTCCCGCAACCCGCGTGATCCTCTGCCCTGAGAGGAAGGCGCC
ATCTCGCCCCTCGACCCAGATCGCTCCCGGCCCGCCGCTCACCTTGAAGTTGACC
GGGTCCACCCGAAGCTTGTGCGCGTGCAGGTCGCTCAGGGCGGACAGCGCGTTG
GGCATGTCGTCCACGTGCGCCACGGCGTTGGTCAGCGCGTCGGCCACCTTCTTGC
CGTGGCCCTTAACCTGGGCAGAGCCGTGGCTCAGGTCGAAGTGCGGGAAGTAGG
TCTTGGTGGTGGGGAAGGACAGGAACATCCTGCGGGGAGAAGCAGAGTGAGGG
GTGGGGTTTGGGTCCGGGGCCAGGACGGTTGAGGGTGGCCTGTGGGTCCGGGCG
GGCGAGGAGCCCGGGTCGGAGCAGGGGAGGGAGCCTCACCTCTCCAGGGCCTCC
GCACCATACTCGCCAGCGTGCGCGCCGACCTTACCCCAGGCGGCCTTGACGTTGG
TCTTGTCGGCAGGAGACAGCACCATGGTGGGTTCTCTCTGAGTCTGTGGGGACCA
GAAGAGTGCCGGCCCGCGAGCGCGCCAGGGTTTATGCTTGGGGCGCGGGGGCAC
GCCCGGCCGGGCGGCGCTCATTGGCTGGCGCGGAGCCCGGGGCGCGGCCTGGAC
CGCAGGGGAGTCCCGGGCGGGGCCTGCGCGGGGCCGGGCGGCGGGCTCGGCCTC
CCCTGGGGGTGCACGCGGGGCGGGGGCCAGGACGTCTCCACCCTCCACCCGCCA
CTACGTaTATGTGTATATATATATATATATTCAGGAAATAATATATTCTAGAATAT
GTCACATTCTGTCTCAGGCATCCATTTTCTTTATGATGCCGTTTGAGGTGGAGTTT
TAGTCAGGTGGTCAGCTTCTCCTTTTTTTTGCCATCTGCCCTGTAAGCATCCTGCT
GGGGACCCAGATAGGAGTCATCACTCTAGGCTGAGAACATCTGGGCACACACCC
TAAGCCTCAGCATGACTCATCATGACTCAGCATTGCTGTGCTTGAGCCAGAAGGT
TTGCTTAGAAGGTTACACAGAACCAGAAGGCGGGGGTGGGGCACTGACCCCGAC
AGGGGCCTGGCCAGAACTGCTCATGCTTGGACTATGGGAGGTCACTAATGGAGA
CACACAGAAATGTAACAGGAACTAAGGAAAAACTGAAGCTTtgggggtataggggagcagt
cccatgtagtagtagaatgaaaaatgctgctatgctgtgcctcccccacctttcccatgtctgccctctactcatggtctatctctcctggct
cctgggagtcatggactccacccagcaccaccaacctgacctaaccacctatctgagcctgccagcctataacccatctgggccctga
tagctggtggccagccctgaccccaccccaccctccctggaacctctgatagacacatctggcacaccagctcgcaaagtcaccgtg
agggtcttgtgtttgctgagtcaaaattccttgaaatccaagtccttagagactcccaggcttggattcaaagctcctgactttctgtctagt
gtatgtgcagtgagccccttttcctctaactgaaagaaggaaaaaaaaatggaacccaaaatattctacatagtttccatgtcacagcca
gggctgggcagtctcctgttatttcttttaaaataaatatatcatttaaatgcataaataagcaaaccctgctcgggaatgggagggagagt
ctctggagtccaccccttctcggccctggctctgcagatagtgctatcaaagccctgacagagccctgcccattgctgggccttggagt
gagtcagcctagtagagaggcagggcaagccatctcatagctgctgagtgggagagagaaaagggctcattgtctataaactcaggt
catggctattcttattaaaagaaaaggggggactggaagggctaattcactcccaacgaagacaagatctgctttttgcttgtactGGG
TCTCTCTGGTTAGACCAGATCTGAGCCTGGGAGCTCTCTGGCTAACTAGGGAACC
CACTGCTTAAGCCTCAATAAAGCTTGCCTTGAGTGCTTCAAGTAGTGTGTTGGTTT
TTTGTGTGTGCATTGTCTGAGTAGGTGTCATTCTATTCTGGGGGGTGGGGTGGGG
CAGCACAGCAAGGGGGAGGATTGGGAAGACAATAGCAGGCATGCTGGGGATGC
GGTGGGCTCTATGGagtagtagttcatgtcatcttattattcagtatttataacttgcaaagaaatgaatatcagagagtgaga
ggaacttgtttattgcagcttataatggttacaaataaagcaatagcatcacaaatttcacaaataaagcatttttttcactgcattctagttgt
ggtttgtccaaactcatcaatgtatcttatcatgtctggctctagctatcccgcccctaactccgcccATCCCGCCCCTAACT
CCGCCCAGTTCCGCCCATTCTCCGCCCCATGGCTCTCACTACTTCTGGAATAGCTC
AGAGGCCGAGGCGGCCTCGGCCTCTGCATAAATAAAAAAAATTAGTCGAGGCTT
TTTTGGAGGCCTAGGgacgtacccaattcgccctatagtgagtcgtattacgcgcgctcactggccgtcgttttacaacgt
cgtgactgggaaaaccctggcgttacccaacttaatcgccttgcagcacatccccctttcgccagctggcgtaatagcgaagaggccc
gcaccgatcgcccttcccaacagttgcgcagcctgaatggcgaatggg
A2-B-UTRS-UV
Sequence ID No: 37
acgcgccctgtagcggcgcattaagcgcggcgggtgtggtggttacgcgcagcgtgaccgctacacttgccagcgccctagcgccc
gctcctttcgctttcttcccttcctttctcgccacgttcgccggctttccccgtcaagctctaaatcgggggctccctttagggttccgattta
gtgctttacggcacctcgaccccaaaaaacttgattagggtgatggttcacgtagtgggccatcgccctgatagacggtttttcgcccttt
gacgttggagtccacgttctttaatagtggactcttgttccaaactggaacaacactcaaccctatctcggtctattcttttgatttataaggg
attttgccgatttcggcctattggttaaaaaatgagctgatttaacaaaaatttaacgcgaattttaacaaaatattaacgcttacaatttaggt
ggcacttttcggggaaatgtgcgcggaacccctatttgtttatttttctaaatacattcaaatatgtatccgctcatgagacaataaccctgat
aaatgcttcaataatagcacctagatcaagagacaggatgaggatcgtttcgcatgattgaacaagatggattgcacgcaggttctccg
gccgcttgggtggagaggctattcggctatgactgggcacaacagacaatcggctgctctgatgccgccgtgttccggctgtcagcgc
aggggcgcccggttctttttgtcaagaccgacctgtccggtgccctgaatgaactgcaagacgaggcagcgcggctatcgtggctgg
ccacgacgggcgttccttgcgcagctgtgctcgacgttgtcactgaagcgggaagggactggctgctattgggcgaagtgccgggg
caggatctcctgtcatctcaccttgctcctgccgagaaagtatccatcatggctgatgcaatgcggcggctgcatacgcttgatccggct
acctgcccattcgaccaccaagcgaaacatcgcatcgagcgagcacgtactcggatggaagccggtcttgtcgatcaggatgatctg
gacgaagagcatcaggggctcgcgccagccgaactgttcgccaggctcaaggcgagcatgcccgacggcgaggatctcgtcgtga
cccatggcgatgcctgcttgccgaatatcatggtggaaaatggccgcttttctggattcatcgactgtggccggctgggtgtggcggac
cgctatcaggacatagcgttggctacccgtgatattgctgaagagcttggcggcgaatgggctgaccgcttcctcgtgctttacggtatc
gccgctcccgattcgcagcgcatcgccttctatcgccttcttgacgagttcttctgaattattaacgcttacaatttcctgatgcggtattttct
ccttacgcatctgtgcggtatttcacaccgcatcaggtggcacttttcggggaaatgtgcgcggaacccctatttgtttatttttctaaatac
attcaaatatgtatccgctcatgaccaaaatcccttaacgtgagttttcgttccactgagcgtcagaccccgtagaaaagatcaaaggatc
ttcttgagatcctttttttctgcgcgtaatctgctgcttgcaaacaaaaaaaccaccgctaccagcggtggtttgtttgccggatcaagagct
accaactctttttccgaaggtaactggcttcagcagagcgcagataccaaatactgttcttctagtgtagccgtagttaggccaccacttc
aagaactctgtagcaccgcctacatacctcgctctgctaatcctgttaccagtggctgctgccagtggcgataagtcgtgtcttaccggg
ttggactcaagacgatagttaccggataaggcgcagcggtcgggctgaacgggggggtcgtgcacacagcccagcttggagcgaa
cgacctacaccgaactgagatacctacagcgtgagctatgagaaagcgccacgcttcccgaagggagaaaggcggacaggtatcc
ggtaagcggcagggtcggaacaggagagcgcacgagggagcttccagggggaaacgcctggtatctttatagtcctgtcgggtttc
gccacctctgacttgagcgtcgatttttgtgatgctcgtcaggggggcggagcctatggaaaaacgccagcaacgcggcctttttacgg
ttcctggccttttgctggccttttgctcacatgttctttcctgcgttatcccctgattctgtggataaccgtattaccgcctttgagtgagctgat
accgctcgccgcagccgaacgaccgagcgcagcgagtcagtgagcgaggaagcggaagagcgcccaatacgcaaaccgcctct
ccccgcgcgttggccgattcattaatgcagctggcacgacaggtttcccgactggaaagcgggcagtgagcgcaacgcaattaatgt
gagttagctcactcattaggcaccccaggctttacactttatgcttccggctcgtatgttgtgtggaattgtgagcggataacaatttcaca
caggaaacagctatgaccatgattacgccaagcgcgcaattaaccctcactaaagggaacaaaagctggagctgcaagcttggccat
tgcatacgttgtatccatatcataatatgtacatttatattggctcatgtccaacattaccgccatgttgacattgattattgactagttattaata
gtaatcaattacggggtcattagttcatagcccatatatggagttccgcgttacataacttacggtaaatggcccgcctggctgaccgccc
aacgacccccgcccattgacgtcaataatgacgtatgttcccatagtaacgccaatagggactttccattgacgtcaatgggtggagtat
ttacggtaaactgcccacttggcagtacatcaagtgtatcatatgccaagtacgccccctattgacgtcaatgacggtaaatggcccgcc
tggcattatgcccagtacatgaccttatgggactttcctacttggcagtacatctacgtattagtcatcgctattaccatggtgatgcggtttt
ggcagtacatcaatgggcgtggatagcggtttgactcacggggatttccaagtctccaccccattgacgtcaatgggagtttgttttggc
accaaaatcaacgggactttccaaaatgtcgtaacaactccgccccattgacgcaaatgggcggtaggcgtgtacggtgggaggtcta
tataagcagagctcgtttagtgaaccgGGGTCTCTCTGGTTAGACCAGATCTGAGCCTGGGAGCT
CTCTGGCTAACTAGGGAACCCACTGCTTAAGCCTCAATAAAGCTTGCCTTGAGTG
CTTCAAGTAGTGTGTGCCCGTCTGTTGTGTGACTCTGGTAACTAGAGATCCCTCA
GACCCTTTTAGTCAGTGTGGAAAATCTCTAGCagtggcgcccgaacagggacttgaaagcgaaaggg
aaaccagaggagctctcTCGACGCAGGACTCGGCTTGCTGAAGCGCGCACGGCAAGAGGC
GAGGGGCGGCGACTGGTGAGTACGCCAAAAATTTTGACTAGCGGAGGCTAGAAG
GAGAGAGATGGGTGCGAGAGCGTCAGTATTAAGCGGGGGAGaattagatcgcgatgggaaa
aaattcggttaaggccagggggaaagaaaaaatataaattaaaacatatagtatgggcaagcagggagctagaacgattcgcagttaa
tcctggcctgttagaaacatcagaaggctgtagacaaatactgggacagctacaaccatcccttcagacaggatcagaagaacttagat
cattatataatacagtagcaaccctctattgtgtgcatcaaaggatagagataaaagacaccaaggaagctttagacaagatagaggaa
gagcaaaacaaaagtaagaccaccgcacagcaagcGGCCGCTgatcttcagacctggaggaggagatatgagggacaattg
gagaagtgaattatataaatataaagtagtaaaaattgaaccattaggagtagcacccaccaaggcaaagagaagagtggtgcagag
agaaaaaagaGCAGTGGGAATaggagctttgttccttgggttcttgggagcagcaggaagcactatgggcgcagcgtcaat
gacgctgacggtacaggccagacaattattgtctggtatagtgcagcagcagaacaatttgctgagggctattgaggcgcaacagcat
ctgttgcaactcacagtctggggcatcaagcagctccaggcaagaatcctggctgtggaaagatacctaaaggatcaacagctcctgg
ggatttggggttgctctggaaaactcatttgcaccactgctgtgccttggaatgctagttggagtaataaatctctggaacagatttggaat
cacacgacctggatggagtgggacagagaaattaacaattacacaagcttaatacactccttaattgaagaatcgcaaaaccagcaag
aaaagaatgaacaagaattattggaattagataaatgggcaagtttgtggaattggtttaacataacaaattggctgtggtatataaaattat
tcataatgatagtaggaggcttggtaggtttaagaatagtttttgctgtactttctatagtgaatagagttaggcagggatattcaccattatc
gtttcagacccacctcccaaccccgaggggacccgacaggcccgaaggaatagaagaagaaggtggagagagagacagagaca
gatccattcgattagtgaacggatCTCGACGGTATCGATCTCGAcacaaatggcagtattcatccacaattttaaaag
aaaaggggggattggggggtacagtgcaggggaaagaatagtagacataatagcaacagacatacaaactaaagaattacaaaaac
aaattacaaaaattcaaaattttcgggtttattacagggacagcagacatgaggacagctaaaacaataagtaatgtaaaatacagcata
gcaaaactttaacctccaaatcaagcctctacttgaatccttttctgagggatgaataaggcataggcatcaggggctgttgccaatgtgc
attagctgtttgcagcctcaccttctttcatggagtttaagatatagtgtattttcccaaggtttgaactagctcttcatttctttatgttttaaatg
cactgacctcccacattccctttttagtaaaatattcagaaataatttaaatacatcattgcaatgaaaataaatgttttttattaggcagaatc
cagatgctcaaggcccttcataatatcccccagtttagtagttggacttagggaacaaaggaacctttaatagaaattggacagcaagaa
agcgagcTTAACGGTATTTGGAGGTCAGCACGGTGCTCACAGAAGCCAGGAACTTGT
CCAGGGAGGCGTGCACCGCAGGGGTGAACTCGGCGGGGAGGTGGGCGGCCAGG
GTCACCAGCAGGCAGTGGCTTAGGAGCTGTGCAGAGAAGAGGGTCAGTGCGGCC
CAGGCCCGCAGCCGCCGCCTGCGCTACACCTCCCGCAACCCGCGTGATCCTCTGC
CCTGAGAGGAAGGCGCCATCTCGCCCCTCGACCCAGATCGCTCCCGGCCCGCCGC
TCACCTTGAAGTTGACCGGGTCCACCCGAAGCTTGTGCGCGTGCAGGTCGCTCAG
GGCGGACAGCGCGTTGGGCATGTCGTCCACGTGCGCCACGGCGTTGGTCAGCGC
GTCGGCCACCTTCTTGCCGTGGCCCTTAACCTGGGCAGAGCCGTGGCTCAGGTCG
AAGTGCGGGAAGTAGGTCTTGGTGGTGGGGAAGGACAGGAACATCCTGCGGGGA
GAAGCAGAGTGAGGGGTGGGGTTTGGGTCCGGGGCCAGGACGGTTGAGGGTGGC
CTGTGGGTCCGGGCGGGCGAGGAGCCCGGGTCGGAGCAGGGGAGGGAGCCTCA
CCTCTCCAGGGCCTCCGCACCATACTCGCCAGCGTGCGCGCCGACCTTACCCCAG
GCGGCCTTGACGTTGGTCTTGTCGGCAGGAGACAGCACCATggtggctgtttgaggttgctagt
gaacacagttgtgtcagaagcaaatgtaagcaatagatggctctgccctgacttttatgcccagccctggctcctgccctccctgctcct
gggagtagattggccaaccctagggtgtggctccacagggtgaggtctaagtgatgacagccgtacctgtccttggctcttctggcact
ggcttaggagttggacttcaaaccctcagccctccctctaagatatatctcttggccccataccatcagtacaaattgctactaaaaacatc
ctcctttgcaagtgtatttacTACGTaTATGTGTATATATATATATATATTCAGGAAATAATATA
TTCTAGAATATGTCACATTCTGTCTCAGGCATCCATTTTCTTTATGATGCCGTTTG
AGGTGGAGTTTTAGTCAGGTGGTCAGCTTCTCCTTTTTTTTGCCATCTGCCCTGTA
AGCATCCTGCTGGGGACCCAGATAGGAGTCATCACTCTAGGCTGAGAACATCTG
GGCACACACCCTAAGCCTCAGCATGACTCATCATGACTCAGCATTGCTGTGCTTG
AGCCAGAAGGTTTGCTTAGAAGGTTACACAGAACCAGAAGGCGGGGGTGGGGCA
CTGACCCCGACAGGGGCCTGGCCAGAACTGCTCATGCTTGGACTATGGGAGGTC
ACTAATGGAGACACACAGAAATGTAACAGGAACTAAGGAAAAACTGAAGCTTtgg
gggtataggggagcagtcccatgtagtagtagaatgaaaaatgctgctatgctgtgcctcccccacctttcccatgtctgccctctactca
tggtctatctctcctggctcctgggagtcatggactccacccagcaccaccaacctgacctaaccacctatctgagcctgccagcctata
acccatctgggccctgatagctggtggccagccctgaccccaccccaccctccctggaacctctgatagacacatctggcacaccag
ctcgcaaagtcaccgtgagggtcttgtgtttgctgagtcaaaattccttgaaatccaagtccttagagactcccaggcttggattcaaagc
tcctgactttctgtctagtgtatgtgcagtgagccccttttcctctaactgaaagaaggaaaaaaaaatggaacccaaaatattctacatag
tttccatgtcacagccagggctgggcagtctcctgttatttcttttaaaataaatatatcatttaaatgcataaataagcaaaccctgctcgg
gaatgggagggagagtctctggagtccaccccttctcggccctggctctgcagatagtgctatcaaagccctgacagagccctgccc
attgctgggccttggagtgagtcagcctagtagagaggcagggcaagccatctcatagctgctgagtgggagagagaaaagggctc
attgtctataaactcaggtcatggctattcttattaaaagaaaaggggggactggaagggctaattcactcccaacgaagacaagatctg
ctttttgcttgtactGGGTCTCTCTGGTTAGACCAGATCTGAGCCTGGGAGCTCTCTGGCTA
ACTAGGGAACCCACTGCTTAAGCCTCAATAAAGCTTGCCTTGAGTGCTTCAAGTA
GTGTGTTGGTTTTTTGTGTGTGCATTGTCTGAGTAGGTGTCATTCTATTCTGGGGG
GTGGGGTGGGGCAGCACAGCAAGGGGGAGGATTGGGAAGACAATAGCAGGCAT
GCTGGGGATGCGGTGGGCTCTATGGagtagtagttcatgtcatcttattattcagtatttataacttgcaaagaaat
gaatatcagagagtgagaggaacttgtttattgcagcttataatggttacaaataaagcaatagcatcacaaatttcacaaataaagcattt
ttttcactgcattctagttgtggtttgtccaaactcatcaatgtatcttatcatgtctggctctagctatcccgcccctaactccgcccATCC
CGCCCCTAACTCCGCCCAGTTCCGCCCATTCTCCGCCCCATGGCTCTCACTACTTC
TGGAATAGCTCAGAGGCCGAGGCGGCCTCGGCCTCTGCATAAATAAAAAAAATT
AGTCGAGGCTTTTTTGGAGGCCTAGGgacgtacccaattcgccctatagtgagtcgtattacgcgcgctcactg
gccgtcgttttacaacgtcgtgactgggaaaaccctggcgttacccaacttaatcgccttgcagcacatccccctttcgccagctggcgt
aatagcgaagaggcccgcaccgatcgcccttcccaacagttgcgcagcctgaatggcgaatggg
A2-cDNA-UV
Sequence ID No: 38
acgcgccctgtagcggcgcattaagcgcggcgggtgtggtggttacgcgcagcgtgaccgctacacttgccagcgccctagcgccc
gctcctttcgctttcttcccttcctttctcgccacgttcgccggctttccccgtcaagctctaaatcgggggctccctttagggttccgattta
gtgctttacggcacctcgaccccaaaaaacttgattagggtgatggttcacgtagtgggccatcgccctgatagacggtttttcgcccttt
gacgttggagtccacgttctttaatagtggactcttgttccaaactggaacaacactcaaccctatctcggtctattcttttgatttataaggg
attttgccgatttcggcctattggttaaaaaatgagctgatttaacaaaaatttaacgcgaattttaacaaaatattaacgcttacaatttaggt
ggcacttttcggggaaatgtgcgcggaacccctatttgtttatttttctaaatacattcaaatatgtatccgctcatgagacaataaccctgat
aaatgcttcaataatagcacctagatcaagagacaggatgaggatcgtttcgcatgattgaacaagatggattgcacgcaggttctccg
gccgcttgggtggagaggctattcggctatgactgggcacaacagacaatcggctgctctgatgccgccgtgttccggctgtcagcgc
aggggcgcccggttctttttgtcaagaccgacctgtccggtgccctgaatgaactgcaagacgaggcagcgcggctatcgtggctgg
ccacgacgggcgttccttgcgcagctgtgctcgacgttgtcactgaagcgggaagggactggctgctattgggcgaagtgccgggg
caggatctcctgtcatctcaccttgctcctgccgagaaagtatccatcatggctgatgcaatgcggcggctgcatacgcttgatccggct
acctgcccattcgaccaccaagcgaaacatcgcatcgagcgagcacgtactcggatggaagccggtcttgtcgatcaggatgatctg
gacgaagagcatcaggggctcgcgccagccgaactgttcgccaggctcaaggcgagcatgcccgacggcgaggatctcgtcgtga
cccatggcgatgcctgcttgccgaatatcatggtggaaaatggccgcttttctggattcatcgactgtggccggctgggtgtggcggac
cgctatcaggacatagcgttggctacccgtgatattgctgaagagcttggcggcgaatgggctgaccgcttcctcgtgctttacggtatc
gccgctcccgattcgcagcgcatcgccttctatcgccttcttgacgagttcttctgaattattaacgcttacaatttcctgatgcggtattttct
ccttacgcatctgtgcggtatttcacaccgcatcaggtggcacttttcggggaaatgtgcgcggaacccctatttgtttatttttctaaatac
attcaaatatgtatccgctcatgaccaaaatcccttaacgtgagttttcgttccactgagcgtcagaccccgtagaaaagatcaaaggatc
ttcttgagatcctttttttctgcgcgtaatctgctgcttgcaaacaaaaaaaccaccgctaccagcggtggtttgtttgccggatcaagagct
accaactctttttccgaaggtaactggcttcagcagagcgcagataccaaatactgttcttctagtgtagccgtagttaggccaccacttc
aagaactctgtagcaccgcctacatacctcgctctgctaatcctgttaccagtggctgctgccagtggcgataagtcgtgtcttaccggg
ttggactcaagacgatagttaccggataaggcgcagcggtcgggctgaacgggggggtcgtgcacacagcccagcttggagcgaa
cgacctacaccgaactgagatacctacagcgtgagctatgagaaagcgccacgcttcccgaagggagaaaggcggacaggtatcc
ggtaagcggcagggtcggaacaggagagcgcacgagggagcttccagggggaaacgcctggtatctttatagtcctgtcgggtttc
gccacctctgacttgagcgtcgatttttgtgatgctcgtcaggggggcggagcctatggaaaaacgccagcaacgcggcctttttacgg
ttcctggccttttgctggccttttgctcacatgttctttcctgcgttatcccctgattctgtggataaccgtattaccgcctttgagtgagctgat
accgctcgccgcagccgaacgaccgagcgcagcgagtcagtgagcgaggaagcggaagagcgcccaatacgcaaaccgcctct
ccccgcgcgttggccgattcattaatgcagctggcacgacaggtttcccgactggaaagcgggcagtgagcgcaacgcaattaatgt
gagttagctcactcattaggcaccccaggctttacactttatgcttccggctcgtatgttgtgtggaattgtgagcggataacaatttcaca
caggaaacagctatgaccatgattacgccaagcgcgcaattaaccctcactaaagggaacaaaagctggagctgcaagcttggccat
tgcatacgttgtatccatatcataatatgtacatttatattggctcatgtccaacattaccgccatgttgacattgattattgactagttattaata
gtaatcaattacggggtcattagttcatagcccatatatggagttccgcgttacataacttacggtaaatggcccgcctggctgaccgccc
aacgacccccgcccattgacgtcaataatgacgtatgttcccatagtaacgccaatagggactttccattgacgtcaatgggtggagtat
ttacggtaaactgcccacttggcagtacatcaagtgtatcatatgccaagtacgccccctattgacgtcaatgacggtaaatggcccgcc
tggcattatgcccagtacatgaccttatgggactttcctacttggcagtacatctacgtattagtcatcgctattaccatggtgatgcggtttt
ggcagtacatcaatgggcgtggatagcggtttgactcacggggatttccaagtctccaccccattgacgtcaatgggagtttgttttggc
accaaaatcaacgggactttccaaaatgtcgtaacaactccgccccattgacgcaaatgggcggtaggcgtgtacggtgggaggtcta
tataagcagagctcgtttagtgaaccgGGGTCTCTCTGGTTAGACCAGATCTGAGCCTGGGAGCT
CTCTGGCTAACTAGGGAACCCACTGCTTAAGCCTCAATAAAGCTTGCCTTGAGTG
CTTCAAGTAGTGTGTGCCCGTCTGTTGTGTGACTCTGGTAACTAGAGATCCCTCA
GACCCTTTTAGTCAGTGTGGAAAATCTCTAGCagtggcgcccgaacagggacttgaaagcgaaaggg
aaaccagaggagctctcTCGACGCAGGACTCGGCTTGCTGAAGCGCGCACGGCAAGAGGC
GAGGGGCGGCGACTGGTGAGTACGCCAAAAATTTTGACTAGCGGAGGCTAGAAG
GAGAGAGATGGGTGCGAGAGCGTCAGTATTAAGCGGGGGAGaattagatcgcgatgggaaa
aaattcggttaaggccagggggaaagaaaaaatataaattaaaacatatagtatgggcaagcagggagctagaacgattcgcagttaa
tcctggcctgttagaaacatcagaaggctgtagacaaatactgggacagctacaaccatcccttcagacaggatcagaagaacttagat
cattatataatacagtagcaaccctctattgtgtgcatcaaaggatagagataaaagacaccaaggaagctttagacaagatagaggaa
gagcaaaacaaaagtaagaccaccgcacagcaagcGGCCGCTgatcttcagacctggaggaggagatatgagggacaattg
gagaagtgaattatataaatataaagtagtaaaaattgaaccattaggagtagcacccaccaaggcaaagagaagagtggtgcagag
agaaaaaagaGCAGTGGGAATaggagctttgttccttgggttcttgggagcagcaggaagcactatgggcgcagcgtcaat
gacgctgacggtacaggccagacaattattgtctggtatagtgcagcagcagaacaatttgctgagggctattgaggcgcaacagcat
ctgttgcaactcacagtctggggcatcaagcagctccaggcaagaatcctggctgtggaaagatacctaaaggatcaacagctcctgg
ggatttggggttgctctggaaaactcatttgcaccactgctgtgccttggaatgctagttggagtaataaatctctggaacagatttggaat
cacacgacctggatggagtgggacagagaaattaacaattacacaagcttaatacactccttaattgaagaatcgcaaaaccagcaag
aaaagaatgaacaagaattattggaattagataaatgggcaagtttgtggaattggtttaacataacaaattggctgtggtatataaaattat
tcataatgatagtaggaggcttggtaggtttaagaatagtttttgctgtactttctatagtgaatagagttaggcagggatattcaccattatc
gtttcagacccacctcccaaccccgaggggacccgacaggcccgaaggaatagaagaagaaggtggagagagagacagagaca
gatccattcgattagtgaacggatCTCGACGGTATCGATCTCGAcacaaatggcagtattcatccacaattttaaaag
aaaaggggggattggggggtacagtgcaggggaaagaatagtagacataatagcaacagacatacaaactaaagaattacaaaaac
aaattacaaaaattcaaaattttcgggtttattacagggacagcagaTGCTGCCCACTCAGACTTTATTCAAAG
ACCAGGAAGGGCCGGTGCAAGGAGGGGAGGAGGGCCCGTTGGGAGGCCCAGCG
GGCAGGAGGAACGGCTACCGAGGCTCCAGCtgtgctgctctgcggcgaTTAACGGTATTTG
GAGGTCAGCACGGTGCTCACAGAAGCCAGGAACTTGTCCAGGGAGGCGTGCACC
GCAGGGGTGAACTCGGCGGGGAGGTGGGCGGCCAGGGTCACCAGCAGGCAGTG
GCTTAGGAGCTTGAAGTTGACCGGGTCCACCCGAAGCTTGTGCGCGTGCAGGTCG
CTCAGGGCGGACAGCGCGTTGGGCATGTCGTCCACGTGCGCCACGGCGTTGGTC
AGCGCGTCGGCCACCTTCTTGCCGTGGCCCTTAACCTGGGCAGAGCCGTGGCTCA
GGTCGAAGTGCGGGAAGTAGGTCTTGGTGGTGGGGAAGGACAGGAACATCCTCT
CCAGGGCCTCCGCACCATACTCGCCAGCGTGCGCGCCGACCTTACCCCAGGCGGC
CTTGACGTTGGTCTTGTCGGCAGGAGACAGCACCATGGTGGGTTCTCTCTGAGTC
TGTGGGGACCAGAAGAGTaagcaatagatggctctgccctgacttttatgcccagccctggctcctgccctccctgct
cctgggagtagattggccaaccctagggtgtggctccacagggtgaggtctaagtgatgacagccgtacctgtccttggctcttctggc
actggcttaggagttggacttcaaaccctcagccctccctctaagatatatctcttggccccataccatcagtacaaattgctactaaaaac
atcctcctttgcaagtgtatttacTACGTaTATGTGTATATATATATATATATTCAGGAAATAATA
TATTCTAGAATATGTCACATTCTGTCTCAGGCATCCATTTTCTTTATGATGCCGTT
TGAGGTGGAGTTTTAGTCAGGTGGTCAGCTTCTCCTTTTTTTTGCCATCTGCCCTG
TAAGCATCCTGCTGGGGACCCAGATAGGAGTCATCACTCTAGGCTGAGAACATCT
GGGCACACACCCTAAGCCTCAGCATGACTCATCATGACTCAGCATTGCTGTGCTT
GAGCCAGAAGGTTTGCTTAGAAGGTTACACAGAACCAGAAGGCGGGGGTGGGGC
ACTGACCCCGACAGGGGCCTGGCCAGAACTGCTCATGCTTGGACTATGGGAGGT
CACTAATGGAGACACACAGAAATGTAACAGGAACTAAGGAAAAACTGAAGCTTt
gggggtataggggagcagtcccatgtagtagtagaatgaaaaatgctgctatgctgtgcctcccccacctttcccatgtctgccctctac
tcatggtctatctctcctggctcctgggagtcatggactccacccagcaccaccaacctgacctaaccacctatctgagcctgccagcct
ataacccatctgggccctgatagctggtggccagccctgaccccaccccaccctccctggaacctctgatagacacatctggcacacc
agctcgcaaagtcaccgtgagggtcttgtgtttgctgagtcaaaattccttgaaatccaagtccttagagactcccaggcttggattcaaa
gctcctgactttctgtctagtgtatgtgcagtgagccccttttcctctaactgaaagaaggaaaaaaaaatggaacccaaaatattctacat
agtttccatgtcacagccagggctgggcagtctcctgttatttcttttaaaataaatatatcatttaaatgcataaataagcaaaccctgctcg
ggaatgggagggagagtctctggagtccaccccttctcggccctggctctgcagatagtgctatcaaagccctgacagagccctgcc
cattgctgggccttggagtgagtcagcctagtagagaggcagggcaagccatctcatagctgctgagtgggagagagaaaagggct
cattgtctataaactcaggtcatggctattcttattaaaagaaaaggggggactggaagggctaattcactcccaacgaagacaagatct
gctttttgcttgtactGGGTCTCTCTGGTTAGACCAGATCTGAGCCTGGGAGCTCTCTGGCTA
ACTAGGGAACCCACTGCTTAAGCCTCAATAAAGCTTGCCTTGAGTGCTTCAAGTA
GTGTGTTGGTTTTTTGTGTGTGCATTGTCTGAGTAGGTGTCATTCTATTCTGGGGG
GTGGGGTGGGGCAGCACAGCAAGGGGGAGGATTGGGAAGACAATAGCAGGCAT
GCTGGGGATGCGGTGGGCTCTATGGagtagtagttcatgtcatcttattattcagtatttataacttgcaaagaaat
gaatatcagagagtgagaggaacttgtttattgcagcttataatggttacaaataaagcaatagcatcacaaatttcacaaataaagcattt
ttttcactgcattctagttgtggtttgtccaaactcatcaatgtatcttatcatgtctggctctagctatcccgcccctaactccgcccATCC
CGCCCCTAACTCCGCCCAGTTCCGCCCATTCTCCGCCCCATGGCTCTCACTACTTC
TGGAATAGCTCAGAGGCCGAGGCGGCCTCGGCCTCTGCATAAATAAAAAAAATT
AGTCGAGGCTTTTTTGGAGGCCTAGGgacgtacccaattcgccctatagtgagtcgtattacgcgcgctcactg
gccgtcgttttacaacgtcgtgactgggaaaaccctggcgttacccaacttaatcgccttgcagcacatccccctttcgccagctggcgt
aatagcgaagaggcccgcaccgatcgcccttcccaacagttgcgcagcctgaatggcgaatggg
A2-DIVS2-GLOBE
Sequence ID No: 39
acgcgccctgtagcggcgcattaagcgcggcgggtgtggtggttacgcgcagcgtgaccgctacacttgccagcgccctagcgccc
gctcctttcgctttcttcccttcctttctcgccacgttcgccggctttccccgtcaagctctaaatcgggggctccctttagggttccgattta
gtgctttacggcacctcgaccccaaaaaacttgattagggtgatggttcacgtagtgggccatcgccctgatagacggtttttcgcccttt
gacgttggagtccacgttctttaatagtggactcttgttccaaactggaacaacactcaaccctatctcggtctattcttttgatttataaggg
attttgccgatttcggcctattggttaaaaaatgagctgatttaacaaaaatttaacgcgaattttaacaaaatattaacgcttacaatttaggt
ggcacttttcggggaaatgtgcgcggaacccctatttgtttatttttctaaatacattcaaatatgtatccgctcatgagacaataaccctgat
aaatgcttcaataatagcacctagatcaagagacaggatgaggatcgtttcgcatgattgaacaagatggattgcacgcaggttctccg
gccgcttgggtggagaggctattcggctatgactgggcacaacagacaatcggctgctctgatgccgccgtgttccggctgtcagcgc
aggggcgcccggttctttttgtcaagaccgacctgtccggtgccctgaatgaactgcaagacgaggcagcgcggctatcgtggctgg
ccacgacgggcgttccttgcgcagctgtgctcgacgttgtcactgaagcgggaagggactggctgctattgggcgaagtgccgggg
caggatctcctgtcatctcaccttgctcctgccgagaaagtatccatcatggctgatgcaatgcggcggctgcatacgcttgatccggct
acctgcccattcgaccaccaagcgaaacatcgcatcgagcgagcacgtactcggatggaagccggtcttgtcgatcaggatgatctg
gacgaagagcatcaggggctcgcgccagccgaactgttcgccaggctcaaggcgagcatgcccgacggcgaggatctcgtcgtga
cccatggcgatgcctgcttgccgaatatcatggtggaaaatggccgcttttctggattcatcgactgtggccggctgggtgtggcggac
cgctatcaggacatagcgttggctacccgtgatattgctgaagagcttggcggcgaatgggctgaccgcttcctcgtgctttacggtatc
gccgctcccgattcgcagcgcatcgccttctatcgccttcttgacgagttcttctgaattattaacgcttacaatttcctgatgcggtattttct
ccttacgcatctgtgcggtatttcacaccgcatcaggtggcacttttcggggaaatgtgcgcggaacccctatttgtttatttttctaaatac
attcaaatatgtatccgctcatgaccaaaatcccttaacgtgagttttcgttccactgagcgtcagaccccgtagaaaagatcaaaggatc
ttcttgagatcctttttttctgcgcgtaatctgctgcttgcaaacaaaaaaaccaccgctaccagcggtggtttgtttgccggatcaagagct
accaactctttttccgaaggtaactggcttcagcagagcgcagataccaaatactgttcttctagtgtagccgtagttaggccaccacttc
aagaactctgtagcaccgcctacatacctcgctctgctaatcctgttaccagtggctgctgccagtggcgataagtcgtgtcttaccggg
ttggactcaagacgatagttaccggataaggcgcagcggtcgggctgaacgggggggtcgtgcacacagcccagcttggagcgaa
cgacctacaccgaactgagatacctacagcgtgagctatgagaaagcgccacgcttcccgaagggagaaaggcggacaggtatcc
ggtaagcggcagggtcggaacaggagagcgcacgagggagcttccagggggaaacgcctggtatctttatagtcctgtcgggtttc
gccacctctgacttgagcgtcgatttttgtgatgctcgtcaggggggcggagcctatggaaaaacgccagcaacgcggcctttttacgg
ttcctggccttttgctggccttttgctcacatgttctttcctgcgttatcccctgattctgtggataaccgtattaccgcctttgagtgagctgat
accgctcgccgcagccgaacgaccgagcgcagcgagtcagtgagcgaggaagcggaagagcgcccaatacgcaaaccgcctct
ccccgcgcgttggccgattcattaatgcagctggcacgacaggtttcccgactggaaagcgggcagtgagcgcaacgcaattaatgt
gagttagctcactcattaggcaccccaggctttacactttatgcttccggctcgtatgttgtgtggaattgtgagcggataacaatttcaca
caggaaacagctatgaccatgattacgccaagcgcgcaattaaccctcactaaagggaacaaaagctggagctgcaagcttggccat
tgcatacgttgtatccatatcataatatgtacatttatattggctcatgtccaacattaccgccatgttgacattgattattgactagttattaata
gtaatcaattacggggtcattagttcatagcccatatatggagttccgcgttacataacttacggtaaatggcccgcctggctgaccgccc
aacgacccccgcccattgacgtcaataatgacgtatgttcccatagtaacgccaatagggactttccattgacgtcaatgggtggagtat
ttacggtaaactgcccacttggcagtacatcaagtgtatcatatgccaagtacgccccctattgacgtcaatgacggtaaatggcccgcc
tggcattatgcccagtacatgaccttatgggactttcctacttggcagtacatctacgtattagtcatcgctattaccatggtgatgcggtttt
ggcagtacatcaatgggcgtggatagcggtttgactcacggggatttccaagtctccaccccattgacgtcaatgggagtttgttttggc
accaaaatcaacgggactttccaaaatgtcgtaacaactccgccccattgacgcaaatgggcggtaggcgtgtacggtgggaggtcta
tataagcagagctcgtttagtgaaccgGGGTCTCTCTGGTTAGACCAGATCTGAGCCTGGGAGCT
CTCTGGCTAACTAGGGAACCCACTGCTTAAGCCTCAATAAAGCTTGCCTTGAGTG
CTTCAAGTAGTGTGTGCCCGTCTGTTGTGTGACTCTGGTAACTAGAGATCCCTCA
GACCCTTTTAGTCAGTGTGGAAAATCTCTAGCagtggcgcccgaacagggacttgaaagcgaaaggg
aaaccagaggagctctcTCGACGCAGGACTCGGCTTGCTGAAGCGCGCACGGCAAGAGGC
GAGGGGCGGCGACTGGTGAGTACGCCAAAAATTTTGACTAGCGGAGGCTAGAAG
GAGAGAGATGGGTGCGAGAGCGTCAGTATTAAGCGGGGGAGaattagatcgcgatgggaaa
aaattcggttaaggccagggggaaagaaaaaatataaattaaaacatatagtatgggcaagcagggagctagaacgattcgcagttaa
tcctggcctgttagaaacatcagaaggctgtagacaaatactgggacagctacaaccatcccttcagacaggatcagaagaacttagat
cattatataatacagtagcaaccctctattgtgtgcatcaaaggatagagataaaagacaccaaggaagctttagacaagatagaggaa
gagcaaaacaaaagtaagaccaccgcacagcaagcGGCCGCTgatcttcagacctggaggaggagatatgagggacaattg
gagaagtgaattatataaatataaagtagtaaaaattgaaccattaggagtagcacccaccaaggcaaagagaagagtggtgcagag
agaaaaaagaGCAGTGGGAATaggagctttgttccttgggttcttgggagcagcaggaagcactatgggcgcagcgtcaat
gacgctgacggtacaggccagacaattattgtctggtatagtgcagcagcagaacaatttgctgagggctattgaggcgcaacagcat
ctgttgcaactcacagtctggggcatcaagcagctccaggcaagaatcctggctgtggaaagatacctaaaggatcaacagctcctgg
ggatttggggttgctctggaaaactcatttgcaccactgctgtgccttggaatgctagttggagtaataaatctctggaacagatttggaat
cacacgacctggatggagtgggacagagaaattaacaattacacaagcttaatacactccttaattgaagaatcgcaaaaccagcaag
aaaagaatgaacaagaattattggaattagataaatgggcaagtttgtggaattggtttaacataacaaattggctgtggtatataaaattat
tcataatgatagtaggaggcttggtaggtttaagaatagtttttgctgtactttctatagtgaatagagttaggcagggatattcaccattatc
gtttcagacccacctcccaaccccgaggggacccgacaggcccgaaggaatagaagaagaaggtggagagagagacagagaca
gatccattcgattagtgaacggatCTCGACGGTATCGATCTCGAcacaaatggcagtattcatccacaattttaaaag
aaaaggggggattggggggtacagtgcaggggaaagaatagtagacataatagcaacagacatacaaactaaagaattacaaaaac
aaattacaaaaattcaaaattttcgggtttattacagggacagcagaTGCTGCCCACTCAGACTTTATTCAAAG
ACCAGGAAGGGCCGGTGCAAGGAGGGGAGGAGGGCCCGTTGGGAGGCCCAGCG
GGCAGGAGGAACGGCTACCGAGGCTCCAGCTTAACGGTATTTGGAGGTCAGCAC
GGTGCTCACAGAAGCCAGGAACTTGTCCAGGGAGGCGTGCACCGCAGGGGTGAA
CTCGGCGGGGAGGTGGGCGGCCAGGGTCACCAGCAGGCAGTGGCTTAGGAGcTT
GAAGTTGACCGGGTCCACCCGAAGCTTGTGCGCGTGCAGGTCGCTCAGGGCGGA
CAGCGCGTTGGGCATGTCGTCCACGTGCGCCACGGCGTTGGTCAGCGCGTCGGCC
ACCTTCTTGCCGTGGCCCTTAACCTGGGCAGAGCCGTGGCTCAGGTCGAAGTGCG
GGAAGTAGGTCTTGGTGGTGGGGAAGGACAGGAACATCCTGCGGGGAGAAGCA
GAGTGAGGGGTGGGGTTTGGGTCCGGGGCCAGGACGGTTGAGGGTGGCCTGTGG
GTCCGGGCGGGCGAGGAGCCCGGGTCGGAGCAGGGGAGGGAGCCTCACCTCTCC
AGGGCCTCCGCACCATACTCGCCAGCGTGCGCGCCGACCTTACCCCAGGCGGCCT
TGACGTTGGTCTTGTCGGCAGGAGACAGCACCATGGTGGGTTCTCTCTGAGTCTG
TGGGGACCAGAAGAGTaagcaatagatggctctgccctgacttttatgcccagccctggctcctgccctccctgctcctg
ggagtagattggccaaccctagggtgtggctccacagggtgaggtctaagtgatgacagccgtacctgtccttggctcttctggcactg
gcttaggagttggacttcaaaccctcagccctccctctaagatatatctcttggccccataccatcagtacaaattgctactaaaaacatcc
tcctttgcaagtgtatttacccttttgccacctagctgtccaggggtgccttaaaatggcaaacaaggtttgttttcttttcctgttttcatgcctt
cctcttccatatccttgtttcatattaatacatgtgtatagatcctaaaaatctatacacatgtattaataaagcctgattctgccgcttctaggt
atagaggccacctgcaagataaatatttgattcacaataactaatcattctatggcaattgataacaacaaatatatatatatatatatatacg
tatatgtgtatatatatatatatatattcaggaaataatatattctagaatatgtcacattctgtctcaggcatccattttctttatgatgccgtttg
aggtggagttttagtcaggtggtcagcttctccttttttttgccatctgccctgtaagcatcctgctggggacccagataggagtcatcact
ctaggctgagaacatctgggcacacaccctaagcctcagcatgactcatcatgactcagcattgctgtgcttgagccagaaggtttgctt
agaaggttacacagaaccagaaggcgggggtggggcactgaccccgacaggggcctggccagaactgctcatgcttggactatgg
gaggtcactaatggagacacacagaaatgtaacaggaactaaggaaaaactgaagcttatttaatcagagatgaggatgctggaagg
gatagagggagctgagcttgtaaaaagtatagtaatcattcagcaaatggttttgaagcacctgctggatgctaaacactattttcagtgct
tgaatcataaataagaataaaacatgtatcttattccccacaagagtccaagtaaaaaataacagttaattataatgtgctctgtcccccag
gctggagtgcagtggcacgatctcagctcactgcaacctccgcctcccgggttcaagcaattctcctgcctcagccaccctaatagctg
ggattacaggtgcacaccaccatgccaggctaatttttgtactttttgtagaggcagggtatcaccatgttgtccaagatggtcttgaactc
ctgagctccaagcagtccacccacctcagcctcccaaagtgctgggattacaggtgtgagacaccatgcccagattttccatatttaata
gaggtatttatgggatgggggaaaagaatgtttctctcactgtggattattttagagagtggagaatggtcaagatttttttaaaaattaaga
aaacataagttggaccttgagaaatgaaaatttatttttttgttggaggatacccattctctatctcccatcagggcaagctgtaaggaactg
gctaagacacagtgagacagagtgacttagtcttagaggccccactggtacgacggtcaccaagctttcattaaaaaaagtctaacca
gctgcattcgactttgactgcagcagctggttagaaggttctactggaggagggtcccagcccattgctaaattaacatcaggctctgag
actggcagtatatctctaacagtggttgatgctatcttctggaacttgcctgctacattgagaccactgacccatacataggaagcccata
gctctgtcctgaactgttaggccactggtccagagagtgtgcatctcctttgatcctcataataaccctatgagatagacacaattattactc
ttactttatagatgatgatcctgaaaacataggagtcaaggcacttgcccctagctgggggtataggggagcagtcccatgtagtagtag
aatgaaaaatgctgctatgctgtgcctcccccacctttcccatgtctgccctctactcatggtctatctctcctggctcctgggagtcatgg
actccacccagcaccaccaacctgacctaaccacctatctgagcctgccagcctataacccatctgggccctgatagctggtggccag
ccctgaccccaccccaccctccctggaacctctgatagacacatctggcacaccagctcgcaaagtcaccgtgagggtcttgtgtttgc
tgagtcaaaattccttgaaatccaagtccttagagactcctgctcccaaatttacagtcatagacttcttcatggctgtctcctttatccacag
aatgattcctttgcttcattgccccatccatctgatcctcctcatcagtgcagcacagggcccatgagcagtagctgcagagtctcacata
ggtctggcactgcctctgacatgtccgaccttaggcaaatgcttgactcttctgagctcagtcttgtcatggcaaaataaagataataata
gtgtttttttatggagttagcgtgaggatggaaaacaatagcaaaattgattagactataaaaggtctcaacaaatagtagtagattttatca
tccattaatccttccctctcctctcttactcatcccatcacgtatgcctcttaattttcccttacctataataagagttattcctcttattatattcttc
ttatagtgattctggatattaaagtgggaatgaggggcaggccactaacgaagaagatgtttctcaaagaagctaaaagaaaaggggg
gactggaagggctaattcactcccaacgaagacaagatctgctttttgcttgtactGGGTCTCTCTGGTTAGACCAG
ATCTGAGCCTGGGAGCTCTCTGGCTAACTAGGGAACCCACTGCTTAAGCCTCAAT
AAAGCTTGCCTTGAGTGCTTCAAGTAGTGTGTTGGTTTTTTGTGTGTGCATTGTCT
GAGTAGGTGTCATTCTATTCTGGGGGGTGGGGTGGGGCAGCACAGCAAGGGGGA
GGATTGGGAAGACAATAGCAGGCATGCTGGGGATGCGGTGGGCTCTATGGagtagta
gttcatgtcatcttattattcagtatttataacttgcaaagaaatgaatatcagagagtgagaggaacttgtttattgcagcttataatggttac
aaataaagcaatagcatcacaaatttcacaaataaagcatttttttcactgcattctagttgtggtttgtccaaactcatcaatgtatcttatcat
gtctggctctagctatcccgcccctaactccgcccATCCCGCCCCTAACTCCGCCCAGTTCCGCCCATT
CTCCGCCCCATGGCTCTCACTACTTCTGGAATAGCTCAGAGGCCGAGGCGGCCTC
GGCCTCTGCATAAATAAAAAAAATTAGTCGAGGCTTTTTTGGAGGCCTAGGgacgta
cccaattcgccctatagtgagtcgtattacgcgcgctcactggccgtcgttttacaacgtgtgactgggaaaaccctggcgttacccaa
cttaatcgccttgcagcacatccccctttcgccagctggcgtaatagcgaagaggcccgcaccgatcgcccttcccaacagttgcgca
gcctgaatggcgaatggg
A2-DIVS2-UV
Sequence ID No: 40
acgcgccctgtagcggcgcattaagcgcggcgggtgtggtggttacgcgcagcgtgaccgctacacttgccagcgccctagcgccc
gctcctttcgctttcttcccttcctttctcgccacgttcgccggctttccccgtcaagctctaaatcgggggctccctttagggttccgattta
gtgctttacggcacctcgaccccaaaaaacttgattagggtgatggttcacgtagtgggccatcgccctgatagacggtttttcgcccttt
gacgttggagtccacgttctttaatagtggactcttgttccaaactggaacaacactcaaccctatctcggtctattcttttgatttataaggg
attttgccgatttcggcctattggttaaaaaatgagctgatttaacaaaaatttaacgcgaattttaacaaaatattaacgcttacaatttaggt
ggcacttttcggggaaatgtgcgcggaacccctatttgtttatttttctaaatacattcaaatatgtatccgctcatgagacaataaccctgat
aaatgcttcaataatagcacctagatcaagagacaggatgaggatcgtttcgcatgattgaacaagatggattgcacgcaggttctccg
gccgcttgggtggagaggctattcggctatgactgggcacaacagacaatcggctgctctgatgccgccgtgttccggctgtcagcgc
aggggcgcccggttctttttgtcaagaccgacctgtccggtgccctgaatgaactgcaagacgaggcagcgcggctatcgtggctgg
ccacgacgggcgttccttgcgcagctgtgctcgacgttgtcactgaagcgggaagggactggctgctattgggcgaagtgccgggg
caggatctcctgtcatctcaccttgctcctgccgagaaagtatccatcatggctgatgcaatgcggcggctgcatacgcttgatccggct
acctgcccattcgaccaccaagcgaaacatcgcatcgagcgagcacgtactcggatggaagccggtcttgtcgatcaggatgatctg
gacgaagagcatcaggggctcgcgccagccgaactgttcgccaggctcaaggcgagcatgcccgacggcgaggatctcgtcgtga
cccatggcgatgcctgcttgccgaatatcatggtggaaaatggccgcttttctggattcatcgactgtggccggctgggtgtggcggac
cgctatcaggacatagcgttggctacccgtgatattgctgaagagcttggcggcgaatgggctgaccgcttcctcgtgctttacggtatc
gccgctcccgattcgcagcgcatcgccttctatcgccttcttgacgagttcttctgaattattaacgcttacaatttcctgatgcggtattttct
ccttacgcatctgtgcggtatttcacaccgcatcaggtggcacttttcggggaaatgtgcgcggaacccctatttgtttatttttctaaatac
attcaaatatgtatccgctcatgaccaaaatcccttaacgtgagttttcgttccactgagcgtcagaccccgtagaaaagatcaaaggatc
ttcttgagatcctttttttctgcgcgtaatctgctgcttgcaaacaaaaaaaccaccgctaccagcggtggtttgtttgccggatcaagagct
accaactctttttccgaaggtaactggcttcagcagagcgcagataccaaatactgttcttctagtgtagccgtagttaggccaccacttc
aagaactctgtagcaccgcctacatacctcgctctgctaatcctgttaccagtggctgctgccagtggcgataagtcgtgtcttaccggg
ttggactcaagacgatagttaccggataaggcgcagcggtcgggctgaacgggggggtcgtgcacacagcccagcttggagcgaa
cgacctacaccgaactgagatacctacagcgtgagctatgagaaagcgccacgcttcccgaagggagaaaggcggacaggtatcc
ggtaagcggcagggtcggaacaggagagcgcacgagggagcttccagggggaaacgcctggtatctttatagtcctgtcgggtttc
gccacctctgacttgagcgtcgatttttgtgatgctcgtcaggggggcggagcctatggaaaaacgccagcaacgcggcctttttacgg
ttcctggccttttgctggccttttgctcacatgttctttcctgcgttatcccctgattctgtggataaccgtattaccgcctttgagtgagctgat
accgctcgccgcagccgaacgaccgagcgcagcgagtcagtgagcgaggaagcggaagagcgcccaatacgcaaaccgcctct
ccccgcgcgttggccgattcattaatgcagctggcacgacaggtttcccgactggaaagcgggcagtgagcgcaacgcaattaatgt
gagttagctcactcattaggcaccccaggctttacactttatgcttccggctcgtatgttgtgtggaattgtgagcggataacaatttcaca
caggaaacagctatgaccatgattacgccaagcgcgcaattaaccctcactaaagggaacaaaagctggagctgcaagcttggccat
tgcatacgttgtatccatatcataatatgtacatttatattggctcatgtccaacattaccgccatgttgacattgattattgactagttattaata
gtaatcaattacggggtcattagttcatagcccatatatggagttccgcgttacataacttacggtaaatggcccgcctggctgaccgccc
aacgacccccgcccattgacgtcaataatgacgtatgttcccatagtaacgccaatagggactttccattgacgtcaatgggtggagtat
ttacggtaaactgcccacttggcagtacatcaagtgtatcatatgccaagtacgccccctattgacgtcaatgacggtaaatggcccgcc
tggcattatgcccagtacatgaccttatgggactttcctacttggcagtacatctacgtattagtcatcgctattaccatggtgatgcggtttt
ggcagtacatcaatgggcgtggatagcggtttgactcacggggatttccaagtctccaccccattgacgtcaatgggagtttgttttggc
accaaaatcaacgggactttccaaaatgtcgtaacaactccgccccattgacgcaaatgggggtaggcgtgtacggtgggaggtcta
tataagcagagctcgtttagtgaaccgGGGTCTCTCTGGTTAGACCAGATCTGAGCCTGGGAGCT
CTCTGGCTAACTAGGGAACCCACTGCTTAAGCCTCAATAAAGCTTGCCTTGAGTG
CTTCAAGTAGTGTGTGCCCGTCTGTTGTGTGACTCTGGTAACTAGAGATCCCTCA
GACCCTTTTAGTCAGTGTGGAAAATCTCTAGCagtggcgcccgaacagggacttgaaagcgaaaggg
aaaccagaggagctctcTCGACGCAGGACTCGGCTTGCTGAAGCGCGCACGGCAAGAGGC
GAGGGGCGGCGACTGGTGAGTACGCCAAAAATTTTGACTAGCGGAGGCTAGAAG
GAGAGAGATGGGTGCGAGAGCGTCAGTATTAAGCGGGGGAGaattagatcgcgatgggaaa
aaattcggttaaggccagggggaaagaaaaaatataaattaaaacatatagtatgggcaagcagggagctagaacgattcgcagttaa
tcctggcctgttagaaacatcagaaggctgtagacaaatactgggacagctacaaccatcccttcagacaggatcagaagaacttagat
cattatataatacagtagcaaccctctattgtgtgcatcaaaggatagagataaaagacaccaaggaagctttagacaagatagaggaa
gagcaaaacaaaagtaagaccaccgcacagcaagcGGCCGCTgatcttcagacctggaggaggagatatgagggacaattg
gagaagtgaattatataaatataaagtagtaaaaattgaaccattaggagtagcacccaccaaggcaaagagaagagtggtgcagag
agaaaaaagaGCAGTGGGAATaggagctttgttccttgggttcttgggagcagcaggaagcactatgggcgcagcgtcaat
gacgctgacggtacaggccagacaattattgtctggtatagtgcagcagcagaacaatttgctgagggctattgaggcgcaacagcat
ctgttgcaactcacagtctggggcatcaagcagctccaggcaagaatcctggctgtggaaagatacctaaaggatcaacagctcctgg
ggatttggggttgctctggaaaactcatttgcaccactgctgtgccttggaatgctagttggagtaataaatctctggaacagatttggaat
cacacgacctggatggagtgggacagagaaattaacaattacacaagcttaatacactccttaattgaagaatcgcaaaaccagcaag
aaaagaatgaacaagaattattggaattagataaatgggcaagtttgtggaattggtttaacataacaaattggctgtggtatataaaattat
tcataatgatagtaggaggcttggtaggtttaagaatagtttttgctgtactttctatagtgaatagagttaggcagggatattcaccattatc
gtttcagacccacctcccaaccccgaggggacccgacaggcccgaaggaatagaagaagaaggtggagagagagacagagaca
gatccattcgattagtgaacggatCTCGACGGTATCGATCTCGAcacaaatggcagtattcatccacaattttaaaag
aaaaggggggattggggggtacagtgcaggggaaagaatagtagacataatagcaacagacatacaaactaaagaattacaaaaac
aaattacaaaaattcaaaattttcgggtttattacagggacagcagaTGCTGCCCACTCAGACTTTATTCAAAG
ACCAGGAAGGGCCGGTGCAAGGAGGGGAGGAGGGCCCGTTGGGAGGCCCAGCG
GGCAGGAGGAACGGCTACCGAGGCTCCAGCTTAACGGTATTTGGAGGTCAGCAC
GGTGCTCACAGAAGCCAGGAACTTGTCCAGGGAGGCGTGCACCGCAGGGGTGAA
CTCGGCGGGGAGGTGGGCGGCCAGGGTCACCAGCAGGCAGTGGCTTAGGAGCTT
GAAGTTGACCGGGTCCACCCGAAGCTTGTGCGCGTGCAGGTCGCTCAGGGCGGA
CAGCGCGTTGGGCATGTCGTCCACGTGCGCCACGGCGTTGGTCAGCGCGTCGGCC
ACCTTCTTGCCGTGGCCCTTAACCTGGGCAGAGCCGTGGCTCAGGTCGAAGTGCG
GGAAGTAGGTCTTGGTGGTGGGGAAGGACAGGAACATCCTGCGGGGAGAAGCA
GAGTGAGGGGTGGGGTTTGGGTCCGGGGCCAGGACGGTTGAGGGTGGCCTGTGG
GTCCGGGCGGGCGAGGAGCCCGGGTCGGAGCAGGGGAGGGAGCCTCACCTCTCC
AGGGCCTCCGCACCATACTCGCCAGCGTGCGCGCCGACCTTACCCCAGGCGGCCT
TGACGTTGGTCTTGTCGGCAGGAGACAGCACCATGGTGGGTTCTCTCTGAGTCTG
TGGGGACCAGAAGAGTaagcaatagatggctctgccctgacttttatgcccagccctggctcctgccctccctgctcctg
ggagtagattggccaaccctagggtgtggctccacagggtgaggtctaagtgatgacagccgtacctgtccttggctcttctggcactg
gcttaggagttggacttcaaaccctcagccctccctctaagatatatctcttggccccataccatcagtacaaattgctactaaaaacatcc
tcctttgcaagtgtatttacTACGTaTATGTGTATATATATATATATATTCAGGAAATAATATAT
TCTAGAATATGTCACATTCTGTCTCAGGCATCCATTTTCTTTATGATGCCGTTTGA
GGTGGAGTTTTAGTCAGGTGGTCAGCTTCTCCTTTTTTTTGCCATCTGCCCTGTAA
GCATCCTGCTGGGGACCCAGATAGGAGTCATCACTCTAGGCTGAGAACATCTGG
GCACACACCCTAAGCCTCAGCATGACTCATCATGACTCAGCATTGCTGTGCTTGA
GCCAGAAGGTTTGCTTAGAAGGTTACACAGAACCAGAAGGCGGGGGTGGGGCAC
TGACCCCGACAGGGGCCTGGCCAGAACTGCTCATGCTTGGACTATGGGAGGTCA
CTAATGGAGACACACAGAAATGTAACAGGAACTAAGGAAAAACTGAAGCTTtggg
ggtataggggagcagtcccatgtagtagtagaatgaaaaatgctgctatgctgtgcctcccccacctttcccatgtctgccctctactcat
ggtctatctctcctggctcctgggagtcatggactccacccagcaccaccaacctgacctaaccacctatctgagcctgccagcctata
acccatctgggccctgatagctggtggccagccctgaccccaccccaccctccctggaacctctgatagacacatctggcacaccag
ctcgcaaagtcaccgtgagggtcttgtgtttgctgagtcaaaattccttgaaatccaagtccttagagactcccaggcttggattcaaagc
tcctgactttctgtctagtgtatgtgcagtgagccccttttcctctaactgaaagaaggaaaaaaaaatggaacccaaaatattctacatag
tttccatgtcacagccagggctgggcagtctcctgttatttcttttaaaataaatatatcatttaaatgcataaataagcaaaccctgctcgg
gaatgggagggagagtctctggagtccaccccttctcggccctggctctgcagatagtgctatcaaagccctgacagagccctgccc
attgctgggccttggagtgagtcagcctagtagagaggcagggcaagccatctcatagctgctgagtgggagagagaaaagggctc
attgtctataaactcaggtcatggctattcttattaaaagaaaaggggggactggaagggctaattcactcccaacgaagacaagatctg
ctttttgcttgtactGGGTCTCTCTGGTTAGACCAGATCTGAGCCTGGGAGCTCTCTGGCTA
ACTAGGGAACCCACTGCTTAAGCCTCAATAAAGCTTGCCTTGAGTGCTTCAAGTA
GTGTGTTGGTTTTTTGTGTGTGCATTGTCTGAGTAGGTGTCATTCTATTCTGGGGG
GTGGGGTGGGGCAGCACAGCAAGGGGGAGGATTGGGAAGACAATAGCAGGCAT
GCTGGGGATGCGGTGGGCTCTATGGagtagtagttcatgtcatcttattattcagtatttataacttgcaaagaaat
gaatatcagagagtgagaggaacttgtttattgcagcttataatggttacaaataaagcaatagcatcacaaatttcacaaataaagcattt
ttttcactgcattctagttgtggtttgtccaaactcatcaatgtatcttatcatgtctggctctagctatcccgcccctaactccgcccATCC
CGCCCCTAACTCCGCCCAGTTCCGCCCATTCTCCGCCCCATGGCTCTCACTACTTC
TGGAATAGCTCAGAGGCCGAGGCGGCCTCGGCCTCTGCATAAATAAAAAAAATT
AGTCGAGGCTTTTTTGGAGGCCTAGGgacgtacccaattcgccctatagtgagtcgtattacgcgcgctcactg
gccgtcgttttacaacgtcgtgactgggaaaaccctggcgttacccaacttaatcgccttgcagcacatccccctttcgccagctggcgt
aatagcgaagaggcccgcaccgatcgcccttcccaacagttgcgcagcctgaatggcgaatggg
A2-GLOBE
Sequence ID No: 41
acgcgccctgtagcggcgcattaagcgcggcgggtgtggtggttacgcgcagcgtgaccgctacacttgccagcgccctagcgccc
gctcctttcgctttcttcccttcctttctcgccacgttcgccggctttccccgtcaagctctaaatcgggggctccctttagggttccgattta
gtgctttacggcacctcgaccccaaaaaacttgattagggtgatggttcacgtagtgggccatcgccctgatagacggtttttcgcccttt
gacgttggagtccacgttctttaatagtggactcttgttccaaactggaacaacactcaaccctatctcggtctattcttttgatttataaggg
attttgccgatttcggcctattggttaaaaaatgagctgatttaacaaaaatttaacgcgaattttaacaaaatattaacgcttacaatttaggt
ggcacttttcggggaaatgtgcgcggaacccctatttgtttatttttctaaatacattcaaatatgtatccgctcatgagacaataaccctgat
aaatgcttcaataatagcacctagatcaagagacaggatgaggatcgtttcgcatgattgaacaagatggattgcacgcaggttctccg
gccgcttgggtggagaggctattcggctatgactgggcacaacagacaatcggctgctctgatgccgccgtgttccggctgtcagcgc
aggggcgcccggttctttttgtcaagaccgacctgtccggtgccctgaatgaactgcaagacgaggcagcgcggctatcgtggctgg
ccacgacgggcgttccttgcgcagctgtgctcgacgttgtcactgaagcgggaagggactggctgctattgggcgaagtgccgggg
caggatctcctgtcatctcaccttgctcctgccgagaaagtatccatcatggctgatgcaatgcggcggctgcatacgcttgatccggct
acctgcccattcgaccaccaagcgaaacatcgcatcgagcgagcacgtactcggatggaagccggtcttgtcgatcaggatgatctg
gacgaagagcatcaggggctcgcgccagccgaactgttcgccaggctcaaggcgagcatgcccgacggcgaggatctcgtcgtga
cccatggcgatgcctgcttgccgaatatcatggtggaaaatggccgcttttctggattcatcgactgtggccggctgggtgtggcggac
cgctatcaggacatagcgttggctacccgtgatattgctgaagagcttggcggcgaatgggctgaccgcttcctcgtgctttacggtatc
gccgctcccgattcgcagcgcatcgccttctatcgccttcttgacgagttcttctgaattattaacgcttacaatttcctgatgcggtattttct
ccttacgcatctgtgcggtatttcacaccgcatcaggtggcacttttcggggaaatgtgcgcggaacccctatttgtttatttttctaaatac
attcaaatatgtatccgctcatgaccaaaatcccttaacgtgagttttcgttccactgagcgtcagaccccgtagaaaagatcaaaggatc
ttcttgagatcctttttttctgcgcgtaatctgctgcttgcaaacaaaaaaaccaccgctaccagcggtggtttgtttgccggatcaagagct
accaactctttttccgaaggtaactggcttcagcagagcgcagataccaaatactgttcttctagtgtagccgtagttaggccaccacttc
aagaactctgtagcaccgcctacatacctcgctctgctaatcctgttaccagtggctgctgccagtggcgataagtcgtgtcttaccggg
ttggactcaagacgatagttaccggataaggcgcagcggtcgggctgaacgggggggtcgtgcacacagcccagcttggagcgaa
cgacctacaccgaactgagatacctacagcgtgagctatgagaaagcgccacgcttcccgaagggagaaaggcggacaggtatcc
ggtaagcggcagggtcggaacaggagagcgcacgagggagcttccagggggaaacgcctggtatctttatagtcctgtcgggtttc
gccacctctgacttgagcgtcgatttttgtgatgctcgtcaggggggcggagcctatggaaaaacgccagcaacgcggcctttttacgg
ttcctggccttttgctggccttttgctcacatgttctttcctgcgttatcccctgattctgtggataaccgtattaccgcctttgagtgagctgat
accgctcgccgcagccgaacgaccgagcgcagcgagtcagtgagcgaggaagcggaagagcgcccaatacgcaaaccgcctct
ccccgcgcgttggccgattcattaatgcagctggcacgacaggtttcccgactggaaagcgggcagtgagcgcaacgcaattaatgt
gagttagctcactcattaggcaccccaggctttacactttatgcttccggctcgtatgttgtgtggaattgtgagcggataacaatttcaca
caggaaacagctatgaccatgattacgccaagcgcgcaattaaccctcactaaagggaacaaaagctggagctgcaagcttggccat
tgcatacgttgtatccatatcataatatgtacatttatattggctcatgtccaacattaccgccatgttgacattgattattgactagttattaata
gtaatcaattacggggtcattagttcatagcccatatatggagttccgcgttacataacttacggtaaatggcccgcctggctgaccgccc
aacgacccccgcccattgacgtcaataatgacgtatgttcccatagtaacgccaatagggactttccattgacgtcaatgggtggagtat
ttacggtaaactgcccacttggcagtacatcaagtgtatcatatgccaagtacgccccctattgacgtcaatgacggtaaatggcccgcc
tggcattatgcccagtacatgaccttatgggactttcctacttggcagtacatctacgtattagtcatcgctattaccatggtgatgcggtttt
ggcagtacatcaatgggcgtggatagcggtttgactcacggggatttccaagtctccaccccattgacgtcaatgggagtttgttttggc
accaaaatcaacgggactttccaaaatgtcgtaacaactccgccccattgacgcaaatgggcggtaggcgtgtacggtgggaggtcta
tataagcagagctcgtttagtgaaccgGGGTCTCTCTGGTTAGACCAGATCTGAGCCTGGGAGCT
CTCTGGCTAACTAGGGAACCCACTGCTTAAGCCTCAATAAAGCTTGCCTTGAGTG
CTTCAAGTAGTGTGTGCCCGTCTGTTGTGTGACTCTGGTAACTAGAGATCCCTCA
GACCCTTTTAGTCAGTGTGGAAAATCTCTAGCagtggcgcccgaacagggacttgaaagcgaaaggg
aaaccagaggagctctcTCGACGCAGGACTCGGCTTGCTGAAGCGCGCACGGCAAGAGGC
GAGGGGCGGCGACTGGTGAGTACGCCAAAAATTTTGACTAGCGGAGGCTAGAAG
GAGAGAGATGGGTGCGAGAGCGTCAGTATTAAGCGGGGGAGaattagatcgcgatgggaaa
aaattcggttaaggccagggggaaagaaaaaatataaattaaaacatatagtatgggcaagcagggagctagaacgattcgcagttaa
tcctggcctgttagaaacatcagaaggctgtagacaaatactgggacagctacaaccatcccttcagacaggatcagaagaacttagat
cattatataatacagtagcaaccctctattgtgtgcatcaaaggatagagataaaagacaccaaggaagctttagacaagatagaggaa
gagcaaaacaaaagtaagaccaccgcacagcaagcGGCCGCTgatcttcagacctggaggaggagatatgagggacaattg
gagaagtgaattatataaatataaagtagtaaaaattgaaccattaggagtagcacccaccaaggcaaagagaagagtggtgcagag
agaaaaaagaGCAGTGGGAATaggagctttgttccttgggttcttgggagcagcaggaagcactatgggcgcagcgtcaat
gacgctgacggtacaggccagacaattattgtctggtatagtgcagcagcagaacaatttgctgagggctattgaggcgcaacagcat
ctgttgcaactcacagtctggggcatcaagcagctccaggcaagaatcctggctgtggaaagatacctaaaggatcaacagctcctgg
ggatttggggttgctctggaaaactcatttgcaccactgctgtgccttggaatgctagttggagtaataaatctctggaacagatttggaat
cacacgacctggatggagtgggacagagaaattaacaattacacaagcttaatacactccttaattgaagaatcgcaaaaccagcaag
aaaagaatgaacaagaattattggaattagataaatgggcaagtttgtggaattggtttaacataacaaattggctgtggtatataaaattat
tcataatgatagtaggaggcttggtaggtttaagaatagtttttgctgtactttctatagtgaatagagttaggcagggatattcaccattatc
gtttcagacccacctcccaaccccgaggggacccgacaggcccgaaggaatagaagaagaaggtggagagagagacagagaca
gatccattcgattagtgaacggatCTCGACGGTATCGATCTCGAcacaaatggcagtattcatccacaattttaaaag
aaaaggggggattggggggtacagtgcaggggaaagaatagtagacataatagcaacagacatacaaactaaagaattacaaaaac
aaattacaaaaattcaaaattttcgggtttattacagggacagcagaTGCTGCCCACTCAGACTTTATTCAAAG
ACCAGGAAGGGCCGGTGCAAGGAGGGGAGGAGGGCCCGTTGGGAGGCCCAGCG
GGCAGGAGGAACGGCTACCGAGGCTCCAGCtgtgctgctctgcggcgaTTAACGGTATTTG
GAGGTCAGCACGGTGCTCACAGAAGCCAGGAACTTGTCCAGGGAGGCGTGCACC
GCAGGGGTGAACTCGGCGGGGAGGTGGGCGGCCAGGGTCACCAGCAGGCAGTG
GCTTAGGAGCTGTGCAGAGAAGAGGGTCAGTGCGGCCCAGGCCCGCAGCCGCCG
CCTGCGCTACACCTCCCGCAACCCGCGTGATCCTCTGCCCTGAGAGGAAGGCGCC
ATCTCGCCCCTCGACCCAGATCGCTCCCGGCCCGCCGCTCACCTTGAAGTTGACC
GGGTCCACCCGAAGCTTGTGCGCGTGCAGGTCGCTCAGGGCGGACAGCGCGTTG
GGCATGTCGTCCACGTGCGCCACGGCGTTGGTCAGCGCGTCGGCCACCTTCTTGC
CGTGGCCCTTAACCTGGGCAGAGCCGTGGCTCAGGTCGAAGTGCGGGAAGTAGG
TCTTGGTGGTGGGGAAGGACAGGAACATCCTGCGGGGAGAAGCAGAGTGAGGG
GTGGGGTTTGGGTCCGGGGCCAGGACGGTTGAGGGTGGCCTGTGGGTCCGGGCG
GGCGAGGAGCCCGGGTCGGAGCAGGGGAGGGAGCCTCACCTCTCCAGGGCCTCC
GCACCATACTCGCCAGCGTGCGCGCCGACCTTACCCCAGGCGGCCTTGACGTTGG
TCTTGTCGGCAGGAGACAGCACCATGGTGGGTTCTCTCTGAGTCTGTGGGGACCA
GAAGAGTaagcaatagatggctctgccctgacttttatgcccagccctggctcctgccctccctgctcctgggagtagattggcc
aaccctagggtgtggctccacagggtgaggtctaagtgatgacagccgtacctgtccttggctcttctggcactggcttaggagttgga
cttcaaaccctcagccctccctctaagatatatctcttggccccataccatcagtacaaattgctactaaaaacatcctcctttgcaagtgta
tttacccttttgccacctagctgtccaggggtgccttaaaatggcaaacaaggtttgttttcttttcctgttttcatgccttcctcttccatatcct
tgtttcatattaatacatgtgtatagatcctaaaaatctatacacatgtattaataaagcctgattctgccgcttctaggtatagaggccacct
gcaagataaatatttgattcacaataactaatcattctatggcaattgataacaacaaatatatatatatatatatatacgtatatgtgtatatat
atatatatatattcaggaaataatatattctagaatatgtcacattctgtctcaggcatccattttctttatgatgccgtttgaggtggagttttag
tcaggtggtcagcttctccttttttttgccatctgccctgtaagcatcctgctggggacccagataggagtcatcactctaggctgagaaca
tctgggcacacaccctaagcctcagcatgactcatcatgactcagcattgctgtgcttgagccagaaggtttgcttagaaggttacacag
aaccagaaggcgggggtggggcactgaccccgacaggggcctggccagaactgctcatgcttggactatgggaggtcactaatgg
agacacacagaaatgtaacaggaactaaggaaaaactgaagcttatttaatcagagatgaggatgctggaagggatagagggagctg
agcttgtaaaaagtatagtaatcattcagcaaatggttttgaagcacctgctggatgctaaacactattttcagtgcttgaatcataaataag
aataaaacatgtatcttattccccacaagagtccaagtaaaaaataacagttaattataatgtgctctgtcccccaggctggagtgcagtg
gcacgatctcagctcactgcaacctccgcctcccgggttcaagcaattctcctgcctcagccaccctaatagctgggattacaggtgca
caccaccatgccaggctaatttttgtacttttttgtagaggcagggtatcaccatgttgtccaagatggtctttgaactcctgagctccaagca
gtccacccacctcagcctcccaaagtgctgggattacaggtgtgagacaccatgcccagattttccatatttaatagaggtatttatggga
tgggggaaaagaatgtttctctcactgtggattattttagagagtggagaatggtcaagatttttttaaaaattaagaaaacataagttggac
cttgagaaatgaaaatttatttttttgttggaggatacccattctctatctcccatcagggcaagctgtaaggaactggctaagacacagtg
agacagagtgacttagtcttagaggccccactggtacgacggtcaccaagctttcattaaaaaaagtctaaccagctgcattcgactttg
actgcagcagctggttagaaggttctactggaggagggtcccagcccattgctaaattaacatcaggctctgagactggcagtatatct
ctaacagtggttgatgctatcttctggaacttgcctgctacattgagaccactgacccatacataggaagcccatagctctgtcctgaact
gttaggccactggtccagagagtgtgcatctcctttgatcctcataataaccctatgagatagacacaattattactcttactttatagatgat
gatcctgaaaacataggagtcaaggcacttgcccctagctgggggtataggggagcagtcccatgtagtagtagaatgaaaaatgctg
ctatgctgtgcctcccccacctttcccatgtctgccctctactcatggtctatctctcctggctcctgggagtcatggactccacccagcac
caccaacctgacctaaccacctatctgagcctgccagcctataacccatctgggccctgatagctggtggccagccctgaccccaccc
caccctccctggaacctctgatagacacatctggcacaccagctcgcaaagtcaccgtgagggtcttgtgtttgctgagtcaaaattcct
tgaaatccaagtccttagagactcctgctcccaaatttacagtcatagacttcttcatggctgtctcctttatccacagaatgattcctttgctt
cattgccccatccatctgatcctcctcatcagtgcagcacagggcccatgagcagtagctgcagagtctcacataggtctggcactgcc
tctgacatgtccgaccttaggcaaatgcttgactcttctgagctcagtcttgtcatggcaaaataaagataataatagtgtttttttatggagtt
agcgtgaggatggaaaacaatagcaaaattgattagactataaaaggtctcaacaaatagtagtagattttatcatccattaatccttccct
ctcctctcttactcatcccatcacgtatgcctcttaattttcccttacctataataagagttattcctcttattatattcttcttatagtgattctggat
attaaagtgggaatgaggggcaggccactaacgaagaagatgtttctcaaagaagctaaaagaaaaggggggactggaagggcta
attcactcccaacgaagacaagatctgctttttgcttgtactGGGTCTCTCTGGTTAGACCAGATCTGAGCCT
GGGAGCTCTCTGGCTAACTAGGGAACCCACTGCTTAAGCCTCAATAAAGCTTGCC
TTGAGTGCTTCAAGTAGTGTGTTGGTTTTTTGTGTGTGCATTGTCTGAGTAGGTGT
CATTCTATTCTGGGGGGTGGGGTGGGGCAGCACAGCAAGGGGGAGGATTGGGAA
GACAATAGCAGGCATGCTGGGGATGCGGTGGGCTCTATGGagtagtagttcatgtcatcttattat
tcagtatttataacttgcaaagaaatgaatatcagagagtgagaggaacttgtttattgcagcttataatggttacaaataaagcaatagca
tcacaaatttcacaaataaagcatttttttcactgcattctagttgtggtttgtccaaactcatcaatgtatcttatcatgtctggctctagctatc
ccgcccctaactccgcccATCCCGCCCCTAACTCCGCCCAGTTCCGCCCATTCTCCGCCCCA
TGGCTCTCACTACTTCTGGAATAGCTCAGAGGCCGAGGCGGCCTCGGCCTCTGCA
TAAATAAAAAAAATTAGTCGAGGCTTTTTTGGAGGCCTAGGgacgtacccaattcgccctata
gtgagtcgtattacgcgcgctcactggccgtcgttttacaacgtcgtgactgggaaaaccctggcgttacccaacttaatcgccttgcag
cacatccccctttcgccagctggcgtaatagcgaagaggcccgcaccgatcgcccttcccaacagttgcgcagcctgaatggcgaat
ggg
A2-HS40-UV
Sequence ID No: 42
acgcgccctgtagcggcgcattaagcgcggcgggtgtggtggttacgcgcagcgtgaccgctacacttgccagcgccctagcgccc
gctcctttcgctttcttcccttcctttctcgccacgttcgccggctttccccgtcaagctctaaatcgggggctccctttagggttccgattta
gtgctttacggcacctcgaccccaaaaaacttgattagggtgatggttcacgtagtgggccatcgccctgatagacggtttttcgcccttt
gacgttggagtccacgttctttaatagtggactcttgttccaaactggaacaacactcaaccctatctcggtctattcttttgatttataaggg
attttgccgatttcggcctattggttaaaaaatgagctgatttaacaaaaatttaacgcgaattttaacaaaatattaacgcttacaatttaggt
ggcacttttcggggaaatgtgcgcggaacccctatttgtttatttttctaaatacattcaaatatgtatccgctcatgagacaataaccctgat
aaatgcttcaataatagcacctagatcaagagacaggatgaggatcgtttcgcatgattgaacaagatggattgcacgcaggttctccg
gccgcttgggtggagaggctattcggctatgactgggcacaacagacaatcggctgctctgatgccgccgtgttccggctgtcagcgc
aggggcgcccggttctttttgtcaagaccgacctgtccggtgccctgaatgaactgcaagacgaggcagcgcggctatcgtggctgg
ccacgacgggcgttccttgcgcagctgtgctcgacgttgtcactgaagcgggaagggactggctgctattgggcgaagtgccgggg
caggatctcctgtcatctcaccttgctcctgccgagaaagtatccatcatggctgatgcaatgcggggctgcatacgcttgatccggct
acctgcccattcgaccaccaagcgaaacatcgcatcgagcgagcacgtactcggatggaagccggtcttgtcgatcaggatgatctg
gacgaagagcatcaggggctcgcgccagccgaactgttcgccaggctcaaggcgagcatgcccgacggcgaggatctcgtcgtga
cccatggcgatgcctgcttgccgaatatcatggtggaaaatggccgcttttctggattcatcgactgtggccggctgggtgtggcggac
cgctatcaggacatagcgttggctacccgtgatattgctgaagagcttggggcgaatgggctgaccgcttcctcgtgctttacggtatc
gccgctcccgattcgcagcgcatcgccttctatcgccttcttgacgagttcttctgaattattaacgcttacaatttcctgatgcggtattttct
ccttacgcatctgtgcggtatttcacaccgcatcaggtggcacttttcggggaaatgtgcgcggaacccctatttgtttatttttctaaatac
attcaaatatgtatccgctcatgaccaaaatcccttaacgtgagttttcgttccactgagcgtcagaccccgtagaaaagatcaaaggatc
ttcttgagatcctttttttctgcgcgtaatctgctgcttgcaaacaaaaaaaccaccgctaccagcggtggtttgtttgccggatcaagagct
accaactctttttccgaaggtaactggcttcagcagagcgcagataccaaatactgttcttctagtgtagccgtagttaggccaccacttc
aagaactctgtagcaccgcctacatacctcgctctgctaatcctgttaccagtggctgctgccagtggcgataagtcgtgtcttaccggg
ttggactcaagacgatagttaccggataaggcgcagcggtcgggctgaacgggggggtcgtgcacacagcccagcttggagcgaa
cgacctacaccgaactgagatacctacagcgtgagctatgagaaagcgccacgcttcccgaagggagaaaggcggacaggtatcc
ggtaagcggcagggtcggaacaggagagcgcacgagggagcttccagggggaaacgcctggtatctttatagtcctgtcgggtttc
gccacctctgacttgagcgtcgatttttgtgatgctcgtcaggggggcggagcctatggaaaaacgccagcaacgcggcctttttacgg
ttcctggccttttgctggccttttgctcacatgttctttcctgcgttatcccctgattctgtggataaccgtattaccgcctttgagtgagctgat
accgctcgccgcagccgaacgaccgagcgcagcgagtcagtgagcgaggaagcggaagagcgcccaatacgcaaaccgcctct
ccccgcgcgttggccgattcattaatgcagctggcacgacaggtttcccgactggaaagcgggcagtgagcgcaacgcaattaatgt
gagttagctcactcattaggcaccccaggctttacactttatgcttccggctcgtatgttgtgtggaattgtgagcggataacaatttcaca
caggaaacagctatgaccatgattacgccaagcgcgcaattaaccctcactaaagggaacaaaagctggagctgcaagcttggccat
tgcatacgttgtatccatatcataatatgtacatttatattggctcatgtccaacattaccgccatgttgacattgattattgactagttattaata
gtaatcaattacggggtcattagttcatagcccatatatggagttccgcgttacataacttacggtaaatggcccgcctggctgaccgccc
aacgacccccgcccattgacgtcaataatgacgtatgttcccatagtaacgccaatagggactttccattgacgtcaatgggtggagtat
ttacggtaaactgcccacttggcagtacatcaagtgtatcatatgccaagtacgccccctattgacgtcaatgacggtaaatggcccgcc
tggcattatgcccagtacatgaccttatgggactttcctacttggcagtacatctacgtattagtcatcgctattaccatggtgatgcggtttt
ggcagtacatcaatgggcgtggatagcggtttgactcacggggatttccaagtctccaccccattgacgtcaatgggagtttgttttggc
accaaaatcaacgggactttccaaaatgtcgtaacaactccgccccattgacgcaaatgggcggtaggcgtgtacggtgggaggtcta
tataagcagagctcgtttagtgaaccgGGGTCTCTCTGGTTAGACCAGATCTGAGCCTGGGAGCT
CTCTGGCTAACTAGGGAACCCACTGCTTAAGCCTCAATAAAGCTTGCCTTGAGTG
CTTCAAGTAGTGTGTGCCCGTCTGTTGTGTGACTCTGGTAACTAGAGATCCCTCA
GACCCTTTTAGTCAGTGTGGAAAATCTCTAGCagtggcgcccgaacagggacttgaaagcgaaaggg
aaaccagaggagctctcTCGACGCAGGACTCGGCTTGCTGAAGCGCGCACGGCAAGAGGC
GAGGGGCGGCGACTGGTGAGTACGCCAAAAATTTTGACTAGCGGAGGCTAGAAG
GAGAGAGATGGGTGCGAGAGCGTCAGTATTAAGCGGGGGAGaattagatcgcgatgggaaa
aaattcggttaaggccagggggaaagaaaaaatataaattaaaacatatagtatgggcaagcagggagctagaacgattcgcagttaa
tcctggcctgttagaaacatcagaaggctgtagacaaatactgggacagctacaaccatcccttcagacaggatcagaagaacttagat
cattatataatacagtagcaaccctctattgtgtgcatcaaaggatagagataaaagacaccaaggaagctttagacaagatagaggaa
gagcaaaacaaaagtaagaccaccgcacagcaagcGGCCGCTgatcttcagacctggaggaggagatatgagggacaattg
gagaagtgaattatataaatataaagtagtaaaaattgaaccattaggagtagcacccaccaaggcaaagagaagagtggtgcagag
agaaaaaagaGCAGTGGGAATaggagctttgttccttgggttcttgggagcagcaggaagcactatgggcgcagcgtcaat
gacgctgacggtacaggccagacaattattgtctggtatagtgcagcagcagaacaatttgctgagggctattgaggcgcaacagcat
ctgttgcaactcacagtctggggcatcaagcagctccaggcaagaatcctggctgtggaaagatacctaaaggatcaacagctcctgg
ggatttggggttgctctggaaaactcatttgcaccactgctgtgccttggaatgctagttggagtaataaatctctggaacagatttggaat
cacacgacctggatggagtgggacagagaaattaacaattacacaagcttaatacactccttaattgaagaatcgcaaaaccagcaag
aaaagaatgaacaagaattattggaattagataaatgggcaagttttgtggaattggttttaacataacaaattggctgtggtatataaaattat
tcataatgatagtaggaggcttggtaggtttaagaatagtttttgctgtactttctatagtgaatagagttaggcagggatattcaccattatc
gtttcagacccacctcccaaccccgaggggacccgacaggcccgaaggaatagaagaagaaggtggagagagagacagagaca
gatccattcgattagtgaacggatCTCGACGGTATCGATCTCGAcacaaatggcagtattcatccacaattttaaaag
aaaaggggggattggggggtacagtgcaggggaaagaatagtagacataatagcaacagacatacaaactaaagaattacaaaaac
aaattacaaaaattcaaaattttcgggtttattacagggacagcagaTGCTGCCCACTCAGACTTTATTCAAAG
ACCAGGAAGGGCCGGTGCAAGGAGGGGAGGAGGGCCCGTTGGGAGGCCCAGCG
GGCAGGAGGAACGGCTACCGAGGCTCCAGCtgtgctgctctgcggcgaTTAACGGTATTTG
GAGGTCAGCACGGTGCTCACAGAAGCCAGGAACTTGTCCAGGGAGGCGTGCACC
GCAGGGGTGAACTCGGCGGGGAGGTGGGCGGCCAGGGTCACCAGCAGGCAGTG
GCTTAGGAGCTGTGCAGAGAAGAGGGTCAGTGCGGCCCAGGCCCGCAGCCGCCG
CCTGCGCTACACCTCCCGCAACCCGCGTGATCCTCTGCCCTGAGAGGAAGGCGCC
ATCTCGCCCCTCGACCCAGATCGCTCCCGGCCCGCCGCTCACCTTGAAGTTGACC
GGGTCCACCCGAAGCTTGTGCGCGTGCAGGTCGCTCAGGGCGGACAGCGCGTTG
GGCATGTCGTCCACGTGCGCCACGGCGTTGGTCAGCGCGTCGGCCACCTTCTTGC
CGTGGCCCTTAACCTGGGCAGAGCCGTGGCTCAGGTCGAAGTGCGGGAAGTAGG
TCTTGGTGGTGGGGAAGGACAGGAACATCCTGCGGGGAGAAGCAGAGTGAGGG
GTGGGGTTTGGGTCCGGGGCCAGGACGGTTGAGGGTGGCCTGTGGGTCCGGGCG
GGCGAGGAGCCCGGGTCGGAGCAGGGGAGGGAGCCTCACCTCTCCAGGGCCTCC
GCACCATACTCGCCAGCGTGCGCGCCGACCTTACCCCAGGCGGCCTTGACGTTGG
TCTTGTCGGCAGGAGACAGCACCATGGTGGGTTCTCTCTGAGTCTGTGGGGACCA
GAAGAGTaagcaatagatggctctgccctgacttttatgcccagccctggctcctgccctccctgctcctgggagtagattggcc
aaccctagggtgtggctccacagggtgaggtctaagtgatgacagccgtacctgtccttggctcttctggcactggcttaggagttgga
cttcaaaccctcagccctccctctaagatatatctcttggccccataccatcagtacaaattgctactaaaaacatcctcctttgcaagtgta
tttacCTCTCAGATAAACAGGAGGGGGAGGCAGCACCAGAGGTTGTAATCAGCAGT
ACCATTCACCCCTTTCCCTTGTCCCACCTCCACCCCCACAGGATGACTCAGCAGTC
CTGTTGGCCTCCAGAAGCACTGAGTCATGGTTGGCCCAGTTATCTGCTCCCTCAA
GTGTTTTCACAGACCCCCACAGCTGCATGCCTCCACCCTTGGCTTGGGCAGATGT
GCTTGCCTGGCTGACTGTGGTCCCTGATAGGTTCCAGAGTACGTaTATGTGTATAT
ATATATATATATTCAGGAAATAATATATTCTAGAATATGTCACATTCTGTCTCAG
GCATCCATTTTCTTTATGATGCCGTTTGAGGTGGAGTTTTAGTCAGGTGGTCAGCT
TCTCCTTTTTTTTGCCATCTGCCCTGTAAGCATCCTGCTGGGGACCCAGATAGGAG
TCATCACTCTAGGCTGAGAACATCTGGGCACACACCCTAAGCCTCAGCATGACTC
ATCATGACTCAGCATTGCTGTGCTTGAGCCAGAAGGTTTGCTTAGAAGGTTACAC
AGAACCAGAAGGCGGGGGTGGGGCACTGACCCCGACAGGGGCCTGGCCAGAAC
TGCTCATGCTTGGACTATGGGAGGTCACTAATGGAGACACACAGAAATGTAACA
GGAACTAAGGAAAAACTGAAGCTTtgggggtataggggagcagtcccatgtagtagtagaatgaaaaatgct
gctatgctgtgcctcccccacctttcccatgtctgccctctactcatggtctatctctcctggctcctgggagtcatggactccacccagca
ccaccaacctgacctaaccacctatctgagcctgccagcctataacccatctgggccctgatagctggtggccagccctgaccccacc
ccaccctccctggaacctctgatagacacatctggcacaccagctcgcaaagtcaccgtgagggtcttgtgtttgctgagtcaaaattcc
ttgaaatccaagtccttagagactcccaggcttggattcaaagctcctgactttctgtctagtgtatgtgcagtgagccccttttcctctaact
gaaagaaggaaaaaaaaatggaacccaaaatattctacatagtttccatgtcacagccagggctgggcagtctcctgttatttcttttaaa
ataaatatatcatttaaatgcataaataagcaaaccctgctcgggaatgggagggagagtctctggagtccaccccttctcggccctgg
ctctgcagatagtgctatcaaagccctgacagagccctgcccattgctgggccttggagtgagtcagcctagtagagaggcagggca
agccatctcatagctgctgagtgggagagagaaaagggctcattgtctataaactcaggtcatggctattcttattaaaagaaaagggg
ggactggaagggctaattcactcccaacgaagacaagatctgctttttgcttgtactGGGTCTCTCTGGTTAGACCAG
ATCTGAGCCTGGGAGCTCTCTGGCTAACTAGGGAACCCACTGCTTAAGCCTCAAT
AAAGCTTGCCTTGAGTGCTTCAAGTAGTGTGTTGGTTTTTTGTGTGTGCATTGTCT
GAGTAGGTGTCATTCTATTCTGGGGGGTGGGGTGGGGCAGCACAGCAAGGGGGA
GGATTGGGAAGACAATAGCAGGCATGCTGGGGATGCGGTGGGCTCTATGGagtagta
gttcatgtcatcttattattcagtatttataacttgcaaagaaatgaatatcagagagtgagaggaacttgtttattgcagcttataatggttac
aaataaagcaatagcatcacaaatttcacaaataaagcatttttttcactgcattctagttgtggtttgtccaaactcatcaatgtatcttatcat
gtctggctctagctatcccgcccctaactccgcccATCCCGCCCCTAACTCCGCCCAGTTCCGCCCATT
CTCCGCCCCATGGCTCTCACTACTTCTGGAATAGCTCAGAGGCCGAGGCGGCCTC
GGCCTCTGCATAAATAAAAAAAATTAGTCGAGGCTTTTTTGGAGGCCTAGGgacgta
cccaattcgccctatagtgagtcgtattacgcgcgctcactggccgtcgttttacaacgtcgtgactgggaaaaccctggcgttacccaa
cttaatcgccttgcagcacatccccctttcgccagctggcgtaatagcgaagaggcccgcaccgatcgcccttcccaacagttgcgca
gcctgaatggcgaatggg
A2-UV
Sequence ID No: 43
acgcgccctgtagcggcgcattaagcgcggcgggtgtggtggttacgcgcagcgtgaccgctacacttgccagcgccctagcgccc
gctcctttcgctttcttcccttcctttctcgccacgttcgccggctttccccgtcaagctctaaatcgggggctccctttagggttccgattta
gtgctttacggcacctcgaccccaaaaaacttgattagggtgatggttcacgtagtgggccatcgccctgatagacggtttttcgcccttt
gacgttggagtccacgttctttaatagtggactcttgttccaaactggaacaacactcaaccctatctcggtctattcttttgatttataaggg
attttgccgatttcggcctattggttaaaaaatgagctgatttaacaaaaatttaacgcgaattttaacaaaatattaacgcttacaatttaggt
ggcacttttcggggaaatgtgcgcggaacccctatttgtttatttttctaaatacattcaaatatgtatccgctcatgagacaataaccctgat
aaatgcttcaataatagcacctagatcaagagacaggatgaggatcgtttcgcatgattgaacaagatggattgcacgcaggttctccg
gccgcttgggtggagaggctattcggctatgactgggcacaacagacaatcggctgctctgatgccgccgtgttccggctgtcagcgc
aggggcgcccggttctttttgtcaagaccgacctgtccggtgccctgaatgaactgcaagacgaggcagcgcggctatcgtggctgg
ccacgacgggcgttccttgcgcagctgtgctcgacgttgtcactgaagcgggaagggactggctgctattgggcgaagtgccgggg
caggatctcctgtcatctcaccttgctcctgccgagaaagtatccatcatggctgatgcaatgcggcggctgcatacgcttgatccggct
acctgcccattcgaccaccaagcgaaacatcgcatcgagcgagcacgtactcggatggaagccggtcttgtcgatcaggatgatctg
gacgaagagcatcaggggctcgcgccagccgaactgttcgccaggctcaaggcgagcatgcccgacggcgaggatctcgtcgtga
cccatggcgatgcctgcttgccgaatatcatggtggaaaatggccgcttttctggattcatcgactgtggccggctgggtgtggcggac
cgctatcaggacatagcgttggctacccgtgatattgctgaagagcttggcggcgaatgggctgaccgcttcctcgtgctttacggtatc
gccgctcccgattcgcagcgcatcgccttctatcgccttcttgacgagttcttctgaattattaacgcttacaatttcctgatgcggtattttct
ccttacgcatctgtgcggtatttcacaccgcatcaggtggcacttttcggggaaatgtgcgcggaacccctatttgtttatttttctaaatac
attcaaatatgtatccgctcatgaccaaaatcccttaacgtgagttttcgttccactgagcgtcagaccccgtagaaaagatcaaaggatc
ttcttgagatcctttttttctgcgcgtaatctgctgcttgcaaacaaaaaaaccaccgctaccagcggtggtttgtttgccggatcaagagct
accaactctttttccgaaggtaactggcttcagcagagcgcagataccaaatactgttcttctagtgtagccgtagttaggccaccacttc
aagaactctgtagcaccgcctacatacctcgctctgctaatcctgttaccagtggctgctgccagtggcgataagtcgtgtcttaccggg
ttggactcaagacgatagttaccggataaggcgcagcggtcgggctgaacgggggggtcgtgcacacagcccagcttggagcgaa
cgacctacaccgaactgagatacctacagcgtgagctatgagaaagcgccacgcttcccgaagggagaaaggcggacaggtatcc
ggtaagcggcagggtcggaacaggagagcgcacgagggagcttccagggggaaacgcctggtatctttatagtcctgtcgggtttc
gccacctctgacttgagcgtcgatttttgtgatgctcgtcaggggggcggagcctatggaaaaacgccagcaacgcggcctttttacgg
ttcctggccttttgctggccttttgctcacatgttctttcctgcgttatcccctgattctgtggataaccgtattaccgcctttgagtgagctgat
accgctcgccgcagccgaacgaccgagcgcagcgagtcagtgagcgaggaagcggaagagcgcccaatacgcaaaccgcctct
ccccgcgcgttggccgattcattaatgcagctggcacgacaggtttcccgactggaaagcgggcagtgagcgcaacgcaattaatgt
gagttagctcactcattaggcaccccaggctttacactttatgcttccggctcgtatgttgtgtggaattgtgagcggataacaatttcaca
caggaaacagctatgaccatgattacgccaagcgcgcaattaaccctcactaaagggaacaaaagctggagctgcaagcttggccat
tgcatacgttgtatccatatcataatatgtacatttatattggctcatgtccaacattaccgccatgttgacattgattattgactagttattaata
gtaatcaattacggggtcattagttcatagcccatatatggagttccgcgttacataacttacggtaaatggcccgcctggctgaccgccc
aacgacccccgcccattgacgtcaataatgacgtatgttcccatagtaacgccaatagggactttccattgacgtcaatgggtggagtat
ttacggtaaactgcccacttggcagtacatcaagtgtatcatatgccaagtacgccccctattgacgtcaatgacggtaaatggcccgcc
tggcattatgcccagtacatgaccttatgggactttcctacttggcagtacatctacgtattagtcatcgctattaccatggtgatgcggtttt
ggcagtacatcaatgggcgtggatagcggtttgactcacggggatttccaagtctccaccccattgacgtcaatgggagtttgttttggc
accaaaatcaacgggactttccaaaatgtcgtaacaactccgccccattgacgcaaatgggcggtaggcgtgtacggtgggaggtcta
tataagcagagctcgtttagtgaaccgGGGTCTCTCTGGTTAGACCAGATCTGAGCCTGGGAGCT
CTCTGGCTAACTAGGGAACCCACTGCTTAAGCCTCAATAAAGCTTGCCTTGAGTG
CTTCAAGTAGTGTGTGCCCGTCTGTTGTGTGACTCTGGTAACTAGAGATCCCTCA
GACCCTTTTAGTCAGTGTGGAAAATCTCTAGCagtggcgcccgaacagggacttgaaagcgaaaggg
aaaccagaggagctctcTCGACGCAGGACTCGGCTTGCTGAAGCGCGCACGGCAAGAGGC
GAGGGGCGGCGACTGGTGAGTACGCCAAAAATTTTGACTAGCGGAGGCTAGAAG
GAGAGAGATGGGTGCGAGAGCGTCAGTATTAAGCGGGGGAGaattagatcgcgatgggaaa
aaattcggttaaggccagggggaaagaaaaaatataaattaaaacatatagtatgggcaagcagggagctagaacgattcgcagttaa
tcctggcctgttagaaacatcagaaggctgtagacaaatactgggacagctacaaccatcccttcagacaggatcagaagaacttagat
cattatataatacagtagcaaccctctattgtgtgcatcaaaggatagagataaaagacaccaaggaagctttagacaagatagaggaa
gagcaaaacaaaagtaagaccaccgcacagcaagcGGCCGCTgatcttcagacctggaggaggagatatgagggacaattg
gagaagtgaattatataaatataaagtagtaaaaattgaaccattaggagtagcacccaccaaggcaaagagaagagtggtgcagag
agaaaaaagaGCAGTGGGAATaggagctttgttccttgggttcttgggagcagcaggaagcactatgggcgcagcgtcaat
gacgctgacggtacaggccagacaattattgtctggtatagtgcagcagcagaacaatttgctgagggctattgaggcgcaacagcat
ctgttgcaactcacagtctggggcatcaagcagctccaggcaagaatcctggctgtggaaagatacctaaaggatcaacagctcctgg
ggatttggggttgctctggaaaactcatttgcaccactgctgtgccttggaatgctagttggagtaataaatctctggaacagatttggaat
cacacgacctggatggagtgggacagagaaattaacaattacacaagcttaatacactccttaattgaagaatcgcaaaaccagcaag
aaaagaatgaacaagaattattggaattagataaatgggcaagtttgtggaattggtttaacataacaaattggctgtggtatataaaattat
tcataatgatagtaggaggcttggtaggtttaagaatagtttttgctgtactttctatagtgaatagagttaggcagggatattcaccattatc
gtttcagacccacctcccaaccccgaggggacccgacaggcccgaaggaatagaagaagaaggtggagagagagacagagaca
gatccattcgattagtgaacggatCTCGACGGTATCGATCTCGAcacaaatggcagtattcatccacaattttaaaag
aaaaggggggattggggggtacagtgcaggggaaagaatagtagacataatagcaacagacatacaaactaaagaattacaaaaac
aaattacaaaaattcaaaattttcgggtttattacagggacagcagaTGCTGCCCACTCAGACTTTATTCAAAG
ACCAGGAAGGGCCGGTGCAAGGAGGGGAGGAGGGCCCGTTGGGAGGCCCAGCG
GGCAGGAGGAACGGCTACCGAGGCTCCAGCtgtgctgctctgcggcgaTTAACGGTATTTG
GAGGTCAGCACGGTGCTCACAGAAGCCAGGAACTTGTCCAGGGAGGCGTGCACC
GCAGGGGTGAACTCGGCGGGGAGGTGGGCGGCCAGGGTCACCAGCAGGCAGTG
GCTTAGGAGCTGTGCAGAGAAGAGGGTCAGTGCGGCCCAGGCCCGCAGCCGCCG
CCTGCGCTACACCTCCCGCAACCCGCGTGATCCTCTGCCCTGAGAGGAAGGCGCC
ATCTCGCCCCTCGACCCAGATCGCTCCCGGCCCGCCGCTCACCTTGAAGTTGACC
GGGTCCACCCGAAGCTTGTGCGCGTGCAGGTCGCTCAGGGCGGACAGCGCGTTG
GGCATGTCGTCCACGTGCGCCACGGCGTTGGTCAGCGCGTCGGCCACCTTCTTGC
CGTGGCCCTTAACCTGGGCAGAGCCGTGGCTCAGGTCGAAGTGCGGGAAGTAGG
TCTTGGTGGTGGGGAAGGACAGGAACATCCTGCGGGGAGAAGCAGAGTGAGGG
GTGGGGTTTGGGTCCGGGGCCAGGACGGTTGAGGGTGGCCTGTGGGTCCGGGCG
GGCGAGGAGCCCGGGTCGGAGCAGGGGAGGGAGCCTCACCTCTCCAGGGCCTCC
GCACCATACTCGCCAGCGTGCGCGCCGACCTTACCCCAGGCGGCCTTGACGTTGG
TCTTGTCGGCAGGAGACAGCACCATGGTGGGTTCTCTCTGAGTCTGTGGGGACCA
GAAGAGTaagcaatagatggctctgccctgacttttatgcccagccctggctcctgccctccctgctcctgggagtagattggcc
aaccctagggtgtggctccacagggtgaggtctaagtgatgacagccgtacctgtccttggctcttctggcactggcttaggagttgga
cttcaaaccctcagccctccctctaagatatatctcttggccccataccatcagtacaaattgctactaaaaacatcctcctttgcaagtgta
tttacTACGTaTATGTGTATATATATATATATATTCAGGAAATAATATATTCTAGAAT
ATGTCACATTCTGTCTCAGGCATCCATTTTCTTTATGATGCCGTTTGAGGTGGAGT
TTTAGTCAGGTGGTCAGCTTCTCCTTTTTTTTGCCATCTGCCCTGTAAGCATCCTG
CTGGGGACCCAGATAGGAGTCATCACTCTAGGCTGAGAACATCTGGGCACACAC
CCTAAGCCTCAGCATGACTCATCATGACTCAGCATTGCTGTGCTTGAGCCAGAAG
GTTTGCTTAGAAGGTTACACAGAACCAGAAGGCGGGGGTGGGGCACTGACCCCG
ACAGGGGCCTGGCCAGAACTGCTCATGCTTGGACTATGGGAGGTCACTAATGGA
GACACACAGAAATGTAACAGGAACTAAGGAAAAACTGAAGCTTtgggggtataggggagc
agtcccatgtagtagtagaatgaaaaatgctgctatgtgtgcctcccccacctttcccatgtctgccctctactcatggtctatctctcctg
gctcctgggagtcatggactccacccagcaccaccaacctgacctaaccacctatctgagcctgccagcctataacccatctgggccc
tgatagctggtggccagccctgaccccaccccaccctccctggaacctctgatagacacatctggcacaccagctcgcaaagtcacc
gtgagggtcttgtgtttgctgagtcaaaattccttgaaatccaagtccttagagactcccaggcttggattcaaagctcctgactttctgtct
agtgtatgtgcagtgagccccttttcctctaactgaaagaaggaaaaaaaaatggaacccaaaatattctacatagtttccatgtcacagc
cagggctgggcagtctcctgttatttcttttaaaataaatatatcatttaaatgcataaataagcaaaccctgctcgggaatgggagggag
agtctctggagtccaccccttctcggccctggctctgcagatagtgctatcaaagccctgacagagccctgcccattgctgggccttgg
agtgagtcagcctagtagagaggcagggcaagccatctcatagctgctgagtgggagagagaaaagggctcattgtctataaactca
ggtcatggctattcttattaaaagaaaaggggggactggaagggctaattcactcccaacgaagacaagatctgctttttgcttgtactG
GGTCTCTCTGGTTAGACCAGATCTGAGCCTGGGAGCTCTCTGGCTAACTAGGGAA
CCCACTGCTTAAGCCTCAATAAAGCTTGCCTTGAGTGCTTCAAGTAGTGTGTTGG
TTTTTTGTGTGTGCATTGTCTGAGTAGGTGTCATTCTATTCTGGGGGGTGGGGTGG
GGCAGCACAGCAAGGGGGAGGATTGGGAAGACAATAGCAGGCATGCTGGGGAT
GCGGTGGGCTCTATGGagtagtagttcatgtcatcttattattcagtatttataacttgcaaagaaatgaatatcagagagtg
agaggaacttgtttattgcagcttataatggttacaaataaagcaatagcatcacaaatttcacaaataaagcatttttttcactgcattctagt
tgtggtttgtccaaactcatcaatgtatcttatcatgtctggctctagctatcccgcccctaactccgcccATCCCGCCCCTAA
CTCCGCCCAGTTCCGCCCATTCTCCGCCCCATGGCTCTCACTACTTCTGGAATAGC
TCAGAGGCCGAGGCGGCCTCGGCCTCTGCATAAATAAAAAAAATTAGTCGAGGC
TTTTTTGGAGGCCTAGGgacgtacccaattcgccctatagtgagtcgtattacgcgcgctcactggccgtcgttttacaa
cgtcgtgactgggaaaaccctggcgttacccaacttaatcgccttgcagcacatccccctttcgccagctggcgtaatagcgaagagg
cccgcaccgatcgcccttcccaacagttgcgcagcctgaatggcgaatggg
Hba-a1 LCR-Globe-TAG
Sequence ID No: 44
acgcgccctgtagcggcgcattaagcgcggcgggtgtggtggttacgcgcagcgtgaccgctacacttgccagcgccctagcgccc
gctcctttcgctttcttcccttcctttctcgccacgttcgccggctttccccgtcaagctctaaatcgggggctccctttagggttccgattta
gtgctttacggcacctcgaccccaaaaaacttgattagggtgatggttcacgtagtgggccatcgccctgatagacggtttttcgcccttt
gacgttggagtccacgttctttaatagtggactcttgttccaaactggaacaacactcaaccctatctcggtctattcttttgatttataaggg
attttgccgatttcggcctattggttaaaaaatgagctgatttaacaaaaatttaacgcgaattttaacaaaatattaacgcttacaatttaggt
ggcacttttcggggaaatgtgcgcggaacccctatttgtttatttttctaaatacattcaaatatgtatccgctcatgagacaataaccctgat
aaatgcttcaataatagcacctagatcaagagacaggatgaggatcgtttcgcatgattgaacaagatggattgcacgcaggttctccg
gccgcttgggtggagaggctattcggctatgactgggcacaacagacaatcggctgctctgatgccgccgtgttccggctgtcagcgc
aggggcgcccggttctttttgtcaagaccgacctgtccggtgccctgaatgaactgcaagacgaggcagcgcggctatcgtggctgg
ccacgacgggcgttccttgcgcagctgtgctcgacgttgtcactgaagcgggaagggactggctgctattgggcgaagtgccgggg
caggatctcctgtcatctcaccttgctcctgccgagaaagtatccatcatggctgatgcaatgcggcggctgcatacgcttgatccggct
acctgcccattcgaccaccaagcgaaacatcgcatcgagcgagcacgtactcggatggaagccggtcttgtcgatcaggatgatctg
gacgaagagcatcaggggctcgcgccagccgaactgttcgccaggctcaaggcgagcatgcccgacggcgaggatctcgtcgtga
cccatggcgatgcctgcttgccgaatatcatggtggaaaatggccgcttttctggattcatcgactgtggccggctgggtgtggcggac
cgctatcaggacatagcgttggctacccgtgatattgctgaagagcttggcggcgaatgggctgaccgcttcctcgtgctttacggtatc
gccgctcccgattcgcagcgcatcgccttctatcgccttcttgacgagttcttctgaattattaacgcttacaatttcctgatgcggtattttct
ccttacgcatctgtgcggtatttcacaccgcatcaggtggcacttttcggggaaatgtgcgcggaacccctatttgtttatttttctaaatac
attcaaatatgtatccgctcatgaccaaaatcccttaacgtgagttttcgttccactgagcgtcagaccccgtagaaaagatcaaaggatc
ttcttgagatcctttttttctgcgcgtaatctgctgcttgcaaacaaaaaaaccaccgctaccagcggtggtttgtttgccggatcaagagct
accaactctttttccgaaggtaactggcttcagcagagcgcagataccaaatactgttcttctagtgtagccgtagttaggccaccacttc
aagaactctgtagcaccgcctacatacctcgctctgctaatcctgttaccagtggctgctgccagtggcgataagtcgtgtcttaccggg
ttggactcaagacgatagttaccggataaggcgcagcggtcgggctgaacgggggggtcgtgcacacagcccagcttggagcgaa
cgacctacaccgaactgagatacctacagcgtgagctatgagaaagcgccacgcttcccgaagggagaaaggcggacaggtatcc
ggtaagcggcagggtcggaacaggagagcgcacgagggagcttccagggggaaacgcctggtatctttatagtcctgtcgggtttc
gccacctctgacttgagcgtcgatttttgtgatgctcgtcaggggggcggagcctatggaaaaacgccagcaacgcggcctttttacgg
ttcctggccttttgctggccttttgctcacatgttctttcctgcgttatcccctgattctgtggataaccgtattaccgcctttgagtgagctgat
accgctcgccgcagccgaacgaccgagcgcagcgagtcagtgagcgaggaagcggaagagcgcccaatacgcaaaccgcctct
ccccgcgcgttggccgattcattaatgcagctggcacgacaggtttcccgactggaaagcgggcagtgagcgcaacgcaattaatgt
gagttagctcactcattaggcaccccaggctttacactttatgcttccggctcgtatgttgtgtggaattgtgagcggataacaatttcaca
caggaaacagctatgaccatgattacgccaagcgcgcaattaaccctcactaaagggaacaaaagctggagctgcaagcttggccat
tgcatacgttgtatccatatcataatatgtacatttatattggctcatgtccaacattaccgccatgttgacattgattattgactagttattaata
gtaatcaattacggggtcattagttcatagcccatatatggagttccgcgttacataacttacggtaaatggcccgcctggctgaccgccc
aacgacccccgcccattgacgtcaataatgacgtatgttcccatagtaacgccaatagggactttccattgacgtcaatgggtggagtat
ttacggtaaactgcccacttggcagtacatcaagtgtatcatatgccaagtacgccccctattgacgtcaatgacggtaaatggcccgcc
tggcattatgcccagtacatgaccttatgggactttcctacttggcagtacatctacgtattagtcatcgctattaccatggtgatgcggtttt
ggcagtacatcaatgggcgtggatagcggtttgactcacggggatttccaagtctccaccccattgacgtcaatgggagtttgttttggc
accaaaatcaacgggactttccaaaatgtcgtaacaactccgccccattgacgcaaatgggcggtaggcgtgtacggtgggaggtcta
tataagcagagctcgtttagtgaaccgGGGTCTCTCTGGTTAGACCAGATCTGAGCCTGGGAGCT
CTCTGGCTAACTAGGGAACCCACTGCTTAAGCCTCAATAAAGCTTGCCTTGAGTG
CTTCAAGTAGTGTGTGCCCGTCTGTTGTGTGACTCTGGTAACTAGAGATCCCTCA
GACCCTTTTAGTCAGTGTGGAAAATCTCTAGCagtggcgcccgaacagggacttgaaagcgaaaggg
aaaccagaggagctctcTCGACGCAGGACTCGGCTTGCTGAAGCGCGCACGGCAAGAGGC
GAGGGGCGGCGACTGGTGAGTACGCCAAAAATTTTGACTAGCGGAGGCTAGAAG
GAGAGAGATGGGTGCGAGAGCGTCAGTATTAAGCGGGGGAGaattagatcgcgatgggaaa
aaattcggttaaggccagggggaaagaaaaaatataaattaaaacatatagtatgggcaagcagggagctagaacgattcgcagttaa
tcctggcctgttagaaacatcagaaggctgtagacaaatactgggacagctacaaccatcccttcagacaggatcagaagaacttagat
cattatataatacagtagcaaccctctattgtgtgcatcaaaggatagagataaaagacaccaaggaagctttagacaagatagaggaa
gagcaaaacaaaagtaagaccaccgcacagcaagcGGCCGCTgatcttcagacctggaggaggagatatgagggacaattg
gagaagtgaattatataaatataaagtagtaaaaattgaaccattaggagtagcacccaccaaggcaaagagaagagtggtgcagag
agaaaaaagaGCAGTGGGAATaggagctttgttccttgggttcttgggagcagcaggaagcactatgggcgcagcgtcaat
gacgctgacggtacaggccagacaattattgtctggtatagtgcagcagcagaacaatttgctgagggctattgaggcgcaacagcat
ctgttgcaactcacagtctggggcatcaagcagctccaggcaagaatcctggctgtggaaagatacctaaaggatcaacagctcctgg
ggatttggggttgctctggaaaactcatttgcaccactgctgtgccttggaatgctagttggagtaataaatctctggaacagatttggaat
cacacgacctggatggagtgggacagagaaattaacaattacacaagcttaatacactccttaattgaagaatcgcaaaaccagcaag
aaaagaatgaacaagaattattggaattagataaatgggcaagtttgtggaattggtttaacataacaaattggctgtggtatataaaattat
tcataatgatagtaggaggcttggtaggtttaagaatagtttttgctgtactttctatagtgaatagagttaggcagggatattcaccattatc
gtttcagacccacctcccaaccccgaggggacccgacaggcccgaaggaatagaagaagaaggtggagagagagacagagaca
gatccattcgattagtgaacggatCTCGACGGTATCGATCTCGAcacaaatggcagtattcatccacaattttaaaag
aaaaggggggattggggggtacagtgcaggggaaagaatagtagacataatagcaacagacatacaaactaaagaattacaaaaac
aaattacaaaaattcaaaattttcgggtttattacagggacagcagaTCAAAGACCAAGAGGTACAGGTGCAA
GGGAGAGAAGAAGGGCATGGCCAGAAGGCAAGCCCCGCAGAAGGCAGCtgtgctgct
ctgcggcgaTTAACGGTACTTGGAGGTCAGCACGGTGCTCACAGAGGCAAGGAATTT
GTCCAGAGAGGCATGCACCGCGGGGGTGAAATCGGCAGGGTGGTGGCTAGCCAA
GGTCACCAGCAGGCAGTGGCTCAGGAGctgcggagacaaagtggacaccctgatgcctctgctccccttcc
tgggaccactatgttccctgccttgggcacgaggacccccaagcccttcctaggggtcccagatgccgcctgccaggtcccagcgca
tacCTTGAAGTTGACGGGATCCACACGCAGCTTGTGGGCATGCAGGTCGCTCAGAG
CAGACAGGGCACCGGGCAGGTCATCGAGGTGGCCTGCAGCATTGGCCAGAGCAT
CGGCGACCTTCTTGCCGTGACCCTTGACCTGGGCAGAGCCGTGGCTTACATCAAA
GTGAGGGAAGTAGGTCTTGGTGGTGGGGAAGCTAGCAAACATCctgggagaaggagagaa
gagaagttgaggtcacagaaaagcatagttagaagctgcccactgagcgagtgccaggtccatattggatcctgtgatccttacagatc
aaggtcCtgttctcacCTTTCCAGGGCTTCAGCTCCATATTCAGCACCATGGCCACCAATCT
TCCCCCAGGCAGCCTTGATGTTGCTTTTGTCTTCCCCAGAGAGCACCATGGTTTCT
TCCTGAGTCTGTCAGAATCAGAAGTGTCTTGGACCTGCAGCAAGTAGCACTTATA
TGCCCTCCAAGGGCGTGTCCTCCAGGCAGGGTGGACACGCCCTTGGAGCAGTTCT
CATTGGCTGGTTTGTGCTTGCTCTACCCAGTAAAACTTGTTAGGGCCCCTGAGATT
CTGGGGCTGGGTTGTGCTTCCCCaagcaatagatggctctgccctgacttttatgcccagccctggctcctgccct
ccctgctcctgggagtagattggccaaccctagggtgtggctccacagggtgaggtctaagtgatgacagccgtacctgtccttggctc
ttctggcactggcttaggagttggacttcaaaccctcagccctccctctaagatatatctcttggccccataccatcagtacaaattgctac
taaaaacatcctcctttgcaagtgtatttacccttttgccacctagctgtccaggggtgccttaaaatggcaaacaaggtttgttttcttttcct
gttttcatgccttcctcttccatatccttgtttcatattaatacatgtgtatagatcctaaaaatctatacacatgtattaataaagcctgattctgc
cgcttctaggtatagaggccacctgcaagataaatatttgattcacaataactaatcattctatggcaattgataacaacaaatatatatatat
atatatatacgtatatgtgtatatatatatatatatattcaggaaataatatattctagaatatgtcacattctgtctcaggcatccattttctttatg
atgccgtttgaggtggagttttagtcaggtggtcagcttctccttttttttgccatctgccctgtaagcatcctgctggggacccagatagga
gtcatcactctaggctgagaacatctgggcacacaccctaagcctcagcatgactcatcatgactcagcattgctgtgctttgagccaga
aggtttgcttagaaggttacacagaaccagaaggcgggggtggggcactgaccccgacaggggcctggccagaactgctcatgctt
ggactatgggaggtcactaatggagacacacagaaatgtaacaggaactaaggaaaaactgaagcttatttaatcagagatgaggatg
ctggaagggatagagggagctgagcttgtaaaaagtatagtaatcattcagcaaatggttttgaagcacctgctggatgctaaacactat
tttcagtgcttgaatcataaataagaataaaacatgtatcttattccccacaagagtccaagtaaaaaataacagttaattataatgtgctctg
tcccccaggctggagtgcagtggcacgatctcagctcactgcaacctccgcctcccgggttcaagcaattctcctgcctcagccaccct
aatagctgggattacaggtgcacaccaccatgccaggctaatttttgtactttttgtagaggcagggtatcaccatgttgtccaagatggt
cttgaactcctgagctccaagcagtccacccacctcagcctcccaaagtgctgggattacaggtgtgagacaccatgcccagattttcc
atatttaatagaggtatttatgggatgggggaaaagaatgtttctctcactgtggattattttagagagtggagaatggtcaagatttttttaa
aaattaagaaaacataagttggaccttgagaaatgaaaatttatttttttgttggaggatacccattctctatctcccatcagggcaagctgt
aaggaactggctaagacacagtgagacagagtgacttagtcttagaggccccactggtacgacggtcaccaagctttcattaaaaaaa
gtctaaccagctgcattcgactttgactgcagcagctggttagaaggttctactggaggagggtcccagcccattgctaaattaacatca
ggctctgagactggcagtatatctctaacagtggttgatgctatcttctggaacttgcctgctacattgagaccactgacccatacatagg
aagcccatagctctgtcctgaactgttaggccactggtccagagagtgtgcatctcctttgatcctcataataaccctatgagatagacac
aattattactcttactttatagatgatgatcctgaaaacataggagtcaaggcacttgcccctagctgggggtataggggagcagtcccat
gtagtagtagaatgaaaaatgctgctatgctgtgcctcccccacctttcccatgtctgccctctactcatggtctatctctcctggctcctgg
gagtcatggactccacccagcaccaccaacctgacctaaccacctatctgagcctgccagcctataacccatctgggccctgatagct
ggtggccagccctgaccccaccccaccctccctggaacctctgatagacacatctggcacaccagctcgcaaagtcaccgtgagggt
cttgtgtttgctgagtcaaaattccttgaaatccaagtccttagagactcctgctcccaaatttacagtcatagacttcttcatggctgtctcct
ttatccacagaatgattcctttgcttcattgccccatccatctgatcctcctcatcagtgcagcacagggcccatgagcagtagctgcaga
gtctcacataggtctggcactgcctctgacatgtccgaccttaggcaaatgcttgactcttctgagctcagtcttgtcatggcaaaataaa
gataataatagtgtttttttatggagttagcgtgaggatggaaaacaatagcaaaattgattagactataaaaggtctcaacaaatagtagt
agattttatcatccattaatccttccctctcctctcttactcatcccatcacgtatgcctcttaattttcccttacctataataagagttattcctctt
attatattcttcttatagtgattctggatattaaagtgggaatgaggggcaggccactaacgaagaagatgtttctcaaagaagctaaaag
aaaaggggggactggaagggctaattcactcccaacgaagacaagatctgctttttgcttgtactGGGTCTCTCTGGTTA
GACCAGATCTGAGCCTGGGAGCTCTCTGGCTAACTAGGGAACCCACTGCTTAAGC
CTCAATAAAGCTTGCCTTGAGTGCTTCAAGTAGTGTGTTGGTTTTTTGTGTGTGCA
TTGTCTGAGTAGGTGTCATTCTATTCTGGGGGGTGGGGTGGGGCAGCACAGCAAG
GGGGAGGATTGGGAAGACAATAGCAGGCATGCTGGGGATGCGGTGGGCTCTATG
Gagtagtagttcatgtcatcttattattcagtatttataacttgcaaagaaatgaatatcagagagtgagaggaacttgtttattgcagcttat
aatggttacaaataaagcaatagcatcacaaatttcacaaataaagcatttttttcactgcattctagttgtggtttgtccaaactcatcaatgt
atcttatcatgtctggctctagctatcccgcccctaactccgcccATCCCGCCCCTAACTCCGCCCAGTTCCG
CCCATTCTCCGCCCCATGGCTCTCACTACTTCTGGAATAGCTCAGAGGCCGAGGC
GGCCTCGGCCTCTGCATAAATAAAAAAAATTAGTCGAGGCTTTTTTGGAGGCCTA
GGgacgtacccaattcgccctatagtgagtcgtattacgcgcgctcactggccgtcgttttacaacgtcgtgactgggaaaaccctgg
cgttacccaacttaatcgccttgcagcacatccccctttcgccagctggcgtaatagcgaagaggcccgcaccgatcgcccttcccaa
cagttgcgcagcctgaatggcgaatggg
Hba-a1 UV-TAG
Sequence ID No: 45
acgcgccctgtagcggcgcattaagcgcggcgggtgtggtggttacgcgcagcgtgaccgctacacttgccagcgccctagcgccc
gctcctttcgctttcttcccttcctttctcgccacgttcgccggctttccccgtcaagctctaaatcgggggctccctttagggttccgattta
gtgctttacggcacctcgaccccaaaaaacttgattagggtgatggttcacgtagtgggccatcgccctgatagacggtttttcgcccttt
gacgttggagtccacgttctttaatagtggactcttgttccaaactggaacaacactcaaccctatctcggtctattcttttgatttataaggg
attttgccgatttcggcctattggttaaaaaatgagctgatttaacaaaaatttaacgcgaattttaacaaaatattaacgcttacaatttaggt
ggcacttttcggggaaatgtgcgcggaacccctatttgtttatttttctaaatacattcaaatatgtatccgctcatgagacaataaccctgat
aaatgcttcaataatagcacctagatcaagagacaggatgaggatcgtttcgcatgattgaacaagatggattgcacgcaggttctccg
gccgcttgggtggagaggctattcggctatgactgggcacaacagacaatcggctgctctgatgccgccgtgttccggctgtcagcgc
aggggcgcccggttctttttgtcaagaccgacctgtccggtgccctgaatgaactgcaagacgaggcagcgcggctatcgtggctgg
ccacgacgggcgttccttgcgcagctgtgctcgacgttgtcactgaagcgggaagggactggctgctattgggcgaagtgccgggg
caggatctcctgtcatctcaccttgctcctgccgagaaagtatccatcatggctgatgcaatgcggcggctgcatacgcttgatccggct
acctgcccattcgaccaccaagcgaaacatcgcatcgagcgagcacgtactcggatggaagccggtcttgtcgatcaggatgatctg
gacgaagagcatcaggggctcgcgccagccgaactgttcgccaggctcaaggcgagcatgcccgacggcgaggatctcgtcgtga
cccatggcgatgcctgcttgccgaatatcatggtggaaaatggccgcttttctggattcatcgactgtggccggctgggtgtggcggac
cgctatcaggacatagcgttggctacccgtgatattgctgaagagcttggcggcgaatgggctgaccgcttcctcgtgctttacggtatc
gccgctcccgattcgcagcgcatcgccttctatcgccttcttgacgagttcttctgaattattaacgcttacaatttcctgatgcggtattttct
ccttacgcatctgtgcggtatttcacaccgcatcaggtggcacttttcggggaaatgtgcgcggaacccctatttgtttatttttctaaatac
attcaaatatgtatccgctcatgaccaaaatcccttaacgtgagttttcgttccactgagcgtcagaccccgtagaaaagatcaaaggatc
ttcttgagatcctttttttctgcgcgtaatctgctgcttgcaaacaaaaaaaccaccgctaccagcggtggtttgtttgccggatcaagagct
accaactctttttccgaaggtaactggcttcagcagagcgcagataccaaatactgttcttctagtgtagccgtagttaggccaccacttc
aagaactctgtagcaccgcctacatacctcgctctgctaatcctgttaccagtggctgctgccagtggcgataagtcgtgtcttaccggg
ttggactcaagacgatagttaccggataaggcgcagcggtcgggctgaacgggggggtcgtgcacacagcccagcttggagcgaa
cgacctacaccgaactgagatacctacagcgtgagctatgagaaagcgccacgcttcccgaagggagaaaggcggacaggtatcc
ggtaagcggcagggtcggaacaggagagcgcacgagggagcttccagggggaaacgcctggtatctttatagtcctgtcgggtttc
gccacctctgacttgagcgtcgatttttgtgatgctcgtcaggggggcggagcctatggaaaaacgccagcaacgcggcctttttacgg
ttcctggccttttgctggccttttgctcacatgttctttcctgcgttatcccctgattctgtggataaccgtattaccgcctttgagtgagctgat
accgctcgccgcagccgaacgaccgagcgcagcgagtcagtgagcgaggaagcggaagagcgcccaatacgcaaaccgcctct
ccccgcgcgttggccgattcattaatgcagctggcacgacaggtttcccgactggaaagcgggcagtgagcgcaacgcaattaatgt
gagttagctcactcattaggcaccccaggctttacactttatgcttccggctcgtatgttgtgtggaattgtgagcggataacaatttcaca
caggaaacagctatgaccatgattacgccaagcgcgcaattaaccctcactaaagggaacaaaagctggagctgcaagcttggccat
tgcatacgttgtatccatatcataatatgtacatttatattggctcatgtccaacattaccgccatgttgacattgattattgactagttattaata
gtaatcaattacggggtcattagttcatagcccatatatggagttccgcgttacataacttacggtaaatggcccgcctggctgaccgccc
aacgacccccgcccattgacgtcaataatgacgtatgttcccatagtaacgccaatagggactttccattgacgtcaatgggtggagtat
ttacggtaaactgcccacttggcagtacatcaagtgtatcatatgccaagtacgccccctattgacgtcaatgacggtaaatggcccgcc
tggcattatgcccagtacatgaccttatgggactttcctacttggcagtacatctacgtattagtcatcgctattaccatggtgatgcggtttt
ggcagtacatcaatgggcgtggatagcggtttgactcacggggatttccaagtctccaccccattgacgtcaatgggagtttgttttggc
accaaaatcaacgggactttccaaaatgtcgtaacaactccgccccattgacgcaaatgggcggtaggcgtgtacggtgggaggtcta
tataagcagagctcgtttagtgaaccgGGGTCTCTCTGGTTAGACCAGATCTGAGCCTGGGAGCT
CTCTGGCTAACTAGGGAACCCACTGCTTAAGCCTCAATAAAGCTTGCCTTGAGTG
CTTCAAGTAGTGTGTGCCCGTCTGTTGTGTGACTCTGGTAACTAGAGATCCCTCA
GACCCTTTTAGTCAGTGTGGAAAATCTCTAGCagtggcgcccgaacagggacttgaaagcgaaaggg
aaaccagaggagctctcTCGACGCAGGACTCGGCTTGCTGAAGCGCGCACGGCAAGAGGC
GAGGGGCGGCGACTGGTGAGTACGCCAAAAATTTTGACTAGCGGAGGCTAGAAG
GAGAGAGATGGGTGCGAGAGCGTCAGTATTAAGCGGGGGAGaattagatcgcgatgggaaa
aaattcggttaaggccagggggaaagaaaaaatataaattaaaacatatagtatgggcaagcagggagctagaacgattcgcagttaa
tcctggcctgttagaaacatcagaaggctgtagacaaatactgggacagctacaaccatcccttcagacaggatcagaagaacttagat
cattatataatacagtagcaaccctctattgtgtgcatcaaaggatagagataaaagacaccaaggaagctttagacaagatagaggaa
gagcaaaacaaaagtaagaccaccgcacagcaagcGGCCGCTgatcttcagacctggaggaggagatatgagggacaattg
gagaagtgaattatataaatataaagtagtaaaaattgaaccattaggagtagcacccaccaaggcaaagagaagagtggtgcagag
agaaaaaagaGCAGTGGGAATaggagctttgttccttgggttcttgggagcagcaggaagcactatgggcgcagcgtcaat
gacgctgacggtacaggccagacaattattgtctggtatagtgcagcagcagaacaatttgctgagggctattgaggcgcaacagcat
ctgttgcaactcacagtctggggcatcaagcagctccaggcaagaatcctggctgtggaaagatacctaaaggatcaacagctcctgg
ggatttggggttgctctggaaaactcatttgcaccactgctgtgccttggaatgctagttggagtaataaatctctggaacagatttggaat
cacacgacctggatggagtgggacagagaaattaacaattacacaagcttaatacactccttaattgaagaatcgcaaaaccagcaag
aaaagaatgaacaagaattattggaattagataaatgggcaagtttgtggaattggtttaacataacaaattggctgtggtatataaaattat
tcataatgatagtaggaggcttggtaggtttaagaatagtttttgctgtactttctatagtgaatagagttaggcagggatattcaccattatc
gtttcagacccacctcccaaccccgaggggacccgacaggcccgaaggaatagaagaagaaggtggagagagagacagagaca
gatccattcgattagtgaacggatCTCGACGGTATCGATCTCGAcacaaatggcagtattcatccacaattttaaaag
aaaaggggggattggggggtacagtgcaggggaaagaatagtagacataatagcaacagacatacaaactaaagaattacaaaaac
aaattacaaaaattcaaaattttcgggtttattacagggacagcagaTCAAAGACCAAGAGGTACAGGTGCAA
GGGAGAGAAGAAGGGCATGGCCAGAAGGCAAGCCCCGCAGAAGGCAGCtgtgctgct
ctgcggcgaTTAACGGTACTTGGAGGTCAGCACGGTGCTCACAGAGGCAAGGAATTT
GTCCAGAGAGGCATGCACCGCGGGGGTGAAATCGGCAGGGTGGTGGCTAGCCAA
GGTCACCAGCAGGCAGTGGCTCAGGAGctgcggagacaaagtggacaccctgatgcctctgctccccttcc
tgggaccactatgttccctgccttgggcacgaggacccccaagcccttcctaggggtcccagatgccgcctgccaggtcccagcgca
tacCTTGAAGTTGACGGGATCCACACGCAGCTTGTGGGCATGCAGGTCGCTCAGAG
CAGACAGGGCACCGGGCAGGTCATCGAGGTGGCCTGCAGCATTGGCCAGAGCAT
CGGCGACCTTCTTGCCGTGACCCTTGACCTGGGCAGAGCCGTGGCTTACATCAAA
GTGAGGGAAGTAGGTCTTGGTGGTGGGGAAGCTAGCAAACATCctgggagaaggagagaa
gagaagttgaggtcacagaaaagcatagttagaagctgcccactgagcgagtgccaggtccatattggatcctgtgatccttacagatc
aaggtcCtgttctcacCTTTCCAGGGCTTCAGCTCCATATTCAGCACCATGGCCACCAATCT
TCCCCCAGGCAGCCTTGATGTTGCTTTTGTCTTCCCCAGAGAGCACCATGGTTTCT
TCCTGAGTCTGTCAGAATCAGAAGTGTCTTGGACCTGCAGCAAGTAGCACTTATA
TGCCCTCCAAGGGCGTGTCCTCCAGGCAGGGTGGACACGCCCTTGGAGCAGTTCT
CATTGGCTGGTTTGTGCTTGCTCTACCCAGTAAAACTTGTTAGGGCCCCTGAGATT
CTGGGGCTGGGTTGTGCTTCCCCaagcaatagatggctctgccctgacttttatgcccagccctggctcctgccct
cccgctcctgggagtagattggccaaccctagggtgtggctccacagggtgaggtctaagtgatgacagccgtacctgtccttggctc
ttctggcactggcttaggagttggacttcaaaccctcagccctccctctaagatatatctcttggccccataccatcagtacaaattgctac
taaaaacatcctcctttgcaagtgtatttacTACGTaTATGTGTATATATATATATATATTCAGGAAAT
AATATATTCTAGAATATGTCACATTCTGTCTCAGGCATCCATTTTCTTTATGATGC
CGTTTGAGGTGGAGTTTTAGTCAGGTGGTCAGCTTCTCCTTTTTTTTGCCATCTGC
CCTGTAAGCATCCTGCTGGGGACCCAGATAGGAGTCATCACTCTAGGCTGAGAA
CATCTGGGCACACACCCTAAGCCTCAGCATGACTCATCATGACTCAGCATTGCTG
TGCTTGAGCCAGAAGGTTTGCTTAGAAGGTTACACAGAACCAGAAGGCGGGGGT
GGGGCACTGACCCCGACAGGGGCCTGGCCAGAACTGCTCATGCTTGGACTATGG
GAGGTCACTAATGGAGACACACAGAAATGTAACAGGAACTAAGGAAAAACTGA
AGCTTtgggggtataggggagcagtcccatgtagtagtagaatgaaaaatgctgctatgtgtgcctcccccacctttcccatgtct
gccctctactcatggtctatctctcctggctcctgggagtcatggactccacccagcaccaccaacctgacctaaccacctatctgagcc
tgccagcctataacccatctgggccctgatagctggtggccagccctgaccccaccccaccctccctggaacctctgatagacacatc
tggcacaccagctcgcaaagtcaccgtgagggtcttgtgtttgctgagtcaaaattccttgaaatccaagtccttagagactcccaggctt
ggattcaaagctcctgactttctgtctagtgtatgtgcagtgagccccttttcctctaactgaaagaaggaaaaaaaaatggaacccaaaa
tattctacatagtttccatgtcacagccagggctgggcagtctcctgttatttcttttaaaataaatatatcatttaaatgcataaataagcaaa
ccctgctcgggaatgggagggagagtctctggagtccaccccttctcggccctggctctgcagatagtgctatcaaagccctgacaga
gccctgcccattgctgggccttggagtgagtcagcctagtagagaggcagggcaagccatctcatagctgctgagtgggagagaga
aaagggctcattgtctataaactcaggtcatggctattcttattaaaagaaaaggggggactggaagggctaattcactcccaacgaag
acaagatctgctttttgcttgtactGGGTCTCTCTGGTTAGACCAGATCTGAGCCTGGGAGCTCTC
TGGCTAACTAGGGAACCCACTGCTTAAGCCTCAATAAAGCTTGCCTTGAGTGCTT
CAAGTAGTGTGTTGGTTTTTTGTGTGTGCATTGTCTGAGTAGGTGTCATTCTATTC
TGGGGGGTGGGGTGGGGCAGCACAGCAAGGGGGAGGATTGGGAAGACAATAGC
AGGCATGCTGGGGATGCGGTGGGCTCTATGGagtagtagttcatgtcatcttattattcagtatttataacttg
caaagaaatgaatatcagagagtgagaggaacttgtttattgcagcttataatggttacaaataaagcaatagcatcacaaatttcacaaa
taaagcatttttttcactgcattctagttgtggtttgtccaaactcatcaatgtatcttatcatgtctggctctagctatcccgcccctaactccg
cccATCCCGCCCCTAACTCCGCCCAGTTCCGCCCATTCTCCGCCCCATGGCTCTCAC
TACTTCTGGAATAGCTCAGAGGCCGAGGCGGCCTCGGCCTCTGCATAAATAAAA
AAAATTAGTCGAGGCTTTTTTGGAGGCCTAGGgacgtacccaattcgccctatagtgagtcgtattacgc
gcgctcactggccgtcgttttacaacgtcgtgactgggaaaaccctggcgttacccaacttaatcgccttgcagcacatccccctttcgc
cagctggcgtaatagcgaagaggcccgcaccgatcgcccttcccaacagttgcgcagcctgaatggcgaatggg

1. A recombinant lentiviral vector (LV) comprising:
an expression cassette comprising a nucleic acid construct comprising:
an alpha-globin (α-globin) gene;
a 5′ UTR upstream from said α-globin gene and a 3′ UTR downstream from said α-globin gene where said 5′ and 3′ UTRs are from β-globin genes or from α-globin genes;
an α-globin promoter or a β globin promoter upstream from said 5′ UTR; and
a locus control region (LCR) upstream from said promoter,
wherein said LCR is selected from the group consisting of core LCR UV set forth in SEQ ID NO:11, GLOBE βLCR (A2-LCR) containing HS2 HS3 set forth in SEQ ID NO:12, and GLOBE βLCR containing HS2 set forth in SEQ ID NO:13, or an LCR having at least 95% or at least 98%, or at least 99% sequence identity to the foregoing LCRs; or
wherein said LCT is selected from the group consisting of:
a core β-LCR comprising reduced length HS4, HS3, and HS2 DNase I hypersensitive sites; or a Globe β-LCR comprising HS3 and HS2 DNase I hypersensitive sites; or a Globe β-LCR comprising HS2 DNase I hypersensitive site and β-LCR comprising reduced length HS4, and HS3, DNase I hypersensitive sites.

2. (canceled)

3. The recombinant lentiviral vector according to claim 1, wherein said α-globin gene comprises
(a) a full-length α-globin gene including exon and introns;
(b) an HBA1 α-globin gene of SEQ ID NO:8;
(c) an HBA2 α-globin gene of SEQ ID NO:9;
(d) an alpha globin gene without intron 2; or
(e) an alpha globin cDNA.

4. (canceled)

5. (canceled)

6. (canceled)

7. (canceled)

8. The recombinant lentiviral vector of claim 3, wherein said alpha globin cDNA is codon optimized or contains a tag, wherein said tag optionally comprises or consists of the sequence tgtgctgctctgcggcga (SEQ ID NO:18).

9. (canceled)

10. (canceled)

11. The recombinant lentiviral vector according to claim 1, wherein said expression cassette comprises
(a) a 5′ UTR and a 3′UTR from a β-globin gene; or
(b) a 5′ UTR and a 3′UTR from an α-globin gene.

12. (canceled)

13. The recombinant lentiviral vector according to claim 1, wherein said expression cassette comprises
(a) a full length β-globin promoter;
(b) a shortened β-globin promoter of SEQ ID NO:1;
(c) a full length α-globin promoter; or
(d) a shortened α-globin promoter of SEQ ID NO:14.

14. (canceled)

15. (canceled)

16. (canceled)

17. The recombinant lentiviral vector according to claim 1, wherein said expression cassette comprises
(a) a core β-LCR comprising reduced length HS4 set forth in SEQ ID NO:17, HS3 set forth in SEQ ID NO:16, and HS2 set forth in SEQ ID NO:15 DNase I hypersensitive sites;
(b) a Globe β-LCR comprising HS3 and HS2 DNase I hypersensitive sites (SEQ ID NO:12);
(c) a Globe β-LCR comprising HS2 DNase I hypersensitive site (SEQ ID NO:13) and a β-LCR comprising reduced length HS4, and HS3, DNase I hypersensitive sites;
(d).

18. (canceled)

19. (canceled)

20. The recombinant lentiviral vector according to claim 1, wherein said expression cassette comprises an α-globin regulatory element (HS40) of SEQ ID NO:10 between the LCR and the α-globin gene.

21. The recombinant lentiviral vector of claim 1, wherein said vector comprises
(a) the features of Vector I shown in FIG. 1, panel B or the features of Vector I shown in FIG. 4, panel A, wherein optionally said vector comprises or consists of the nucleic acid sequence of the expression cassette of vector I in SEQ ID NO:27;
(b) the features of Vector II shown in FIG. 1, panel B or the features of Vector II shown in FIG. 4, panel B, wherein optionally said vector comprises or consists of the nucleic acid sequence of the expression cassette of vector I in SEQ ID NO:28;
(c) the features of Vector III shown in FIG. 1, panel B or the features of Vector III shown in FIG. 4, panel C, wherein optionally said vector comprises or consists of the nucleic acid sequence of the expression cassette of vector I in SEQ ID NO:29;
(d) the features of Vector IV shown in FIG. 1, panel B or the features of Vector IV shown in FIG. 4, panel D; wherein optionally said vector comprises or consists of the nucleic acid sequence of the expression cassette of vector I in SEQ ID NO:30;
(e) the features of Vector V shown in FIG. 1, panel B or the features of Vector V shown in FIG. 4, panel E, wherein optionally said vector comprises or consists of the nucleic acid sequence of the expression cassette of vector I in SEQ ID NO:31;
(f) the features of Vector VI shown in FIG. 1, panel B or the features of Vector VI shown in FIG. 4, panel F, wherein optionally said vector comprises or consists of the nucleic acid sequence of the expression cassette of vector I in SEQ ID NO:32;
(g) the features of Vector VII shown in FIG. 1, panel B or comprises the features of Vector VII shown in FIG. 4, panel G, wherein optionally said vector comprises or consists of the nucleic acid sequence of the expression cassette of vector I in SEQ ID NO:33;
(h) the features of Vector VIII (A2-cDNA-UV) shown in FIG. 5 or wherein said vector comprises the nucleic acid sequence of the expression cassette of vector VIII (A2-cDNA-UV) in SEQ ID NO:38, wherein optionally said vector comprises or consists of the nucleic acid sequence of SEQ ID NO:38;
(i) the features of Vector IX (A2-ΔIVS2-UV) shown in FIG. 5 or the nucleic acid sequence of the expression cassette of vector IX (A2-ΔIVS2-UV) in SEQ ID NO:40, wherein optionally said vector comprises or consists of the nucleic acid sequence of SEQ ID NO:40; or
(j) the features of Vector X (A2-ΔIVS2-Globe) shown in FIG. 5 or said vector comprises the nucleic acid sequence of the expression cassette of vector X (A2-ΔIVS2-Globe) in SEQ ID NO:39, wherein optionally said vector comprises or consists of the nucleic acid sequence of SEQ ID NO:39.

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. A host cell transduced with a vector according to claim 1.

59. The host cell of claim 58, wherein the cell is
(a) a stem cell, wherein optionally, said stem cell is derived from bone marrow, and/or from umbilical cord blood, and/or from peripheral blood;
(b) a 293T cell; or
(c) a human hematopoietic progenitor cell, wherein said human hematopoietic progenitor cell is optionally a CD34+ cell.

60. (canceled)

61. (canceled)

62. (canceled)

63. (canceled)

64. An expression cassette comprising a nucleic acid construct comprising:
an alpha-globin (α-globin) gene;
a 5′ UTR upstream from said α-globin gene and a 3′ UTR downstream from said α-globin gene where said 5′ and 3′ UTRs are from β-globin genes or from α-globin genes;
an α-globin promoter or a β globin promoter upstream from said 5′ UTR; and
a locus control region (LCR) upstream from said promoter, wherein said LCR is selected from
(a) the group consisting of core LCR UV (SEQ ID NO:11), GLOBE βLCR (A2-LCR) containing HS2 HS3 (SEQ ID NO:12), and GLOBE βLCR containing HS2 (SEQ ID NO:13), or an LCR having at least 95% or at least 98%, or at least 99% sequence identity to the foregoing LCRs; or
(b) the group consisting of: a core β-LCR comprising reduced length HS4, HS3, and HS2 DNase I hypersensitive sites; or a Globe β-LCR comprising HS3 and HS2 DNase I hypersensitive sites; or a Globe β-LCR comprising HS2 DNase I hypersensitive site and β-LCR comprising reduced length HS4, and HS3, DNase I hypersensitive sites.

65. (canceled)

66. The expression cassette according to claim 64, wherein said α-globin gene comprises
(a) a full-length α-globin gene including exon and introns;
(b) an HBA1 α-globin gene of SEQ ID NO:8;
(c) an HBA2 α-globin gene of SEQ ID NO:9;
(d) an alpha globin gene without intron 2; or
(e) an alpha globin cDNA, wherein optionally said alpha globin cDNA is codon optimized.

67. (canceled)

68. (canceled)

69. (canceled)

70. (canceled)

71. (canceled)

72. The recombinant lentiviral vector according to claim 66, wherein said alpha globin gene contains a tag, wherein optionally said tag comprises or consists of the sequence tgtgctgctctgcggcga (SEQ ID NO:18).

73. (canceled)

74. The expression cassette according to claim 64, wherein said expression cassette comprises
(a) a 5′ UTR and a 3′UTR from a β-globin gene; or
(b) a 5′ UTR and a 3′UTR from an α-globin gene.

75. (canceled)

76. The expression cassette according to claim 64, wherein said expression cassette comprises
(a) a full length β-globin promoter;
(b) a shortened β-globin promoter of SEQ ID NO:1;
(c) a full length α-globin promoter; or
(d) a shortened α-globin promoter of SEQ ID NO:14.

77. (canceled)

78. (canceled)

79. (canceled)

80. The expression cassette according to claim 64, wherein said expression cassette comprises
(a) a core β-LCR comprising reduced length HS4 of SEQ ID NO:17, HS3 of SEQ ID NO:16, and HS2 of SEQ ID NO:15 DNase I hypersensitive sites;
(b) a Globe β-LCR comprising HS3 and HS2 DNase I hypersensitive sites of SEQ ID NO: 12; or
(c) a Globe β-LCR comprising HS2 DNase I hypersensitive site of SEQ ID NO:13 and a β-LCR comprising reduced length HS4, and HS3, DNase I hypersensitive sites.

81. (canceled)

82. (canceled)

83. The expression cassette according to claim 64, wherein said expression cassette comprises an α-globin regulatory element (HS40) of SEQ ID NO:10 between the LCR and the α-globin gene.

84. The expression cassette of claim 64,
(a) wherein said expression cassette comprises or consists of the nucleic acid sequence of the expression cassette of vector I in SEQ ID NO:27;
(b) wherein said expression cassette comprises or consists of the nucleic acid sequence of the expression cassette of vector II in SEQ ID NO:28;
(c) wherein said expression cassette comprises or consists of the nucleic acid sequence of the expression cassette of vector III in SEQ ID NO:29;
(d) wherein said expression cassette comprises or consists of the nucleic acid sequence of the expression cassette of vector IV in SEQ ID NO:30;
(e) wherein said expression cassette comprises or consists of the nucleic acid sequence of expression cassette of vector V in SEQ ID NO:31;
(f) wherein said expression cassette comprises or consists of the nucleic acid sequence of the expression cassette of vector VI in SEQ ID NO:32;
(g) wherein said expression cassette comprises or consists of the nucleic acid sequence of the expression cassette of vector VII in SEQ ID NO:33;
(h) wherein said expression cassette comprises or consists of the nucleic acid sequence of the expression cassette of vector VIII (A2-cDNA-UV) in SEQ ID NO:38;
(i) wherein said expression cassette comprises or consists of the nucleic acid sequence of the expression cassette of vector IX (A2-ΔIVS2-UV) in SEQ ID NO:40; or
(j) wherein said expression cassette comprises or consists of the nucleic acid sequence of the expression cassette of vector X (A2-ΔIVS2-Globe) in SEQ ID NO:39.

85. (canceled)

86. (canceled)

87. (canceled)

88. (canceled)

89. (canceled)

90. (canceled)

91. (canceled)

92. (canceled)

93. (canceled)

94. A lentiviral particle comprising the expression cassette recited in claim 64.

95. (canceled)

96. A method of treating α-thalassemia in a subject, said method comprising:
transducing a stem cell and/or progenitor cell from said subject with a vector according to claim 1; and
transplanting said transduced cell or cells derived therefrom into said subject where said cells or derivatives therefrom express an alpha globin.

97. The method of claim 96, wherein the cell
(a) is a stem cell, wherein optionally stem cell derived from bone marrow;
(b) is a human hematopoietic progenitor cell, wherein optionally the human hematopoietic progenitor cell is a CD34⁺ cell.

98. (canceled)

99. (canceled)

100. (canceled)
